(19) United States
(12) Patent Application Publication
(10) Pub. No.: US 2025/0376464 A1
(43) Pub. Date: Dec. 11, 2025

(54) Compounds for treatment a coronavirus infection

(71) Applicant: Trawsfynydd Therapeutics, Inc., San Diego, CA (US)

(72) Inventors: Boris Rogovoy, Needham, MA (US); Volodymyr Kysil, San Diego, CA (US); Vladimir Berishvili, Mölndal (SE); Charles David Pauza, Gaithersburg, MD (US); Juan Carlos Zapata, Ijamsville, MD (US); Sandra Margarita Medina Moreno, Rockville, MD (US); Haishan Li, North Potomac, MD (US); Andrew Orry, San Diego, CA (US); Polo Chun-Hung Lam, San Diego, CA (US); Ruben Abagyan, La Jolla, CA (US); Nikolay Savchuk, San Diego, CA (US)

(21) Appl. No.: 18/875,691

(22) PCT Filed: Jun. 16, 2023
(86) PCT No.: PCT/US2023/068573

§ 371 Date: Dec. 16, 2024

Related U.S. Application Data
(60) Provisional application No. 63/353,354, filed on Jun. 17, 2022.
(60) Provisional application No. 63/414,287, filed on Oct. 7, 2022.

(51) Int. Cl. C07D 403/12; A61K 31/4439; A61K 31/4709; C07D 401/12; C07D 403/14; C07D 471/04

(52) U.S. Cl. CPC C07D 403/12; A61K 31/4439; A61K 31/4709; C07D 401/12; C07D 403/14; C07D 471/04

(57) ABSTRACT

The present invention is generally directed to inhibitors of SARS-CoV-2-related 3C-like protease (Mpro) useful in the treatment of coronavirus infection and having the Formula (A):

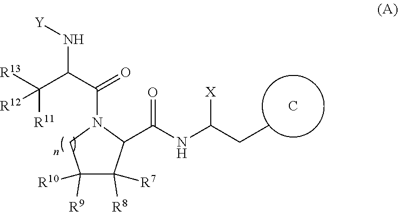

### CROSS-REFERENCE TO RELATED APPLICATIONS

This application claims priority to and benefit of U.S. Provisional Patent Application Ser. No. 63/353,354 filed Jun. 17, 2022, entitled “Compounds for treatment of a coronavirus infection”, and U.S. Provisional Patent Application Ser. No. 63/414,287 filed Oct. 7, 2022, entitled “Compounds for treatment of a coronavirus infection” the disclosure of which are incorporated by reference in its entirety for all purposes.

### FIELD OF INVENTION

The present invention is directed to inhibitors of coronavirus replication activity. The inhibitors described herein can be useful for the treatment of disease caused by coronavirus infection including COVID-19 resulting from SARS-CoV-2 infection. In particular, the invention is concerned with compounds and pharmaceutical compositions that inhibit the SARS-CoV-2-related 3C-like protease, methods of treating coronavirus infection, and methods of synthesizing these compounds.

### BACKGROUND

Coronaviruses are a large family of viruses that usually cause mild to moderate upper-respiratory tract illnesses in humans. However, three coronaviruses have caused more serious and fatal diseases in people: SARS coronavirus (SARS-CoV), which emerged in November 2002 and causes severe acute respiratory syndrome (SARS); MERS coronavirus (MERS-CoV), which emerged in 2012 and causes Middle East respiratory syndrome (MERS); and SARS-CoV-2, which emerged in 2019 and causes coronavirus disease 2019 (COVID-19).

Infection with severe acute respiratory syndrome coronavirus 2, or SARS-CoV-2, causes coronavirus disease 2019 (COVID-19).

The virus that causes COVID-19 spreads easily among people. Data has shown that the COVID-19 virus spreads mainly from person to person among those in close contact (within about 6 feet, or 2 meters). The COVID-19 virus can spread from someone who is infected but has no symptoms. This is called asymptomatic transmission. The COVID-19 virus can also spread from someone who is infected but hasn't developed symptoms yet. This is called presymptomatic transmission. It's possible to contract COVID-19 upon reinfection with SARS-CoV-2 and this may happen more than once.

Whole genome sequencing showed that SARS-CoV-2 shares 79.6% sequence identify with SARS-CoV. SARS-CoV-2 appears to have relatively high transmission rate among humans and causes severe and fatal pneumonia and other damages, threatening people at all ages, especially seniors and persons with pre-existing conditions including immune deficiency, lung disease, metabolic disease and others. As the number of infections and deaths is increasing rapidly, there is an urgent call for drug development against COVID-19.

SARS-CoV-2 is a complex virus consisting of 3 open reading frames expressing polyproteins. The polyproteins undergo proteolytic processing into their mature, active forms. While the spike glycoprotein is subject to cleavage by host proteases Cathepsin L and TMPRSS2, the remaining proteolytic processing steps are mediated by one of two virally-encoded proteases designated Main protease (Mpro or 3C-like protease (3CLpro)) and the papain-like protease PLpro. Mpro plays a vital role in maturation of viral proteins require for RNA replication and is a preferred target for the development of direct acting antiviral drugs capable of inhibiting SARS-CoV-2 infection. The Mpro enzyme of SARS-CoV-2 is similar to SARS-CoV by about 96%.

In view of the ongoing SARS-CoV-2 spread that has caused the current worldwide COVID-19 outbreak, it is desirable to have new methods of inhibiting SARS-CoV-2 viral replication and of treating COVID-19 in patients.

### SUMMARY

In general, the invention relates to compounds of Formula (A):

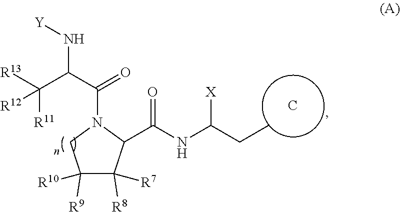

or a pharmaceutically acceptable salt, stereoisomer, solvate, prodrug, or tautomer thereof, wherein:
C is a heterocyclic ring selected from:

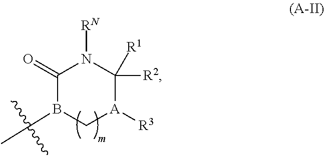

wherein: m is an integer selected from 0, 1, 2; A is selected from CR⁴ and N; B is selected from CR⁶ and N;

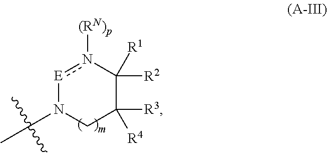

wherein: the bond [symbol] is selected from a single bond and a double bond; p is an integer selected from 0, 1; E is selected from C(O) and N; provided that when the bond [symbol] is a single bond p is 1 and E is C(O), and when the bond [symbol] is a double bond p is 0 and E is N; m is an integer selected from 0, 1, 2;

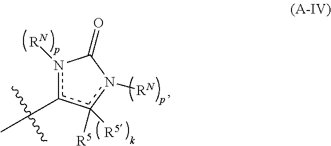

wherein: each bond [symbol] and [symbol] is a single bond or double bond; when [symbol] is a double bond each bond [symbol] is a single bond, when [symbol] is a single bond each bond [symbol] is independently selected from a single bond or double bond; each p is an integer independently selected from 0 and 1; provided that when [symbol] is a single bond then p is 1 and when [symbol] is a double bond then p is 0; k is an integer selected from 0, 1; provided that when the bond

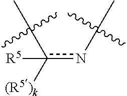

is a single bond and the bond [symbol] is a single bond then k is 1 and when the bond

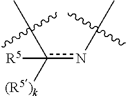

or the bond [symbol] is a double bond then k is 0;
and

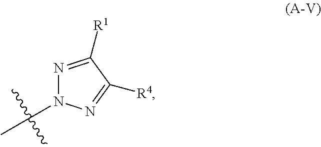

wherein:
R^{N} is selected from H, C₁-C₆ alkyl, cycloalkyl;
R¹ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R² is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R¹ and R² together is oxo;
or R² and R³ together form a double bond;
R³ is selected from hydrogen, C₁-C₆ alkyl, C₂-C₆ alkenyl, C₂-C₆ alkynyl, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R⁴ is selected from hydrogen, halogen, —OH, —CN, —NO₂, —NR¹³R¹⁴, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R¹ and R⁴ together with the atoms to which they are attached and any intervening atoms, form a 3-10 membered cycloalkyl, a 6-10 membered aryl, a 3-14 membered heterocycle, or a 5-6 membered heteroaryl, wherein the cycloalkyl, aryl, heterocycle or heteroaryl is optionally substituted with one or more R¹⁶;
R⁵ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹³, —C(O)N—NR¹⁴R¹⁵, cycloalkyl, —O-cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R^{5′} is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)N—NR¹⁴R¹⁵, cycloalkyl, —O-cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R⁵ and R^{5′} form oxo;
R⁶ is selected from H, C₁-C₆ alkyl, cycloalkyl, halogen, —CN, —OH;
R⁷, R⁸, R⁹ and R¹⁰ are each independently selected from H, alkyl;
or R⁸ and R⁹ together with the atoms to which they are attached and any intervening atoms, form cycloalkyl optionally substituted with one or more halogen, —OH, —CN, C₁-C₆ alkyl;
or R⁹ and R¹⁰ together with the atoms to which they are attached and any intervening atoms, form cycloalkyl optionally substituted with one or more halogen, —OH, —CN, C₁-C₆ alkyl;
R¹¹, R¹², R¹³ are each independently selected from C₁-C₆ alkyl, cycloalkyl, halogen, —CN;
R¹⁴ and R¹⁵ are each independently selected from H, C₁-C₆ alkyl;
or R¹⁴ and R¹⁵ together with the atoms to which they are attached and any intervening atoms, form heterocyclyl optionally substituted with one or more halogen, —OH, —CN, C₁-C₆ alkyl;
R¹⁶ is independently selected from halogen, —OH, —OH, —CN, —NO₂, —NR¹³R¹⁴, C₁-C₆ alkyl, C₂-C₆ alkenyl, C₂-C₆ alkynyl, C₁-C₆ alkoxy, —C(O)OR¹³, —C(O)N—NR¹³R¹⁴, cycloalkyl, —O-cycloalkyl, aryl, heterocyclyl, and heteroaryl;
X is selected from hydrogen, halogen, OH, CN, NO₂, CONH₂, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl wherein alkyl optionally substituted with cycloalkyl, heterocyclyl, aryl, and heteroaryl;
Y is selected from C₁-C₆ alkyl-S(O)ᵤ—, C₁-C₆ alkyl-C(O)—, heteroaryl-C(O)—, wherein alkyl or heteroaryl is optionally substituted with one or more halogen, —OH, —CN;
n is integer selected from 0, 1, 2;
u is integer selected from 0, 1, 2;

wherein,
cycloalkyl is a mono or polycyclic saturated carbon rings containing 3-18 carbon atoms;
aryl is a cyclic, aromatic hydrocarbon groups that have 1 to 3 aromatic rings;
heterocyclyl is a saturated or partially unsaturated 3-10 membered monocyclic, 7-12 membered bicyclic (fused, bridged, or spiro rings), or 11-14 membered tricyclic ring system (fused, bridged, or spiro rings) having one or more heteroatoms selected from O, N, S, P, Se, or B;
heteroaryl is a monovalent monocyclic or a polycyclic aromatic radical of 5 to 24 ring atoms, containing one or more ring heteroatoms selected from N, O, S, P, or B, the remaining ring atoms being C;
provided that when in heterocycle (A-II) m is 0; A and B are CH; R¹ and R² are CH₃, H, deuterium, or R¹ is OH, or R¹ and R² together is oxo; n is 1; R⁸ and R⁹ are C₁-C₆ alkyl or R⁸ and R⁹ together with the atoms to which they are attached and any intervening atoms, form cyclopropyl or cyclohexyl, or R⁹ and R¹⁰ together with the atoms to which they are attached and any intervening atoms, form cyclobutyl, cyclohexyl, or R⁹ is cyclopentyl; R¹¹, R¹², R¹³ are each CH₃; and X is CN then Y is not CF₃C(O)—, (CH₃)₃CS(O)₂—, ClCH₂C(O)—, (CH₃)₂CHC(O)—, CH₃CH₂C(O)—, CH₃C(O)—;
provided that when in heterocycle (A-II) m is 1; A and B are CH; R¹ and R² are CH₃ or H; n is 1; R⁸ and R⁹ together with the atoms to which they are attached and any intervening atoms, form cyclopropyl, cyclopentyl or cyclohexyl, or R⁹ and R¹⁰ together with the atoms to which they are attached and any intervening atoms, form cyclobutyl, cyclohexyl, or R⁹ is cyclopentyl, then Y is not CF₃C(O)—;
and provided that when in heterocycle (A-II) m is 0; A and B are CH; R¹ and R² are both H; n is 2; R⁹ or R¹⁰ is C₁-C₆ alkyl, then Y is not CF₃C(O)—, CH₃S(O)₂.

The first aspect of the invention relates to compounds of Formula (II):

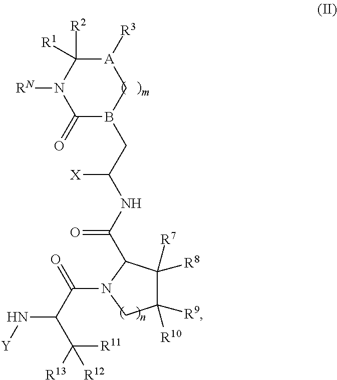

or a pharmaceutically acceptable salt, stereoisomer, solvate, prodrug, or tautomer thereof,

wherein:
A is selected from CR⁴ and N;
B is selected from CR⁶ and N;
R^{N} is selected from H, C₁-C₆ alkyl, cycloalkyl;
R¹ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R² is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R¹ and R² together is oxo;
R³ is selected from hydrogen, C₁-C₆ alkyl, C₂-C₆ alkenyl, C₂-C₆ alkynyl, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R⁴ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R¹ and R⁴ together with the atoms to which they are attached and any intervening atoms, form a 3-10 membered cycloalkyl, a 6-10 membered aryl, a 3-14 membered heterocycle, or a 5-6 membered heteroaryl, wherein the cycloalkyl, aryl, heterocycle or heteroaryl is optionally substituted with one or more R¹⁶;
or R³ and R⁴ together is oxo;
R⁶ is selected from H, C₁-C₆ alkyl, cycloalkyl, halogen, —CN, —OH;
R⁷, R⁸, R⁹ and R¹⁰ are each independently selected from H, C₁-C₆ alkyl, cycloalkyl;
or R⁸ and R⁹ together with the atoms to which they are attached and any intervening atoms, form cycloalkyl optionally substituted with one or more halogen, —OH, —CN, C₁-C₆ alkyl;
or R⁹ and R¹⁰ together with the atoms to which they are attached and any intervening atoms, form cycloalkyl optionally substituted with one or more halogen, —OH, —CN, C₁-C₆ alkyl;
R¹¹, R¹², R¹³ are each independently selected from C₁-C₆ alkyl, cycloalkyl, halogen, —CN;
R¹⁴ and R¹⁵ are each independently selected from H, C₁-C₆ alkyl;
or R¹⁴ and R¹⁵ together with the atoms to which they are attached and any intervening atoms, form heterocyclyl optionally substituted with one or more halogen, —OH, —CN, C₁-C₆ alkyl;
R¹⁶ is independently selected from halogen, —OH, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)N—NR¹⁴R¹⁵, cycloalkyl, —O-cycloalkyl, aryl, heterocyclyl, and heteroaryl;
X is selected from hydrogen, halogen, OH, CN, NO₂, CONH₂, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl wherein alkyl optionally substituted with cycloalkyl, heterocyclyl, aryl, and heteroaryl;
Y is selected from C₁-C₆ alkyl-S(O)ᵤ—, C₁-C₆ alkyl-C(O)—, heteroaryl-C(O)—, wherein alkyl or heteroaryl is optionally substituted with one or more halogen, —OH, —CN;
m is an integer selected from 0, 1, 2;
n is an integer selected from 0, 1, 2;
u is integer selected from 0, 1, 2;

wherein,
cycloalkyl is a mono or polycyclic saturated carbon rings containing 3-18 carbon atoms;
aryl is a cyclic, aromatic hydrocarbon groups that have 1 to 3 aromatic rings;
heterocyclyl is a saturated or partially unsaturated 3-10 membered monocyclic, 7-12 membered bicyclic (fused, bridged, or spiro rings), or 11-14 membered tricyclic ring system (fused, bridged, or spiro rings) having one or more heteroatoms selected from O, N, S, P, Se, or B;
heteroaryl is a monovalent monocyclic or a polycyclic aromatic radical of 5 to 24 ring atoms, containing one or more ring heteroatoms selected from N, O, S, P, or B, the remaining ring atoms being C;
provided that when m is 0; A and B are CH; R¹ and R² are CH₃, H, or R¹ is OH, or R¹ and R² together is oxo; n is 1; R⁸ and R⁹ are C₁-C₆ alkyl or R⁸ and R⁹ together with the atoms to which they are attached and any intervening atoms, form cyclopropyl or cyclohexyl, or R⁹ and R¹⁰ together with the atoms to which they are attached and any intervening atoms, form cyclobutyl, cyclohexyl, or R⁹ is cyclopentyl; R¹¹, R¹², R¹³ are each CH₃; and X is CN then Y is not CF₃C(O)—, (CH₃)₃CS(O)₂—, ClCH₂C(O)—, (CH₃)₂CHC(O)—, CH₃CH₂C(O)—, CH₃C(O)—;
provided that when m is 1; A and B are CH; R¹ and R² are CH₃ or H; n is 1; R⁸ and R⁹ together with the atoms to which they are attached and any intervening atoms, form cyclopropyl, cyclopentyl or cyclohexyl, or R⁹ and R¹⁰ together with the atoms to which they are attached and any intervening atoms, form cyclobutyl, cyclohexyl, or R⁹ is cyclopentyl, then Y is not CF₃C(O)—;
and provided that when m is 0; A and B are CH; R¹ and R² are both H; n is 2; R⁹ or R¹⁰ is C₁-C₆ alkyl, then Y is not CF₃C(O)—, CH₃S(O)₂.

The second aspect of the invention relates to compounds of Formula (III):

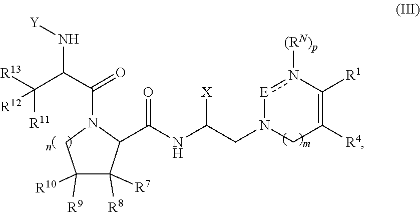

or a pharmaceutically acceptable salt, stereoisomer, solvate, prodrug, or tautomer thereof,

wherein:
the bond [symbol] is selected from a single bond and a double bond;
p is an integer selected from 0, 1;
E is selected from C(O) and N;
provided that when the bond [symbol] is a single bond p is 1, E is C(O) and when the bond [symbol] is a double bond p is 0, E is N;
R^{N} is selected from H, C₁-C₆ alkyl, cycloalkyl;
R¹ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R⁴ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R¹ and R⁴ together with the atoms to which they are attached and any intervening atoms, form a 3-10 membered cycloalkyl, a 6-10 membered aryl, a 3-14 membered heterocycle, or a 5-6 membered heteroaryl, wherein the cycloalkyl, aryl, heterocycle or heteroaryl is optionally substituted with one or more R¹⁶;
R⁷, R⁸, R⁹ and R¹⁰ are each independently selected from H, C₁-C₆ alkyl, cycloalkyl;
or R⁸ and R⁹ together with the atoms to which they are attached and any intervening atoms, form cycloalkyl optionally substituted with one or more halogen, —OH, —CN, C₁-C₆ alkyl;
or R⁹ and R¹⁰ together with the atoms to which they are attached and any intervening atoms, form cycloalkyl optionally substituted with one or more halogen, —OH, —CN, C₁-C₆ alkyl;
R¹¹, R¹², R¹³ are each independently selected from C₁-C₆ alkyl, cycloalkyl, halogen, —CN;
R¹⁴ and R¹⁵ are each independently selected from H, C₁-C₆ alkyl;
or R¹⁴ and R¹⁵ together with the atoms to which they are attached and any intervening atoms, form heterocyclyl optionally substituted with one or more halogen, —OH, —CN, C₁-C₆ alkyl;
R¹⁶ is independently selected from halogen, —OH, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)N—NR¹⁴R¹⁵, cycloalkyl, —O-cycloalkyl, aryl, heterocyclyl, and heteroaryl;
X is selected from hydrogen, halogen, OH, CN, NO₂, CONH₂, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl wherein alkyl optionally substituted with cycloalkyl, heterocyclyl, aryl, and heteroaryl;
Y is selected from C₁-C₆ alkyl-S(O)ᵤ—, C₁-C₆ alkyl-C(O)—, heteroaryl-C(O)—, wherein alkyl or heteroaryl is optionally substituted with one or more halogen, —OH, —CN;
m is an integer selected from 0, 1, 2;
n is an integer selected from 0, 1, 2;
u is integer selected from 0, 1, 2;
   wherein,
cycloalkyl is a mono or polycyclic saturated carbon rings containing 3-18 carbon atoms;
aryl is a cyclic, aromatic hydrocarbon groups that have 1 to 3 aromatic rings;
heterocyclyl is a saturated or partially unsaturated 3-10 membered monocyclic, 7-12 membered bicyclic (fused, bridged, or spiro rings), or 11-14 membered tricyclic ring system (fused, bridged, or spiro rings) having one or more heteroatoms selected from O, N, S, P, Se, or B;
heteroaryl is a monovalent monocyclic or a polycyclic aromatic radical of 5 to 24 ring atoms, containing one or more ring heteroatoms selected from N, O, S, P, or B, the remaining ring atoms being C.

The third aspect of the invention relates to compounds of Formula (IV):

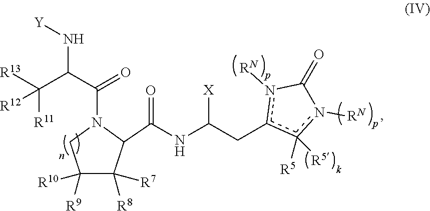

or a pharmaceutically acceptable salt, stereoisomer, solvate, prodrug, or tautomer thereof,

wherein:
each bond [symbol] and [symbol] is a single bond or double bond;
when [symbol] is a double bond each bond [symbol] is a single bond, when [symbol] is a single bond each bond [symbol] is independently selected from a single bond or double bond;
each p is an integer independently selected from 0 and 1;
provided that when [symbol] is a single bond then p is 1 and when [symbol] is a double bond then p is 0;
k is an integer selected from 0, 1;
provided that when the bond

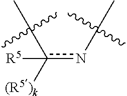

is a single bond and the bond [symbol] is a single k is 1 and when the bond

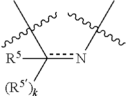

or the bond [symbol] is a double bond k is 0;
each R^{N} is independently selected from H, C₁-C₆ alkyl, cycloalkyl;
R⁵ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹³, —C(O)N—NR¹⁴R¹⁵, cycloalkyl, —O-cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R^{5′} is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)N—NR¹⁴R¹⁵, cycloalkyl, —O-cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R⁵ and R^{5′} form oxo;
R⁷, R⁸, R⁹ and R¹⁰ are each independently selected from H, C₁-C₆ alkyl, cycloalkyl;
or R⁸ and R⁹ together with the atoms to which they are attached and any intervening atoms, form cycloalkyl optionally substituted with one or more halogen, —OH, —CN, C₁-C₆ alkyl;
or R⁹ and R¹⁰ together with the atoms to which they are attached and any intervening atoms, form cycloalkyl optionally substituted with one or more halogen, —OH, —CN, C₁-C₆ alkyl;
R¹¹, R¹², R¹³ are each independently selected from C₁-C₆ alkyl, cycloalkyl, halogen, —CN;
R¹⁴ and R¹⁵ are each independently selected from H, C₁-C₆ alkyl;
or R¹⁴ and R¹⁵ together with the atoms to which they are attached and any intervening atoms, form heterocyclyl optionally substituted with one or more halogen, —OH, —CN, C₁-C₆ alkyl;
R¹⁶ is independently selected from halogen, —OH, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)N—NR¹⁴R¹⁵, cycloalkyl, —O-cycloalkyl, aryl, heterocyclyl, and heteroaryl;
X is selected from hydrogen, halogen, OH, CN, NO₂, CONH₂, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl wherein alkyl optionally substituted with cycloalkyl, heterocyclyl, aryl, and heteroaryl;
Y is selected from C₁-C₆ alkyl-S(O)ᵤ—, C₁-C₆ alkyl-C(O)—, heteroaryl-C(O)—, wherein alkyl or heteroaryl is optionally substituted with one or more halogen, —OH, —CN;
n is an integer selected from 0, 1, 2;
u is an integer selected from 0, 1, 2;
   wherein,
cycloalkyl is a mono or polycyclic saturated carbon rings containing 3-18 carbon atoms;
aryl is a cyclic, aromatic hydrocarbon groups that have 1 to 3 aromatic rings;
heterocyclyl is a saturated or partially unsaturated 3-10 membered monocyclic, 7-12 membered bicyclic (fused, bridged, or spiro rings), or 11-14 membered tricyclic ring system (fused, bridged, or spiro rings) having one or more heteroatoms selected from O, N, S, P, Se, or B;
heteroaryl is a monovalent monocyclic or a polycyclic aromatic radical of 5 to 24 ring atoms, containing one or more ring heteroatoms selected from N, O, S, P, or B, the remaining ring atoms being C.

The fourth aspect of the invention relates to compounds of Formula (V):

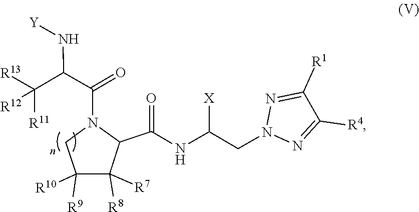

or a pharmaceutically acceptable salt, stereoisomer, solvate, prodrug, or tautomer thereof,

wherein:
R¹ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R⁴ is selected from hydrogen, halogen, —OH, —CN, —NO₂, —NR¹³R¹⁴, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R¹ and R⁴ together with the atoms to which they are attached and any intervening atoms, form a 3-10 membered cycloalkyl, a 6-10 membered aryl, a 3-14 membered heterocycle, or a 5-6 membered heteroaryl, wherein the cycloalkyl, aryl, heterocycle or heteroaryl is optionally substituted with one or more R¹⁶;
R⁷, R⁸, R⁹ and R¹⁰ are each independently selected from H, C₁-C₆ alkyl, cycloalkyl;
or R⁸ and R⁹ together with the atoms to which they are attached and any intervening atoms, form cycloalkyl optionally substituted with one or more halogen, —OH, —CN, C₁-C₆ alkyl;
or R⁹ and R¹⁰ together with the atoms to which they are attached and any intervening atoms, form cycloalkyl optionally substituted with one or more halogen, —OH, —CN, C₁-C₆ alkyl;
R¹¹, R¹², R¹³ are each independently selected from C₁-C₆ alkyl, cycloalkyl, halogen, —CN;
R¹⁴ and R¹⁵ are each independently selected from H, C₁-C₆ alkyl;
or R¹⁴ and R¹⁵ together with the atoms to which they are attached and any intervening atoms, form heterocyclyl optionally substituted with one or more halogen, —OH, —CN, C₁-C₆ alkyl;
R¹⁶ is independently selected from halogen, —OH, —OH, —CN, —NO₂, —NR¹³R¹⁴, C₁-C₆ alkyl, C₂-C₆ alkenyl, C₂-C₆ alkynyl, C₁-C₆ alkoxy, —C(O)OR¹³, —C(O)N—NR¹³R¹⁴, cycloalkyl, —O-cycloalkyl, aryl, heterocyclyl, and heteroaryl;
X is selected from hydrogen, halogen, OH, CN, NO₂, CONH₂, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl wherein alkyl optionally substituted with cycloalkyl, heterocyclyl, aryl, and heteroaryl;
Y is selected from C₁-C₆ alkyl-S(O)ᵤ—, C₁-C₆ alkyl-C(O)—, heteroaryl-C(O)—, wherein alkyl or heteroaryl is optionally substituted with one or more halogen, —OH, —CN;
n is integer selected from 0, 1, 2;
u is integer selected from 0, 1, 2;
   wherein,
cycloalkyl is a mono or polycyclic saturated carbon rings containing 3-18 carbon atoms;
aryl is a cyclic, aromatic hydrocarbon groups that have 1 to 3 aromatic rings;
heterocyclyl is a saturated or partially unsaturated 3-10 membered monocyclic, 7-12 membered bicyclic (fused, bridged, or spiro rings), or 11-14 membered tricyclic ring system (fused, bridged, or spiro rings) having one or more heteroatoms selected from O, N, S, P, Se, or B;
heteroaryl is a monovalent monocyclic or a polycyclic aromatic radical of 5 to 24 ring atoms, containing one or more ring heteroatoms selected from N, O, S, P, or B, the remaining ring atoms being C.

Another aspect of the invention is directed to pharmaceutical compositions comprising a compound of Formula (II), or a pharmaceutically acceptable salt, hydrate, solvate, prodrug, stereoisomer, or tautomer thereof and a pharmaceutically acceptable carrier. The pharmaceutical acceptable carrier may further include an excipient, diluent, or surfactant.

Another aspect of the invention is directed to pharmaceutical compositions comprising a compound of Formula (III), or a pharmaceutically acceptable salt, hydrate, solvate, prodrug, stereoisomer, or tautomer thereof and a pharmaceutically acceptable carrier. The pharmaceutical acceptable carrier may further include an excipient, diluent, or surfactant.

Another aspect of the invention is directed to pharmaceutical compositions comprising a compound of Formula (IV), or a pharmaceutically acceptable salt, hydrate, solvate, prodrug, stereoisomer, or tautomer thereof and a pharmaceutically acceptable carrier. The pharmaceutical acceptable carrier may further include an excipient, diluent, or surfactant.

Another aspect of the invention is directed to pharmaceutical compositions comprising a compound of Formula (V), or a pharmaceutically acceptable salt, hydrate, solvate, prodrug, stereoisomer, or tautomer thereof and a pharmaceutically acceptable carrier. The pharmaceutical acceptable carrier may further include an excipient, diluent, or surfactant.

Another aspect of the invention relates to a method of treating coronavirus infection such as COVID-19. The method comprises administering to a patient in need of a treatment coronavirus infection such as COVID-19 an effective amount of a compound of Formula (II), Formula (III), Formula (IV), Formula (V) or a pharmaceutically acceptable salt, hydrate, solvate, prodrug, stereoisomer, tautomer, or pharmaceutical composition thereof.

Another aspect of the invention is directed to a method of inhibiting or preventing SARS-CoV-2 viral replication. The method involves administering to a patient in need thereof an effective amount of a compound of Formula (II), Formula (III), Formula (IV), Formula (V) or a pharmaceutically acceptable salt, hydrate, solvate, prodrug, stereoisomer, tautomer, or pharmaceutical composition thereof.

Another aspect of the present invention relates to compounds of Formula (II), Formula (III), Formula (IV), Formula (V) or pharmaceutically acceptable salts, hydrates, solvates, prodrugs, stereoisomers, tautomers, or pharmaceutical compositions thereof, for use in the manufacture of a medicament for inhibiting or preventing SARS-CoV-2 viral replication.

Another aspect of the present invention relates to the use of compounds of Formula (II), Formula (III), Formula (IV), Formula (V) or pharmaceutically acceptable salts, hydrates, solvates, prodrugs, stereoisomers, tautomers, or pharmaceutical compositions thereof, in the treatment of a disease or disorder associated with SARS-CoV-2 viral infection.

Another aspect of the present invention relates to compounds of Formula (II), Formula (III), Formula (IV), Formula (V) or pharmaceutically acceptable salts, hydrates, solvates, prodrugs, stereoisomers, tautomers, or pharmaceutical compositions thereof, for use in the manufacture of a medicament for treating or preventing a viral infection disclosed herein.

Another aspect of the invention is directed to a method of treating or preventing a coronavirus infection such as COVID-19 in a subject in need thereof. The method involves administering to a patient in need of the treatment an effective amount of a compound of Formula (II), or of a compound of Formula (III), or of a compound of Formula (IV), or of a compound of Formula (V), or a pharmaceutically acceptable salt, hydrate, solvate, prodrug, stereoisomer, tautomer, or pharmaceutical composition thereof.

Another aspect of the present invention relates to the use of compounds of Formula (II), or pharmaceutically acceptable salts, hydrates, solvates, prodrugs, stereoisomers, tautomers, or pharmaceutical compositions thereof, in the treatment of a coronavirus infection.

Another aspect of the present invention relates to the use of compounds of Formula (III), or pharmaceutically acceptable salts, hydrates, solvates, prodrugs, stereoisomers, tautomers, or pharmaceutical compositions thereof, in the treatment of a coronavirus infection.

Another aspect of the present invention relates to the use of compounds of Formula (IV), or pharmaceutically acceptable salts, hydrates, solvates, prodrugs, stereoisomers, tautomers, or pharmaceutical compositions thereof, in the treatment of a coronavirus infection.

Another aspect of the present invention relates to the use of compounds of Formula (V), or pharmaceutically acceptable salts, hydrates, solvates, prodrugs, stereoisomers, tautomers, or pharmaceutical compositions thereof, in the treatment of a coronavirus infection.

Another aspect of the present invention relates to the use of compounds of Formula (II)-(V), or pharmaceutically acceptable salts, hydrates, solvates, prodrugs, stereoisomers, tautomers, or pharmaceutical compositions thereof, in the treatment of a coronavirus infection.

The present invention further provides methods of treating a coronavirus infection, comprising administering to a patient suffering from a coronavirus infection a compound of Formula (II)-(V), or a pharmaceutically acceptable salt, hydrate, solvate, prodrug, stereoisomer, tautomer, or pharmaceutical composition thereof.

The present invention provides inhibitors of viral replication activity that are therapeutic agents in the treatment of a coronavirus infection.

The present invention provides SARS-CoV-2-related 3C-like protease inhibitors that are therapeutic agents in the treatment of a coronavirus infection.

The present invention provides SARS-CoV-2-related Mpro protease inhibitors that are therapeutic agents in the treatment of a coronavirus infection.

The present invention further provides compounds and compositions with an improved efficacy and safety profile relative to known inhibitors SARS-CoV-2-related 3C-like protease.

The present invention further provides methods of treating a disease or disorder associated with coronavirus infection, comprising administering to a patient suffering from coronavirus infection a compound of Formula (II)-(V), or a pharmaceutically acceptable salt, hydrate, solvate, prodrug, stereoisomer, tautomer, or pharmaceutical composition thereof.

The present invention provides inhibitors SARS-CoV-2-related 3C-like protease that are therapeutic agents in the treatment of coronavirus infection.

The present invention provides inhibitors SARS-CoV-2-related Mpro protease that are therapeutic agents in the treatment of coronavirus infection.

The present invention further provides compounds and compositions with an improved efficacy and safety profile relative to known SARS-CoV-2-related 3C-like protease inhibitors. The present disclosure also provides agents with novel mechanisms of action toward SARS-CoV-2-related 3C-like protease in the treatment of coronavirus infection.

The present invention further provides compounds and compositions with an improved efficacy and safety profile relative to known SARS-CoV-2-related Mpro protease inhibitors. The present disclosure also provides agents with novel mechanisms of action toward SARS-CoV-2-related Mpro protease in the treatment of coronavirus infection.

The present invention further provides methods of preventing, treating, or ameliorating a coronavirus infection.

The present invention further provides methods of treating a disease, disorder, or condition selected from cold; pneumonia (either direct viral pneumonia or secondary bacterial pneumonia); bronchitis (either direct viral bronchitis or secondary bacterial bronchitis); severe acute respiratory syndrome (SARS); Middle East respiratory syndrome (MERS); Coronavirus disease 2019 (COVID-19); Coronavirus HuPn-2018 comprising administering to a patient suffering from at least one of said diseases or disorders a compound of Formula (II)-(V), or a pharmaceutically acceptable salt, hydrate, solvate, prodrug, stereoisomer, tautomer, or pharmaceutical composition thereof.

In some aspects, the present disclosure provides a compound obtainable by, or obtained by, a method for preparing compounds described herein (e.g., a method comprising one or more steps described in General Procedures).

In some aspects, the present disclosure provides an intermediate as described herein, being suitable for use in a method for preparing a compound as described herein (e.g., the intermediate is selected from the intermediates described in Preparative part—P1-P65).

In some aspects, the present disclosure provides a method of preparing compounds of the present disclosure.

In some aspects, the present disclosure provides a method of preparing compounds of the present disclosure, comprising one or more steps described herein.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims

### DETAILED DESCRIPTION

The present disclosure provides methods of treating, preventing, or ameliorating a viral infection associated with coronavirus by administering to a subject in need thereof a therapeutically effective amount of a compound as disclosed herein.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### Definitions

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The term “optionally substituted” is understood to mean that a given chemical moiety (e.g., an alkyl group) can (but is not required to) be bonded other substituents (e.g., heteroatoms). For instance, an alkyl group that is optionally substituted can be a fully saturated alkyl chain (i.e., a pure hydrocarbon). Alternatively, the same optionally substituted alkyl group can have one or more substituents different from hydrogen. For instance, it can, at any point along the chain be bonded to a halogen atom, a hydroxyl group, or any other substituent described herein. Thus, the term “optionally substituted” means that a given chemical moiety has the potential to contain other functional groups but does not necessarily have any further functional groups. Suitable substituents used in the optional substitution of the described groups include, without limitation, halogen, oxo, —OH, —CN, —NH₂, —NO₂, —COOH, —CH₂CN, —O—(C₁-C₆) alkyl, (C₁-C₆) alkyl, (C₁-C₆) alkoxy, (C₁-C₆) haloalkyl, (C₁-C₆) haloalkoxy, —O—(C₂-C₆) alkenyl, —O—(C₂-C₆) alkynyl, (C₂-C₆) alkenyl, (C₂-C₆) alkynyl, —OP(O)(OH)₂, —OC(O)(C₁-C₆) alkyl, —C(O)(C₁-C₆) alkyl, —OC(O)O(C₁-C₆) alkyl, —NH((C₁-C₆) alkyl), —N((C₁-C₆) alkyl)₂, —NHC(O)(C₁-C₆) alkyl, —C(O)NH(C₁-C₆) alkyl, —S(O)₂(C₁-C₆) alkyl, —S(O)NH(C₁-C₆)alkyl, and —S(O)N((C₁-C₆)alkyl)₂. The substituents can themselves be optionally substituted. “Optionally substituted” as used herein also refers to substituted or unsubstituted whose meaning is described below.

As used herein, the term “substituted” means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

As used herein, the term “unsubstituted” means that the specified group bears no substituents.

Unless otherwise specifically defined, the term “aryl” refers to cyclic, aromatic hydrocarbon groups that have 1 to 3 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. Exemplary substituents include, but are not limited to, —H, -halogen, —O—(C₁-C₆)alkyl, (C₁-C₆)alkyl, —O—(C₂-C₆)alkenyl, —O—(C₂-C₆) alkynyl, (C₂-C₆)alkenyl, (C₂-C₆)alkynyl, —OH, —OP(O)(OH)₂, —OC(O)(C₁-C₆)alkyl, —C(O)(C₁-C₆) alkyl, —OC(O)O(C₁-C₆)alkyl, —NH₂, —NH((C₁-C₆)alkyl), —N((C₁-C₆)alkyl)₂, —S(O)₂—(C₁-C₆) alkyl, —S(O)NH(C₁-C₆)alkyl, and —S(O)N((C₁-C₆)alkyl)₂. The substituents can themselves be optionally substituted. Furthermore, when containing two fused rings the aryl groups herein defined may have one or more saturated or partially unsaturated ring fused with a fully unsaturated aromatic ring. Exemplary ring systems of these aryl groups include, but are not limited to, phenyl, biphenyl, naphthyl, anthracenyl, phenalenyl, phenanthrenyl, indanyl, indenyl, tetrahydronaphthalenyl, tetrahydrobenzoannulenyl, and the like.

Unless otherwise specifically defined, “heteroaryl” means a monovalent monocyclic or a polycyclic aromatic radical of 5 to 24 ring atoms, containing one or more ring heteroatoms selected from N, O, S, P, or B, the remaining ring atoms being C. A polycyclic aromatic radical includes two or more fused rings and may further include two or more spiro-fused rings, e.g., bicyclic, tricyclic, tetracyclic, and the like. Unless otherwise specifically defined, “fused” means two rings sharing two ring atoms. Unless otherwise specifically defined, “spiro-fused” means two rings sharing one ring atom. Heteroaryl as herein defined also means a bicyclic heteroaromatic group wherein the heteroatom is selected from N, O, S, P, or B. Heteroaryl as herein defined also means a tricyclic heteroaromatic group containing one or more ring heteroatoms selected from N, O, S, P, or B. Heteroaryl as herein defined also means a tetracyclic heteroaromatic group containing one or more ring heteroatoms selected from N, O, S, P, or B. The aromatic radical is optionally substituted independently with one or more substituents described herein. Examples include, but are not limited to, furyl, thienyl, pyrrolyl, pyridyl, pyrazolyl, pyrimidinyl, imidazolyl, isoxazolyl, oxazolyl, oxadiazolyl, pyrazinyl, indolyl, thiophen-2-yl, quinolyl, benzopyranyl, isothiazolyl, thiazolyl, thiadiazole, indazole, benzimidazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuran, chromanyl, thiochromanyl, tetrahydroquinolinyl, dihydrobenzothiazine, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydro pyrrolo[1,2-a]pyrimidinyl, 3,4-dihydro-2H-1-pyrrolo[2,1-b]pyrimidine, dibenzo[b,d]thiophene, pyridin-2-one, furo[3,2-c]pyridinyl, furo[2,3-c]pyridinyl, 1H-pyrido[3,4-b][1,4]thiazinyl, benzooxazolyl, benzoisoxazolyl, furo[2,3-b]pyridinyl, benzothiophenyl, 1,5-naphthyridinyl, furo[3,2-b]pyridine, [1,2,4]triazolo[1,5-a]pyridinyl, benzo[1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo [1,5-b][1,2]oxazinyl, 4,5,6,7-tetrahydropyrazolo[1,5-a]pyridinyl, thiazolo[5,4-d]thiazolyl, imidazo[2,1-b][1,3,4]thiadiazolyl, thieno[2,3-b]pyrrolyl, 3H-indolyl, and derivatives thereof. Furthermore, when containing two or more fused rings, the heteroaryl groups defined herein may have one or more saturated or partially unsaturated ring fused with one or more fully unsaturated aromatic ring. In heteroaryl ring systems containing more than two fused rings, a saturated or partially unsaturated ring may further be fused with a saturated or partially unsaturated ring described herein. Furthermore, when containing three or more fused rings, the heteroaryl groups defined herein may have one or more saturated or partially unsaturated ring spiro-fused. Any saturated or partially unsaturated ring described herein is optionally substituted with one or more oxo. Exemplary ring systems of these heteroaryl groups include, for example, indolinyl, indolinonyl, dihydrobenzothiophenyl, dihydrobenzofuran, chromanyl, thiochromanyl, tetrahydroquinolinyl, dihydrobenzothiazine, 3,4-dihydro-1H-isoquinolinyl, 2,3-dihydrobenzofuranyl, benzofuranonyl, indolinyl, oxindolyl, indolyl, 1,6-dihydro-7H-pyrazolo[3,4-c]pyridin-7-onyl, 7,8-dihydro-6H-pyrido[3,2-b]pyrrolizinyl, 8H-pyrido[3,2-b]pyrrolizinyl, 1,5,6,7-tetrahydrocyclopenta[b]pyrazolo[4,3-e]pyridinyl, 7,8-dihydro-6H-pyrido[3,2-b]pyrrolizine, pyrazolo[1,5-a]pyrimidin-7(4H)-only, 3,4-dihydropyrazino[1,2-a]indol-1(2H)-onyl, benzo[c][1,2]oxaborol-1(3H)-olyl, 6,6a,7,8-tetrahydro-9H-pyrido[2,3-b]puyrrolo[1,2-d][1,4]oxazin-9-onyl, or 6a′,7′-dihydro-6′H,9′H-spiro[cyclopropane-1,8′-pyrido[2,3-b]pyrrolo[1,2-d][1,4]oxazin]-9′-onyl.

Halogen or “halo” refers to fluorine, chlorine, bromine, or iodine.

Alkyl refers to a straight or branched chain saturated hydrocarbon containing 1-12 carbon atoms. Examples of a (C₁-C₆) alkyl group include, but are not limited to, methyl, ethyl, propyl, butyl, pentyl, hexyl, isopropyl, isobutyl, sec-butyl, tert-butyl, isopentyl, neopentyl, and isohexyl.

“Alkoxy” refers to a straight or branched chain saturated hydrocarbon containing 1-12 carbon atoms containing a terminal “O” in the chain, i.e., —O(alkyl). Examples of alkoxy groups include without limitation, methoxy, ethoxy, propoxy, butoxy, tert-butoxy, or pentoxy groups.

“Alkenyl” refers to a straight or branched chain unsaturated hydrocarbon containing 2-12 carbon atoms. The “alkenyl” group contains at least one double bond in the chain. The double bond of an alkenyl group can be unconjugated or conjugated to another unsaturated group. Examples of alkenyl groups include ethenyl, propenyl, n-butenyl, iso-butenyl, pentenyl, or hexenyl. An alkenyl group can be unsubstituted or substituted. Alkenyl, as herein defined, may be straight or branched.

“Alkynyl” refers to a straight or branched chain unsaturated hydrocarbon containing 2-12 carbon atoms. The “alkynyl” group contains at least one triple bond in the chain. Examples of alkynyl groups include ethynyl, propargyl, n-butynyl, iso-butynyl, pentynyl, or hexynyl. An alkynyl group can be unsubstituted or substituted.

The term “alkylene” or “alkylenyl” refers to a divalent alkyl radical. Any of the above mentioned monovalent alkyl groups may be an alkylene by abstraction of a second hydrogen atom from the alkyl. As herein defined, alkylene may also be a C₁-C₆ alkylene. An alkylene may further be a C₁-C₄ alkylene. Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

“Cycloalkyl” means mono or polycyclic saturated carbon rings containing 3-18 carbon atoms. Polycyclic cycloalkyl may be fused bicyclic cycloalkyl, bridged bicyclic cycloalkyl, or spiro-fused bicyclic cycloalkyl. A polycyclic cycloalkyl comprises at least one non-aromatic ring. Examples of cycloalkyl groups include, without limitations, cyclopropyl, cyclobutyl, cyclopentyl, cyclohexyl, cycloheptanyl, cyclooctanyl, norbornyl, norbornenyl, 1,2,3,4-tetrahydronaphthyl, 2,3-dihydro-1H-indenyl, spiro[3.5]nonyl, spiro[5.5]undecyl, bicyclo[1.1.1]pentanyl, bicyclo[2.2.2]octanyl, or bicyclo[2.2.2]octenyl.

“Heterocyclyl”, “heterocycle” or “heterocycloalkyl” mono or polycyclic rings containing 3-24 atoms which include carbon and one or more heteroatoms selected from N, O, S, P, or B and wherein the rings are not aromatic. The heterocycloalkyl ring structure may be substituted by one or more substituents. The substituents can themselves be optionally substituted. Examples of heterocyclyl rings include, but are not limited to, oxetanyl, azetidinyl, tetrahydrofuranyl, tetrahydropyranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, oxazolidinonyl, and homotropanyl.

The term “aromatic” means a planar ring having 4n+2 electrons in a conjugated system. As used herein, “conjugated system” means a system of connected p-orbitals with delocalized electrons, and the system may include lone electron pairs.

The term “halogenalkyl” as used herein refers to an alkyl group, as defined herein, which is substituted one or more halogen. Examples of halogenalkyl groups include, but are not limited to, trifluoromethyl, difluoromethyl, pentafluoroethyl, trichloromethyl, etc.

The term “halogenalkoxy” as used herein refers to an alkoxy group, as defined herein, which is substituted with one or more halogen. Examples of haloalkyl groups include, but are not limited to, trifluoromethoxy, difluoromethoxy, pentafluoroethoxy, trichloromethoxy, etc.

The term “cyano” as used herein means a substituent having a carbon atom joined to a nitrogen atom by a triple bond, i.e., C≡N.

“Spirocycloalkyl” or “spirocyclyl” means carbogenic bicyclic ring systems with both rings connected through a single atom. The ring can be different in size and nature, or identical in size and nature. Examples include spiropentane, spirohexane, spiroheptane, spirooctane, spirononane, or spirodecane. One or both of the rings in a spirocycle can be fused to another ring carbocyclic, heterocyclic, aromatic, or heteroaromatic ring. One or more of the carbon atoms in the spirocycle can be substituted with a heteroatom (e.g., O, N, S, or P). A (C₃-C₁₂) spirocycloalkyl is a spirocycle containing between 3 and 12 carbon atoms. One or more of the carbon atoms can be substituted with a heteroatom.

The term “spiroheterocycloalkyl”, “spiroheterocycle”, or “spiroheterocyclyl” is understood to mean a spirocycle wherein at least one of the rings is a heterocycle (e.g., at least one of the rings is furanyl, morpholinyl, or piperidinyl).

The term “solvate” refers to a complex of variable stoichiometry formed by a solute and solvent. Such solvents for the purpose of the disclosure may not interfere with the biological activity of the solute. Examples of suitable solvents include, but are not limited to, water, MeOH, EtOH, and AcOH. Solvates wherein water is the solvent molecule are typically referred to as hydrates. Hydrates include compositions containing stoichiometric amounts of water, as well as compositions containing variable amounts of water.

The term “isomer” refers to compounds that have the same composition and molecular weight but differ in physical and/or chemical properties. The structural difference may be in constitution (geometric isomers) or in the ability to rotate the plane of polarized light (stereoisomers). With regard to stereoisomers, the compounds of Formula (A) may have one or more asymmetric carbon atom and may occur as racemates, racemic mixtures and as individual enantiomers or diastereomers.

The present disclosure also contemplates isotopically-labelled compounds of Formula (A) (e.g., those labeled with ²H and ¹⁴C). Deuterated (i.e., ²H or D) and carbon-14 (i.e., ¹⁴C) isotopes are particularly preferred for their ease of preparation and detectability. Further, substitution with heavier isotopes such as deuterium may afford certain therapeutic advantages resulting from greater metabolic stability (e.g., increased in vivo half-life or reduced dosage requirements) and hence may be preferred in some circumstances. Isotopically labelled compounds of Formula (A) can generally be prepared by following procedures analogous to those disclosed in the Schemes and/or in the Examples herein below, by substituting an appropriate isotopically labelled reagent for a non-isotopically labelled reagent.

The disclosure also includes pharmaceutical compositions comprising a therapeutically effective amount of a disclosed compound and a pharmaceutically acceptable carrier. Representative “pharmaceutically acceptable salts” include, e.g., water-soluble and water-insoluble salts, such as the acetate, amsonate (4,4-diaminostilbene-2,2-disulfonate), benzenesulfonate, benzoate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate, pantothenate, phosphate/diphosphate, picrate, polygalacturonate, propionate, p-toluenesulfonate, salicylate, stearate, subacetate, succinate, sulfate, sulfosalicylate, tannate, tartrate, teoclate, tosylate, triethiodide, and valerate salts.

A “patient” or “subject” is a mammal, e.g., a human, mouse, rat, guinea pig, dog, cat, horse, cow, pig, or non-human primate, such as a monkey, chimpanzee, baboon, or rhesus.

An “effective amount” when used in connection with a compound is an amount effective for treating or preventing a disease in a subject as described herein.

The term “carrier”, as used in this disclosure, encompasses carriers, excipients, and diluents, and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “treating” with regard to a subject, refers to improving at least one symptom of the subject's disorder. Treating includes curing, improving, or at least partially ameliorating the disorder.

The term “disorder” is used in this disclosure to mean, and is used interchangeably with, the terms disease, condition, or illness, unless otherwise indicated.

The term “administer”, “administering”, or “administration” as used in this disclosure refers to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body.

The term “prodrug,” as used in this disclosure, means a compound which is convertible in vivo by metabolic means (e.g., by hydrolysis) to a disclosed compound.

The term “salt’ refers to pharmaceutically acceptable salts.

The term “pharmaceutically acceptable salt” also refers to a salt of the compositions of the present disclosure having an acidic functional group, such as a carboxylic acid functional group, and a base.

“SARS-CoV-2-related 3C-like protease inhibitor” as used herein refer to compounds of Formula (II), Formula (III), Formula (IV), or Formula (V) and/or compositions comprising a compound of Formula (II), Formula (III), Formula (IV), or Formula (V) which inhibit the SARS-CoV-2-related 3C-like protease.

“SARS-CoV-2 viral replication inhibitor” as used herein refer to compounds of Formula (II), Formula (III), Formula (IV), or Formula (V) and/or compositions comprising a compound of Formula (II), Formula (III), Formula (IV), or Formula (V) which inhibit the SARS-CoV-2 viral replication.

The term “SARS-CoV-2 inhibiting agent” means any SARS-CoV-2-related coronavirus 3C-like protease inhibitor compound described herein or a pharmaceutically acceptable salt, hydrate, prodrug, active metabolite or solvate thereof or a compound which inhibits replication of SARS-CoV-2 in any manner.

The amount of compound or composition described herein needed for achieving a therapeutic effect may be determined empirically in accordance with conventional procedures for the particular purpose. Generally, for administering therapeutic agents (e.g. compounds or compositions of Formula (II), Formula (III), Formula (IV) or Formula (V) (and/or additional agents) described herein) for therapeutic purposes, the therapeutic agents are given at a pharmacologically effective dose.

A “pharmacologically effective amount”, “pharmacologically effective dose”, “therapeutically effective amount”, or “effective amount” refers to an amount sufficient to produce the desired physiological effect or amount capable of achieving the desired result, particularly for treating the coronavirus infection. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

### Compounds of the Present Disclosure

In one aspect, the present disclosure provides compounds of Formula (II) and pharmaceutical acceptable salts, stereoisomers, solvates, prodrugs, isotopic derivatives, and tautomers thereof:

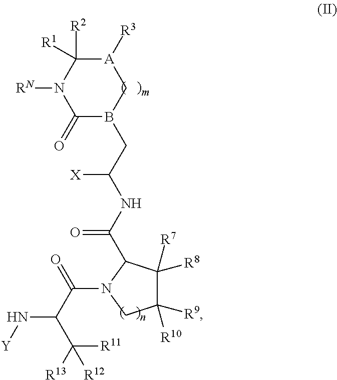

Wherein R^{N}, R¹, R², R³, R⁷, R⁸, R⁹, R¹⁰, R¹¹, R¹², R¹³, A, B, X, Y, m and n are as described herein.

It is understood that, for a compound of Formula (II), R^{N}, R¹, R², R³, R⁷, R⁸, R⁹, R¹⁰, R¹¹, R¹², R¹³, A, B, X, Y, m and n can each be, where applicable, selected from the groups described herein, and any group described herein for any of R^{N}, R¹, R², R³, R⁷, R⁸, R⁹, R¹⁰, R¹¹, R¹², R¹³, A, B, X, Y, m and n can be combined, where applicable, with any group described herein for one or more of the remainder of R^{N}, R¹, R², R³, R⁷, R⁸, R⁹, R¹⁰, R¹¹, R¹², R¹³, A, B, X, Y, m and n.

In some embodiments,
A is selected from CR⁴ and N;
B is selected from CR⁶ and N;
R^{N} is selected from H, C₁-C₆ alkyl, cycloalkyl;
R¹ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R² is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R¹ and R² together is oxo;
R³ is selected from hydrogen, C₁-C₆ alkyl, C₂-C₆ alkenyl, C₂-C₆ alkynyl, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R⁴ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R¹ and R⁴ together with the atoms to which they are attached and any intervening atoms, form a 3-10 membered cycloalkyl, a 6-10 membered aryl, a 3-14 membered heterocycle, or a 5-6 membered heteroaryl, wherein the cycloalkyl, aryl, heterocycle or heteroaryl is optionally substituted with one or more R¹⁶;
or R³ and R⁴ together is oxo;
R⁶ is selected from H, C₁-C₆ alkyl, cycloalkyl, halogen, —CN, —OH;
R⁷, R⁸, R⁹ and R¹⁰ are each independently selected from H, C₁-C₆ alkyl, cycloalkyl;
or R⁸ and R⁹ together with the atoms to which they are attached and any intervening atoms, form cycloalkyl optionally substituted with one or more halogen, —OH, —CN, C₁-C₆ alkyl;
or R⁹ and R¹⁰ together with the atoms to which they are attached and any intervening atoms, form cycloalkyl optionally substituted with one or more halogen, —OH, —CN, C₁-C₆ alkyl;
R¹¹, R¹², R¹³ are each independently selected from C₁-C₆ alkyl, cycloalkyl, halogen, —CN;
R¹⁴ and R¹⁵ are each independently selected from H, C₁-C₆ alkyl;
or R¹⁴ and R¹⁵ together with the atoms to which they are attached and any intervening atoms, form heterocyclyl optionally substituted with one or more halogen, —OH, —CN, C₁-C₆ alkyl;
R¹⁶ is independently selected from halogen, —OH, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)N—NR¹⁴R¹⁵, cycloalkyl, —O-cycloalkyl, aryl, heterocyclyl, and heteroaryl;
X is selected from hydrogen, halogen, OH, CN, NO₂, CONH₂, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl wherein alkyl optionally substituted with cycloalkyl, heterocyclyl, aryl, and heteroaryl;
Y is selected from C₁-C₆ alkyl-S(O)ᵤ—, C₁-C₆ alkyl-C(O)—, heteroaryl-C(O)—, wherein alkyl or heteroaryl is optionally substituted with one or more halogen, —OH, —CN;
m is an integer selected from 0, 1, 2;
n is an integer selected from 0, 1, 2;
u is integer selected from 0, 1, 2.

In some embodiments, A is CR⁴.

In some embodiments, A is CH.

In some embodiments, A is CCH₃.

In some embodiments, A is N.

In some embodiments, B is CR⁶.

In some embodiments, B is CH.

In some embodiments, B is N.

In some embodiments, R^{N} is selected from H, C₁-C₆ alkyl, cycloalkyl.

In some embodiments, R^{N} is H.

In some embodiments, R^{N} is C₁-C₆ alkyl.

In some embodiments, R^{N} is methyl.

In some embodiments, R^{N} is ethyl.

In some embodiments, R^{N} is cycloalkyl.

In some embodiments, R^{N} is cyclopropyl.

In some embodiments, R^{N} is cyclobutyl.

In some embodiments, R^{N} is cyclopentyl.

In some embodiments, R^{N} is cyclohexyl.

In some embodiments, R¹ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶.

In some embodiments, R¹ is hydrogen.

In some embodiments, R¹ is deuterium.

In some embodiments, R¹ and R⁴ together with the atoms to which they are attached and any intervening atoms, form a 3-10 membered cycloalkyl, a 6-10 membered aryl, a 3-14 membered heterocycle, or a 5-6 membered heteroaryl, wherein the cycloalkyl, aryl, heterocycle or heteroaryl is optionally substituted with one or more R¹⁶.

In some embodiments, R¹ and R⁴ together with the atoms to which they are attached and any intervening atoms, form a 6 membered aryl is optionally substituted with one or more R¹⁶.

In some embodiments, R¹ and R⁴ together with the atoms to which they are attached and any intervening atoms, form a 6 membered unsubstituted aryl.

In some embodiments, R¹ and R⁴ together with the atoms to which they are attached and any intervening atoms, form a 6 membered aryl substituted with one or more halogen.

In some embodiments, R¹ and R⁴ together with the atoms to which they are attached and any intervening atoms, form a 6 membered aryl substituted with one or more F.

In some embodiments, R¹ and R⁴ together with the atoms to which they are attached and any intervening atoms, form a 6 membered aryl substituted with one F.

In some embodiments, R¹ and R⁴ together with the atoms to which they are attached and any intervening atoms, form a 6 membered aryl substituted with one or more Cl.

In some embodiments, R¹ and R⁴ together with the atoms to which they are attached and any intervening atoms, form a 6 membered aryl substituted with one Cl.

In some embodiments, R¹ and R⁴ together with the atoms to which they are attached and any intervening atoms, form a 6 membered aryl optionally substituted with one or more C₁-C₆ alkyl.

In some embodiments, R¹ and R⁴ together with the atoms to which they are attached and any intervening atoms, form a 6 membered aryl substituted with one or more methyl.

In some embodiments, R¹ and R⁴ together with the atoms to which they are attached and any intervening atoms, form a 6 membered aryl substituted with one methyl.

In some embodiments, R¹ and R⁴ together with the atoms to which they are attached and any intervening atoms, form a 6 membered aryl optionally with one or more C₁-C₆ alkoxy.

In some embodiments, R¹ and R⁴ together with the atoms to which they are attached and any intervening atoms, form a 6 membered aryl substituted with one or more methoxy.

In some embodiments, R¹ and R⁴ together with the atoms to which they are attached and any intervening atoms, form a 6 membered aryl substituted with one methoxy.

In some embodiments, R² is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶.

In some embodiments, R² is H.

In some embodiments, R² and R³ together form a double bond.

In some embodiments, R³ is selected from hydrogen, C₁-C₆ alkyl, C₂-C₆ alkenyl, C₂-C₆ alkynyl, cycloalkyl, aryl, heterocyclyl, and heteroaryl, wherein the alkyl, alkenyl, alkynyl, cycloalkyl, aryl, heterocyclyl, or heteroaryl is optionally substituted with one or more R¹⁶.

In some embodiments, R³ and R² together form a double bond.

In some embodiments, R³ is H.

In some embodiments, R⁴ is selected from hydrogen, halogen, —OH, —CN, —NO₂, —NR¹³R¹⁴, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶.

In some embodiments, R⁴ is H.

In some embodiments, R⁴ and R¹ together with the atoms to which they are attached and any intervening atoms, form a 3-10 membered cycloalkyl, a 6-10 membered aryl, a 3-14 membered heterocycle, or a 5-6 membered heteroaryl, wherein the cycloalkyl, aryl, heterocycle or heteroaryl is optionally substituted with one or more R¹⁶.

In some embodiments, R⁴ and R¹ together with the atoms to which they are attached and any intervening atoms, form a 6 membered aryl optionally substituted with one or more R¹⁶.

In some embodiments, R⁴ and R¹ together with the atoms to which they are attached and any intervening atoms, form a 6 membered unsubstituted aryl.

In some embodiments, R⁴ and R¹ together with the atoms to which they are attached and any intervening atoms, form a 6 membered aryl substituted with one or more halogen.

In some embodiments, R⁴ and R¹ together with the atoms to which they are attached and any intervening atoms, form a 6 membered aryl substituted with one or more F.

In some embodiments, R⁴ and R¹ together with the atoms to which they are attached and any intervening atoms, form a 6 membered aryl substituted with one F.

In some embodiments, R⁴ and R¹ together with the atoms to which they are attached and any intervening atoms, form a 6 membered aryl substituted with one or more Cl.

In some embodiments, R⁴ and R¹ together with the atoms to which they are attached and any intervening atoms, form a 6 membered aryl substituted with one Cl.

In some embodiments, R⁴ and R¹ together with the atoms to which they are attached and any intervening atoms, form a 6 membered aryl optionally substituted with one or more C₁-C₆ alkyl.

In some embodiments, R⁴ and R¹ together with the atoms to which they are attached and any intervening atoms, form a 6 membered aryl substituted with one or more methyl.

In some embodiments, R⁴ and R¹ together with the atoms to which they are attached and any intervening atoms, form a 6 membered aryl substituted with one methyl.

In some embodiments, R⁴ and R¹ together with the atoms to which they are attached and any intervening atoms, form a 6 membered aryl optionally substituted with one or more C₁-C₆ alkoxy.

In some embodiments, R⁴ and R¹ together with the atoms to which they are attached and any intervening atoms, form a 6 membered aryl substituted with one or more methoxy.

In some embodiments, R⁴ and R¹ together with the atoms to which they are attached and any intervening atoms, form a 6 membered aryl substituted with one methoxy.

In some embodiments, m is integer selected from 0, 1, 2.

In some embodiments, m is 0.

In some embodiments, m is 1.

In some embodiments, m is 2.

In some embodiments, n is integer selected from 0, 1, 2.

In some embodiments, n is 2.

In some embodiments, n is 1.

In some embodiments, n is 0.

In some embodiments, R⁷, R⁸, R⁹ and R¹⁰ are each independently selected from H, C₁-C₆ alkyl.

In some embodiments, R⁸ and R⁹ together with the atoms to which they are attached and any intervening atoms, form cycloalkyl optionally substituted with one or more halogen, —OH, —CN, C₁-C₆ alkyl.

In some embodiments, R⁸ and R⁹ together with the atoms to which they are attached and any intervening atoms, form a cyclopropane ring optionally substituted with one or more halogen, —OH, —CN, C₁-C₆ alkyl.

In some embodiments, R⁹ and R¹⁰ together with the atoms to which they are attached and any intervening atoms, form cycloalkyl optionally substituted with one or more halogen, —OH, —CN, C₁-C₆ alkyl.

In some embodiments, R⁷ and R⁸ each is H, R⁹ and R¹⁰ are each C₁-C₆ alkyl.

In some embodiments, R⁷ and R⁸ each is H, R⁹ and R¹⁰ are each CH₃.

In some embodiments, n is 2, R⁷ and R⁸ each is H, R⁹ and R¹⁰ are each C₁-C₆ alkyl.

In some embodiments, n is 2, R⁷ and R⁸ each is H, R⁹ and R¹⁰ are each CH₃.

In some embodiments, n is 2, R⁹ and R¹⁰ together with the atoms to which they are attached and any intervening atoms, form cycloalkyl optionally substituted with one or more halogen, —OH, —CN, C₁-C₆ alkyl.

In some embodiments, n is 2, R⁹ and R¹⁰ together with the atoms to which they are attached and any intervening atoms, form cyclopropane ring optionally substituted with one or more halogen, —OH, —CN, C₁-C₆ alkyl.

In some embodiments, n is 1, R⁸ and R⁹ together with the atoms to which they are attached and any intervening atoms, form cycloalkyl optionally substituted with one or more halogen, —OH, —CN, C₁-C₆ alkyl.

In some embodiments, n is 1, R⁸ and R⁹ together with the atoms to which they are attached and any intervening atoms, form a cyclopropane ring optionally substituted with one or more halogen, —OH, —CN, C₁-C₆ alkyl.

In some embodiments, n is 1, R⁸ and R⁹ together with the atoms to which they are attached and any intervening atoms, form a cyclopropane ring optionally substituted with one or more halogen, —OH, —CN, C₁-C₆ alkyl.

In some embodiments, n is 1, R⁸ and R⁹ together with the atoms to which they are attached and any intervening atoms, form a cyclopropane ring optionally substituted with one or more C₁-C₆ alkyl.

In some embodiments, n is 1, R⁸ and R⁹ together with the atoms to which they are attached and any intervening atoms, form a cyclopropane ring optionally substituted with two C₁-C₆ alkyl.

In some embodiments, n is 1, R⁸ and R⁹ together with the atoms to which they are attached and any intervening atoms, form a cyclopropane ring optionally geminal substituted with two C₁-C₆ alkyl.

In some embodiments, n is 1, R⁸ and R⁹ together with the atoms to which they are attached and any intervening atoms, form a cyclopropane ring optionally geminal substituted with two methyl.

In some embodiments, R¹¹, R¹², R¹³ are each independently selected from H, C₁-C₆ alkyl, cycloalkyl, halogen, —CN.

In some embodiments, R¹¹, R¹², R¹³ are each C₁-C₆ alkyl.

In some embodiments, R¹¹, R¹², R¹³ are each methyl.

In some embodiments, R¹¹, R¹², R¹³ are each halogen.

In some embodiments, R¹¹, R¹², R¹³ are each F.

In some embodiments, R¹¹ is methyl, R¹² and R¹³ are each F.

In some embodiments, R¹⁴ and R¹⁵ are each independently selected from H, C₁-C₆ alkyl.

In some embodiments, R¹⁴ and R¹⁵ are each H.

In some embodiments, R¹⁴ and R¹⁵ are each methyl.

In some embodiments, R¹⁴ and R¹⁵ together with the atoms to which they are attached and any intervening atoms, form heterocyclyl optionally substituted with one or more halogen, —OH, —CN, C₁-C₆ alkyl.

In some embodiments, R¹⁶ is independently selected from halogen, —OH, —OH, —CN, —NO₂, —NR¹³R¹⁴, C₁-C₆ alkyl, C₂-C₆ alkenyl, C₂-C₆ alkynyl, C₁-C₆ alkoxy, —C(O)OR¹³, —C(O)N—NR¹³R¹⁴, cycloalkyl, —O-cycloalkyl, aryl, heterocyclyl, and heteroaryl.

In some embodiments, X is selected from hydrogen, halogen, OH, CN, NO₂, CONR¹³R¹⁴, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl wherein alkyl optionally substituted with cycloalkyl, heterocyclyl, aryl, and heteroaryl.

In some embodiments, X is —CN.

In some embodiments, X is CONR¹³R¹⁴.

In some embodiments, X is CONH₂.

In some embodiments, X is selected from C₁-C₆ alkyl optionally substituted with cycloalkyl, heterocyclyl, aryl, and heteroaryl.

In some embodiments, X is selected from C₁-C₆ alkyl substituted with heterocyclyl.

In some embodiments, X is

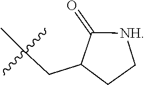 [SMILES: *CC1CCNC1=O]

In some embodiments, X is

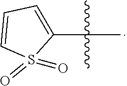 [SMILES: *C1=CC=CS1(=O)=O]

In some embodiments, Y is selected from C₁-C₆ alkyl-S(O)ᵤ—, C₁-C₆ alkyl-C(O)—, heteroaryl-C(O)—, wherein alkyl or heteroaryl is optionally substituted with one or more halogen, —OH, —CN.

In some embodiments, u is integer selected from 0, 1, 2.

In some embodiments, u is 0.

In some embodiments, u is 1.

In some embodiments, u is 2.

In some embodiments, Y is CF₃C(O)—.

In some embodiments, Y is CH₃S(O)₂—.

In some embodiments, Y is CF₃S(O)₂—.

In some embodiments, Y is CF₃CH₂S(O)₂—.

In some embodiments, Y is

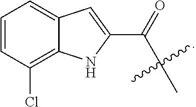 [SMILES: *C(=O)c1cc2cccc(Cl)c2[nH]1]

In another aspect, the present disclosure provides compounds of Formula (III) and pharmaceutical acceptable salts, stereoisomers, solvates, prodrugs, isotopic derivatives, and tautomers thereof:

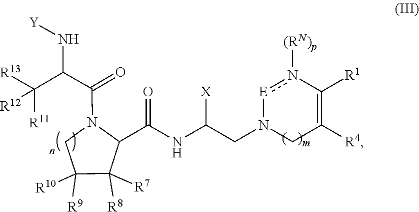

Wherein R^{N}, R¹, R⁴, R⁷, R⁸, R⁹, R¹⁰, R¹¹, R¹², R¹³, E, X, Y, m, n and p are as described herein.

It is understood that, for a compound of Formula (III), R^{N}, R¹, R⁴, R⁷, R⁸, R⁹, R¹⁰, R¹¹, R¹², R¹³, E, X, Y, m, n and p can each be, where applicable, selected from the groups described herein, and any group described herein for any of R^{N}, R¹, R⁴, R⁷, R⁸, R⁹, R¹⁰, R¹¹, R¹², R¹³, E, X, Y, m, n and p can be combined, where applicable, with any group described herein for one or more of the remainder of R^{N}, R¹, R⁴, R⁷, R⁸, R⁹, R¹⁰, R¹¹, R¹², R¹³, E, X, Y, m, n and p.

In some embodiments,
the bond [symbol] is selected from a single bond and a double bond;
p is an integer selected from 0, 1;
E is selected from C(O) and N;
   provided that when the bond [symbol] is a single bond p is 1 and when the bond [symbol] is a double bond p is 0;
and provided that when the bond [symbol] is a double bond E is N and the bond [symbol] is a single bond E is C(O);
R^{N} is selected from H, C₁-C₆ alkyl, cycloalkyl;
R¹ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R⁴ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R¹ and R⁴ together with the atoms to which they are attached and any intervening atoms, form a 3-10 membered cycloalkyl, a 6-10 membered aryl, a 3-14 membered heterocycle, or a 5-6 membered heteroaryl, wherein the cycloalkyl, aryl, heterocycle or heteroaryl is optionally substituted with one or more R¹⁶;
R⁷, R⁸, R⁹ and R¹⁰ are each independently selected from H, C₁-C₆ alkyl, cycloalkyl;
or R⁸ and R⁹ together with the atoms to which they are attached and any intervening atoms, form cycloalkyl optionally substituted with one or more halogen, —OH, —CN, C₁-C₆ alkyl;
or R⁹ and R¹⁰ together with the atoms to which they are attached and any intervening atoms, form cycloalkyl optionally substituted with one or more halogen, —OH, —CN, C₁-C₆ alkyl;
R¹¹, R¹², R¹³ are each independently selected from C₁-C₆ alkyl, cycloalkyl, halogen, —CN;
R¹⁴ and R¹⁵ are each independently selected from H, C₁-C₆ alkyl;
or R¹⁴ and R¹⁵ together with the atoms to which they are attached and any intervening atoms, form heterocyclyl optionally substituted with one or more halogen, —OH, —CN, C₁-C₆ alkyl;
R¹⁶ is independently selected from halogen, —OH, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)N—NR¹⁴R¹⁵, cycloalkyl, —O-cycloalkyl, aryl, heterocyclyl, and heteroaryl;
X is selected from hydrogen, halogen, OH, CN, NO₂, CONH₂, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl wherein alkyl optionally substituted with cycloalkyl, heterocyclyl, aryl, and heteroaryl;
Y is selected from C₁-C₆ alkyl-S(O)ᵤ—, C₁-C₆ alkyl-C(O)—, wherein alkyl is optionally substituted with one or more halogen, —OH, —CN;
m is an integer selected from 0, 1, 2;
n is an integer selected from 0, 1, 2;
u is integer selected from 0, 1, 2.

In some embodiments, the bond [symbol] is a single bond, p is 1 and E is C═O.

In some embodiments, the bond [symbol] is a double bond, p is 0 and E is N.

In some embodiments, m is an integer selected from 0, 1, 2.

In some embodiments, m is 0.

In some embodiments, m is 1.

In some embodiments, m is 2.

In some embodiments, R^{N} is selected from H, C₁-C₆ alkyl, cycloalkyl.

In some embodiments, R^{N} is selected from H.

In some embodiments, R^{N} is selected from C₁-C₆ alkyl.

In some embodiments, R^{N} is selected from methyl.

In some embodiments, R^{N} is selected from cycloalkyl.

In some embodiments, R^{N} is selected from cyclopropyl.

In some embodiments, R¹ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶.

In some embodiments, R¹ is H.

In some embodiments, R¹ is C₁-C₆ alkyl.

In some embodiments, R¹ is methyl.

In some embodiments, R¹ and R⁴ together with the atoms to which they are attached and any intervening atoms, form a 3-10 membered cycloalkyl, a 6-10 membered aryl, a 3-14 membered heterocycle, or a 5-6 membered heteroaryl, wherein the cycloalkyl, aryl, heterocycle or heteroaryl is optionally substituted with one or more R¹⁶.

In some embodiments, R¹ and R⁴ together with the atoms to which they are attached and any intervening atoms, form a 6 membered aryl is optionally substituted with one or more R¹⁶.

In some embodiments, R¹ and R⁴ together with the atoms to which they are attached and any intervening atoms, form a 6 membered unsubstituted aryl.

In some embodiments, R¹ and R⁴ together with the atoms to which they are attached and any intervening atoms, form a 6 membered aryl is substituted with one or more halogen.

In some embodiments, R¹ and R⁴ together with the atoms to which they are attached and any intervening atoms, form a 6 membered aryl is substituted with one or more F.

In some embodiments, R¹ and R⁴ together with the atoms to which they are attached and any intervening atoms, form a 6 membered aryl is substituted with one F.

In some embodiments, R¹ and R⁴ together with the atoms to which they are attached and any intervening atoms, form a 6 membered aryl is substituted with one or more Cl.

In some embodiments, R¹ and R⁴ together with the atoms to which they are attached and any intervening atoms, form a 6 membered aryl is substituted with one Cl.

In some embodiments, R¹ and R⁴ together with the atoms to which they are attached and any intervening atoms, form a 6 membered aryl is optionally with one or more C₁-C₆ alkyl.

In some embodiments, R¹ and R⁴ together with the atoms to which they are attached and any intervening atoms, form a 6 membered aryl is substituted with one or more methyl.

In some embodiments, R¹ and R⁴ together with the atoms to which they are attached and any intervening atoms, form a 6 membered aryl is substituted with one methyl.

In some embodiments, R¹ and R⁴ together with the atoms to which they are attached and any intervening atoms, form a 6 membered aryl is optionally with one or more C₁-C₆ alkoxy.

In some embodiments, R¹ and R⁴ together with the atoms to which they are attached and any intervening atoms, form a 6 membered aryl is substituted with one or more methoxy.

In some embodiments, R¹ and R⁴ together with the atoms to which they are attached and any intervening atoms, form a 6 membered aryl is substituted with one methoxy.

In some embodiments, R² is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶.

In some embodiments, R⁴ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶.

In some embodiments, R⁴ is H.

In some embodiments, R⁴ is C₁-C₆ alkyl.

In some embodiments, R⁴ is methyl.

In some embodiments, R⁴ and R¹ together with the atoms to which they are attached and any intervening atoms, form a 3-10 membered cycloalkyl, a 6-10 membered aryl, a 3-14 membered heterocycle, or a 5-6 membered heteroaryl, wherein the cycloalkyl, aryl, heterocycle or heteroaryl is optionally substituted with one or more R¹⁶.

In some embodiments, R⁴ and R¹ together with the atoms to which they are attached and any intervening atoms, form a 6 membered aryl is optionally substituted with one or more R¹⁶.

In some embodiments, R⁴ and R¹ together with the atoms to which they are attached and any intervening atoms, form a 6 membered unsubstituted aryl.

In some embodiments, R⁴ and R¹ together with the atoms to which they are attached and any intervening atoms, form a 6 membered aryl is substituted with one or more halogen.

In some embodiments, R⁴ and R¹ together with the atoms to which they are attached and any intervening atoms, form a 6 membered aryl is substituted with one or more F.

In some embodiments, R⁴ and R¹ together with the atoms to which they are attached and any intervening atoms, form a 6 membered aryl is substituted with one F.

In some embodiments, R⁴ and R¹ together with the atoms to which they are attached and any intervening atoms, form a 6 membered aryl is substituted with one or more Cl.

In some embodiments, R⁴ and R¹ together with the atoms to which they are attached and any intervening atoms, form a 6 membered aryl is substituted with one Cl.

In some embodiments, R⁴ and R¹ together with the atoms to which they are attached and any intervening atoms, form a 6 membered aryl is optionally with one or more C₁-C₆ alkyl.

In some embodiments, R⁴ and R¹ together with the atoms to which they are attached and any intervening atoms, form a 6 membered aryl is substituted with one or more methyl.

In some embodiments, R⁴ and R¹ together with the atoms to which they are attached and any intervening atoms, form a 6 membered aryl is substituted with one methyl.

In some embodiments, R⁴ and R¹ together with the atoms to which they are attached and any intervening atoms, form a 6 membered aryl is optionally with one or more C₁-C₆ alkoxy.

In some embodiments, R⁴ and R¹ together with the atoms to which they are attached and any intervening atoms, form a 6 membered aryl is substituted with one or more methoxy.

In some embodiments, R⁴ and R¹ together with the atoms to which they are attached and any intervening atoms, form a 6 membered aryl is substituted with one methoxy.

In some embodiments, n is integer selected from 0, 1, 2.

In some embodiments, n is 2.

In some embodiments, n is 1.

In some embodiments, n is 0.

In some embodiments, R⁷, R⁸, R⁹ and R¹⁰ are each independently selected from H, C₁-C₆ alkyl, cycloalkyl.

In some embodiments, R⁸ and R⁹ together with the atoms to which they are attached and any intervening atoms, form cycloalkyl optionally substituted with one or more halogen, —OH, —CN, C₁-C₆ alkyl.

In some embodiments, R⁸ and R⁹ together with the atoms to which they are attached and any intervening atoms, form a cyclopropane ring optionally substituted with one or more halogen, —OH, —CN, C₁-C₆ alkyl.

In some embodiments, R⁹ and R¹⁰ together with the atoms to which they are attached and any intervening atoms, form cycloalkyl optionally substituted with one or more halogen, —OH, —CN, C₁-C₆ alkyl.

In some embodiments, R⁷ and R⁸ each is H, R⁹ and R¹⁰ are each C₁-C₆ alkyl.

In some embodiments, R⁷ and R⁸ each is H, R⁹ and R¹⁰ are each CH₃.

In some embodiments, n is 2, R⁷ and R⁸ each is H, R⁹ and R¹⁰ are each C₁-C₆ alkyl.

In some embodiments, n is 2, R⁷ and R⁸ each is H, R⁹ and R¹⁰ are each CH₃.

In some embodiments, n is 2, R⁹ and R¹⁰ together with the atoms to which they are attached and any intervening atoms, form cycloalkyl optionally substituted with one or more halogen, —OH, —CN, C₁-C₆ alkyl.

In some embodiments, n is 2, R⁹ and R¹⁰ together with the atoms to which they are attached and any intervening atoms, form cyclopropane ring optionally substituted with one or more halogen, —OH, —CN, C₁-C₆ alkyl.

In some embodiments, n is 1, R⁸ and R⁹ together with the atoms to which they are attached and any intervening atoms, form cycloalkyl optionally substituted with one or more halogen, —OH, —CN, C₁-C₆ alkyl.

In some embodiments, n is 1, R⁸ and R⁹ together with the atoms to which they are attached and any intervening atoms, form a cyclopropane ring optionally substituted with one or more halogen, —OH, —CN, C₁-C₆ alkyl.

In some embodiments, n is 1, R⁸ and R⁹ together with the atoms to which they are attached and any intervening atoms, form a cyclopropane ring optionally substituted with one or more halogen, —OH, —CN, C₁-C₆ alkyl.

In some embodiments, n is 1, R⁸ and R⁹ together with the atoms to which they are attached and any intervening atoms, form a cyclopropane ring optionally substituted with one or more C₁-C₆ alkyl.

In some embodiments, n is 1, R⁸ and R⁹ together with the atoms to which they are attached and any intervening atoms, form a cyclopropane ring optionally substituted with two C₁-C₆ alkyl.

In some embodiments, n is 1, R⁸ and R⁹ together with the atoms to which they are attached and any intervening atoms, form a cyclopropane ring optionally geminal substituted with two C₁-C₆ alkyl.

In some embodiments, n is 1, R⁸ and R⁹ together with the atoms to which they are attached and any intervening atoms, form a cyclopropane ring optionally geminal substituted with two methyl.

In some embodiments, R¹¹, R¹², R¹³ are each independently selected from H, C₁-C₆ alkyl, cycloalkyl, halogen, —CN.

In some embodiments, R¹¹, R¹², R¹³ are each C₁-C₆ alkyl.

In some embodiments, R¹¹, R¹², R¹³ are each methyl.

In some embodiments, R¹¹, R¹², R¹³ are each halogen.

In some embodiments, R¹¹, R¹², R¹³ are each F.

In some embodiments, R¹¹ is methyl, R¹² and R¹³ are each F.

In some embodiments, R¹⁴ and R¹⁵ are each independently selected from H, C₁-C₆ alkyl.

In some embodiments, R¹⁴ and R¹⁵ are each H.

In some embodiments, R¹⁴ and R¹⁵ are each methyl.

In some embodiments, R¹⁴ and R¹⁵ together with the atoms to which they are attached and any intervening atoms, form heterocyclyl optionally substituted with one or more halogen, —OH, —CN, C₁-C₆ alkyl.

In some embodiments, R¹⁶ is independently selected from halogen, —OH, —OH, —CN, —NO₂, —NR¹³R¹⁴, C₁-C₆ alkyl, C₂-C₆ alkenyl, C₂-C₆ alkynyl, C₁-C₆ alkoxy, —C(O)OR¹³, —C(O)N—NR¹³R¹⁴, cycloalkyl, —O-cycloalkyl, aryl, heterocyclyl, and heteroaryl.

In some embodiments, X is selected from hydrogen, halogen, OH, CN, NO₂, CONR¹³R¹⁴, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl wherein alkyl optionally substituted with cycloalkyl, heterocyclyl, aryl, and heteroaryl.

In some embodiments, X is —CN.

In some embodiments, X is CONR¹³R¹⁴.

In some embodiments, X is CONH₂.

In some embodiments, X is selected from C₁-C₆ alkyl optionally substituted with cycloalkyl, heterocyclyl, aryl, and heteroaryl.

In some embodiments, X is selected from C₁-C₆ alkyl substituted with heterocyclyl.

In some embodiments, X is

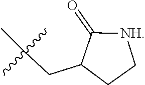 [SMILES: *CC1CCNC1=O]

In some embodiments, X is

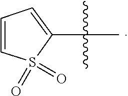 [SMILES: *C1=CC=CS1(=O)=O]

In some embodiments, Y is selected from C₁-C₆ alkyl-S(O)ᵤ—, C₁-C₆ alkyl-C(O)—, wherein alkyl is optionally substituted with one or more halogen, —OH, —CN.

In some embodiments, u is integer selected from 0, 1, 2.

In some embodiments, u is 0.

In some embodiments, u is 1.

In some embodiments, u is 2.

In some embodiments, Y is CF₃C(O)—.

In some embodiments, Y is CH₃S(O)₂—.

In some embodiments, Y is CF₃S(O)₂—.

In some embodiments, Y is CF₃CH₂S(O)₂—.

In another aspect, the present disclosure provides compounds of Formula (IV) and pharmaceutical acceptable salts, stereoisomers, solvates, prodrugs, isotopic derivatives, and tautomers thereof:

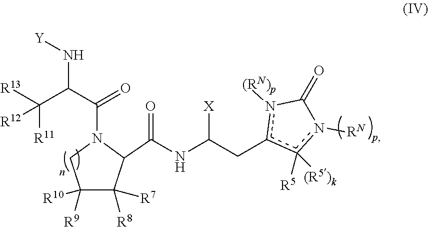

Wherein R⁵, R^{5′}, R⁷, R⁸, R⁹, R¹⁰, R¹¹, R¹², R¹³, R^{N}, X, Y, k, n and p are as described herein.

It is understood that, for a compound of Formula (IV), R⁵, R^{5′}, R⁷, R⁸, R⁹, R¹⁰, R¹¹, R¹², R¹³, R^{N}, X, Y, k, n and p can each be, where applicable, selected from the groups described herein, and any group described herein for any of R⁵, R^{5′}, R⁷, R⁸, R⁹, R¹⁰, R¹¹, R¹², R¹³, R^{N}, X, Y, k, n and p can be combined, where applicable, with any group described herein for one or more of the remainder of R⁵, R^{5′}, R⁷, R⁸, R⁹, R¹⁰, R¹¹, R¹², R¹³, R^{N}, X, Y, k, n and p.

In some embodiments,
each bond [symbol] and [symbol] is a single bond or double bond;
when bond [symbol] is a double bond each bond [symbol] is a single bond, when [symbol] is a single bond each bond [symbol] is independently selected from a single bond or double bond;
each p is an integer independently selected from 0 and 1;
provided that when [symbol] is a single bond then p is 1 and when [symbol] is a double bond then p is 0;
k is an integer selected from 0, 1;
provided that when the bond [symbol] is a single bond and the bond [symbol] is a single k is 1 and when the bond [symbol] or the bond [symbol] is a double bond k is 0;
R⁵ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹³, —C(O)N—NR¹⁴R¹⁵, cycloalkyl, —O-cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R^{5′} is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)N—NR¹⁴R¹⁵, cycloalkyl, —O-cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R⁵ and R^{5′} form oxo;
R⁷, R⁸, R⁹ and R¹⁰ are each independently selected from H, alkyl;
or R⁸ and R⁹ together with the atoms to which they are attached and any intervening atoms, form cycloalkyl optionally substituted with one or more halogen, —OH, —CN, C₁-C₆ alkyl;
or R⁹ and R¹⁰ together with the atoms to which they are attached and any intervening atoms, form cycloalkyl optionally substituted with one or more halogen, —OH, —CN, C₁-C₆ alkyl;
R¹¹, R¹², R¹³ are each independently selected from C₁-C₆ alkyl, cycloalkyl, halogen, —CN;
R¹⁴ and R¹⁵ are each independently selected from H, C₁-C₆ alkyl;
or R¹⁴ and R¹⁵ together with the atoms to which they are attached and any intervening atoms, form heterocyclyl optionally substituted with one or more halogen, —OH, —CN, C₁-C₆ alkyl;
R¹⁶ is independently selected from halogen, —OH, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)N—NR¹⁴R¹⁵, cycloalkyl, —O-cycloalkyl, aryl, heterocyclyl, and heteroaryl;
each R^{N} is independently selected from H, C₁-C₆ alkyl, cycloalkyl;
X is selected from hydrogen, halogen, OH, CN, NO₂, CONH₂, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl wherein alkyl optionally substituted with cycloalkyl, heterocyclyl, aryl, and heteroaryl;
Y is selected from C₁-C₆ alkyl-S(O)ᵤ—, C₁-C₆ alkyl-C(O)—, wherein alkyl is optionally substituted with one or more halogen, —OH, —CN;
n is an integer selected from 0, 1, 2;
u is an integer selected from 0, 1, 2.

In some embodiments, bond [symbol] is a double bond, each bond [symbol] is a single bond, k is 0 and each p is 1.

In some embodiments, bond [symbol] is a single bond, each bond [symbol] is a double bond, k is 0 and each p is 0.

In some embodiments, bond [symbol] is a single bond, each bond [symbol] is a single bond, k is 1 and each p is 1.

In some embodiments, R⁵ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹³, —C(O)N—NR¹⁴R¹⁵, cycloalkyl, —O-cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶.

In some embodiments, R⁵ is H.

In some embodiments, R^{5′} is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)N—NR¹⁴R¹⁵, cycloalkyl, —O-cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶.

In some embodiments, R^{5′} is H.

In some embodiments, R⁵ and R^{5′} form oxo.

In some embodiments, each R^{N} is independently selected from H, C₁-C₆ alkyl, cycloalkyl.

In some embodiments, each R^{N} is independently selected from H and C₁-C₆ alkyl.

In some embodiments, the compound of Formula (IV) is compound of Formula (IV′):

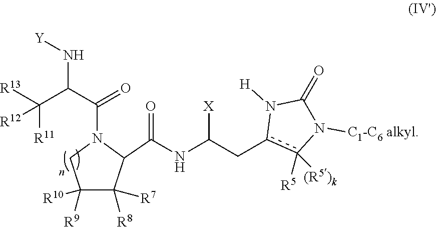

In some embodiments, the compound of Formula (IV) is compound of Formula (IV″):

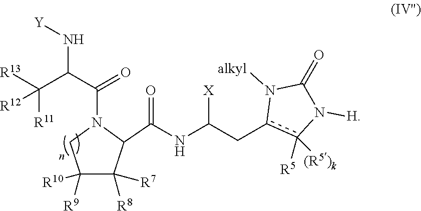

In some embodiments, the compound of Formula (IV) is compound of Formula (IV′″):

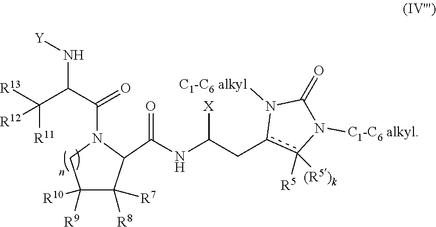

In some embodiments, n is integer selected from 0, 1, 2.

In some embodiments, n is 2.

In some embodiments, n is 1.

In some embodiments, n is 0.

In some embodiments, R⁷, R⁸, R⁹ and R¹⁰ are each independently selected from H, C₁-C₆ alkyl.

In some embodiments, R⁸ and R⁹ together with the atoms to which they are attached and any intervening atoms, form cycloalkyl optionally substituted with one or more halogen, —OH, —CN, C₁-C₆ alkyl.

In some embodiments, R⁸ and R⁹ together with the atoms to which they are attached and any intervening atoms, form a cyclopropane ring optionally substituted with one or more halogen, —OH, —CN, C₁-C₆ alkyl.

In some embodiments, R⁹ and R¹⁰ together with the atoms to which they are attached and any intervening atoms, form cycloalkyl optionally substituted with one or more halogen, —OH, —CN, C₁-C₆ alkyl.

In some embodiments, R⁷ and R⁸ each is H, R⁹ and R¹⁰ are each C₁-C₆ alkyl.

In some embodiments, R⁷ and R⁸ each is H, R⁹ and R¹⁰ are each CH₃.

In some embodiments, n is 2, R⁷ and R⁸ each is H, R⁹ and R¹⁰ are each C₁-C₆ alkyl.

In some embodiments, n is 2, R⁷ and R⁸ each is H, R⁹ and R¹⁰ are each CH₃.

In some embodiments, n is 2, R⁹ and R¹⁰ together with the atoms to which they are attached and any intervening atoms, form cycloalkyl optionally substituted with one or more halogen, —OH, —CN, C₁-C₆ alkyl.

In some embodiments, n is 2, R⁹ and R¹⁰ together with the atoms to which they are attached and any intervening atoms, form cyclopropane ring optionally substituted with one or more halogen, —OH, —CN, C₁-C₆ alkyl.

In some embodiments, n is 1, R⁸ and R⁹ together with the atoms to which they are attached and any intervening atoms, form cycloalkyl optionally substituted with one or more halogen, —OH, —CN, C₁-C₆ alkyl.

In some embodiments, n is 1, R⁸ and R⁹ together with the atoms to which they are attached and any intervening atoms, form a cyclopropane ring optionally substituted with one or more halogen, —OH, —CN, C₁-C₆ alkyl.

In some embodiments, n is 1, R⁸ and R⁹ together with the atoms to which they are attached and any intervening atoms, form a cyclopropane ring optionally substituted with one or more halogen, —OH, —CN, C₁-C₆ alkyl.

In some embodiments, n is 1, R⁸ and R⁹ together with the atoms to which they are attached and any intervening atoms, form a cyclopropane ring optionally substituted with one or more C₁-C₆ alkyl.

In some embodiments, n is 1, R⁸ and R⁹ together with the atoms to which they are attached and any intervening atoms, form a cyclopropane ring optionally substituted with two C₁-C₆ alkyl.

In some embodiments, n is 1, R⁸ and R⁹ together with the atoms to which they are attached and any intervening atoms, form a cyclopropane ring optionally geminal substituted with two C₁-C₆ alkyl.

In some embodiments, n is 1, R⁸ and R⁹ together with the atoms to which they are attached and any intervening atoms, form a cyclopropane ring optionally geminal substituted with two methyl.

In some embodiments, R¹¹, R¹², R¹³ are each independently selected from H, C₁-C₆ alkyl, cycloalkyl, halogen, —CN.

In some embodiments, R¹¹, R¹², R¹³ are each C₁-C₆ alkyl.

In some embodiments, R¹¹, R¹², R¹³ are each methyl.

In some embodiments, R¹¹, R¹², R¹³ are each halogen.

In some embodiments, R¹¹, R¹², R¹³ are each F.

In some embodiments, R¹¹ is methyl, R¹² and R¹³ are each F.

In some embodiments, R¹⁴ and R¹⁵ are each independently selected from H, C₁-C₆ alkyl.

In some embodiments, R¹⁴ and R¹⁵ are each H.

In some embodiments, R¹⁴ and R¹⁵ are each methyl.

In some embodiments, R¹⁴ and R¹⁵ together with the atoms to which they are attached and any intervening atoms, form heterocyclyl optionally substituted with one or more halogen, —OH, —CN, C₁-C₆ alkyl.

In some embodiments, R¹⁶ is independently selected from halogen, —OH, —OH, —CN, —NO₂, —NR¹³R¹⁴, C₁-C₆ alkyl, C₂-C₆ alkenyl, C₂-C₆ alkynyl, C₁-C₆ alkoxy, —C(O)OR¹³, —C(O)N—NR¹³R¹⁴, cycloalkyl, —O-cycloalkyl, aryl, heterocyclyl, and heteroaryl.

In some embodiments, X is selected from hydrogen, halogen, OH, CN, NO₂, CONR¹³R¹⁴, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl wherein alkyl optionally substituted with cycloalkyl, heterocyclyl, aryl, and heteroaryl.

In some embodiments, X is —CN.

In some embodiments, X is CONR¹³R¹⁴.

In some embodiments, X is CONH₂.

In some embodiments, X is selected from C₁-C₆ alkyl optionally substituted with cycloalkyl, heterocyclyl, aryl, and heteroaryl.

In some embodiments, X is selected from C₁-C₆ alkyl substituted with heterocyclyl.

In some embodiments, X is

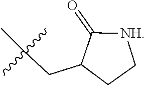 [SMILES: *CC1CCNC1=O]

In some embodiments, X is

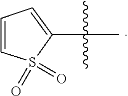 [SMILES: *C1=CC=CS1(=O)=O]

In some embodiments, Y is selected from C₁-C₆ alkyl-S(O)ᵤ—, C₁-C₆ alkyl-C(O)—, wherein alkyl is optionally substituted with one or more halogen, —OH, —CN.

In some embodiments, u is integer selected from 0, 1, 2.

In some embodiments, u is 0.

In some embodiments, u is 1.

In some embodiments, u is 2.

In some embodiments, Y is CF₃C(O)—.

In some embodiments, Y is CH₃S(O)₂—.

In some embodiments, Y is CF₃S(O)₂—.

In some embodiments, Y is CF₃CH₂S(O)₂—.

In another aspect, the present disclosure provides compounds of Formula (V) and pharmaceutical acceptable salts, stereoisomers, solvates, prodrugs, isotopic derivatives, and tautomers thereof:

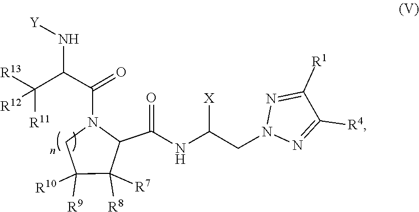

Wherein R¹, R⁴, R⁷, R⁸, R⁹, R¹⁰, R¹¹, R¹², R¹³, X, Y, and n are as described herein.

It is understood that, for a compound of Formula (V), R¹, R⁴, R⁷, R⁸, R⁹, R¹⁰, R¹¹, R¹², R¹³, X, Y, and n can each be, where applicable, selected from the groups described herein, and any group described herein for any of R¹, R⁴, R⁷, R⁸, R⁹, R¹⁰, R¹¹, R¹², R¹³, X, Y, and n can be combined, where applicable, with any group described herein for one or more of the remainder of R¹, R⁴, R⁷, R⁸, R⁹, R¹⁰, R¹¹, R¹², R¹³, X, Y, and n.

In some embodiments,
R¹ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R⁴ is selected from hydrogen, halogen, —OH, —CN, —NO₂, —NR¹³R¹⁴, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R¹ and R⁴ together with the atoms to which they are attached and any intervening atoms, form a 3-10 membered cycloalkyl, a 6-10 membered aryl, a 3-14 membered heterocycle, or a 5-6 membered heteroaryl, wherein the cycloalkyl, aryl, heterocycle or heteroaryl is optionally substituted with one or more R¹⁶;
R⁷, R⁸, R⁹ and R¹⁰ are each independently selected from H, alkyl;
or R⁸ and R⁹ together with the atoms to which they are attached and any intervening atoms, form cycloalkyl optionally substituted with one or more halogen, —OH, —CN, C₁-C₆ alkyl;
or R⁹ and R¹⁰ together with the atoms to which they are attached and any intervening atoms, form cycloalkyl optionally substituted with one or more halogen, —OH, —CN, C₁-C₆ alkyl;
R¹¹, R¹², R¹³ are each independently selected from C₁-C₆ alkyl, cycloalkyl, halogen, —CN;
R¹⁴ and R¹⁵ are each independently selected from H, C₁-C₆ alkyl;
or R¹⁴ and R¹⁵ together with the atoms to which they are attached and any intervening atoms, form heterocyclyl optionally substituted with one or more halogen, —OH, —CN, C₁-C₆ alkyl;
R¹⁶ is independently selected from halogen, —OH, —OH, —CN, —NO₂, —NR¹³R¹⁴, C₁-C₆ alkyl, C₂-C₆ alkenyl, C₂-C₆ alkynyl, C₁-C₆ alkoxy, —C(O)OR¹³, —C(O)N—NR¹³R¹⁴, cycloalkyl, —O-cycloalkyl, aryl, heterocyclyl, and heteroaryl;
X is selected from hydrogen, halogen, OH, CN, NO₂, CONH₂, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl wherein alkyl optionally substituted with cycloalkyl, heterocyclyl, aryl, and heteroaryl;
Y is selected from C₁-C₆ alkyl-S(O)ᵤ—, C₁-C₆ alkyl-C(O)—, wherein alkyl is optionally substituted with one or more halogen, —OH, —CN;
n is integer selected from 0, 1, 2;
u is integer selected from 0, 1, 2.

In some embodiments, R¹ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶.

In some embodiments, R¹ is H.

In some embodiments, R¹ is C₁-C₆ alkyl.

In some embodiments, R¹ is methyl.

In some embodiments, R¹ and R⁴ together with the atoms to which they are attached and any intervening atoms, form a 3-10 membered cycloalkyl, a 6-10 membered aryl, a 3-14 membered heterocycle, or a 5-6 membered heteroaryl, wherein the cycloalkyl, aryl, heterocycle or heteroaryl is optionally substituted with one or more R¹⁶.

In some embodiments, R¹ and R⁴ together with the atoms to which they are attached and any intervening atoms, form a 6 membered aryl is optionally substituted with one or more R¹⁶.

In some embodiments, R¹ and R⁴ together with the atoms to which they are attached and any intervening atoms, form a 6 membered unsubstituted aryl.

In some embodiments, R¹ and R⁴ together with the atoms to which they are attached and any intervening atoms, form a 6 membered aryl is substituted with one or more halogen.

In some embodiments, R¹ and R⁴ together with the atoms to which they are attached and any intervening atoms, form a 6 membered aryl is substituted with one or more F.

In some embodiments, R¹ and R⁴ together with the atoms to which they are attached and any intervening atoms, form a 6 membered aryl is substituted with one F.

In some embodiments, R¹ and R⁴ together with the atoms to which they are attached and any intervening atoms, form a 6 membered aryl is substituted with one or more Cl.

In some embodiments, R¹ and R⁴ together with the atoms to which they are attached and any intervening atoms, form a 6 membered aryl is substituted with one Cl.

In some embodiments, R¹ and R⁴ together with the atoms to which they are attached and any intervening atoms, form a 6 membered aryl is optionally with one or more C₁-C₆ alkyl.

In some embodiments, R¹ and R⁴ together with the atoms to which they are attached and any intervening atoms, form a 6 membered aryl is substituted with one or more methyl.

In some embodiments, R¹ and R⁴ together with the atoms to which they are attached and any intervening atoms, form a 6 membered aryl is substituted with one methyl.

In some embodiments, R¹ and R⁴ together with the atoms to which they are attached and any intervening atoms, form a 6 membered aryl is optionally with one or more C₁-C₆ alkoxy.

In some embodiments, R¹ and R⁴ together with the atoms to which they are attached and any intervening atoms, form a 6 membered aryl is substituted with one or more methoxy.

In some embodiments, R¹ and R⁴ together with the atoms to which they are attached and any intervening atoms, form a 6 membered aryl is substituted with one methoxy.

In some embodiments, R⁴ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶.

In some embodiments, R⁴ is H.

In some embodiments, R⁴ is C₁-C₆ alkyl.

In some embodiments, R⁴ is methyl.

In some embodiments, R⁴ and R¹ together with the atoms to which they are attached and any intervening atoms, form a 3-10 membered cycloalkyl, a 6-10 membered aryl, a 3-14 membered heterocycle, or a 5-6 membered heteroaryl, wherein the cycloalkyl, aryl, heterocycle or heteroaryl is optionally substituted with one or more R¹⁶.

In some embodiments, R⁴ and R¹ together with the atoms to which they are attached and any intervening atoms, form a 6 membered aryl is optionally substituted with one or more R¹⁶.

In some embodiments, R⁴ and R¹ together with the atoms to which they are attached and any intervening atoms, form a 6 membered unsubstituted aryl.

In some embodiments, R⁴ and R¹ together with the atoms to which they are attached and any intervening atoms, form a 6 membered aryl is substituted with one or more halogen.

In some embodiments, R⁴ and R¹ together with the atoms to which they are attached and any intervening atoms, form a 6 membered aryl is substituted with one or more F.

In some embodiments, R⁴ and R¹ together with the atoms to which they are attached and any intervening atoms, form a 6 membered aryl is substituted with one F.

In some embodiments, R⁴ and R¹ together with the atoms to which they are attached and any intervening atoms, form a 6 membered aryl is substituted with one or more Cl.

In some embodiments, R⁴ and R¹ together with the atoms to which they are attached and any intervening atoms, form a 6 membered aryl is substituted with one Cl.

In some embodiments, R⁴ and R¹ together with the atoms to which they are attached and any intervening atoms, form a 6 membered aryl is optionally with one or more C₁-C₆ alkyl.

In some embodiments, R⁴ and R¹ together with the atoms to which they are attached and any intervening atoms, form a 6 membered aryl is substituted with one or more methyl.

In some embodiments, R⁴ and R¹ together with the atoms to which they are attached and any intervening atoms, form a 6 membered aryl is substituted with one methyl.

In some embodiments, R⁴ and R¹ together with the atoms to which they are attached and any intervening atoms, form a 6 membered aryl is optionally with one or more C₁-C₆ alkoxy.

In some embodiments, R⁴ and R¹ together with the atoms to which they are attached and any intervening atoms, form a 6 membered aryl is substituted with one or more methoxy.

In some embodiments, R⁴ and R¹ together with the atoms to which they are attached and any intervening atoms, form a 6 membered aryl is substituted with one methoxy.

In some embodiments, n is integer selected from 0, 1, 2.

In some embodiments, n is 2.

In some embodiments, n is 1.

In some embodiments, n is 0.

In some embodiments, R⁷, R⁸, R⁹ and R¹⁰ are each independently selected from H, C₁-C₆ alkyl, cycloalkyl.

In some embodiments, R⁸ and R⁹ together with the atoms to which they are attached and any intervening atoms, form cycloalkyl optionally substituted with one or more halogen, —OH, —CN, C₁-C₆ alkyl.

In some embodiments, R⁸ and R⁹ together with the atoms to which they are attached and any intervening atoms, form a cyclopropane ring optionally substituted with one or more halogen, —OH, —CN, C₁-C₆ alkyl.

In some embodiments, R⁹ and R¹⁰ together with the atoms to which they are attached and any intervening atoms, form cycloalkyl optionally substituted with one or more halogen, —OH, —CN, C₁-C₆ alkyl.

In some embodiments, R⁷ and R⁸ each is H, R⁹ and R¹⁰ are each C₁-C₆ alkyl.

In some embodiments, R⁷ and R⁸ each is H, R⁹ and R¹⁰ are each CH₃.

In some embodiments, n is 2, R⁷ and R⁸ each is H, R⁹ and R¹⁰ are each C₁-C₆ alkyl.

In some embodiments, n is 2, R⁷ and R⁸ each is H, R⁹ and R¹⁰ are each CH₃.

In some embodiments, n is 2, R⁹ and R¹⁰ together with the atoms to which they are attached and any intervening atoms, form cycloalkyl optionally substituted with one or more halogen, —OH, —CN, C₁-C₆ alkyl.

In some embodiments, n is 2, R⁹ and R¹⁰ together with the atoms to which they are attached and any intervening atoms, form cyclopropane ring optionally substituted with one or more halogen, —OH, —CN, C₁-C₆ alkyl.

In some embodiments, n is 1, R⁸ and R⁹ together with the atoms to which they are attached and any intervening atoms, form cycloalkyl optionally substituted with one or more halogen, —OH, —CN, C₁-C₆ alkyl.

In some embodiments, n is 1, R⁸ and R⁹ together with the atoms to which they are attached and any intervening atoms, form a cyclopropane ring optionally substituted with one or more halogen, —OH, —CN, C₁-C₆ alkyl.

In some embodiments, n is 1, R⁸ and R⁹ together with the atoms to which they are attached and any intervening atoms, form a cyclopropane ring optionally substituted with one or more halogen, —OH, —CN, C₁-C₆ alkyl.

In some embodiments, n is 1, R⁸ and R⁹ together with the atoms to which they are attached and any intervening atoms, form a cyclopropane ring optionally substituted with one or more C₁-C₆ alkyl.

In some embodiments, n is 1, R⁸ and R⁹ together with the atoms to which they are attached and any intervening atoms, form a cyclopropane ring optionally substituted with two C₁-C₆ alkyl.

In some embodiments, n is 1, R⁸ and R⁹ together with the atoms to which they are attached and any intervening atoms, form a cyclopropane ring optionally geminal substituted with two C₁-C₆ alkyl.

In some embodiments, n is 1, R⁸ and R⁹ together with the atoms to which they are attached and any intervening atoms, form a cyclopropane ring optionally geminal substituted with two methyl.

In some embodiments, R¹¹, R¹², R¹³ are each independently selected from H, C₁-C₆ alkyl, cycloalkyl, halogen, —CN.

In some embodiments, R¹¹, R¹², R¹³ are each C₁-C₆ alkyl.

In some embodiments, R¹¹, R¹², R¹³ are each methyl.

In some embodiments, R¹¹, R¹², R¹³ are each halogen.

In some embodiments, R¹¹, R¹², R¹³ are each F.

In some embodiments, R¹¹ is methyl, R¹² and R¹³ are each F.

In some embodiments, R¹⁴ and R¹⁵ are each independently selected from H, C₁-C₆ alkyl.

In some embodiments, R¹⁴ and R¹⁵ are each H.

In some embodiments, R¹⁴ and R¹⁵ are each methyl.

In some embodiments, R¹⁴ and R¹⁵ together with the atoms to which they are attached and any intervening atoms, form heterocyclyl optionally substituted with one or more halogen, —OH, —CN, C₁-C₆ alkyl.

In some embodiments, R¹⁶ is independently selected from halogen, —OH, —OH, —CN, —NO₂, —NR¹³R¹⁴, C₁-C₆ alkyl, C₂-C₆ alkenyl, C₂-C₆ alkynyl, C₁-C₆ alkoxy, —C(O)OR¹³, —C(O)N—NR¹³R¹⁴, cycloalkyl, —O-cycloalkyl, aryl, heterocyclyl, and heteroaryl.

In some embodiments, X is selected from hydrogen, halogen, OH, CN, NO₂, CONR¹³R¹⁴, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl wherein alkyl optionally substituted with cycloalkyl, heterocyclyl, aryl, and heteroaryl.

In some embodiments, X is —CN.

In some embodiments, X is CONR¹³R¹⁴.

In some embodiments, X is CONH₂.

In some embodiments, X is selected from C₁-C₆ alkyl optionally substituted with cycloalkyl, heterocyclyl, aryl, and heteroaryl.

In some embodiments, X is selected from C₁-C₆ alkyl substituted with heterocyclyl.

In some embodiments, X is

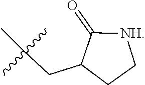 [SMILES: *CC1CCNC1=O]

In some embodiments, X is

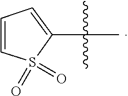 [SMILES: *C1=CC=CS1(=O)=O]

In some embodiments, Y is selected from C₁-C₆ alkyl-S(O)ᵤ—, C₁-C₆ alkyl-C(O)—, wherein alkyl is optionally substituted with one or more halogen, —OH, —CN.

In some embodiments, u is integer selected from 0, 1, 2.

In some embodiments, u is 0.

In some embodiments, u is 1.

In some embodiments, u is 2.

In some embodiments, Y is CF₃C(O)—.

In some embodiments, Y is CH₃S(O)₂—.

In some embodiments, Y is CF₃S(O)₂—.

In some embodiments, Y is CF₃CH₂S(O)₂—.

In some embodiments, the compound is of Formula (II-I):

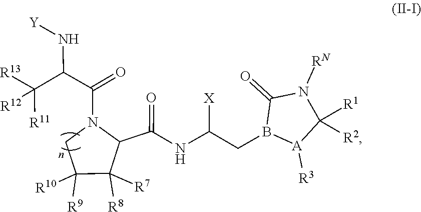

or a pharmaceutically acceptable salt, stereoisomer, solvate, prodrug, isotopic derivative, or tautomer thereof, wherein all variables are as defined herein.

In some embodiments, the compound is of Formula (II-II):

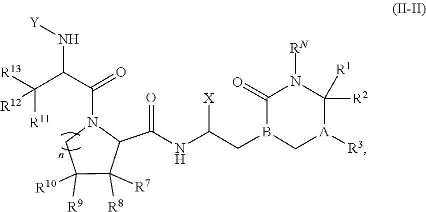

or a pharmaceutically acceptable salt, stereoisomer, solvate, prodrug, isotopic derivative, or tautomer thereof, wherein all variables are as defined herein.

In some embodiments, the compound is of Formula (II-III):

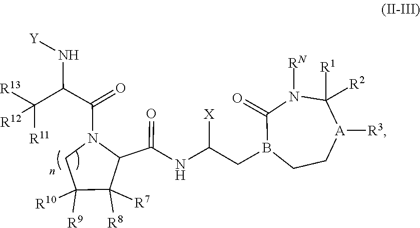

or a pharmaceutically acceptable salt, stereoisomer, solvate, prodrug, isotopic derivative, or tautomer thereof, wherein all variables are as defined herein.

In some embodiments, the compound is of Formula (II-A):

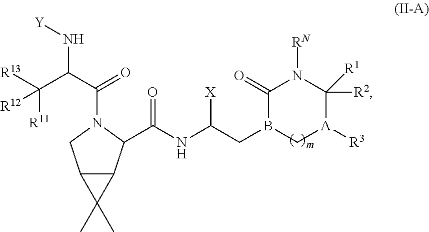

or a pharmaceutically acceptable salt, stereoisomer, solvate, prodrug, isotopic derivative, or tautomer thereof, wherein all variables are as defined herein.

In some embodiments, the compound is of Formula (II-B):

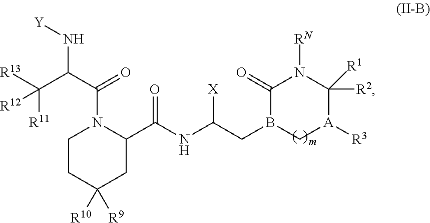

or a pharmaceutically acceptable salt, stereoisomer, solvate, prodrug, isotopic derivative, or tautomer thereof, wherein all variables are as defined herein.

In some embodiments, the compound is of Formula (II-B-a) or Formula (II-B-b):

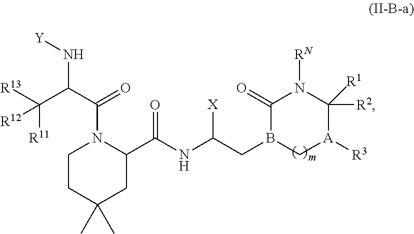

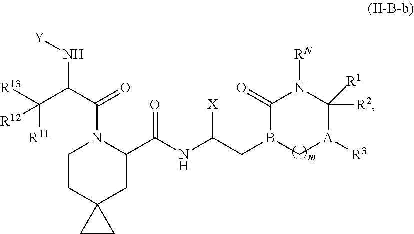

or a pharmaceutically acceptable salt, stereoisomer, solvate, prodrug, isotopic derivative, or tautomer thereof, wherein all variables are as defined herein.

In some embodiments, the compound is of Formula (II-I-A):

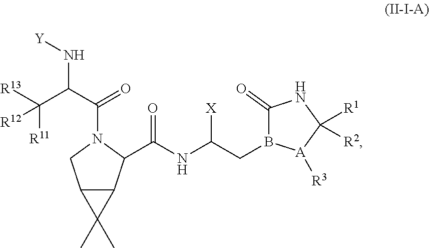

or a pharmaceutically acceptable salt, stereoisomer, solvate, prodrug, isotopic derivative, or tautomer thereof, wherein all variables are as defined herein.

In some embodiments, the compound is of Formula (II-I-A*):

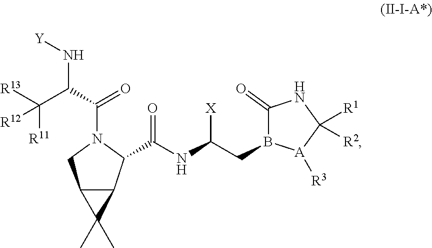

or a pharmaceutically acceptable salt, stereoisomer, solvate, prodrug, isotopic derivative, or tautomer thereof, wherein all variables are as defined herein.

In some embodiments, the compound is of Formula (II-I-A-A):

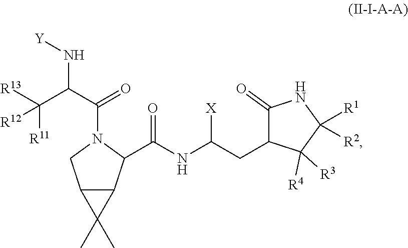

or a pharmaceutically acceptable salt, stereoisomer, solvate, prodrug, isotopic derivative, or tautomer thereof, wherein all variables are as defined herein.

In some embodiments, the compound is of Formula (II-I-A-A*):

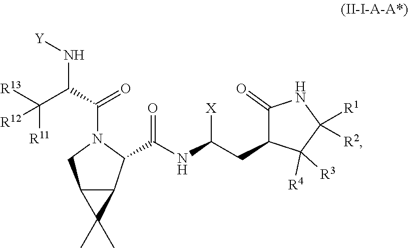

or a pharmaceutically acceptable salt, stereoisomer, solvate, prodrug, isotopic derivative, or tautomer thereof, wherein all variables are as defined herein.

In some embodiments, the compound is of Formula (II-I-A-A-A):

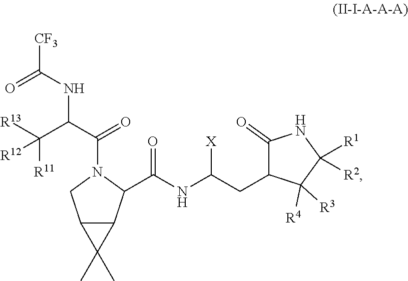

or a pharmaceutically acceptable salt, stereoisomer, solvate, prodrug, isotopic derivative, or tautomer thereof, wherein all variables are as defined herein.

In some embodiments, the compound is of Formula (II-I-A-A-A*):

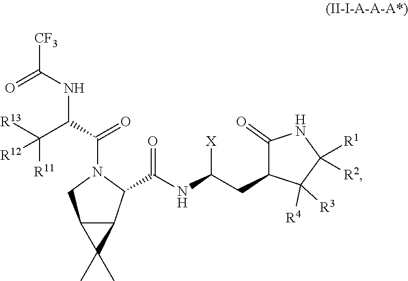

or a pharmaceutically acceptable salt, stereoisomer, solvate, prodrug, isotopic derivative, or tautomer thereof, wherein all variables are as defined herein.

In some embodiments, the compound is of Formula (II-I-A-A-A-I):

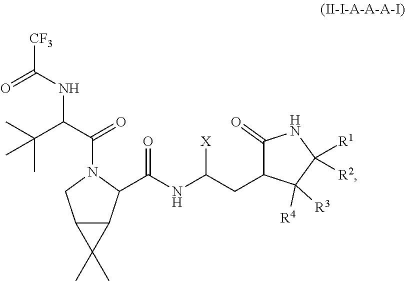

or a pharmaceutically acceptable salt, stereoisomer, solvate, prodrug, isotopic derivative, or tautomer thereof, wherein all variables are as defined herein.

In some embodiments, the compound is of Formula (II-I-A-A-A-I*):

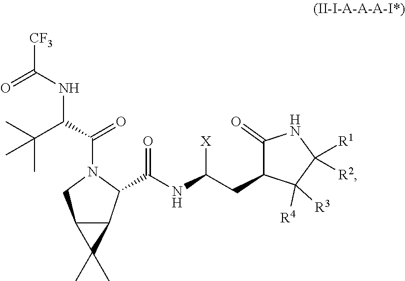

or a pharmaceutically acceptable salt, stereoisomer, solvate, prodrug, isotopic derivative, or tautomer thereof, wherein all variables are as defined herein.

In some embodiments, the compound is of Formula (II-I-A-A-A-I-A):

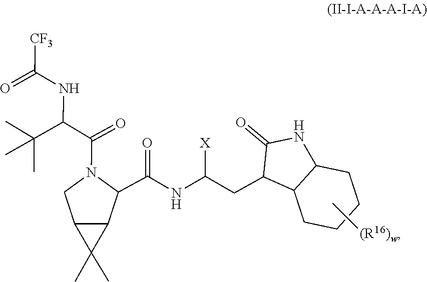

or a pharmaceutically acceptable salt, stereoisomer, solvate, prodrug, isotopic derivative, or tautomer thereof, wherein w is an integer selected from 0, 1, 2, 3, 4, 5, 6, 7, 8; and all other variables are as defined herein.

In some embodiments, the compound is of Formula (II-I-A-A-A-I-A*):

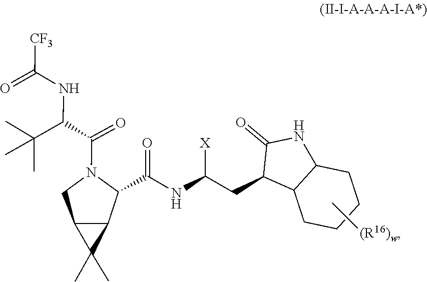

or a pharmaceutically acceptable salt, stereoisomer, solvate, prodrug, isotopic derivative, or tautomer thereof, wherein w is an integer selected from 0, 1, 2, 3, 4, 5, 6, 7, 8; and all other variables are as defined herein.

In some embodiments, the compound is of Formula (II-I-A-A-A-I-A-1) or Formula (II-I-A-A-A-I-A-2):

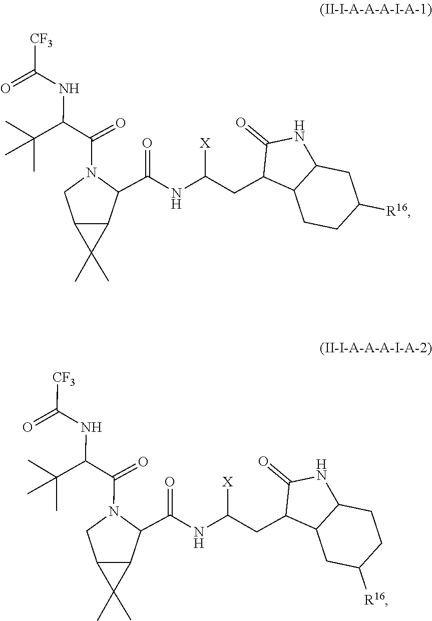

or a pharmaceutically acceptable salt, stereoisomer, solvate, prodrug, isotopic derivative, or tautomer thereof, wherein X is selected from —C(O)NH₂, —CN and all other variables are as defined herein.

In some embodiments, the compound is of Formula (II-I-A-A-A-I-A-1*) or Formula (II-I-A-A-A-I-A-2*):

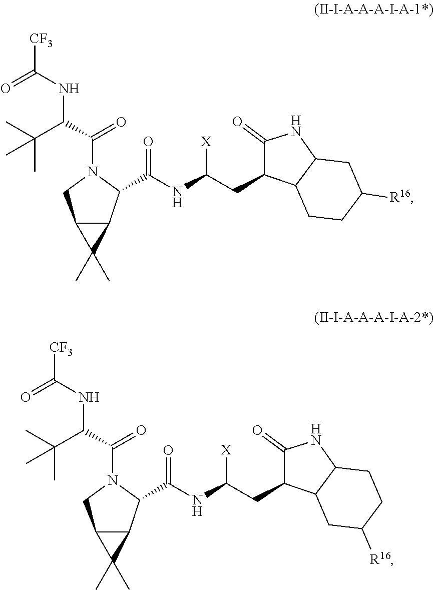

or a pharmaceutically acceptable salt, stereoisomer, solvate, prodrug, isotopic derivative, or tautomer thereof, wherein X is selected from —C(O)NH₂, —CN and all other variables are as defined herein.

In some embodiments, the compound is of Formula (II-I-A-A-A-I-B):

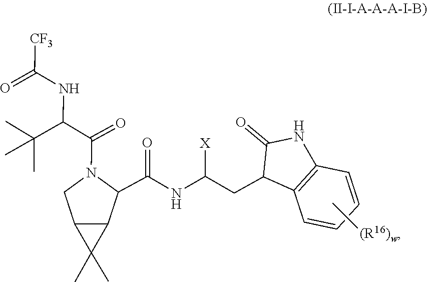

or a pharmaceutically acceptable salt, stereoisomer, solvate, prodrug, isotopic derivative, or tautomer thereof, wherein w is an integer selected from 0, 1, 2, 3, 4; and all other variables are as defined herein.

In some embodiments, the compound is of Formula (II-I-A-A-A-I-B*):

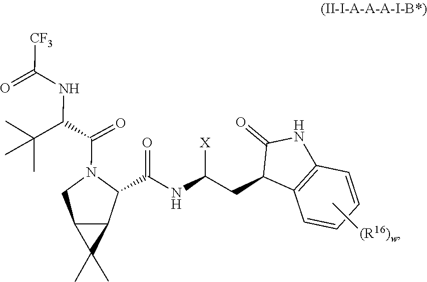

or a pharmaceutically acceptable salt, stereoisomer, solvate, prodrug, isotopic derivative, or tautomer thereof, wherein w is an integer selected from 0, 1, 2, 3, 4; and all other variables are as defined herein.

In some embodiments, the compound is of Formula (II-I-A-A-A-I-B-1) or Formula (II-I-A-A-A-I-B-2):

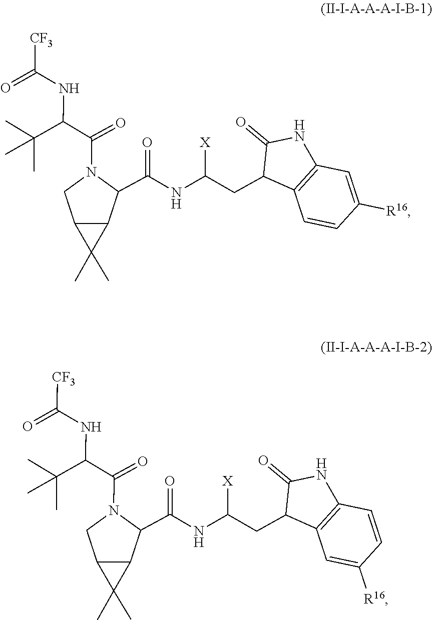

or a pharmaceutically acceptable salt, stereoisomer, solvate, prodrug, isotopic derivative, or tautomer thereof, wherein X is selected from —C(O)NH₂, —CN and all other variables are as defined herein.

In some embodiments, the compound is of Formula (II-I-A-A-A-I-B-1*) or Formula (II-I-A-A-A-I-B-2*):

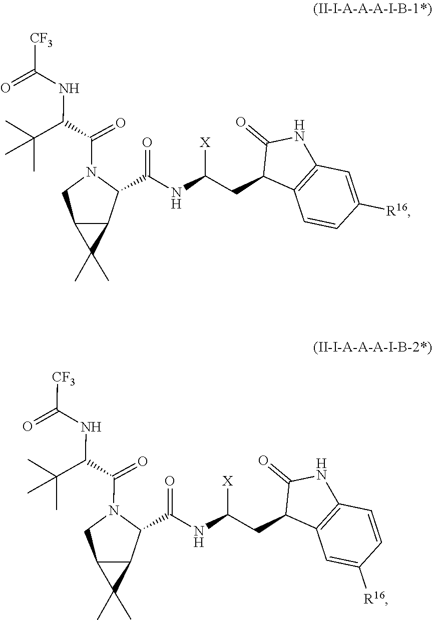

or a pharmaceutically acceptable salt, stereoisomer, solvate, prodrug, isotopic derivative, or tautomer thereof, wherein X is selected from —C(O)NH₂, —CN and all other variables are as defined herein.

In some embodiments, the compound is of Formula (II-3):

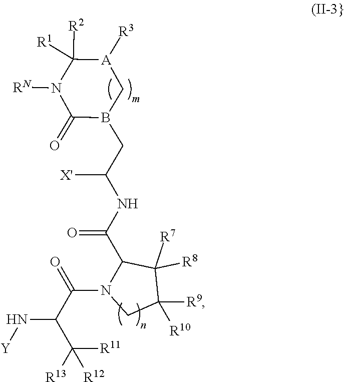

or a pharmaceutically acceptable salt, stereoisomer, solvate, prodrug, isotopic derivative, or tautomer thereof, wherein X′ is selected from cycloalkyl, heterocyclyl, aryl, and heteroaryl and all other variables are as defined herein.

In some embodiments, the compound is of Formula (II-I-3):

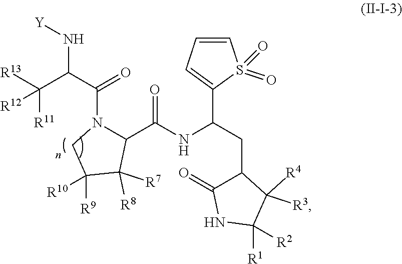

or a pharmaceutically acceptable salt, stereoisomer, solvate, prodrug, isotopic derivative, or tautomer thereof, wherein all variables are as defined herein.

In some embodiments, the compound is of Formula (II-I-A-A-A-I-3):

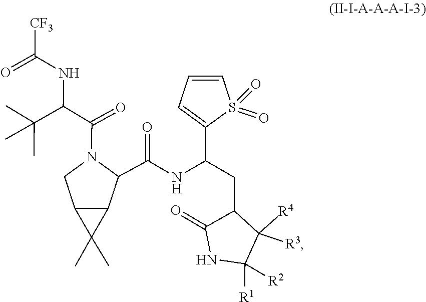

or a pharmaceutically acceptable salt, stereoisomer, solvate, prodrug, isotopic derivative, or tautomer thereof, wherein all variables are as defined herein.

In some embodiments, the compound is of Formula (II-I-A-A-A-I-3*):

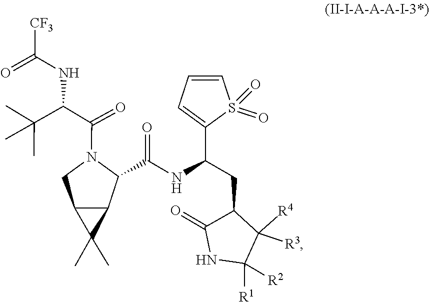

or a pharmaceutically acceptable salt, stereoisomer, solvate, prodrug, isotopic derivative, or tautomer thereof, wherein all variables are as defined herein.

In some embodiments, the compound is of Formula (II-I-A-A-A-II):

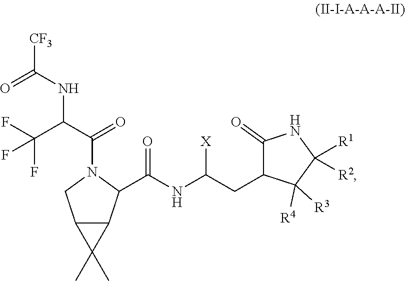

or a pharmaceutically acceptable salt, stereoisomer, solvate, prodrug, isotopic derivative, or tautomer thereof, wherein all variables are as defined herein.

In some embodiments, the compound is of Formula (II-I-A-A-A-II*):

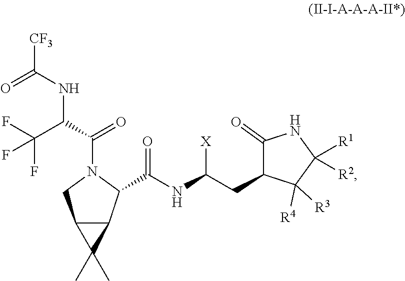

or a pharmaceutically acceptable salt, stereoisomer, solvate, prodrug, isotopic derivative, or tautomer thereof, wherein all variables are as defined herein.

In some embodiments, the compound is of Formula (II-I-A-A-A-II-1) or Formula (II-I-A-A-A-II-2):

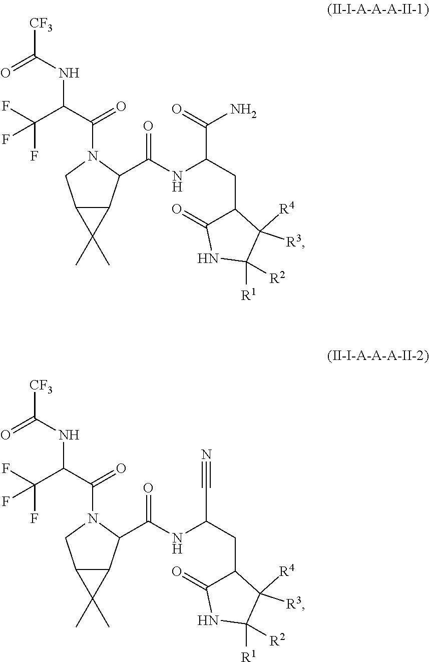

or a pharmaceutically acceptable salt, stereoisomer, solvate, prodrug, isotopic derivative, or tautomer thereof, wherein all variables are as defined herein.

In some embodiments, the compound is of Formula (II-I-A-A-A-II-1*) or Formula (II-I-A-A-A-II-2*):

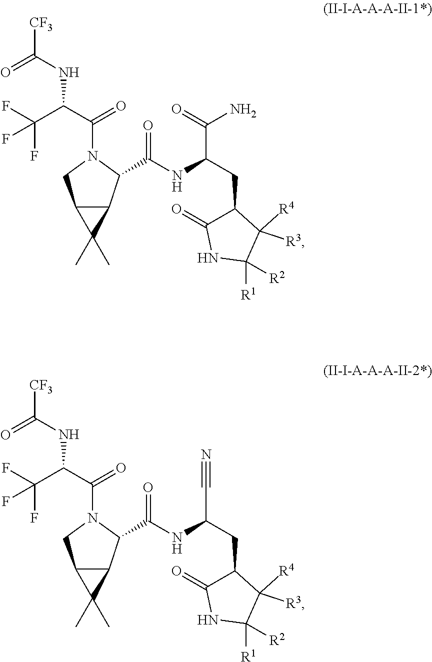

or a pharmaceutically acceptable salt, stereoisomer, solvate, prodrug, isotopic derivative, or tautomer thereof, wherein all variables are as defined herein.

In some embodiments, the compound is of Formula (II-I-A-A-A-III):

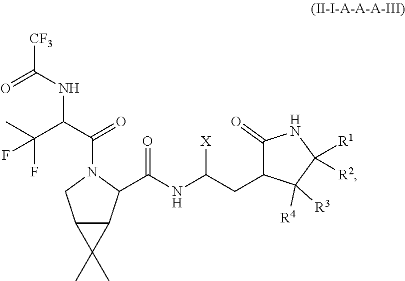

or a pharmaceutically acceptable salt, stereoisomer, solvate, prodrug, isotopic derivative, or tautomer thereof, wherein all variables are as defined herein.

In some embodiments, the compound is of Formula (II-I-A-A-A-III*):

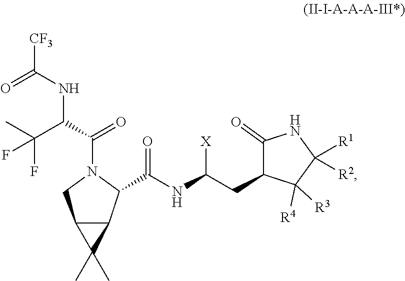

or a pharmaceutically acceptable salt, stereoisomer, solvate, prodrug, isotopic derivative, or tautomer thereof, wherein all variables are as defined herein.

In some embodiments, the compound is of Formula (II-I-A-A-A-III-1) or Formula (II-I-A-A-A-III-2):

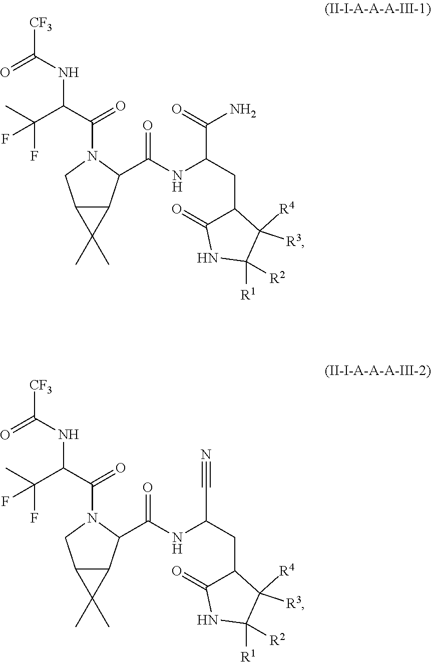

or a pharmaceutically acceptable salt, stereoisomer, solvate, prodrug, isotopic derivative, or tautomer thereof, wherein all variables are as defined herein.

In some embodiments, the compound is of Formula (II-I-A-A-A-III-1*) or Formula (II-I-A-A-A-III-2*):

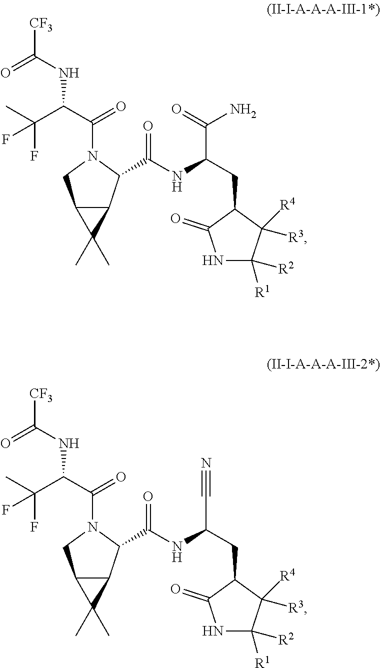

or a pharmaceutically acceptable salt, stereoisomer, solvate, prodrug, isotopic derivative, or tautomer thereof, wherein all variables are as defined herein.

In some embodiments, the compound is of Formula (II-I-A-A-B):

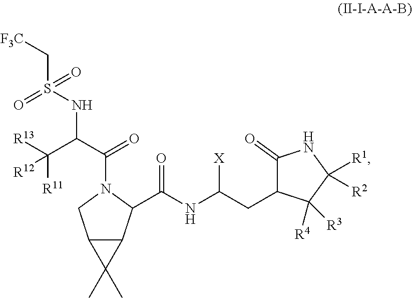

or a pharmaceutically acceptable salt, stereoisomer, solvate, prodrug, isotopic derivative, or tautomer thereof, wherein all variables are as defined herein.

In some embodiments, the compound is of Formula (II-I-A-A-B*):

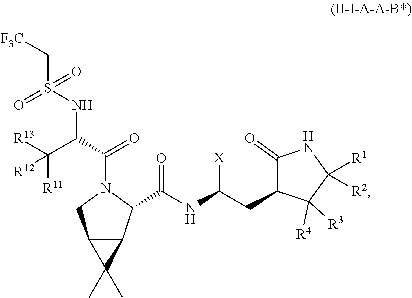

or a pharmaceutically acceptable salt, stereoisomer, solvate, prodrug, isotopic derivative, or tautomer thereof, wherein all variables are as defined herein.

In some embodiments, the compound is of Formula (II-I-A-A-B-I):

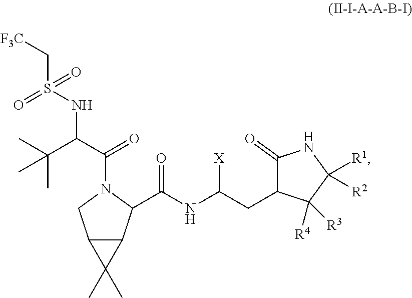

or a pharmaceutically acceptable salt, stereoisomer, solvate, prodrug, isotopic derivative, or tautomer thereof, wherein all variables are as defined herein.

In some embodiments, the compound is of Formula (II-I-A-A-B-I*):

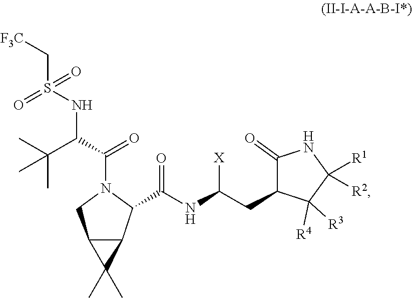

or a pharmaceutically acceptable salt, stereoisomer, solvate, prodrug, isotopic derivative, or tautomer thereof, wherein all variables are as defined herein.

In some embodiments, the compound is of Formula (II-I-A-A-B-I-1) or Formula (II-I-A-A-B-I-2):

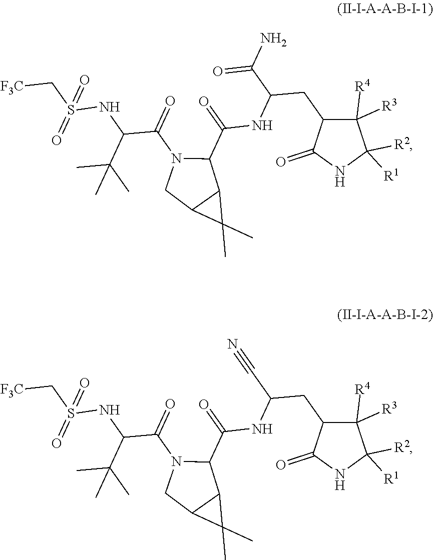

or a pharmaceutically acceptable salt, stereoisomer, solvate, prodrug, isotopic derivative, or tautomer thereof, wherein all variables are as defined herein.

In some embodiments, the compound is of Formula (II-I-A-A-B-I-1*) or Formula (II-I-A-A-B-I-2*):

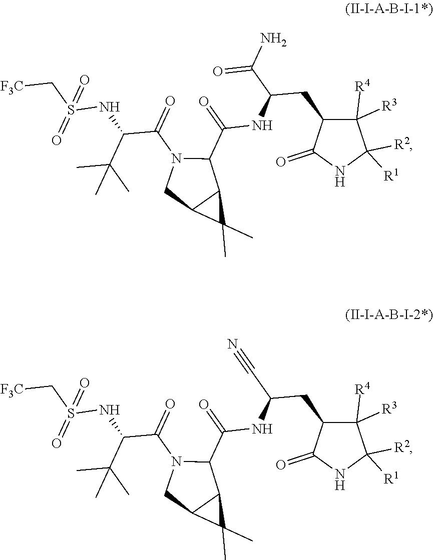

or a pharmaceutically acceptable salt, stereoisomer, solvate, prodrug, isotopic derivative, or tautomer thereof, wherein all variables are as defined herein.

In some embodiments, the compound is of Formula (II-I-A-A-C):

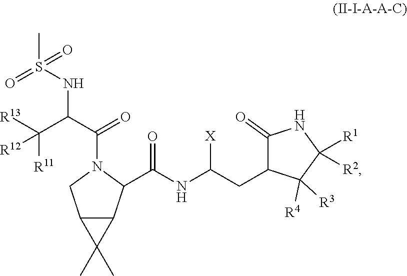

or a pharmaceutically acceptable salt, stereoisomer, solvate, prodrug, isotopic derivative, or tautomer thereof, wherein all variables are as defined herein.

In some embodiments, the compound is of Formula (II-I-A-A-C*):

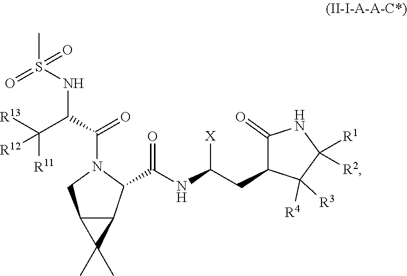

or a pharmaceutically acceptable salt, stereoisomer, solvate, prodrug, isotopic derivative, or tautomer thereof, wherein all variables are as defined herein.

In some embodiments, the compound is of Formula (II-I-A-A-C-I):

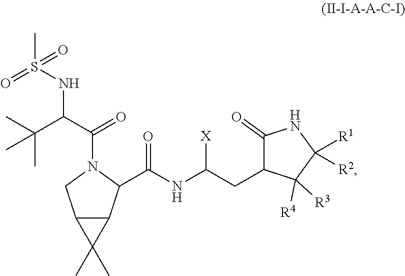

or a pharmaceutically acceptable salt, stereoisomer, solvate, prodrug, isotopic derivative, or tautomer thereof, wherein all variables are as defined herein.

In some embodiments, the compound is of Formula (II-I-A-A-C-I*).

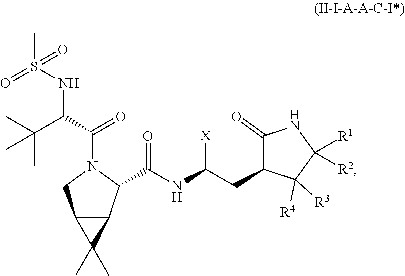

or a pharmaceutically acceptable salt, stereoisomer, solvate, prodrug, isotopic derivative, or tautomer thereof, wherein all variables are as defined herein.

In some embodiments, the compound is of Formula (II-I-A-A-C-I-1) or Formula (II-I-A-A-C-I-2):

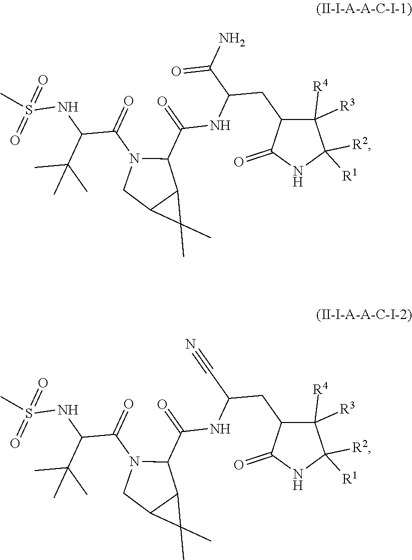

or a pharmaceutically acceptable salt, stereoisomer, solvate, prodrug, isotopic derivative, or tautomer thereof, wherein all variables are as defined herein.

In some embodiments, the compound is of Formula (II-I-A-A-C-I-1*) or Formula (II-I-A-A-C-I-2*):

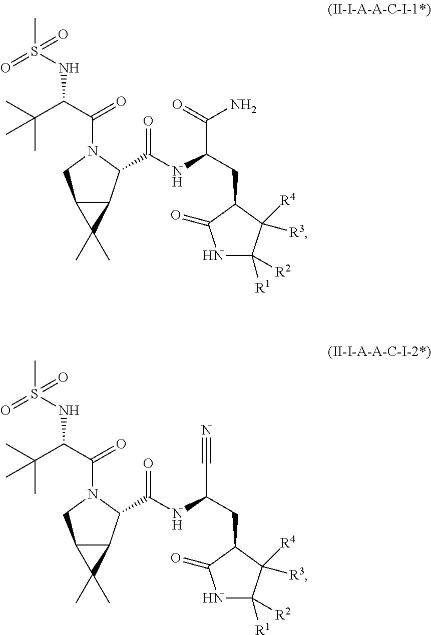

or a pharmaceutically acceptable salt, stereoisomer, solvate, prodrug, isotopic derivative, or tautomer thereof, wherein all variables are as defined herein.

In some embodiments, the compound is of Formula (II-I-A-A-D):

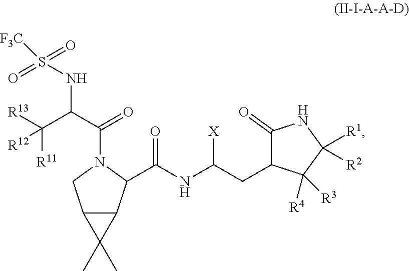

or a pharmaceutically acceptable salt, stereoisomer, solvate, prodrug, isotopic derivative, or tautomer thereof, wherein all variables are as defined herein.

In some embodiments, the compound is of Formula (II-I-A-A-D*):

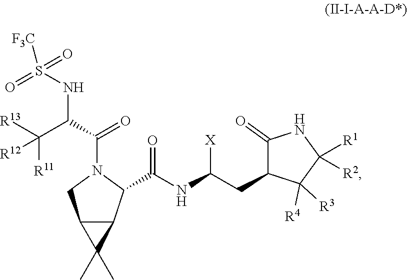

or a pharmaceutically acceptable salt, stereoisomer, solvate, prodrug, isotopic derivative, or tautomer thereof, wherein all variables are as defined herein.

In some embodiments, the compound is of Formula (II-I-A-A-D-I):

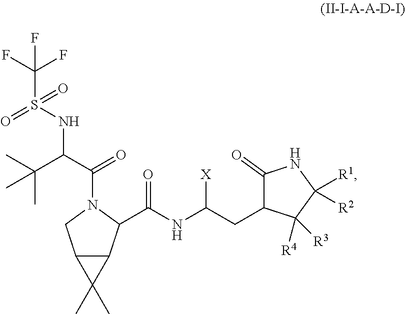

or a pharmaceutically acceptable salt, stereoisomer, solvate, prodrug, isotopic derivative, or tautomer thereof, wherein all variables are as defined herein.

In some embodiments, the compound is of Formula (II-I-A-A-D-I*):

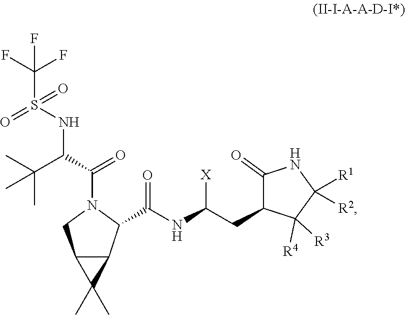

or a pharmaceutically acceptable salt, stereoisomer, solvate, prodrug, isotopic derivative, or tautomer thereof, wherein all variables are as defined herein.

In some embodiments, the compound is of Formula (II-I-A-A-D-I-1) or Formula (II-I-A-A-D-I-2):

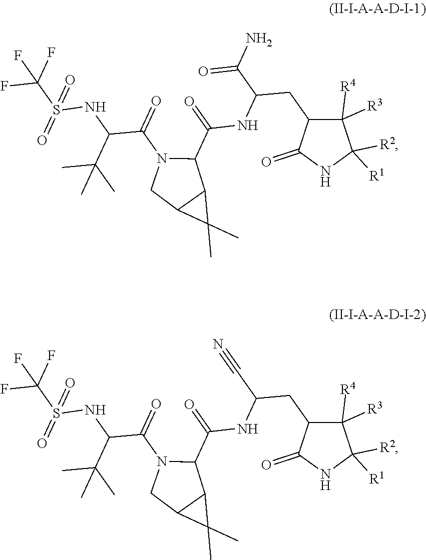

or a pharmaceutically acceptable salt, stereoisomer, solvate, prodrug, isotopic derivative, or tautomer thereof, wherein all variables are as defined herein.

In some embodiments, the compound is of Formula (II-I-A-A-D-I-1*) or Formula (II-I-A-A-D-I-2*):

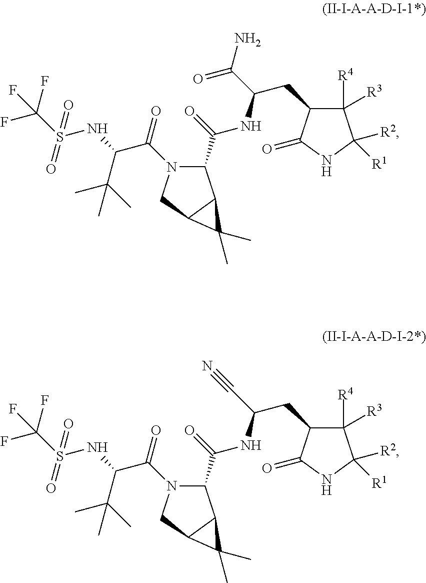

or a pharmaceutically acceptable salt, stereoisomer, solvate, prodrug, isotopic derivative, or tautomer thereof, wherein all variables are as defined herein.

In some embodiments, the compound is of Formula (II-I-A-B):

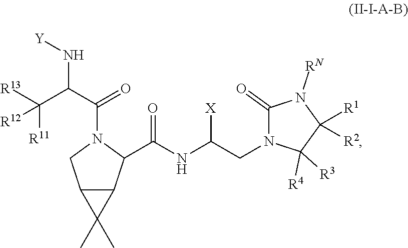

or a pharmaceutically acceptable salt, stereoisomer, solvate, prodrug, isotopic derivative, or tautomer thereof, wherein all variables are as defined herein.

In some embodiments, the compound is of Formula (II-I-A-B*):

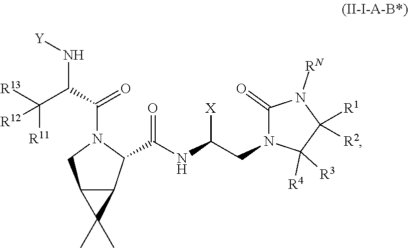

or a pharmaceutically acceptable salt, stereoisomer, solvate, prodrug, isotopic derivative, or tautomer thereof, wherein all variables are as defined herein.

In some embodiments, the compound is of Formula (II-I-A-B-A):

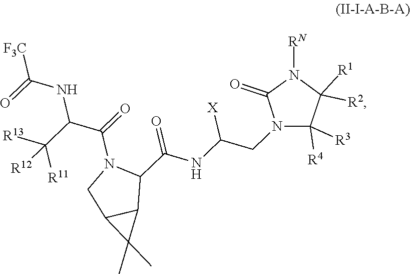

or a pharmaceutically acceptable salt, stereoisomer, solvate, prodrug, isotopic derivative, or tautomer thereof, wherein all variables are as defined herein.

In some embodiments, the compound is of Formula (II-I-A-B-A*):

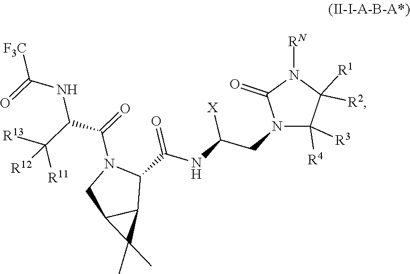

or a pharmaceutically acceptable salt, stereoisomer, solvate, prodrug, isotopic derivative, or tautomer thereof, wherein all variables are as defined herein.

In some embodiments, the compound is of Formula (II-I-A-B-A-I):

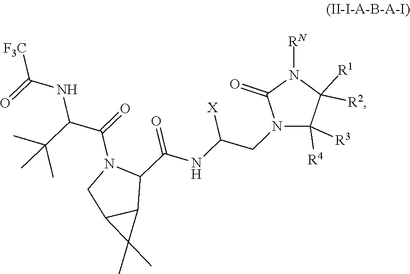

or a pharmaceutically acceptable salt, stereoisomer, solvate, prodrug, isotopic derivative, or tautomer thereof, wherein all variables are as defined herein.

In some embodiments, the compound is of Formula (II-I-A-B-A-I*):

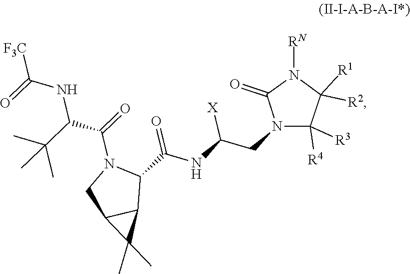

or a pharmaceutically acceptable salt, stereoisomer, solvate, prodrug, isotopic derivative, or tautomer thereof, wherein all variables are as defined herein.

In some embodiments, the compound is of Formula (II-I-B):

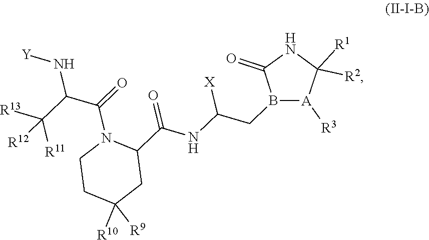

or a pharmaceutically acceptable salt, stereoisomer, solvate, prodrug, isotopic derivative, or tautomer thereof, wherein all variables are as defined herein.

In some embodiments, the compound is of Formula (II-I-B*):

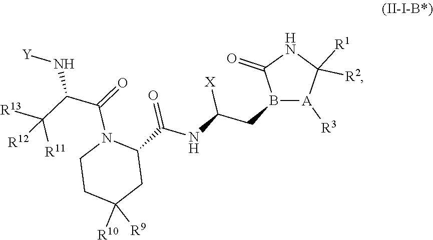

or a pharmaceutically acceptable salt, stereoisomer, solvate, prodrug, isotopic derivative, or tautomer thereof, wherein all variables are as defined herein.

In some embodiments, the compound is of Formula (II-I-B-A):

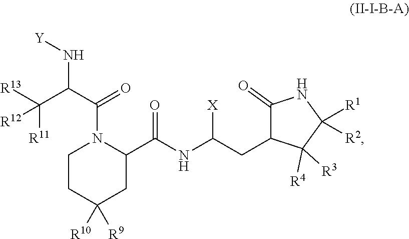

or a pharmaceutically acceptable salt, stereoisomer, solvate, prodrug, isotopic derivative, or tautomer thereof, wherein all variables are as defined herein.

In some embodiments, the compound is of Formula (II-I-B-A*):

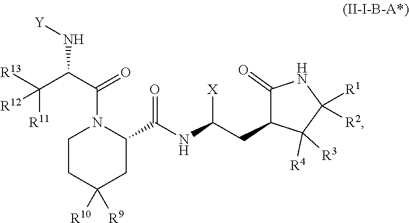

or a pharmaceutically acceptable salt, stereoisomer, solvate, prodrug, isotopic derivative, or tautomer thereof, wherein all variables are as defined herein.

In some embodiments, the compound is of Formula (II-I-B-A-a) or Formula (II-I-B-A-b):

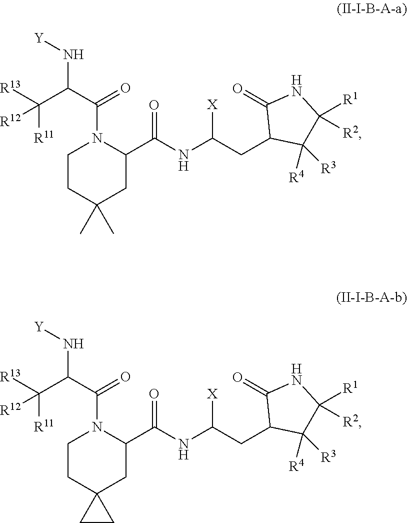

or a pharmaceutically acceptable salt, stereoisomer, solvate, prodrug, isotopic derivative, or tautomer thereof, wherein all variables are as defined herein.

In some embodiments, the compound is of Formula (II-I-B-A-a*) or Formula (II-I-B-A-b*):

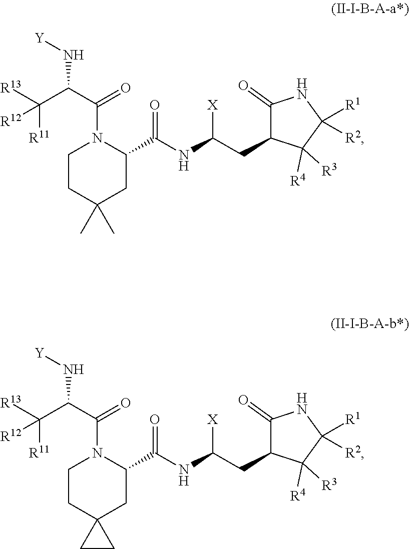

or a pharmaceutically acceptable salt, stereoisomer, solvate, prodrug, isotopic derivative, or tautomer thereof, wherein all variables are as defined herein.

In some embodiments, the compound is of Formula (II-I-B-A-A):

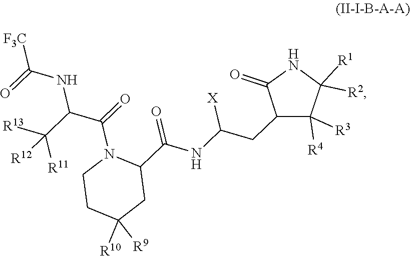

or a pharmaceutically acceptable salt, stereoisomer, solvate, prodrug, isotopic derivative, or tautomer thereof, wherein all variables are as defined herein.

In some embodiments, the compound is of Formula (II-I-B-A-A*):

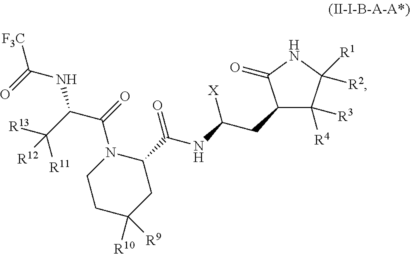

or a pharmaceutically acceptable salt, stereoisomer, solvate, prodrug, isotopic derivative, or tautomer thereof, wherein all variables are as defined herein.

In some embodiments, the compound is of Formula (II-I-B-A-A-a) or Formula (II-I-B-A-A-b):

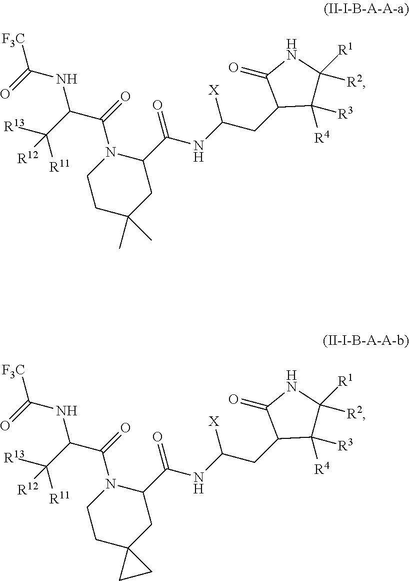

or a pharmaceutically acceptable salt, stereoisomer, solvate, prodrug, isotopic derivative, or tautomer thereof, wherein all variables are as defined herein.

In some embodiments, the compound is of Formula (II-I-B-A-A-a*) or Formula (II-I-B-A-A-b*):

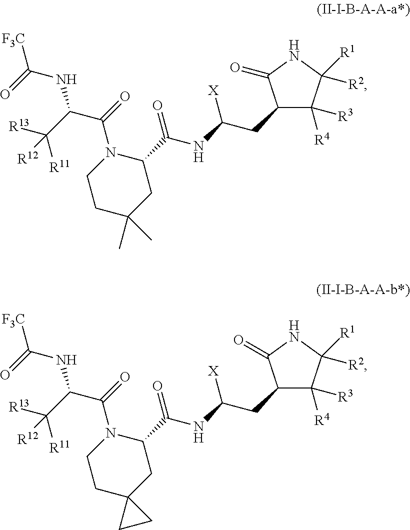

or a pharmaceutically acceptable salt, stereoisomer, solvate, prodrug, isotopic derivative, or tautomer thereof, wherein all variables are as defined herein.

In some embodiments, the compound is of Formula (II-I-B-A-A-I):

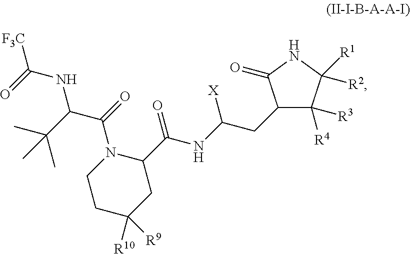

or a pharmaceutically acceptable salt, stereoisomer, solvate, prodrug, isotopic derivative, or tautomer thereof, wherein all variables are as defined herein.

In some embodiments, the compound is of Formula (II-I-B-A-A-I*):

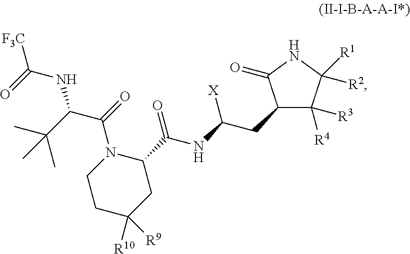

or a pharmaceutically acceptable salt, stereoisomer, solvate, prodrug, isotopic derivative, or tautomer thereof, wherein all variables are as defined herein.

In some embodiments, the compound is of Formula (II-I-B-A-A-I-a) or Formula (II-I-B-A-A-I-b):

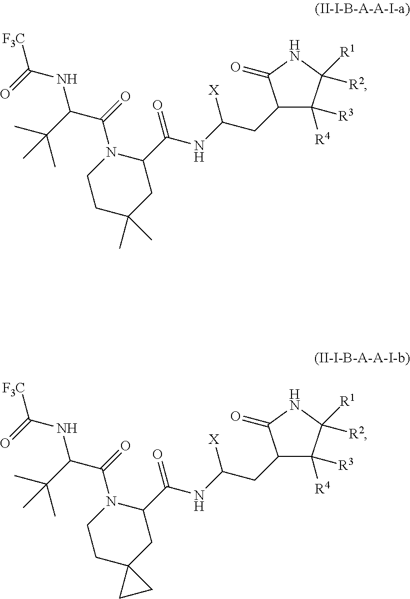

or a pharmaceutically acceptable salt, stereoisomer, solvate, prodrug, isotopic derivative, or tautomer thereof, wherein X is selected from —C(O)NH₂ and —CN and all other variables are as defined herein.

In some embodiments, the compound is of Formula (II-I-B-A-A-I-a*) or Formula (II-I-B-A-A-I-b*):

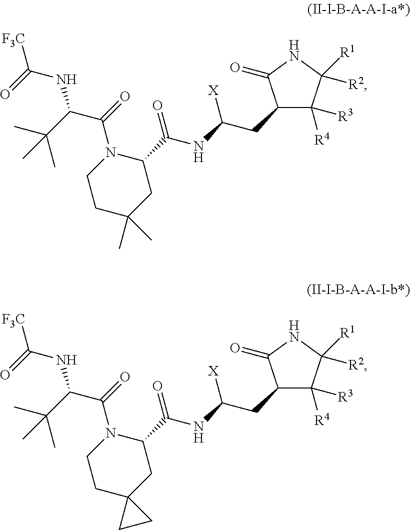

or a pharmaceutically acceptable salt, stereoisomer, solvate, prodrug, isotopic derivative, or tautomer thereof, wherein X is selected from —C(O)NH₂ and —CN and all other variables are as defined herein.

In some embodiments, the compound is of Formula (II-II-A):

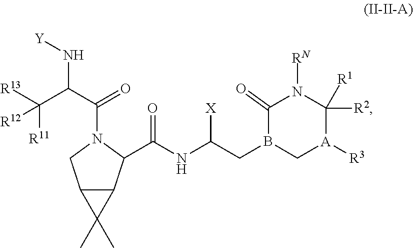

or a pharmaceutically acceptable salt, stereoisomer, solvate, prodrug, isotopic derivative, or tautomer thereof, wherein all variables are as defined herein.

In some embodiments, the compound is of Formula (II-II-A*):

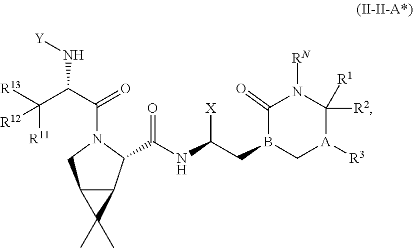

or a pharmaceutically acceptable salt, stereoisomer, solvate, prodrug, isotopic derivative, or tautomer thereof, wherein all variables are as defined herein.

In some embodiments, the compound is of Formula (II-II-A-A):

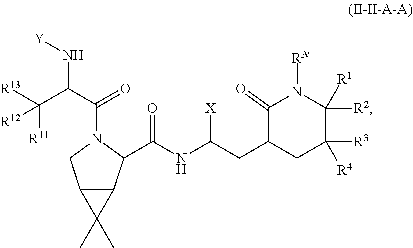

or a pharmaceutically acceptable salt, stereoisomer, solvate, prodrug, isotopic derivative, or tautomer thereof, wherein all variables are as defined herein.

In some embodiments, the compound is of Formula (II-II-A-A*):

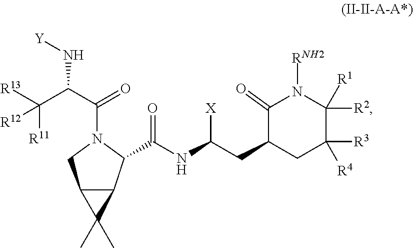

or a pharmaceutically acceptable salt, stereoisomer, solvate, prodrug, isotopic derivative, or tautomer thereof, wherein all variables are as defined herein.

In some embodiments, the compound is of Formula (II-II-A-A-A):

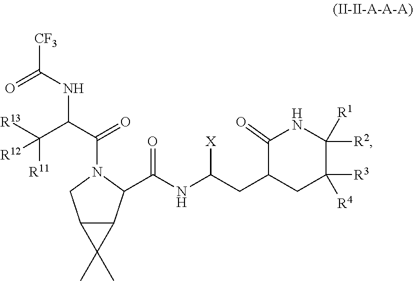

or a pharmaceutically acceptable salt, stereoisomer, solvate, prodrug, isotopic derivative, or tautomer thereof, wherein all variables are as defined herein.

In some embodiments, the compound is of Formula (II-II-A-A-A*):

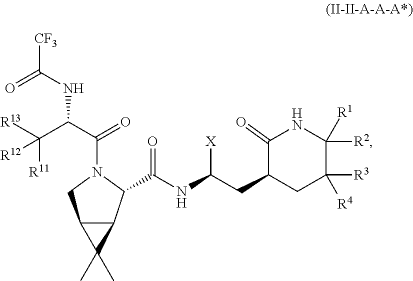

or a pharmaceutically acceptable salt, stereoisomer, solvate, prodrug, isotopic derivative, or tautomer thereof, wherein all variables are as defined herein.

In some embodiments, the compound is of Formula (II-II-A-A-A-I):

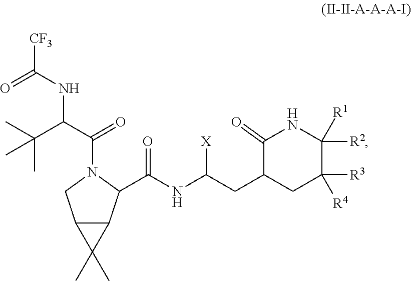

or a pharmaceutically acceptable salt, stereoisomer, solvate, prodrug, isotopic derivative, or tautomer thereof, wherein all variables are as defined herein.

In some embodiments, the compound is of Formula (II-II-A-A-A-I*):

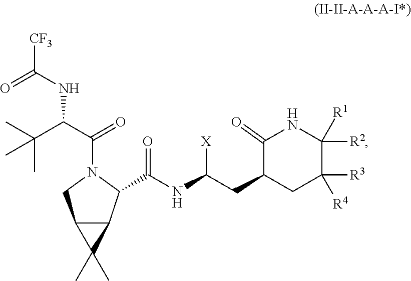

or a pharmaceutically acceptable salt, stereoisomer, solvate, prodrug, isotopic derivative, or tautomer thereof, wherein all variables are as defined herein.

In some embodiments, the compound is of Formula (II-II-A-A-A-I-1) or Formula (II-II-A-A-A-I-2):

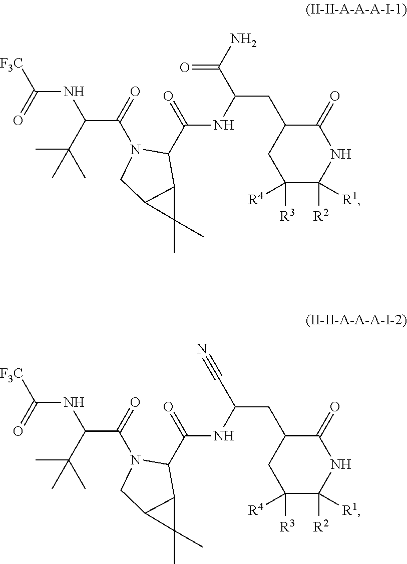

or a pharmaceutically acceptable salt, stereoisomer, solvate, prodrug, isotopic derivative, or tautomer thereof, wherein all variables are as defined herein.

In some embodiments, the compound is of Formula (II-II-A-A-A-I-1*) or Formula (II-II-A-A-A-I-2*):

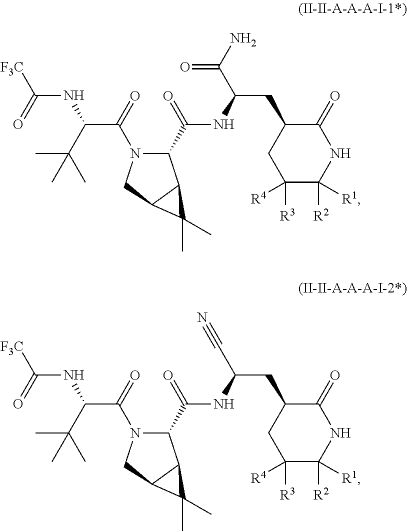

or a pharmaceutically acceptable salt, stereoisomer, solvate, prodrug, isotopic derivative, or tautomer thereof, wherein all variables are as defined herein.

In some embodiments, the compound is of Formula (II-II-A-B):

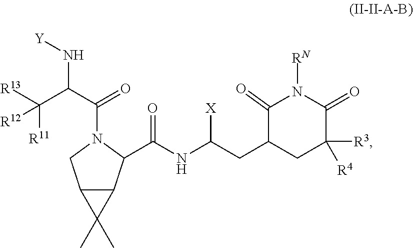

or a pharmaceutically acceptable salt, stereoisomer, solvate, prodrug, isotopic derivative, or tautomer thereof, wherein all variables are as defined herein.

In some embodiments, the compound is of Formula (II-II-A-B*):

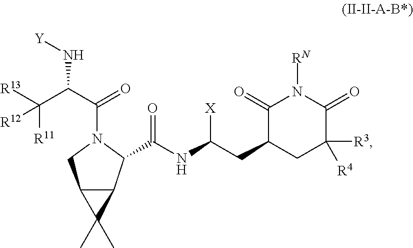

or a pharmaceutically acceptable salt, stereoisomer, solvate, prodrug, isotopic derivative, or tautomer thereof, wherein all variables are as defined herein.

In some embodiments, the compound is of Formula (II-II-A-B-A):

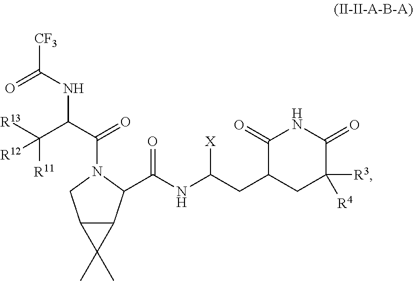

or a pharmaceutically acceptable salt, stereoisomer, solvate, prodrug, isotopic derivative, or tautomer thereof, wherein all variables are as defined herein.

In some embodiments, the compound is of Formula (II-II-A-B-A*):

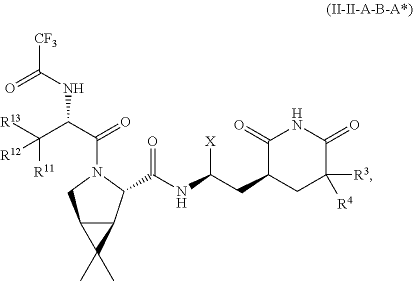

or a pharmaceutically acceptable salt, stereoisomer, solvate, prodrug, isotopic derivative, or tautomer thereof, wherein all variables are as defined herein.

In some embodiments, the compound is of Formula (II-II-A-B-A-I):

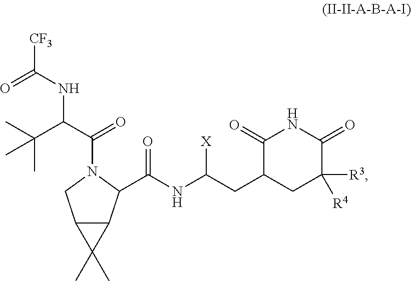

or a pharmaceutically acceptable salt, stereoisomer, solvate, prodrug, isotopic derivative, or tautomer thereof, wherein all variables are as defined herein.

In some embodiments, the compound is of Formula (II-II-A-B-A-I*):

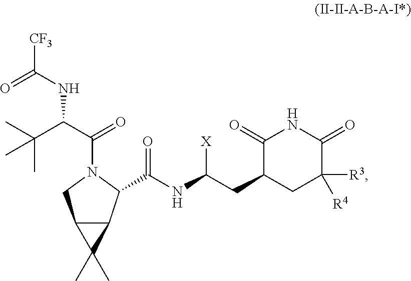

or a pharmaceutically acceptable salt, stereoisomer, solvate, prodrug, isotopic derivative, or tautomer thereof, wherein all variables are as defined herein.

In some embodiments, the compound is of Formula (II-II-A-B-A-I-1) or Formula (II-II-A-B-A-I-2):

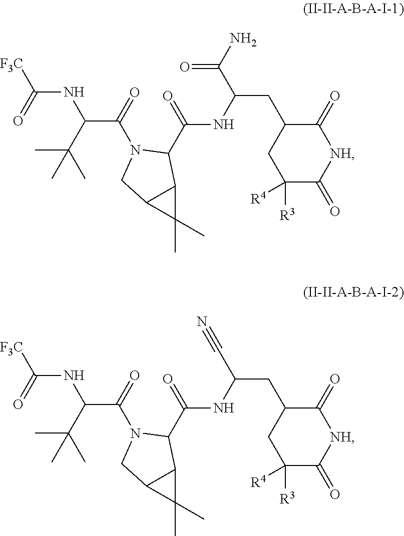

or a pharmaceutically acceptable salt, stereoisomer, solvate, prodrug, isotopic derivative, or tautomer thereof, wherein all variables are as defined herein.

In some embodiments, the compound is of Formula (II-II-A-B-A-I-1*) or Formula (II-II-A-B-A-I-2*):

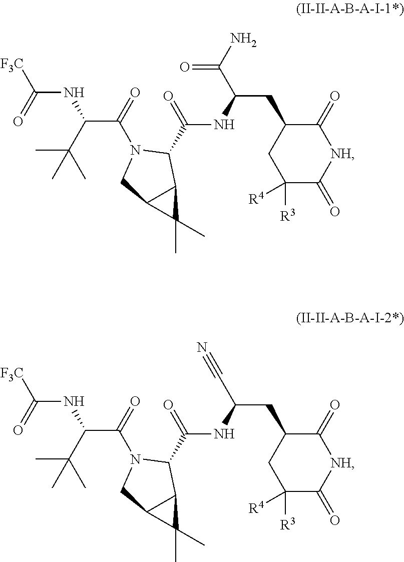

or a pharmaceutically acceptable salt, stereoisomer, solvate, prodrug, isotopic derivative, or tautomer thereof, wherein all variables are as defined herein.

In some embodiments, the compound is of Formula (II-II-A-C):

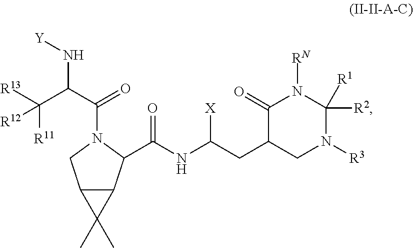

or a pharmaceutically acceptable salt, stereoisomer, solvate, prodrug, isotopic derivative, or tautomer thereof, wherein all variables are as defined herein.

In some embodiments, the compound is of Formula (II-II-A-C*):

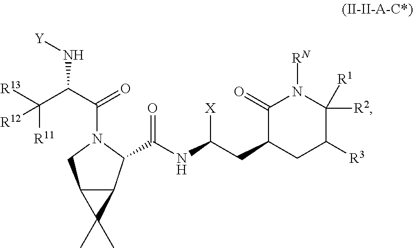

or a pharmaceutically acceptable salt, stereoisomer, solvate, prodrug, isotopic derivative, or tautomer thereof, wherein all variables are as defined herein.

In some embodiments, the compound is of Formula (II-II-A-C-A):

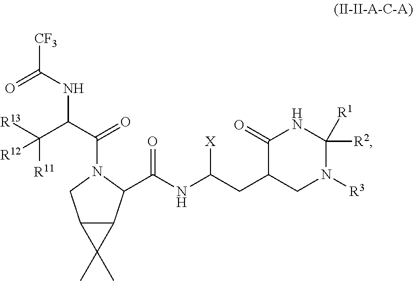

or a pharmaceutically acceptable salt, stereoisomer, solvate, prodrug, isotopic derivative, or tautomer thereof, wherein all variables are as defined herein.

In some embodiments, the compound is of Formula (II-II-A-C-A*):

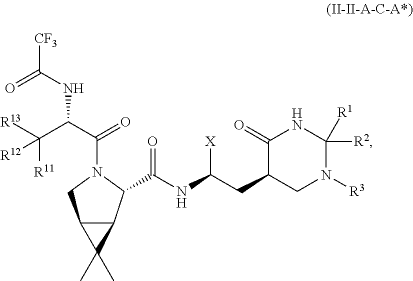

or a pharmaceutically acceptable salt, stereoisomer, solvate, prodrug, isotopic derivative, or tautomer thereof, wherein all variables are as defined herein.

In some embodiments, the compound is of Formula (II-II-A-C-A-I):

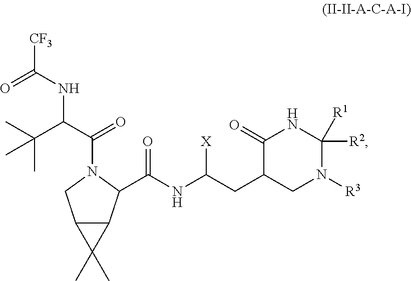

or a pharmaceutically acceptable salt, stereoisomer, solvate, prodrug, isotopic derivative, or tautomer thereof, wherein all variables are as defined herein.

In some embodiments, the compound is of Formula (II-II-A-C-A-I*):

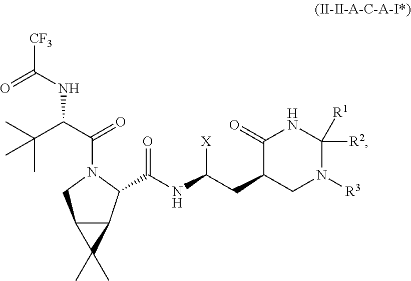

or a pharmaceutically acceptable salt, stereoisomer, solvate, prodrug, isotopic derivative, or tautomer thereof, wherein all variables are as defined herein.

In some embodiments, the compound is of Formula (II-II-A-C-A-I-1) or Formula (II-II-A-C-A-I-2):

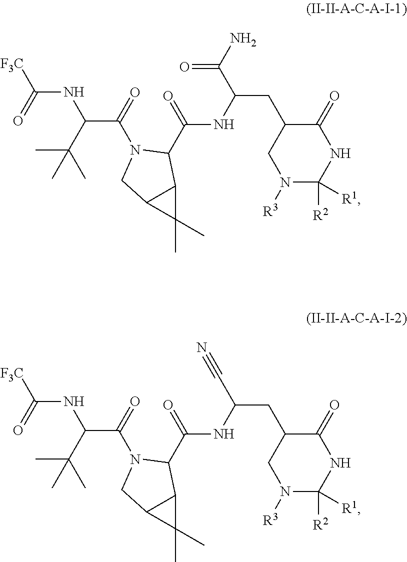

or a pharmaceutically acceptable salt, stereoisomer, solvate, prodrug, isotopic derivative, or tautomer thereof, wherein all variables are as defined herein.

In some embodiments, the compound is of Formula (II-II-A-C-A-I-1*) or Formula (II-II-A-C-A-I-2*):

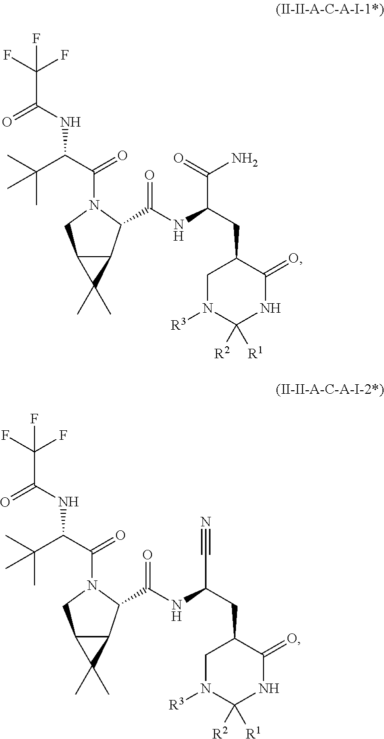

or a pharmaceutically acceptable salt, stereoisomer, solvate, prodrug, isotopic derivative, or tautomer thereof, wherein all variables are as defined herein.

In some embodiments, the compound is of Formula (II-I-W):

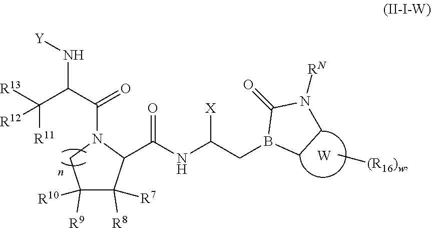

or a pharmaceutically acceptable salt, stereoisomer, solvate, prodrug, isotopic derivative, or tautomer thereof, wherein ring W is selected from 3-10 membered cycloalkyl, a 6-10 membered aryl, a 3-14 membered heterocycle, or a 5-6 membered heteroaryl; w is an integer selected from 0, 1, 2, 3, and 4; and all other variables are as defined herein.

In some embodiments, the compound is of Formula (II-I-W-A):

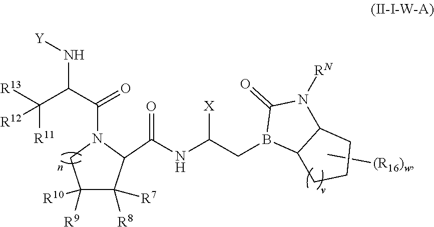

or a pharmaceutically acceptable salt, stereoisomer, solvate, prodrug, isotopic derivative, or tautomer thereof, wherein w is an integer selected from 0, 1, 2, 3, and 4; v is an integer selected from 1, 2 and 3; and all other variables are as defined herein.

In some embodiments, the compound is of Formula (II-I-W-B):

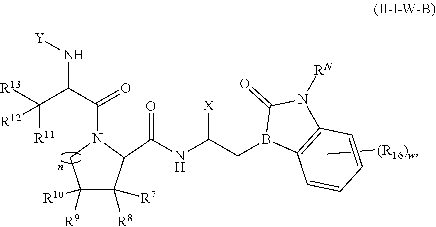

or a pharmaceutically acceptable salt, stereoisomer, solvate, prodrug, isotopic derivative, or tautomer thereof, wherein w is an integer selected from 0, 1, 2, 3, and 4 and all other variables are as defined herein.

In some embodiments, the compound is of Formula (II-I-W-C):

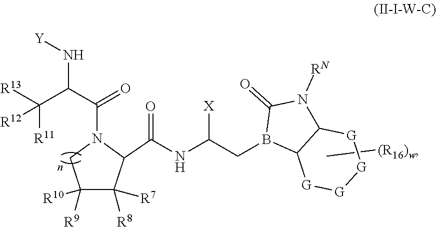

or a pharmaceutically acceptable salt, stereoisomer, solvate, prodrug, isotopic derivative, or tautomer thereof, wherein w is an integer selected from 0, 1, 2, 3, and 4; each G is independently selected from CH₂, NH, O, S, S(O), S(O)₂ and at least two G are CH₂; and all other variables are as defined herein.

In some embodiments, the compound is of Formula (II-I-W-D):

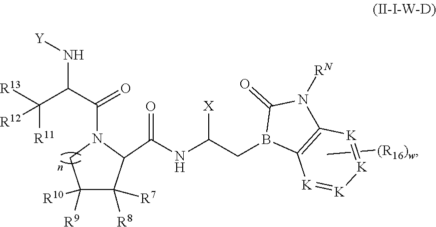

or a pharmaceutically acceptable salt, stereoisomer, solvate, prodrug, isotopic derivative, or tautomer thereof, wherein w is an integer selected from 0, 1, and 2; each K is independently selected from CH and N, and at least two K are CH; and all other variables are as defined herein.

In some embodiments, the compound is of Formula (II-I-W-A-I):

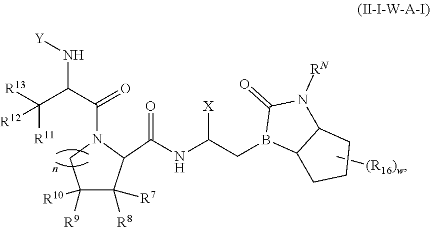

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-A-II):

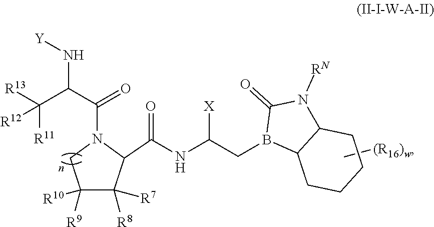

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-A-III):

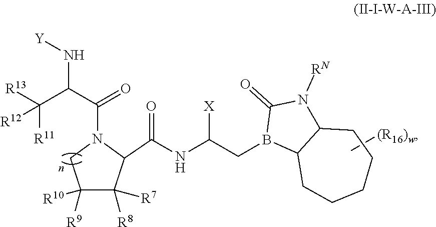

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-B-I):

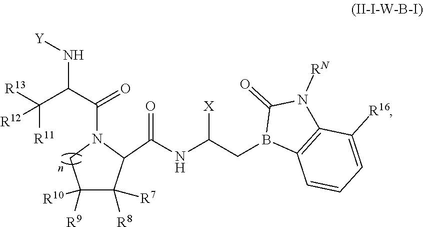

or a pharmaceutically acceptable salt, stereoisomer, solvate, prodrug, isotopic derivative, or tautomer thereof, wherein all variables are as defined herein.

In some embodiments, the compound is of Formula (II-I-W-B-II):

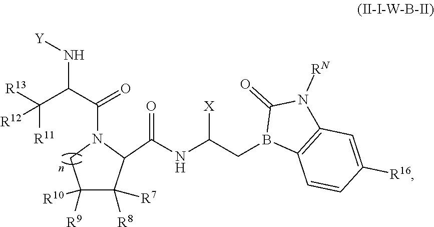

or a pharmaceutically acceptable salt, stereoisomer, solvate, prodrug, isotopic derivative, or tautomer thereof, wherein all variables are as defined herein.

In some embodiments, the compound is of Formula (II-I-W-B-III):

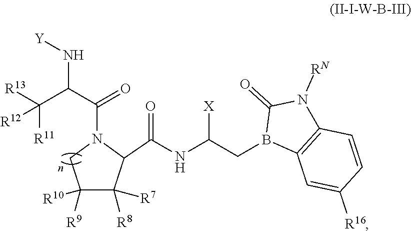

or a pharmaceutically acceptable salt, stereoisomer, solvate, prodrug, isotopic derivative, or tautomer thereof, wherein all variables are as defined herein.

In some embodiments, the compound is of Formula (II-I-W-B-IV):

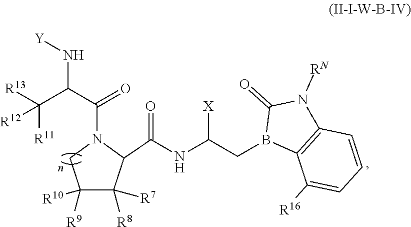

or a pharmaceutically acceptable salt, stereoisomer, solvate, prodrug, isotopic derivative, or tautomer thereof, wherein all variables are as defined herein.

In some embodiments, the compound is of Formula (II-I-W-B-V):

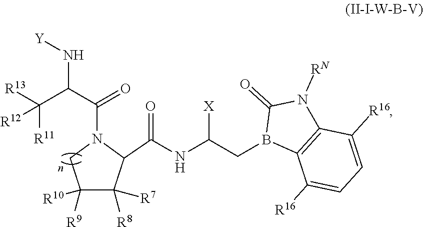

or a pharmaceutically acceptable salt, stereoisomer, solvate, prodrug, isotopic derivative, or tautomer thereof, wherein all variables are as defined herein.

In some embodiments, the compound is of Formula (II-I-W-B-VI):

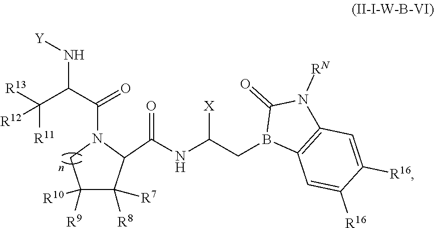

or a pharmaceutically acceptable salt, stereoisomer, solvate, prodrug, isotopic derivative, or tautomer thereof, wherein all variables are as defined herein.

In some embodiments, the compound is of Formula (II-I-W-C-I):

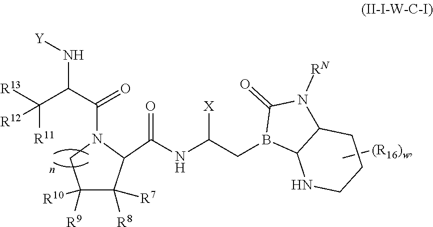

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-II):

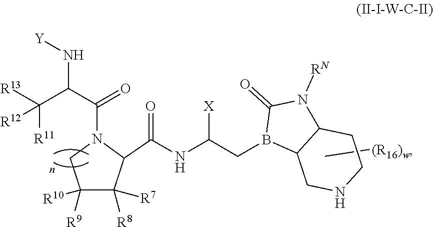

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-III):

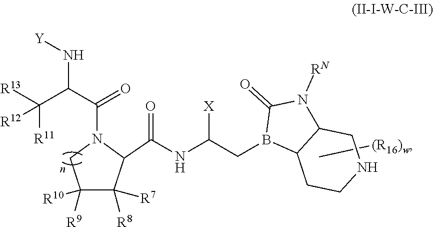

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-IV):

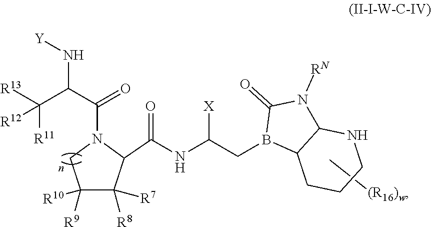

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-V):

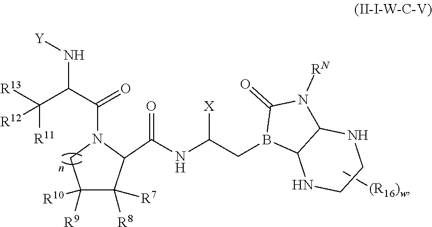

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-VI):

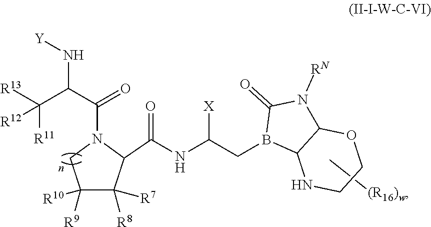

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-VII):

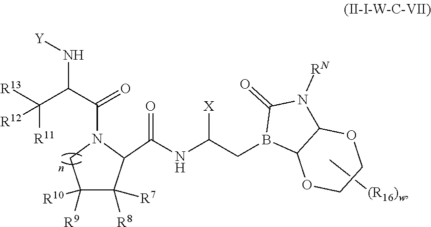

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-VIII):

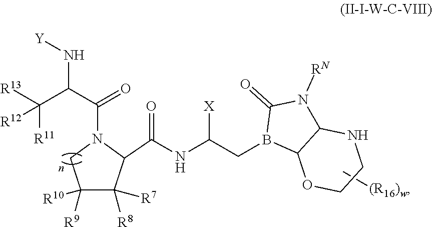

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-IX):

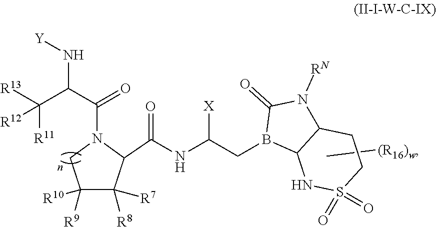

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-X):

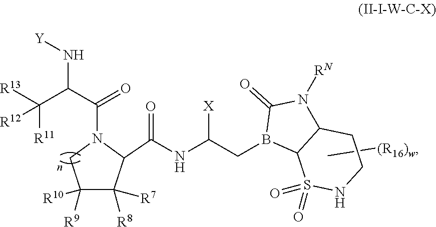

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-D-I):

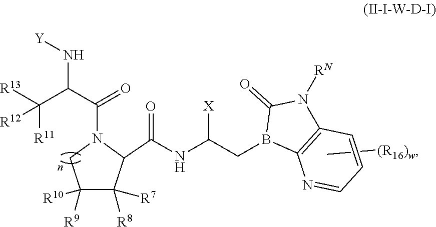

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-II):

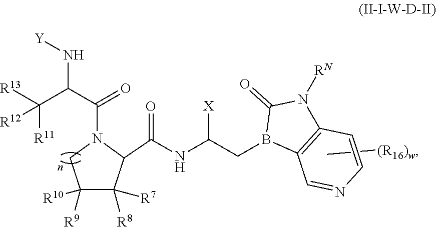

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-III):

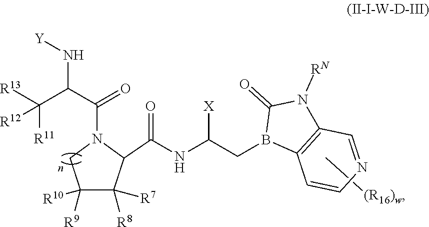

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-IV):

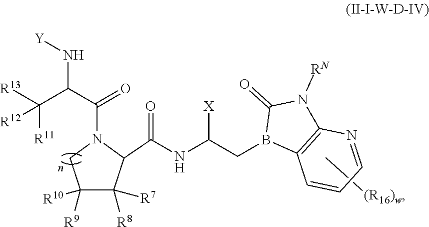

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-V):

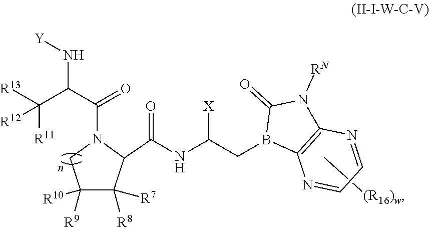

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-VI):

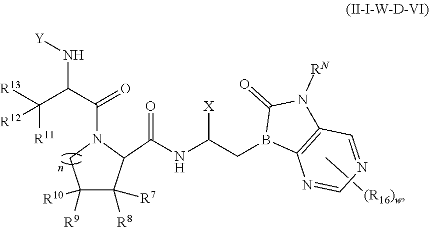

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-VII):

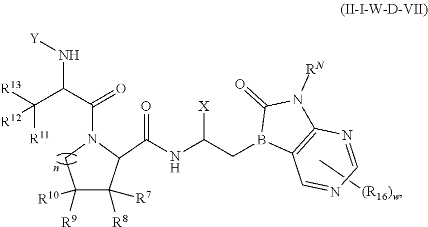

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-A-I-a):

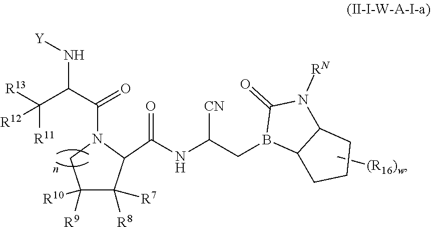

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-A-II-a):

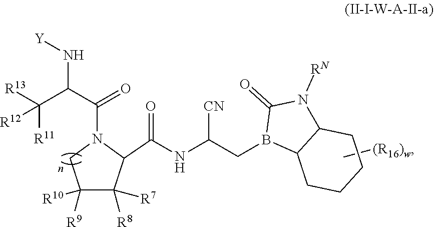

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-A-III-a):

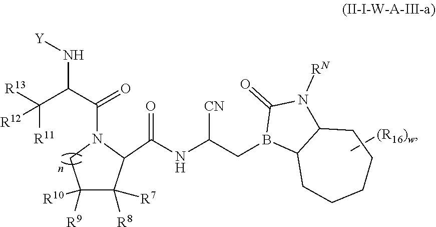

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-B-I-a):

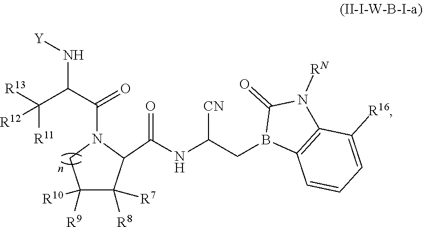

or a pharmaceutically acceptable salt, stereoisomer, solvate, prodrug, isotopic derivative, or tautomer thereof, wherein all variables are as defined herein.

In some embodiments, the compound is of Formula (II-I-W-B-II-a):

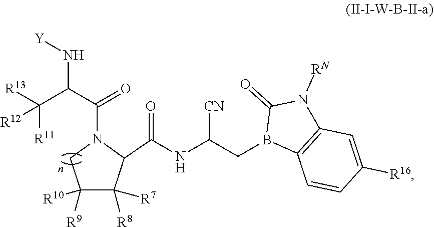

or a pharmaceutically acceptable salt, stereoisomer, solvate, prodrug, isotopic derivative, or tautomer thereof, wherein all variables are as defined herein.

In some embodiments, the compound is of Formula (II-I-W-B-III-a):

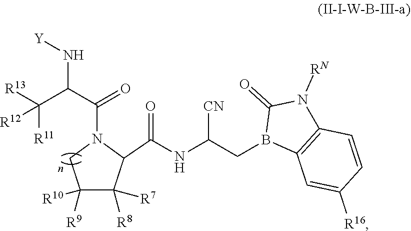

or a pharmaceutically acceptable salt, stereoisomer, solvate, prodrug, isotopic derivative, or tautomer thereof, wherein all variables are as defined herein.

In some embodiments, the compound is of Formula (II-I-W-B-IV-a):

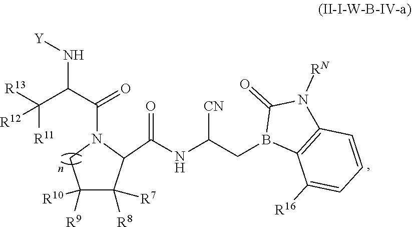

or a pharmaceutically acceptable salt, stereoisomer, solvate, prodrug, isotopic derivative, or tautomer thereof, wherein all variables are as defined herein.

In some embodiments, the compound is of Formula (II-I-W-B-V-a):

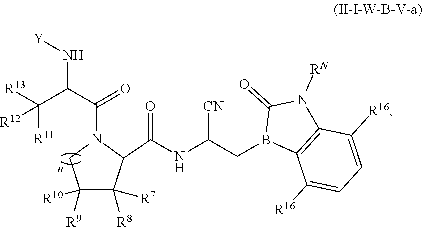

or a pharmaceutically acceptable salt, stereoisomer, solvate, prodrug, isotopic derivative, or tautomer thereof, wherein all variables are as defined herein.

In some embodiments, the compound is of Formula (II-I-W-B-VI-a):

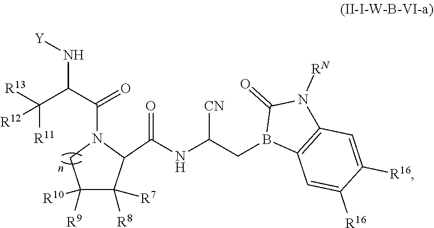

or a pharmaceutically acceptable salt, stereoisomer, solvate, prodrug, isotopic derivative, or tautomer thereof, wherein all variables are as defined herein.

In some embodiments, the compound is of Formula (II-I-W-C-I-a):

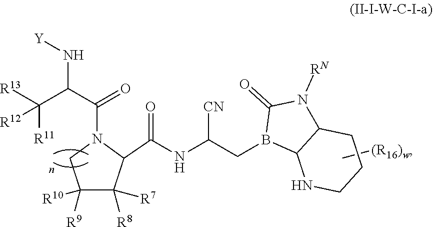

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-II-a):

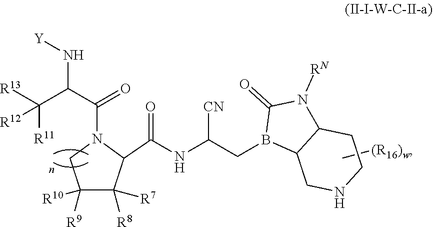

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-III-a):

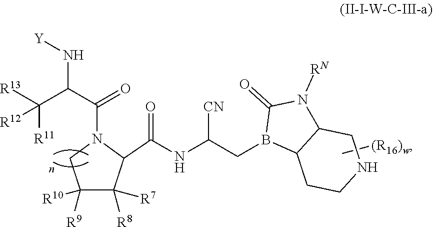

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-IV-a):

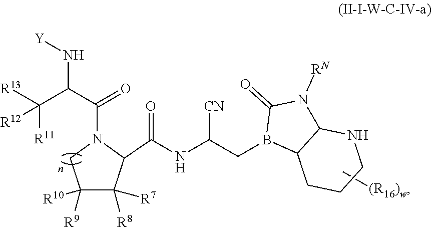

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-V-a):

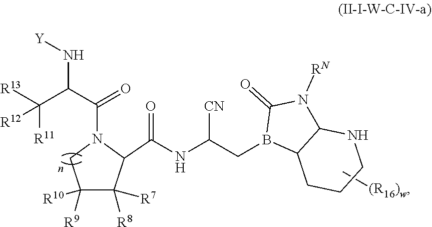

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-VI-a):

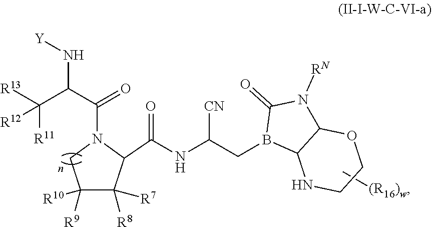

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-VII-a):

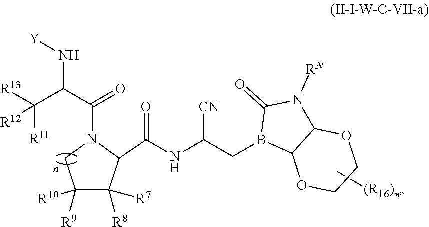

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-VIII-a):

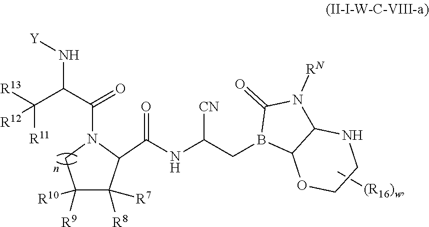

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-IX-a):

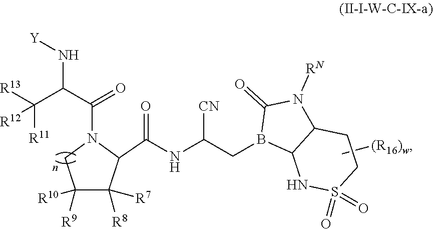

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-X-a):

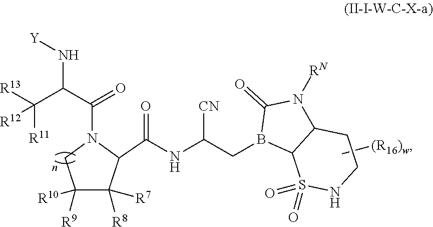

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-D-I-a):

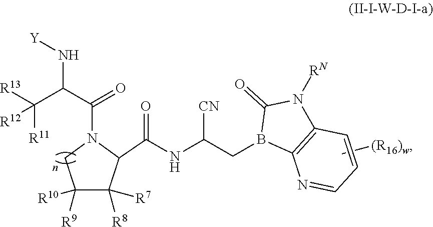

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-II-a):

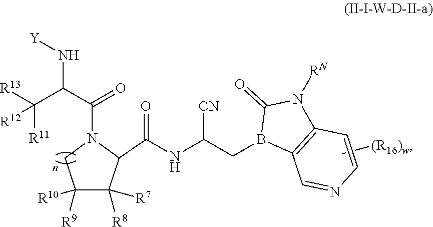

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-III-a):

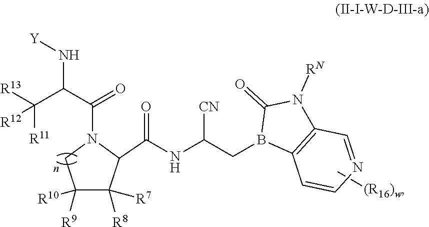

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-IV-a):

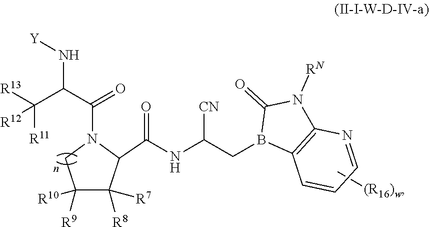

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-V-a):

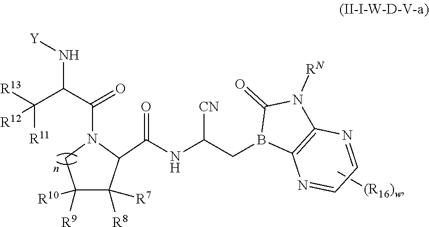

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-VI-a):

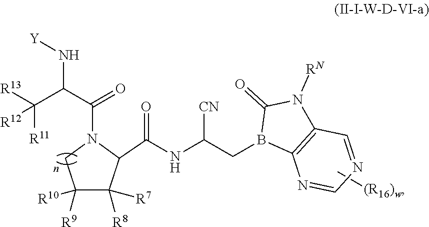

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-VII-a):

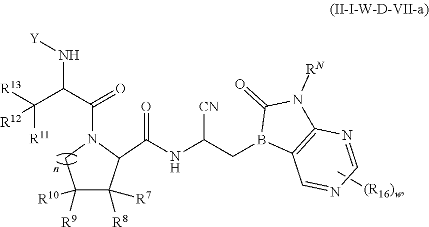

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-A-I-a-1):

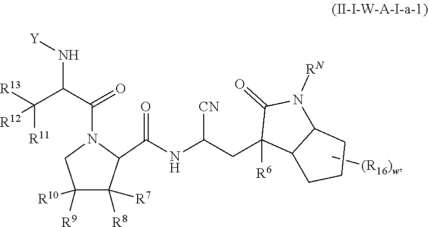

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-A-I-a-2):

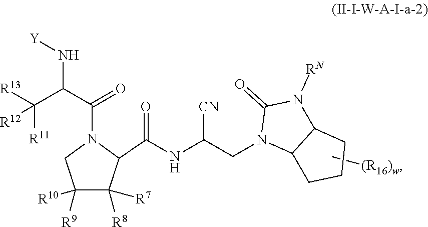

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-A-II-a-1):

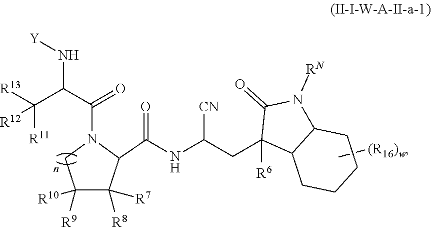

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-A-II-a-2):

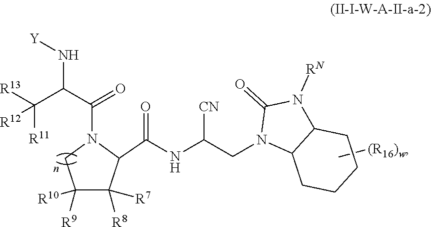

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-A-III-a-1):

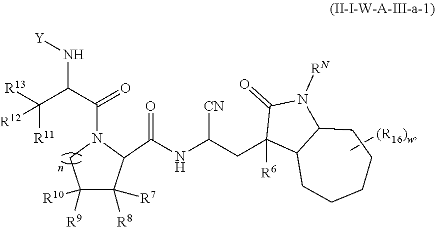

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-A-III-a-2):

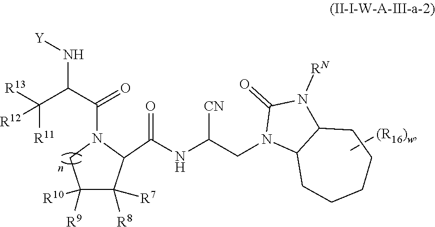

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-B-I-a-1):

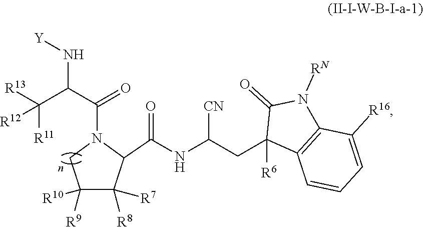

or a pharmaceutically acceptable salt, stereoisomer, solvate, prodrug, isotopic derivative, or tautomer thereof, wherein all variables are as defined herein.

In some embodiments, the compound is of Formula (II-I-W-B-I-a-2):

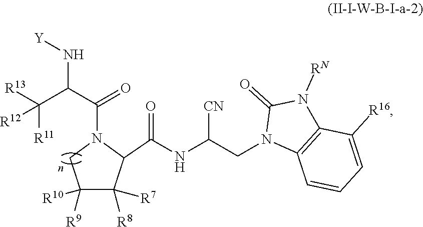

or a pharmaceutically acceptable salt, stereoisomer, solvate, prodrug, isotopic derivative, or tautomer thereof, wherein all variables are as defined herein.

In some embodiments, the compound is of Formula (II-I-W-B-II-a-1):

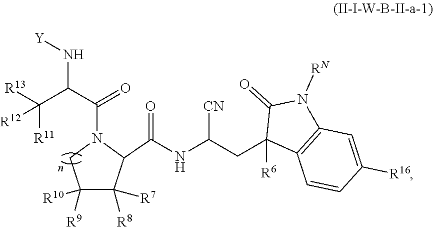

or a pharmaceutically acceptable salt, stereoisomer, solvate, prodrug, isotopic derivative, or tautomer thereof, wherein all variables are as defined herein.

In some embodiments, the compound is of Formula (II-I-W-B-II-a-2):

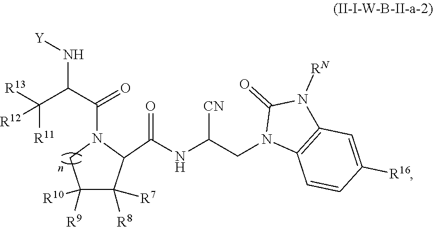

or a pharmaceutically acceptable salt, stereoisomer, solvate, prodrug, isotopic derivative, or tautomer thereof, wherein all variables are as defined herein.

In some embodiments, the compound is of Formula (II-I-W-B-III-a-1):

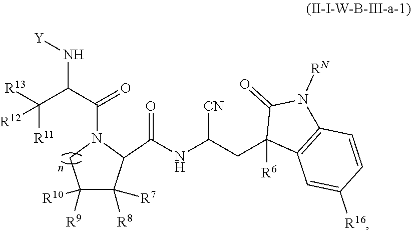

or a pharmaceutically acceptable salt, stereoisomer, solvate, prodrug, isotopic derivative, or tautomer thereof, wherein all variables are as defined herein.

In some embodiments, the compound is of Formula (II-I-W-B-III-a-2):

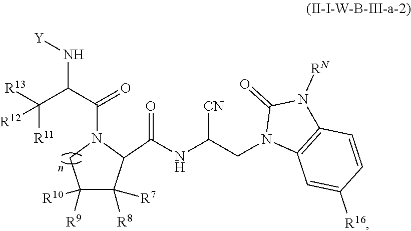

or a pharmaceutically acceptable salt, stereoisomer, solvate, prodrug, isotopic derivative, or tautomer thereof, wherein all variables are as defined herein.

In some embodiments, the compound is of Formula (II-I-W-B-IV-a-1):

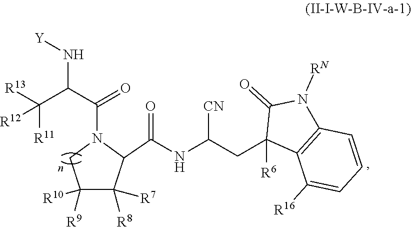

or a pharmaceutically acceptable salt, stereoisomer, solvate, prodrug, isotopic derivative, or tautomer thereof, wherein all variables are as defined herein.

In some embodiments, the compound is of Formula (II-I-W-B-IV-a-2):

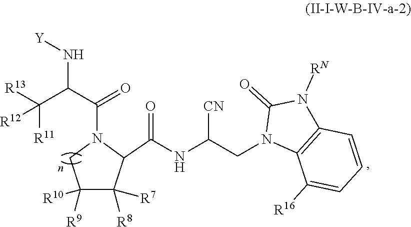

or a pharmaceutically acceptable salt, stereoisomer, solvate, prodrug, isotopic derivative, or tautomer thereof, wherein all variables are as defined herein.

In some embodiments, the compound is of Formula (II-I-W-B-V-a-1):

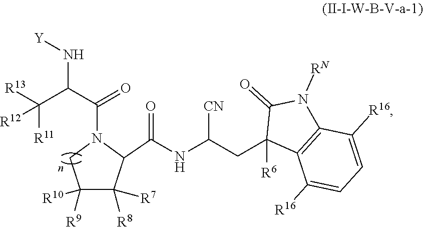

or a pharmaceutically acceptable salt, stereoisomer, solvate, prodrug, isotopic derivative, or tautomer thereof, wherein all variables are as defined herein.

In some embodiments, the compound is of Formula (II-I-W-B-V-a-2):

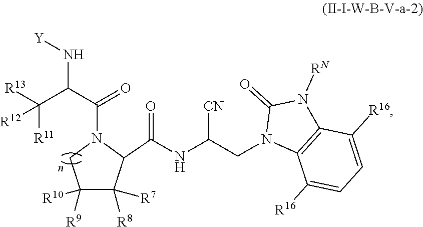

or a pharmaceutically acceptable salt, stereoisomer, solvate, prodrug, isotopic derivative, or tautomer thereof, wherein all variables are as defined herein.

In some embodiments, the compound is of Formula (II-I-W-B-VI-a-1):

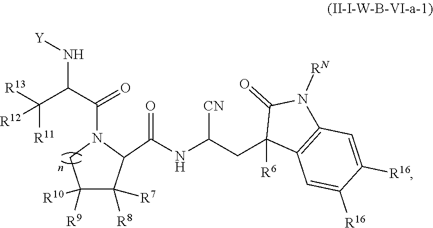

or a pharmaceutically acceptable salt, stereoisomer, solvate, prodrug, isotopic derivative, or tautomer thereof, wherein all variables are as defined herein.

In some embodiments, the compound is of Formula (II-I-W-B-VI-a-2):

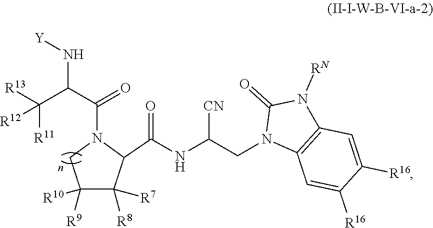

or a pharmaceutically acceptable salt, stereoisomer, solvate, prodrug, isotopic derivative, or tautomer thereof, wherein all variables are as defined herein.

In some embodiments, the compound is of Formula (II-I-W-C-I-a-1):

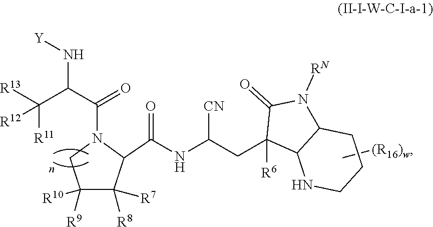

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-I-a-2):

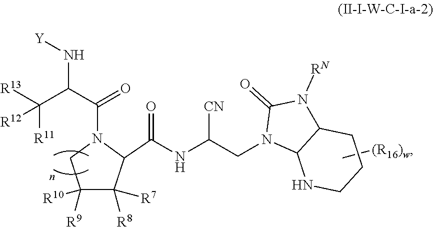

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-II-a-1):

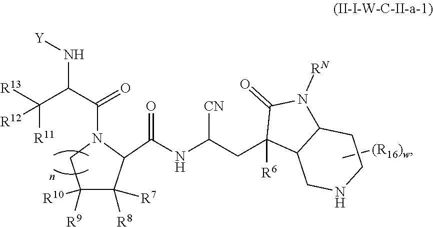

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-II-a-2):

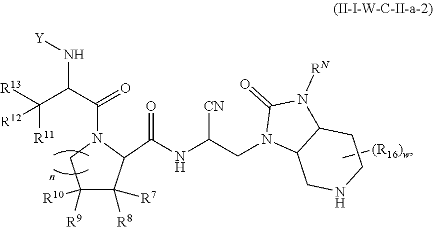

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-III-a-1):

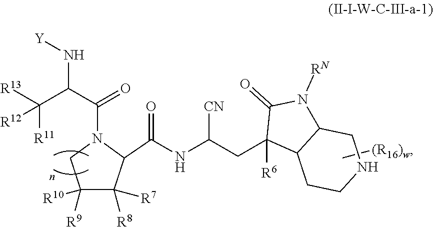

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-III-a-2):

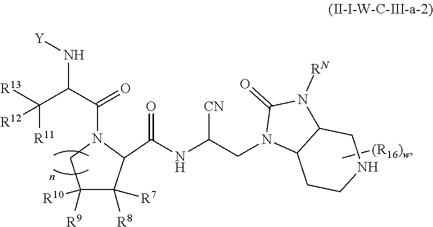

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-IV-a-1):

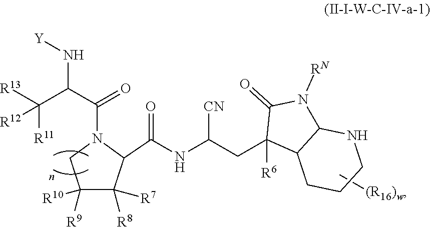

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-IV-a-2):

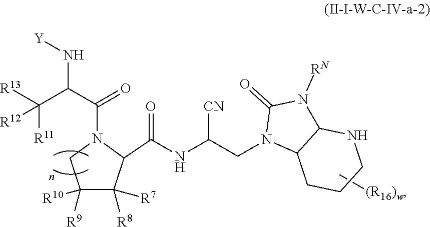

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-V-a-1):

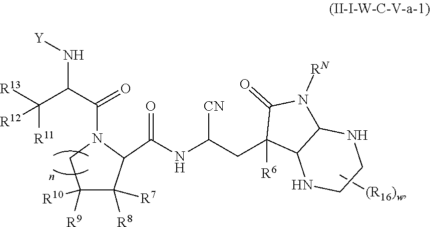

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-V-a-2):

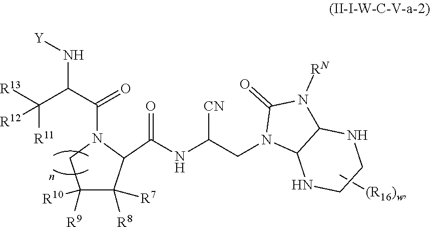

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-VI-a-1):

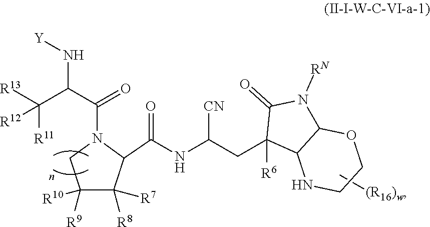

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-VI-a-2):

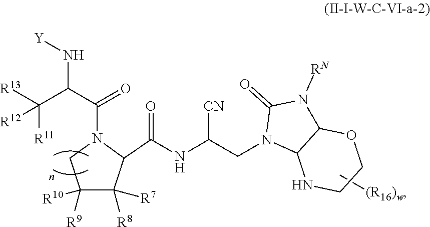

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-VII-a-1):

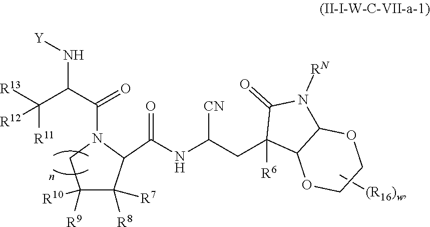

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-VII-a-2):

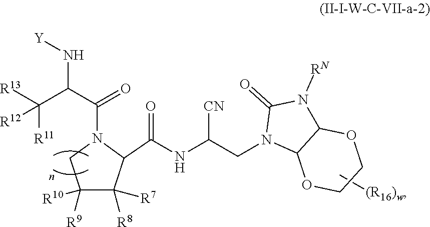

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-VIII-a-1):

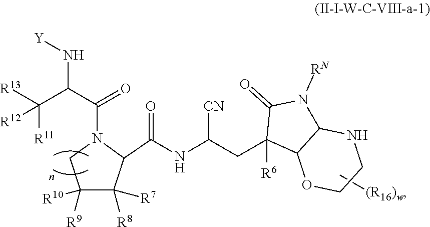

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-VIII-a-2):

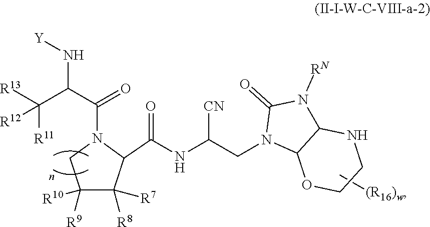

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-IX-a-1):

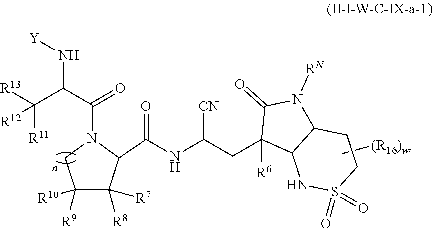

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-IX-a-2):

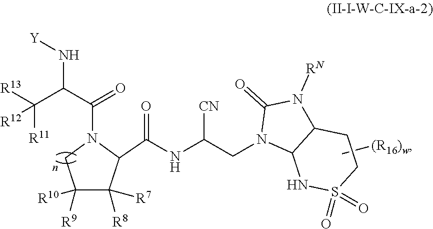

or a pharmaceutically acceptable salt, stereoisomer, solvate, prodrug, isotopic derivative, or tautomer thereof, wherein w is an integer selected from 0, 1, 2, 3, and 4; and all other variables are as defined herein

In some embodiments, the compound is of Formula (II-I-W-C-X-a-1):

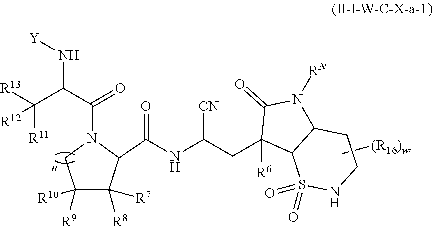

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-X-a-2):

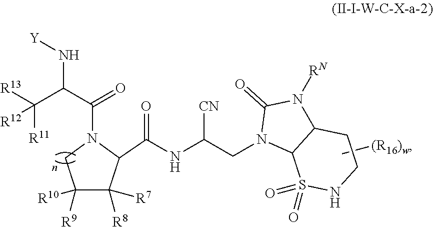

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-D-I-a-1):

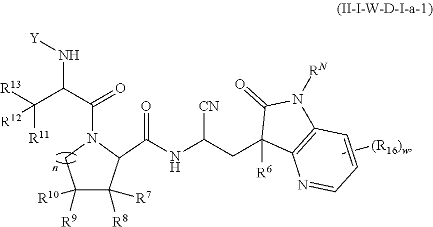

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-I-a-2):

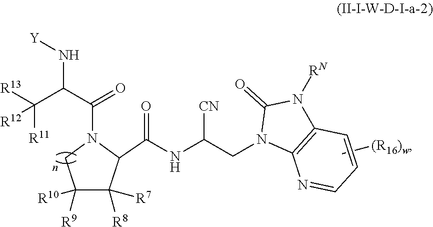

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-II-a-1):

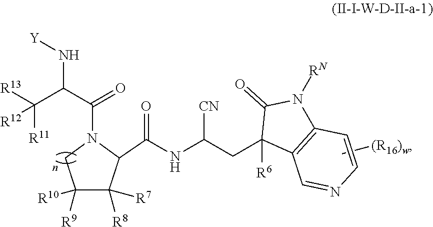

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-II-a-2):

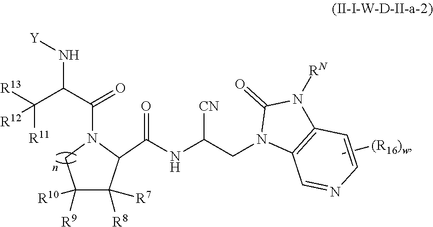

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-III-a-1):

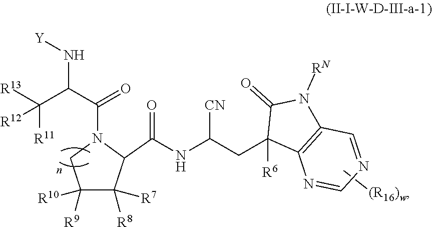

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-III-a-2):

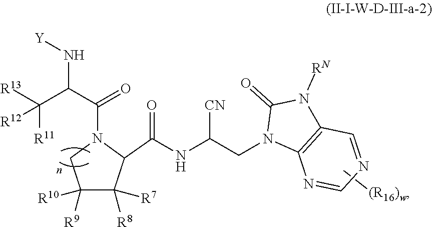

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-IV-a-1):

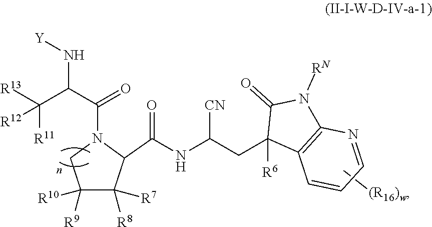

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-IV-a-2):

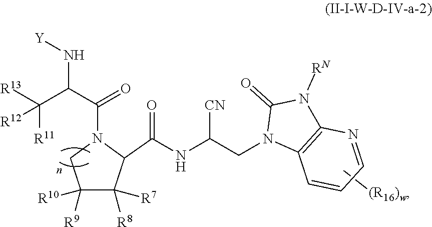

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-V-a-1):

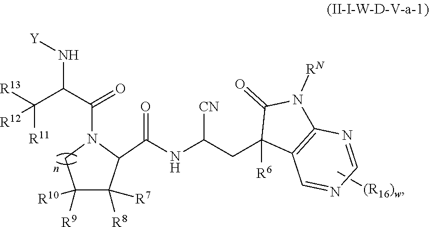

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-V-a-2):

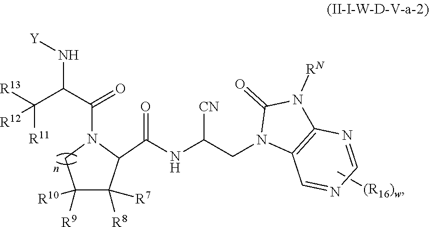

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-VI-a-1):

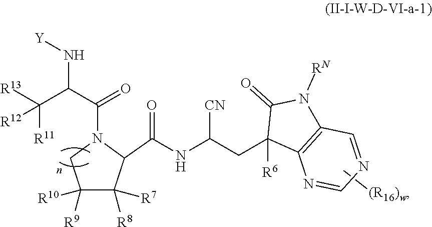

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-VI-a-2):

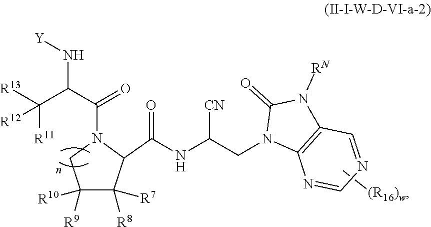

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-VII-a-1):

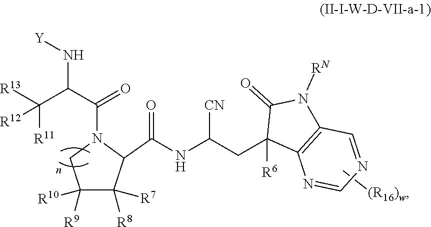

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-VII-a-2):

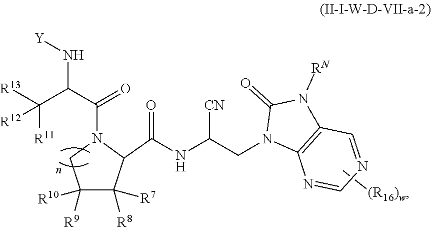

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-A-I-a-1-A):

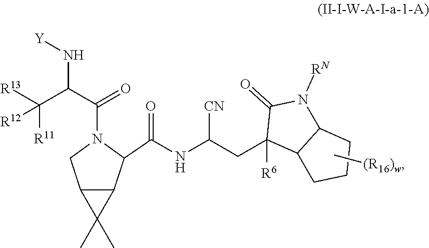

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-A-I-a-1-B):

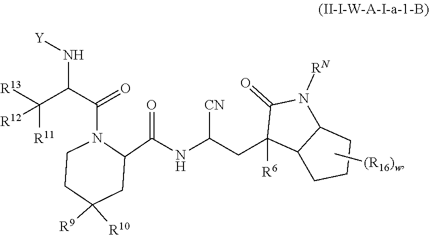

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-A-II-a-1-A):

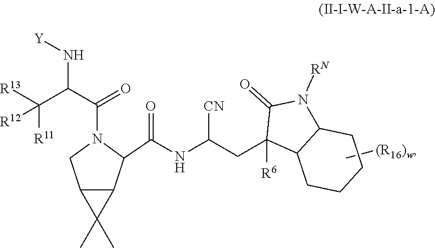

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-A-II-a-1-B):

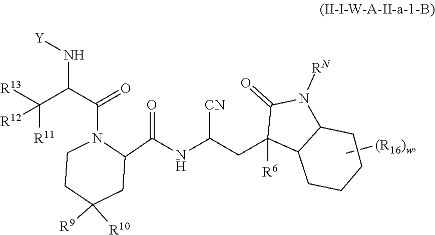

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-A-III-a-1-A):

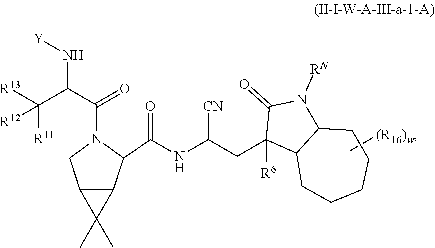

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-A-III-a-1-B):

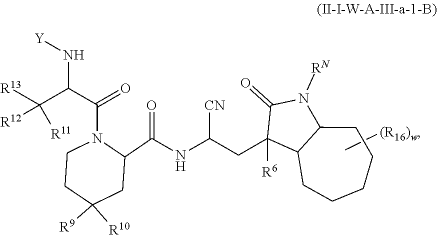

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-A-III-a-2-A):

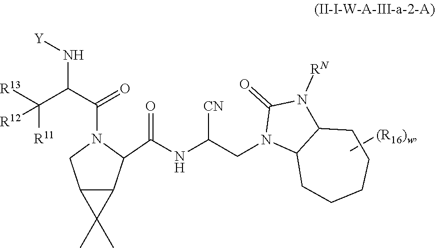

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-A-III-a-2-B):

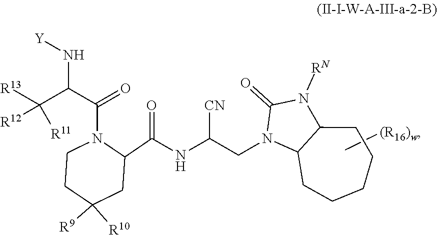

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-B-I-a-1-A):

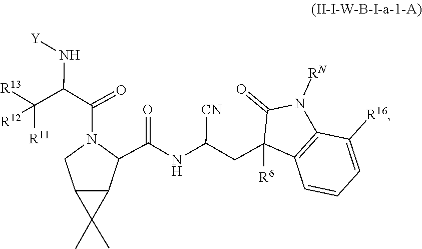

or a pharmaceutically acceptable salt, stereoisomer, solvate, prodrug, isotopic derivative, or tautomer thereof, wherein all variables are as defined herein.

In some embodiments, the compound is of Formula (II-I-W-B-I-a-1-B):

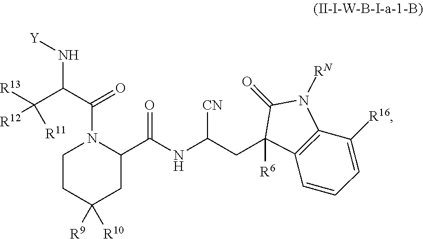

or a pharmaceutically acceptable salt, stereoisomer, solvate, prodrug, isotopic derivative, or tautomer thereof, wherein all variables are as defined herein.

In some embodiments, the compound is of Formula (II-I-W-B-I-a-2-A):

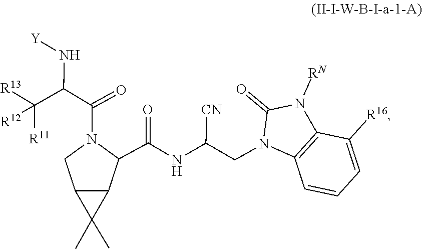

or a pharmaceutically acceptable salt, stereoisomer, solvate, prodrug, isotopic derivative, or tautomer thereof, wherein all variables are as defined herein.

In some embodiments, the compound is of Formula (II-I-W-B-I-a-2-B):

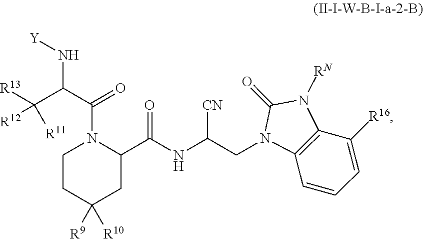

or a pharmaceutically acceptable salt, stereoisomer, solvate, prodrug, isotopic derivative, or tautomer thereof, wherein all variables are as defined herein.

In some embodiments, the compound is of Formula (II-I-W-B-II-a-1-A):

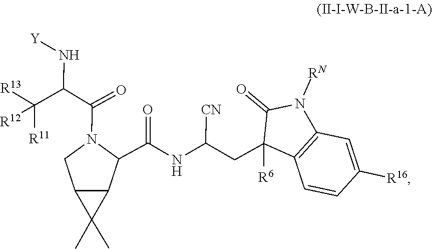

or a pharmaceutically acceptable salt, stereoisomer, solvate, prodrug, isotopic derivative, or tautomer thereof, wherein all variables are as defined herein.

In some embodiments, the compound is of Formula (II-I-W-B-II-a-1-B):

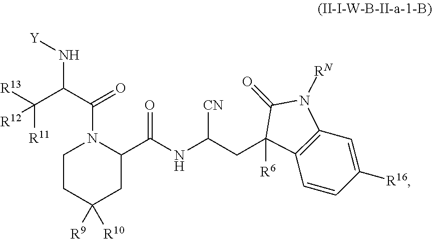

or a pharmaceutically acceptable salt, stereoisomer, solvate, prodrug, isotopic derivative, or tautomer thereof, wherein all variables are as defined herein.

In some embodiments, the compound is of Formula (II-I-W-B-II-a-2-A):

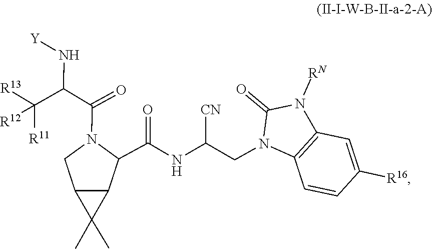

or a pharmaceutically acceptable salt, stereoisomer, solvate, prodrug, isotopic derivative, or tautomer thereof, wherein all variables are as defined herein.

In some embodiments, the compound is of Formula (II-I-W-B-II-a-2-B):

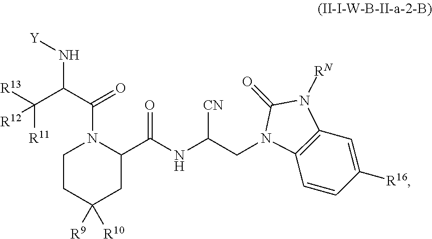

or a pharmaceutically acceptable salt, stereoisomer, solvate, prodrug, isotopic derivative, or tautomer thereof, wherein all variables are as defined herein.

In some embodiments, the compound is of Formula (II-I-W-B-III-a-1-A):

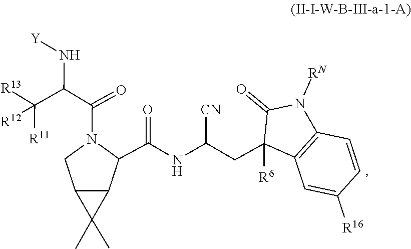

or a pharmaceutically acceptable salt, stereoisomer, solvate, prodrug, isotopic derivative, or tautomer thereof, wherein all variables are as defined herein.

In some embodiments, the compound is of Formula (II-I-W-B-III-a-1-B):

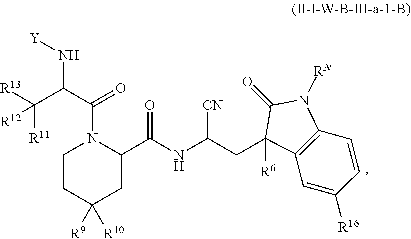

or a pharmaceutically acceptable salt, stereoisomer, solvate, prodrug, isotopic derivative, or tautomer thereof, wherein all variables are as defined herein.

In some embodiments, the compound is of Formula (II-I-W-B-III-a-2-A):

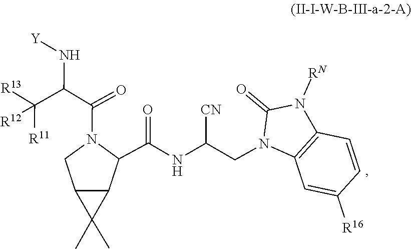

or a pharmaceutically acceptable salt, stereoisomer, solvate, prodrug, isotopic derivative, or tautomer thereof, wherein all variables are as defined herein.

In some embodiments, the compound is of Formula (II-I-W-B-III-a-2-B):

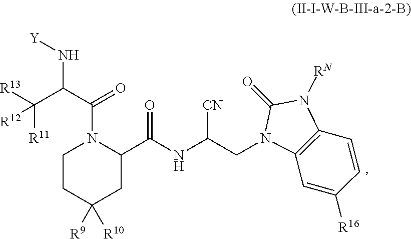

or a pharmaceutically acceptable salt, stereoisomer, solvate, prodrug, isotopic derivative, or tautomer thereof, wherein all variables are as defined herein.

In some embodiments, the compound is of Formula (II-I-W-B-IV-a-1-A):

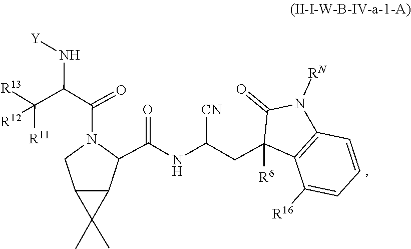

or a pharmaceutically acceptable salt, stereoisomer, solvate, prodrug, isotopic derivative, or tautomer thereof, wherein all variables are as defined herein.

In some embodiments, the compound is of Formula (II-I-W-B-IV-a-1-B):

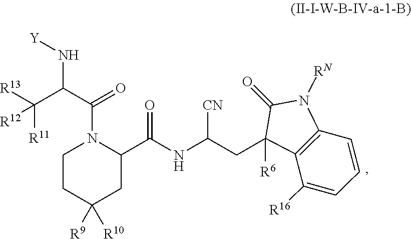

or a pharmaceutically acceptable salt, stereoisomer, solvate, prodrug, isotopic derivative, or tautomer thereof, wherein all variables are as defined herein.

In some embodiments, the compound is of Formula (II-I-W-B-IV-a-2-A):

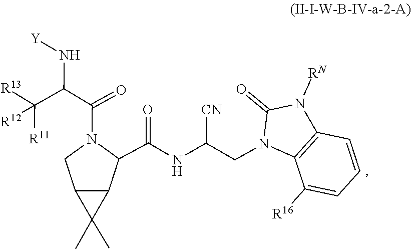

or a pharmaceutically acceptable salt, stereoisomer, solvate, prodrug, isotopic derivative, or tautomer thereof, wherein all variables are as defined herein.

In some embodiments, the compound is of Formula (II-I-W-B-IV-a-2-B):

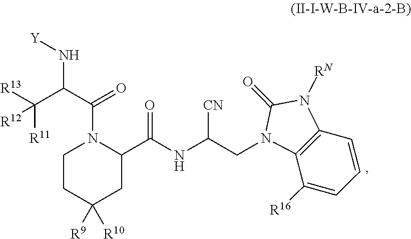

or a pharmaceutically acceptable salt, stereoisomer, solvate, prodrug, isotopic derivative, or tautomer thereof, wherein all variables are as defined herein.

In some embodiments, the compound is of Formula (II-I-W-B-V-a-1-A):

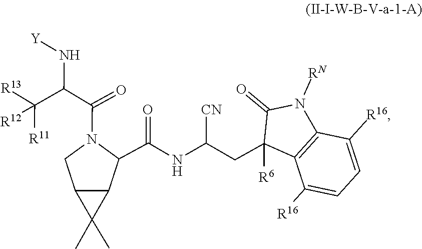

or a pharmaceutically acceptable salt, stereoisomer, solvate, prodrug, isotopic derivative, or tautomer thereof, wherein all variables are as defined herein.

In some embodiments, the compound is of Formula (II-I-W-B-V-a-1-B):

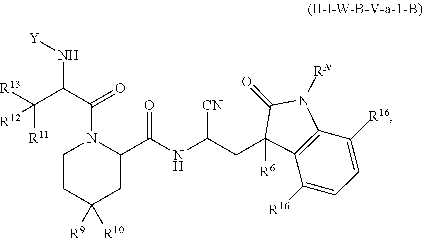

or a pharmaceutically acceptable salt, stereoisomer, solvate, prodrug, isotopic derivative, or tautomer thereof, wherein all variables are as defined herein.

In some embodiments, the compound is of Formula (II-I-W-B-V-a-2-A):

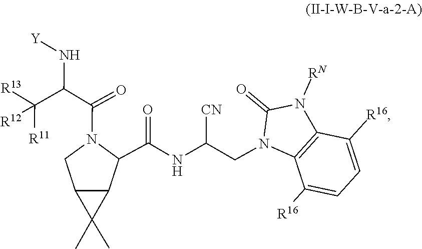

or a pharmaceutically acceptable salt, stereoisomer, solvate, prodrug, isotopic derivative, or tautomer thereof, wherein all variables are as defined herein.

In some embodiments, the compound is of Formula (II-I-W-B-V-a-2-B):

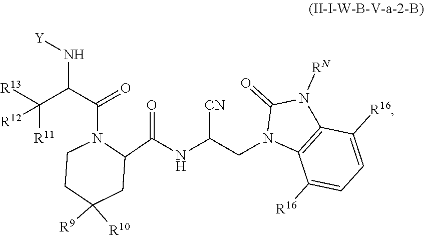

or a pharmaceutically acceptable salt, stereoisomer, solvate, prodrug, isotopic derivative, or tautomer thereof, wherein all variables are as defined herein.

In some embodiments, the compound is of Formula (II-I-W-B-VI-a-1-A):

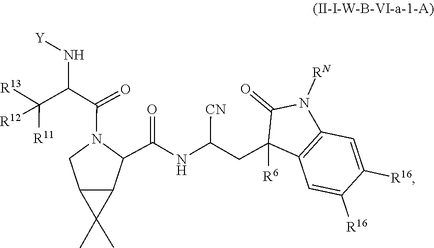

or a pharmaceutically acceptable salt, stereoisomer, solvate, prodrug, isotopic derivative, or tautomer thereof, wherein all variables are as defined herein.

In some embodiments, the compound is of Formula (II-I-W-B-VI-a-1-B):

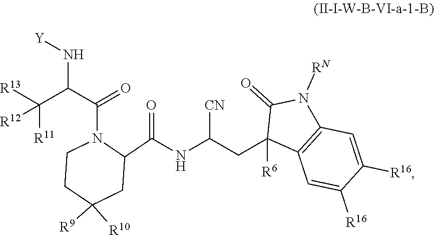

or a pharmaceutically acceptable salt, stereoisomer, solvate, prodrug, isotopic derivative, or tautomer thereof, wherein all variables are as defined herein.

In some embodiments, the compound is of Formula (II-I-W-B-VI-a-2-A):

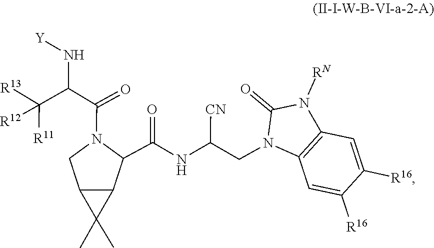

or a pharmaceutically acceptable salt, stereoisomer, solvate, prodrug, isotopic derivative, or tautomer thereof, wherein all variables are as defined herein.

In some embodiments, the compound is of Formula (II-I-W-B-VI-a-2-B):

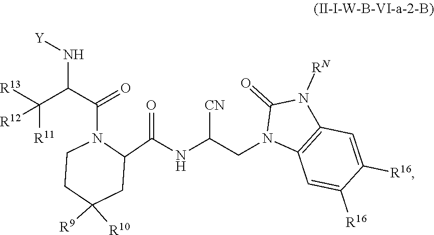

or a pharmaceutically acceptable salt, stereoisomer, solvate, prodrug, isotopic derivative, or tautomer thereof, wherein all variables are as defined herein.

In some embodiments, the compound is of Formula (II-I-W-C-I-a-1-A):

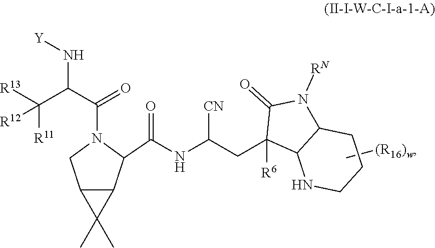

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-I-a-1-B):

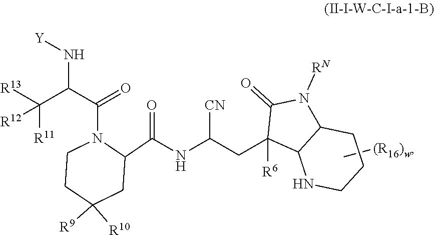

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-I-a-2-A):

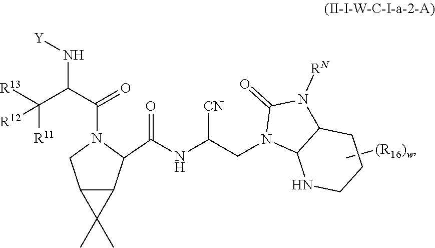

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-I-a-2-B):

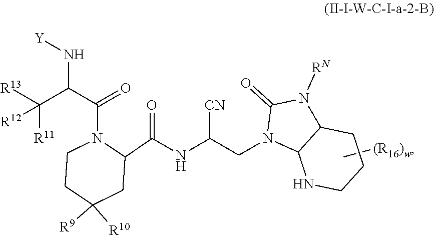

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-II-a-1-A):

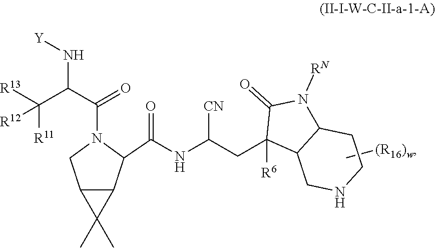

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-II-a-1-B):

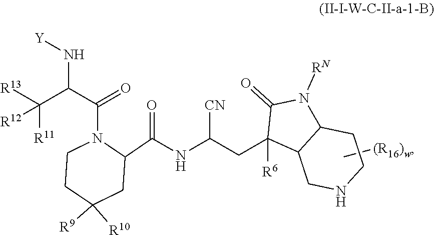

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-II-a-2-A):

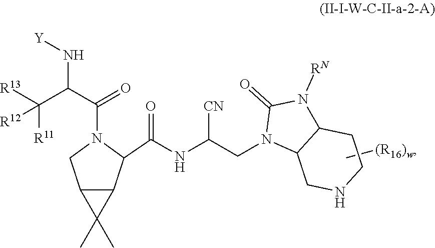

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-II-a-2-B):

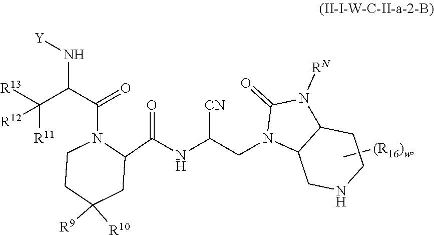

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-III-a-1-A):

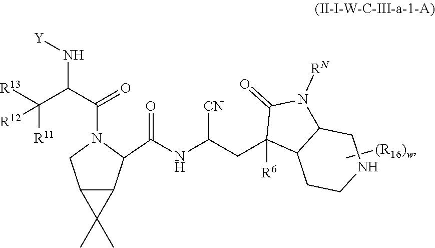

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-III-a-1-B):

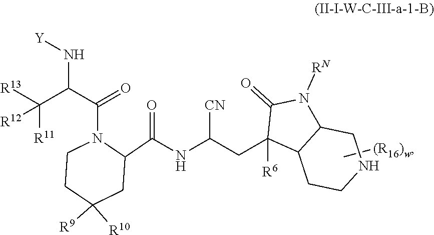

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-III-a-2-A):

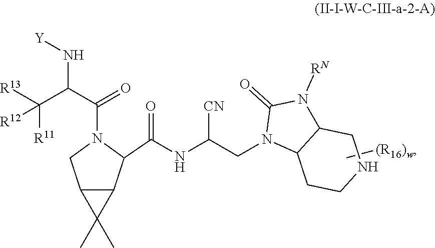

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-III-a-2-B):

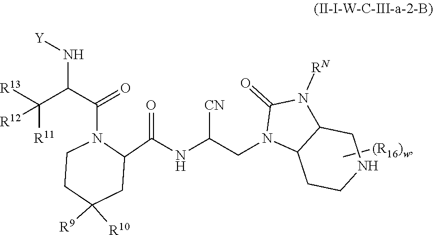

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-IV-a-1-A):

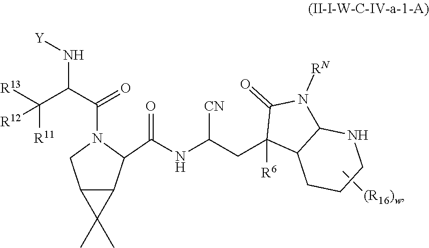

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-IV-a-1-B):

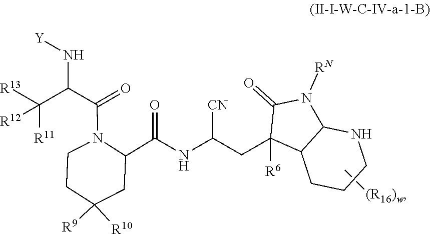

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-IV-a-2-A):

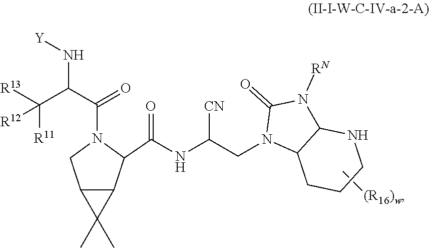

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-IV-a-2-B):

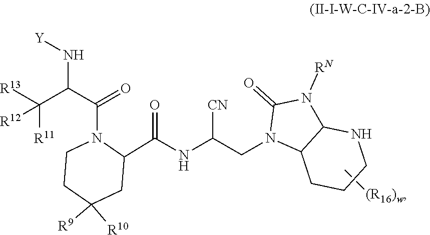

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-V-a-1-A):

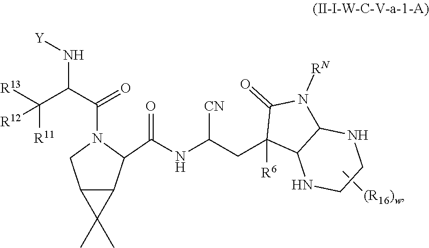

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-V-a-1-B):

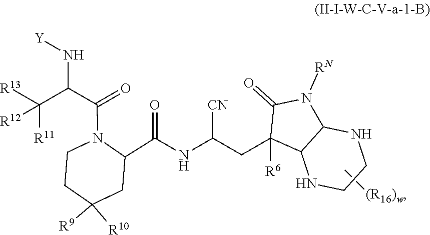

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-V-a-2-A):

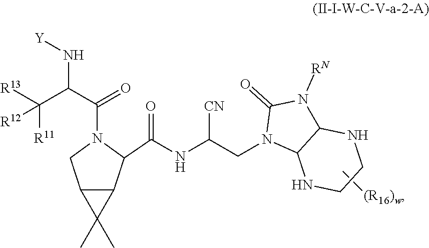

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-V-a-2-B):

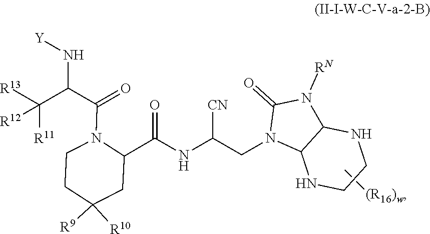

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-VI-a-1-A):

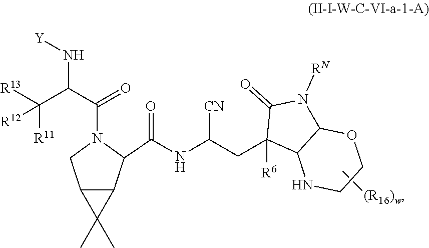

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-VI-a-1-B):

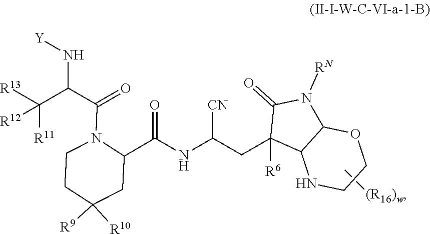

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-VI-a-2-A):

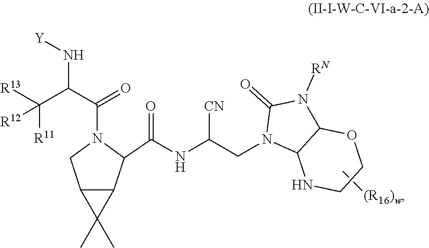

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-VI-a-2-B):

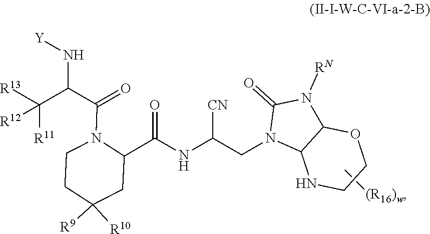

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-VII-a-1-A):

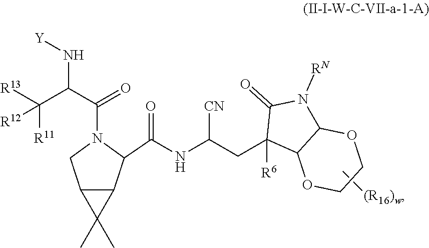

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-VII-a-1-B):

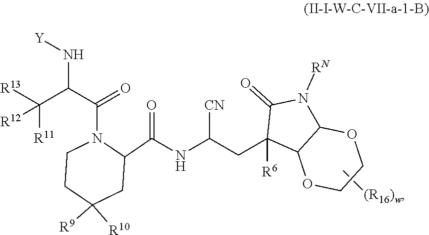

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-VII-a-2-A):

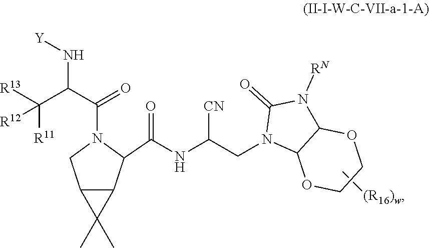

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-VII-a-2-B):

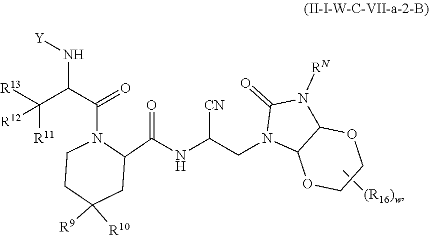

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-VIII-a-1-A):

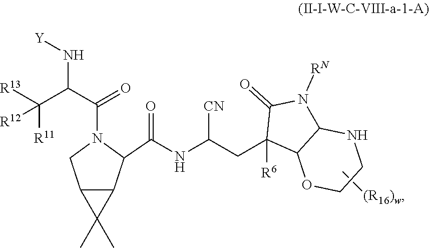

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-VIII-a-1-B):

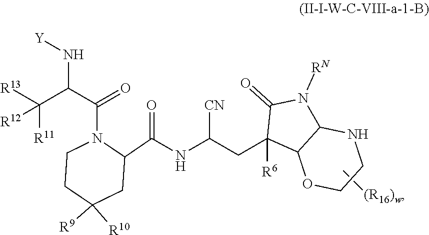

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-VIII-a-2-A):

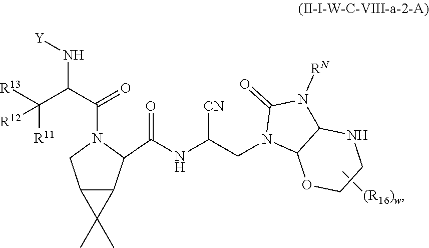

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-VIII-a-2-B):

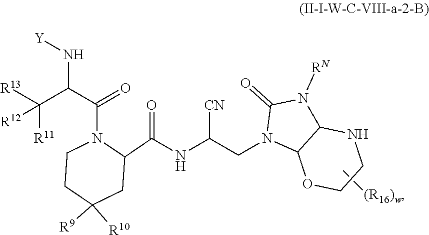

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-IX-a-1-A):

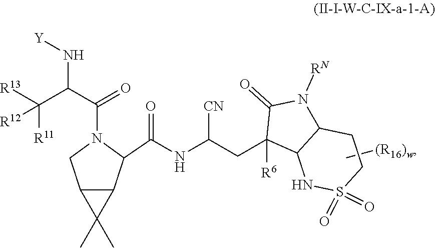

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-IX-a-1-B):

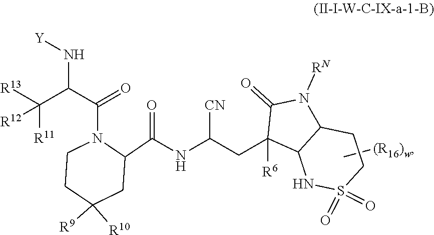

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-IX-a-2-A):

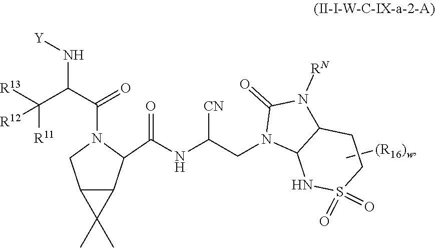

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-IX-a-2-B):

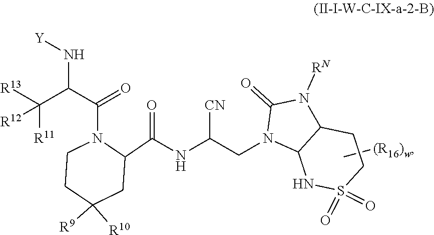

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-X-a-1-A):

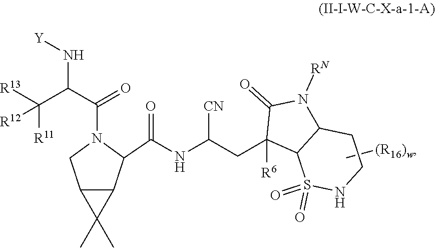

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-X-a-1-B):

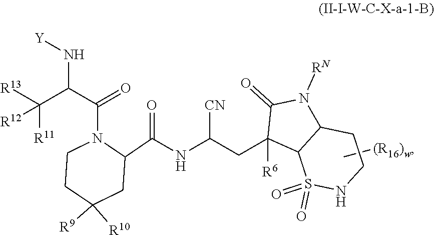

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-X-a-2-A):

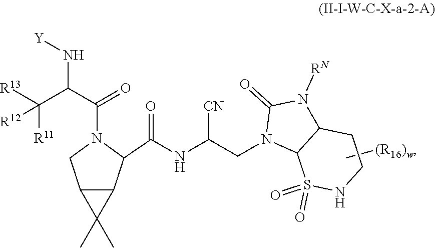

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-C-X-a-2-B):

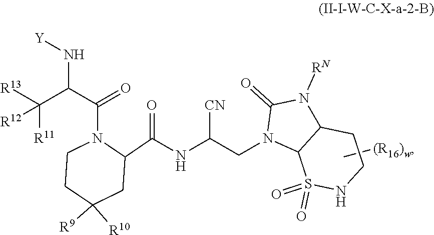

or a pharmaceutically acceptable salt, stereoisomer, solvate, prodrug, isotopic derivative, or tautomer thereof, wherein w is an integer selected from 0, 1, 2, 3, and 4; and all other variables are as defined herein.

In some embodiments, the compound is of Formula (II-I-W-D-I-a-1-A):

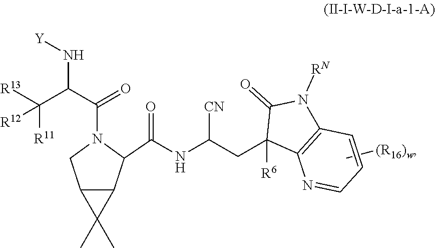

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-I-a-1-B):

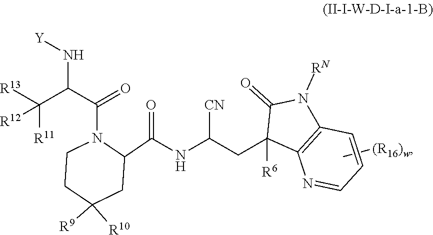

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-I-a-2-A):

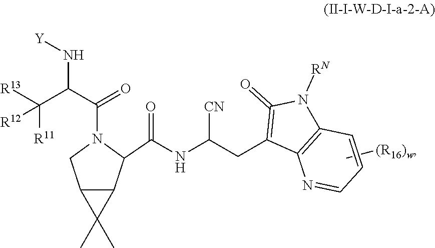

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-I-a-2-B):

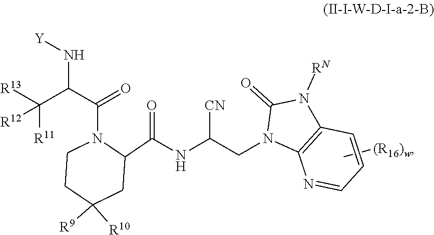

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-II-a-1-A):

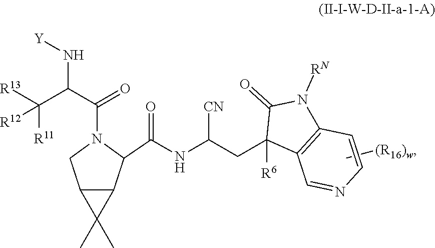

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-II-a-1-B):

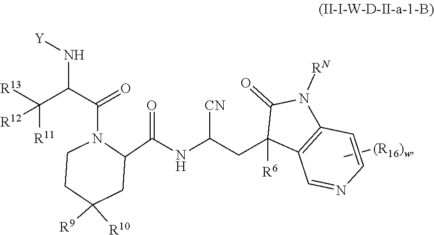

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-II-a-2-A):

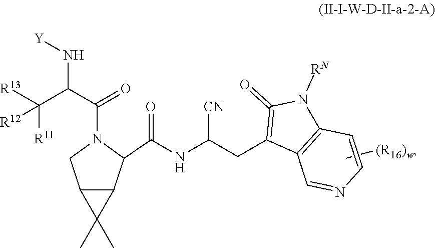

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-II-a-2-B):

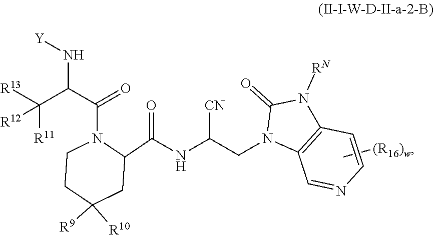

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-III-a-1-A):

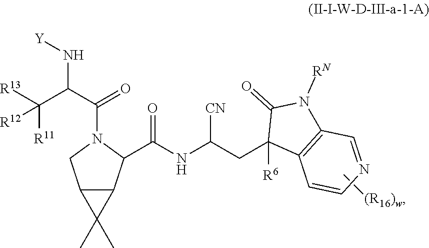

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-III-a-1-B):

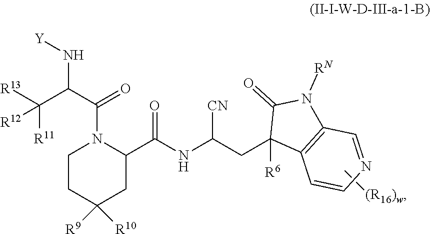

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-III-a-2-A):

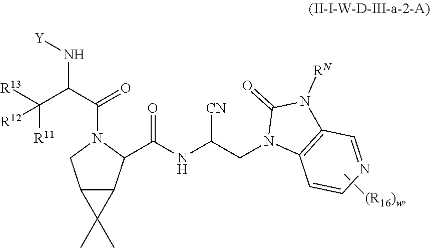

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-III-a-2-B):

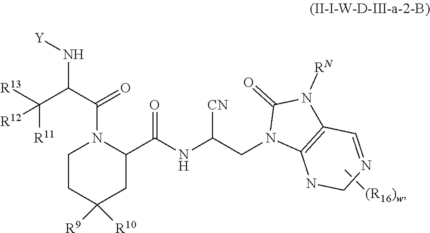

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-IV-a-1-A):

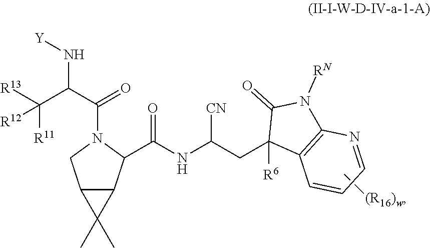

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-IV-a-1-B):

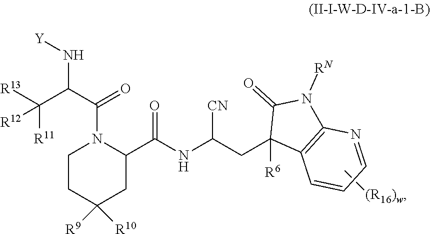

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-IV-a-2-A):

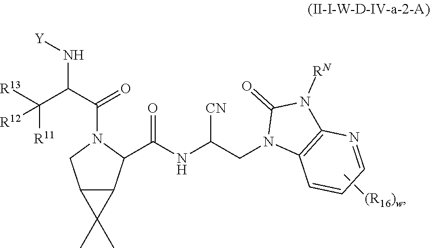

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-IV-a-2-B):

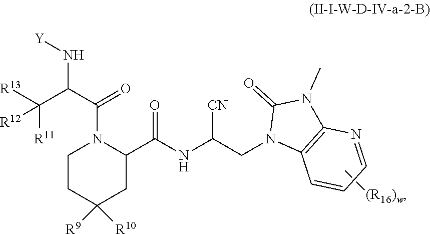

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-V-a-1-A):

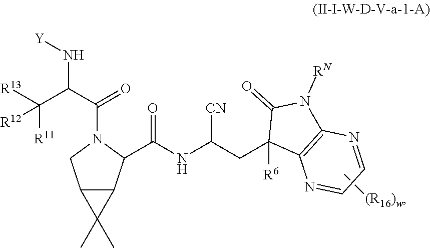

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-V-a-1-B):

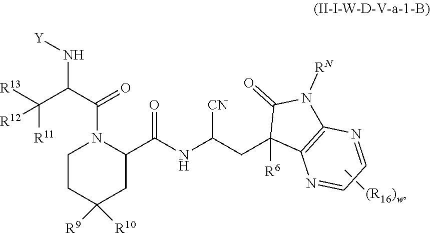

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-V-a-2-A):

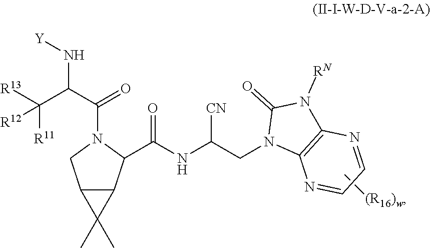

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-V-a-2-B):

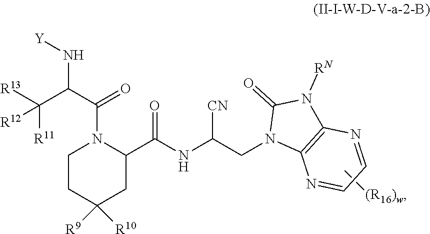

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-VI-a-1-A):

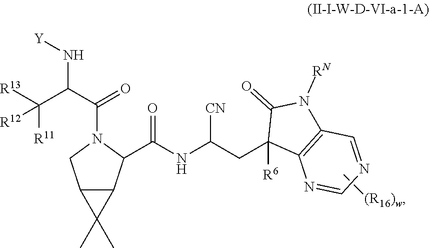

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-VI-a-1-B):

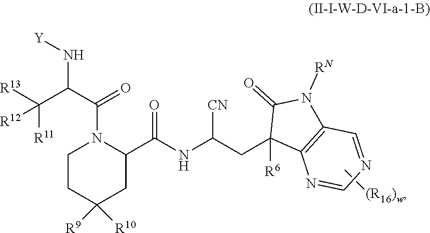

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-VI-a-2-A):

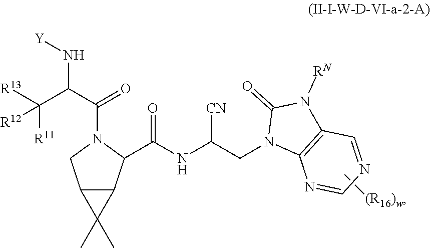

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-VI-a-2-B):

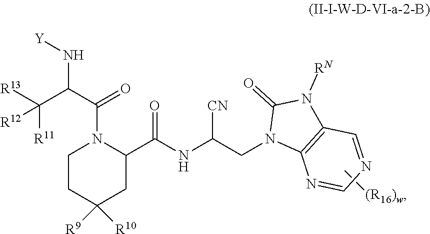

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-VII-a-1-A):

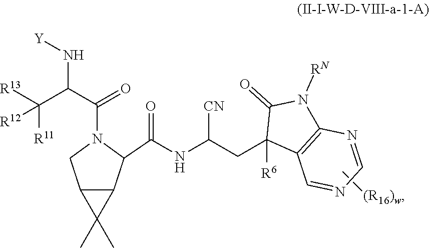

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-VII-a-1-B):

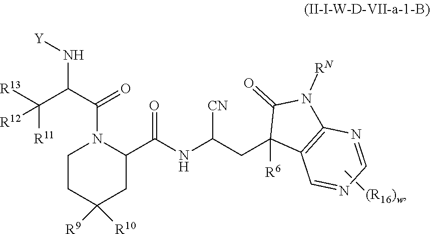

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-VII-a-2-A):

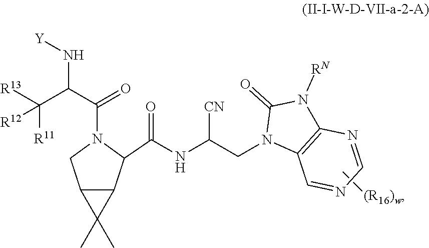

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (II-I-W-D-VII-a-2-B):

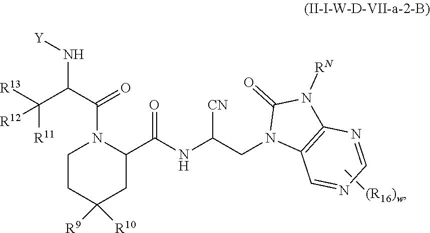

or a pharmaceutically acceptable salt, stereoisomer, solvate, prodrug, isotopic derivative, or tautomer thereof, wherein w is an integer selected from 0, 1, and 2; and all other variables are as defined herein.

In some embodiments, the compound is of Formula (III-I):

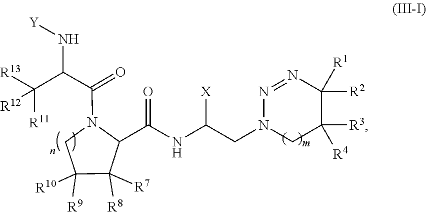

or a pharmaceutically acceptable salt, stereoisomer, solvate, prodrug, isotopic derivative, or tautomer thereof, wherein all variables are as defined herein.

In some embodiments, the compound is of Formula (III-I-I):

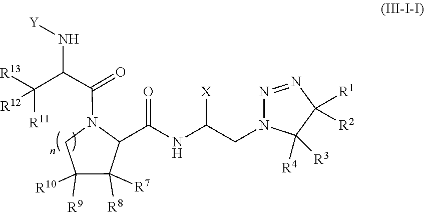

or a pharmaceutically acceptable salt, stereoisomer, solvate, prodrug, isotopic derivative, or tautomer thereof, wherein all variables are as defined herein.

In some embodiments, the compound is of Formula (III-I-I-A):

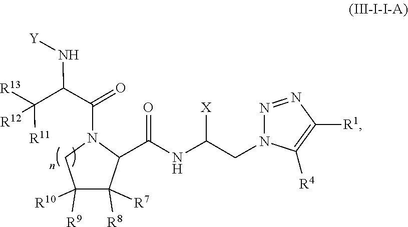

or a pharmaceutically acceptable salt, stereoisomer, solvate, prodrug, isotopic derivative, or tautomer thereof, wherein all variables are as defined herein.

In some embodiments, the compound is of Formula (III-I-I-A-A):

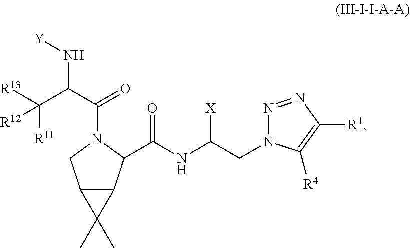

or a pharmaceutically acceptable salt, stereoisomer, solvate, prodrug, isotopic derivative, or tautomer thereof, wherein all variables are as defined herein.

In some embodiments, the compound is of Formula (III-I-I-A-A*):

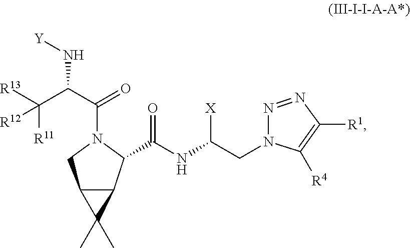

or a pharmaceutically acceptable salt, stereoisomer, solvate, prodrug, isotopic derivative, or tautomer thereof, wherein all variables are as defined herein.

In some embodiments, the compound is of Formula (III-I-I-A-A-A):

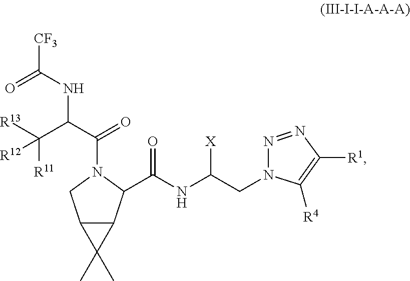

or a pharmaceutically acceptable salt, stereoisomer, solvate, prodrug, isotopic derivative, or tautomer thereof, wherein all variables are as defined herein.

In some embodiments, the compound is of Formula (III-I-I-A-A-A*):

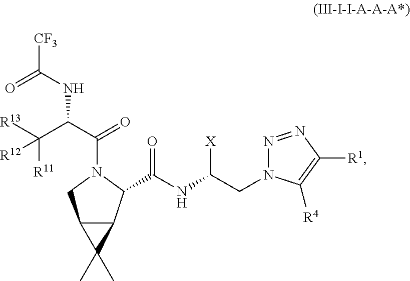

or a pharmaceutically acceptable salt, stereoisomer, solvate, prodrug, isotopic derivative, or tautomer thereof, wherein all variables are as defined herein.

In some embodiments, the compound is of Formula (III-I-I-A-A-A-I):

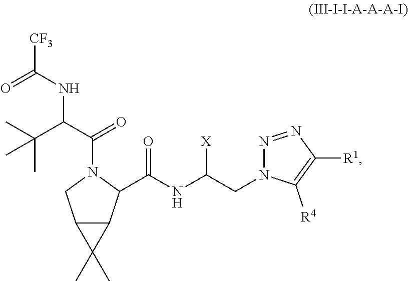

or a pharmaceutically acceptable salt, stereoisomer, solvate, prodrug, isotopic derivative, or tautomer thereof, wherein X is selected from —C(O)NH₂ and —CN and all other variables are as defined herein.

In some embodiments, the compound is of Formula (III-I-I-A-A-A-I*):

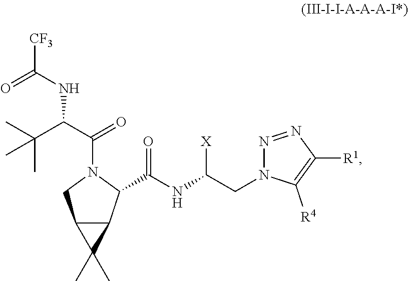

or a pharmaceutically acceptable salt, stereoisomer, solvate, prodrug, isotopic derivative, or tautomer thereof, wherein X is selected from —C(O)NH₂ and —CN and all other variables are as defined herein.

In some embodiments, the compound is of Formula (III-I-I-A-A-A-I-a):

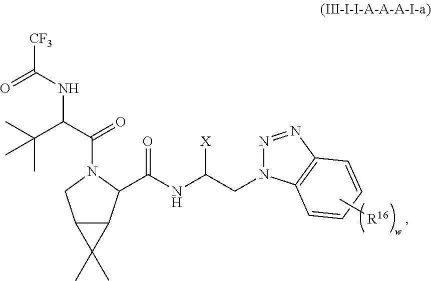

or a pharmaceutically acceptable salt, stereoisomer, solvate, prodrug, isotopic derivative, or tautomer thereof, wherein w is integer selected from 0, 1, 2, 3, 4; X is selected from —C(O)NH₂ and —CN and all other variables are as defined herein.

In some embodiments, the compound is of Formula (III-I-I-A-A-A-I-a*):

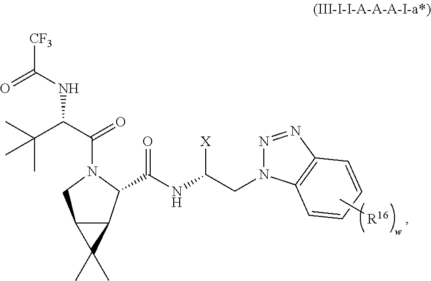

or a pharmaceutically acceptable salt, stereoisomer, solvate, prodrug, isotopic derivative, or tautomer thereof, wherein w is integer selected from 0, 1, 2, 3, 4; X is selected from —C(O)NH₂ and —CN and all other variables are as defined herein.

In some embodiments, the compound is of Formula (III-II):

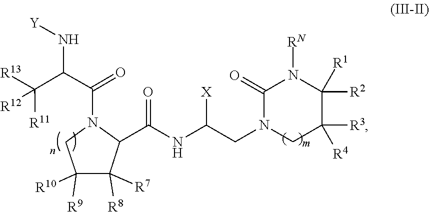

or a pharmaceutically acceptable salt, stereoisomer, solvate, prodrug, isotopic derivative, or tautomer thereof, wherein all variables are as defined herein.

In some embodiments, the compound is of Formula (III-II-I):

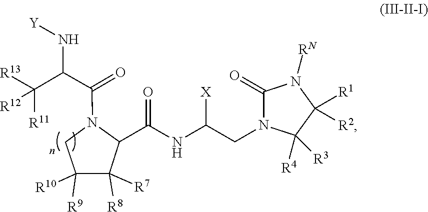

or a pharmaceutically acceptable salt, stereoisomer, solvate, prodrug, isotopic derivative, or tautomer thereof, wherein all variables are as defined herein.

In some embodiments, the compound is of Formula (III-II-I-A):

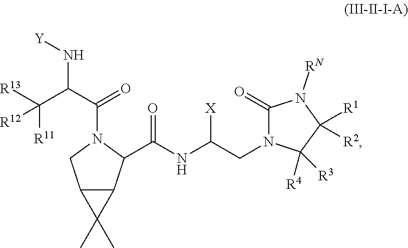

or a pharmaceutically acceptable salt, stereoisomer, solvate, prodrug, isotopic derivative, or tautomer thereof, wherein all variables are as defined herein.

In some embodiments, the compound is of Formula (III-II-I-A*):

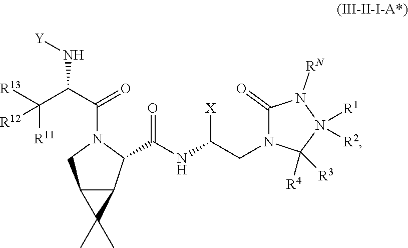

or a pharmaceutically acceptable salt, stereoisomer, solvate, prodrug, isotopic derivative, or tautomer thereof, wherein all variables are as defined herein.

In some embodiments, the compound is of Formula (III-II-I-A-A):

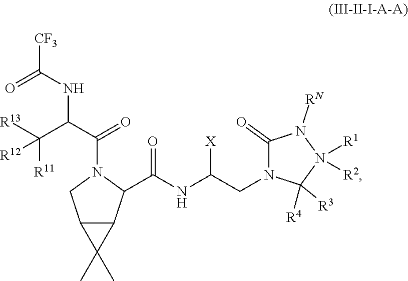

or a pharmaceutically acceptable salt, stereoisomer, solvate, prodrug, isotopic derivative, or tautomer thereof, wherein all variables are as defined herein.

In some embodiments, the compound is of Formula (III-II-I-A-A*):

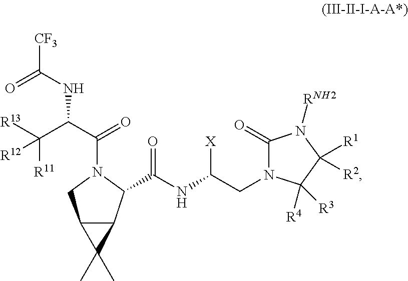

or a pharmaceutically acceptable salt, stereoisomer, solvate, prodrug, isotopic derivative, or tautomer thereof, wherein all variables are as defined herein.

In some embodiments, the compound is of Formula (III-II-I-A-A-I):

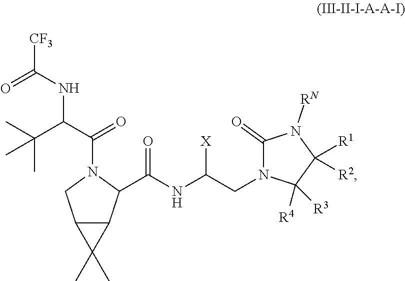

or a pharmaceutically acceptable salt, stereoisomer, solvate, prodrug, isotopic derivative, or tautomer thereof, wherein X is selected from —C(O)NH₂ and —CN and all other variables are as defined herein.

In some embodiments, the compound is of Formula (III-II-I-A-A-I*):

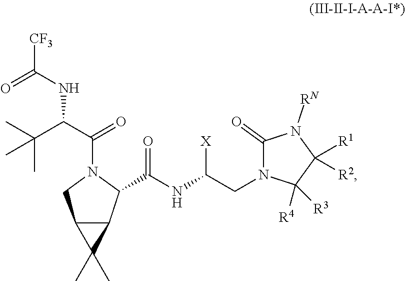

or a pharmaceutically acceptable salt, stereoisomer, solvate, prodrug, isotopic derivative, or tautomer thereof, wherein X is selected from —C(O)NH₂ and —CN and all other variables are as defined herein.

In some embodiments, the compound is of Formula (III-II-I-A-A-I-B):

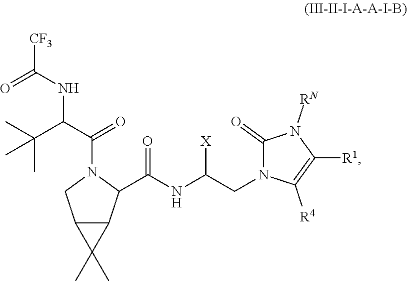

or a pharmaceutically acceptable salt, stereoisomer, solvate, prodrug, isotopic derivative, or tautomer thereof, wherein X is selected from —C(O)NH₂ and —CN and all other variables are as defined herein.

In some embodiments, the compound is of Formula (III-II-I-A-A-I-B*):

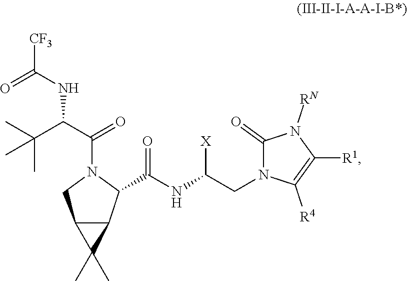

or a pharmaceutically acceptable salt, stereoisomer, solvate, prodrug, isotopic derivative, or tautomer thereof, wherein X is selected from —C(O)NH₂ and —CN and all other variables are as defined herein.

In some embodiments, the compound is of Formula (III-II-I-A-A-I-B-1):

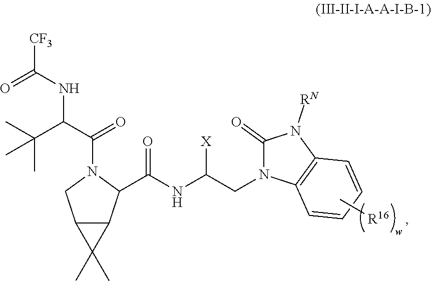

or a pharmaceutically acceptable salt, stereoisomer, solvate, prodrug, isotopic derivative, or tautomer thereof, wherein w is an integer selected from 0, 1, 2, 3, 4; X is selected from —C(O)NH₂ and —CN and all other variables are as defined herein.

In some embodiments, the compound is of Formula (III-II-I-A-A-I-B-1*):

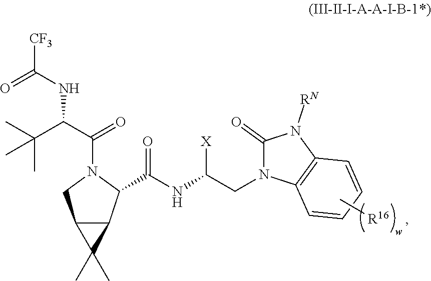

or a pharmaceutically acceptable salt, stereoisomer, solvate, prodrug, isotopic derivative, or tautomer thereof, wherein w is an integer selected from 0, 1, 2, 3, 4; X is selected from —C(O)NH₂ and —CN and all other variables are as defined herein.

In some embodiments, the compound is of Formula (IV-I):

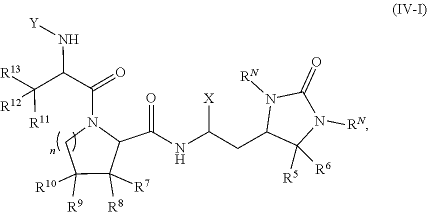

or a pharmaceutically acceptable salt, stereoisomer, solvate, prodrug, isotopic derivative, or tautomer thereof, wherein all variables are as defined herein.

In some embodiments, the compound is of Formula (IV-II):

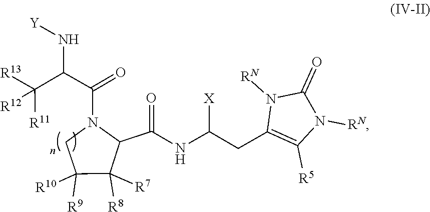

or a pharmaceutically acceptable salt, stereoisomer, solvate, prodrug, isotopic derivative, or tautomer thereof, wherein all variables are as defined herein.

In some embodiments, the compound is of Formula (IV-III):

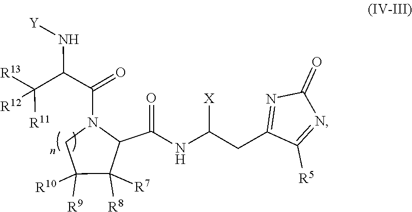

or a pharmaceutically acceptable salt, stereoisomer, solvate, prodrug, isotopic derivative, or tautomer thereof, wherein all variables are as defined herein.

In some embodiments, the compound is of Formula (IV-I-A):

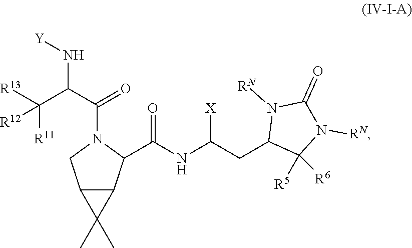

or a pharmaceutically acceptable salt, stereoisomer, solvate, prodrug, isotopic derivative, or tautomer thereof, wherein all variables are as defined herein.

In some embodiments, the compound is of Formula (IV-I-A*):

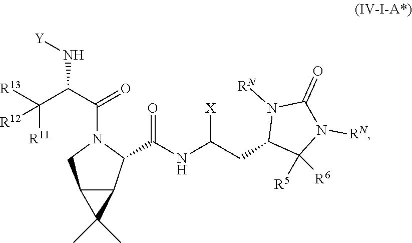

or a pharmaceutically acceptable salt, stereoisomer, solvate, prodrug, isotopic derivative, or tautomer thereof, wherein all variables are as defined herein.

In some embodiments, the compound is of Formula (IV-II-A):

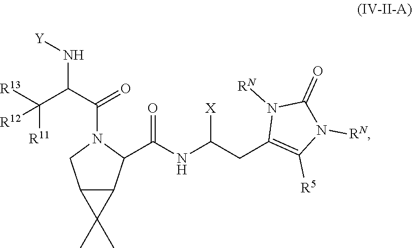

or a pharmaceutically acceptable salt, stereoisomer, solvate, prodrug, isotopic derivative, or tautomer thereof, wherein all variables are as defined herein.

In some embodiments, the compound is of Formula (IV-II-A*):

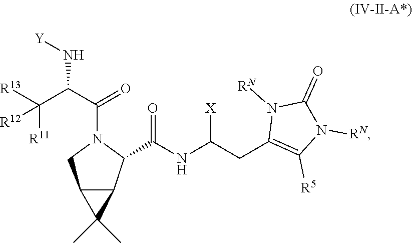

or a pharmaceutically acceptable salt, stereoisomer, solvate, prodrug, isotopic derivative, or tautomer thereof, wherein all variables are as defined herein.

In some embodiments, the compound is of Formula (IV-III-A):

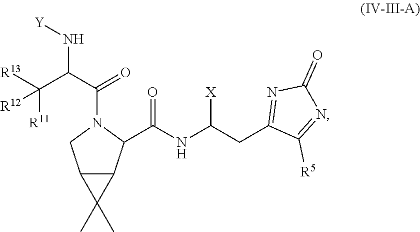

or a pharmaceutically acceptable salt, stereoisomer, solvate, prodrug, isotopic derivative, or tautomer thereof, wherein all variables are as defined herein.

In some embodiments, the compound is of Formula (IV-III-A*):

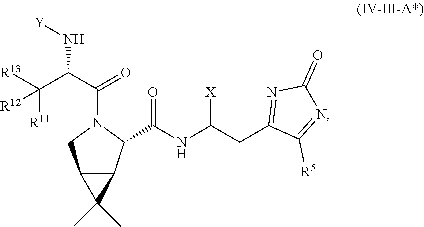

or a pharmaceutically acceptable salt, stereoisomer, solvate, prodrug, isotopic derivative, or tautomer thereof, wherein all variables are as defined herein.

In some embodiments, the compound is of Formula (IV-I-A-A):

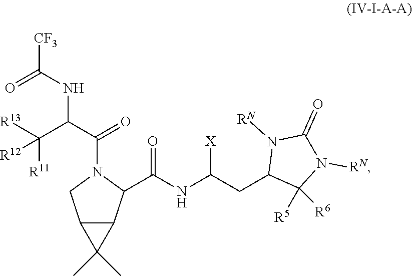

or a pharmaceutically acceptable salt, stereoisomer, solvate, prodrug, isotopic derivative, or tautomer thereof, wherein all variables are as defined herein.

In some embodiments, the compound is of Formula (IV-I-A-A*):

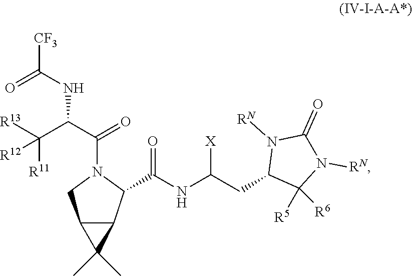

or a pharmaceutically acceptable salt, stereoisomer, solvate, prodrug, isotopic derivative, or tautomer thereof, wherein all variables are as defined herein.

In some embodiments, the compound is of Formula (IV-II-A-A):

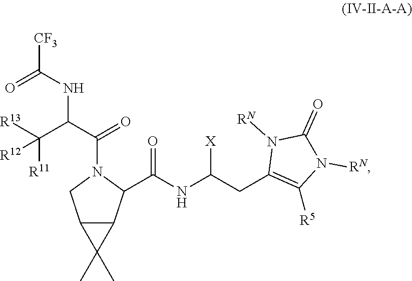

or a pharmaceutically acceptable salt, stereoisomer, solvate, prodrug, isotopic derivative, or tautomer thereof, wherein all variables are as defined herein.

In some embodiments, the compound is of Formula (IV-II-A-A*):

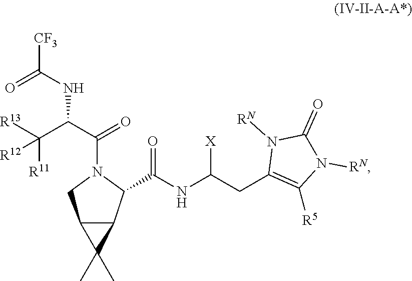

or a pharmaceutically acceptable salt, stereoisomer, solvate, prodrug, isotopic derivative, or tautomer thereof, wherein all variables are as defined herein.

In some embodiments, the compound is of Formula (IV-III-A-A):

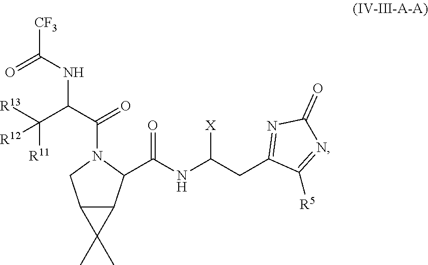

or a pharmaceutically acceptable salt, stereoisomer, solvate, prodrug, isotopic derivative, or tautomer thereof, wherein all variables are as defined herein.

In some embodiments, the compound is of Formula (IV-III-A-A*):

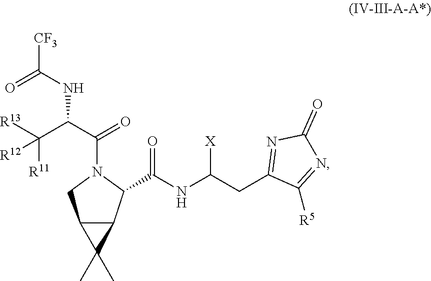

or a pharmaceutically acceptable salt, stereoisomer, solvate, prodrug, isotopic derivative, or tautomer thereof, wherein all variables are as defined herein.

In some embodiments, the compound is of Formula (IV-I-A-A-I):

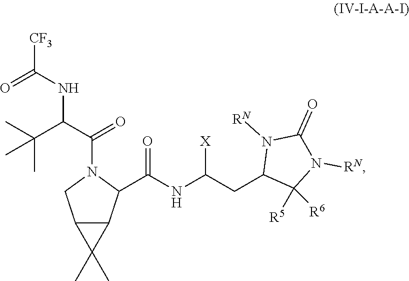

or a pharmaceutically acceptable salt, stereoisomer, solvate, prodrug, isotopic derivative, or tautomer thereof, wherein X is selected from —C(O)NH₂ and —CN and all other variables are as defined herein.

In some embodiments, the compound is of Formula (IV-I-A-A-I*):

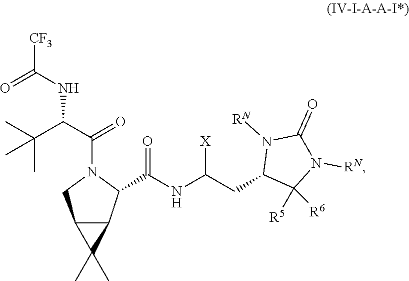

or a pharmaceutically acceptable salt, stereoisomer, solvate, prodrug, isotopic derivative, or tautomer thereof, wherein X is selected from —C(O)NH₂ and —CN and all other variables are as defined herein.

In some embodiments, the compound is of Formula (IV-II-A-A-I):

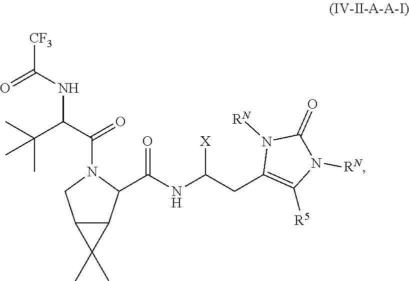

or a pharmaceutically acceptable salt, stereoisomer, solvate, prodrug, isotopic derivative, or tautomer thereof, wherein X is selected from —C(O)NH₂ and —CN and all other variables are as defined herein.

In some embodiments, the compound is of Formula (IV-II-A-A-I*):

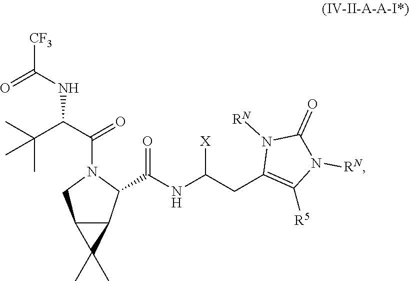

or a pharmaceutically acceptable salt, stereoisomer, solvate, prodrug, isotopic derivative, or tautomer thereof, wherein X is selected from —C(O)NH₂ and —CN and all other variables are as defined herein.

In some embodiments, the compound is of Formula (IV-III-A-A-I):

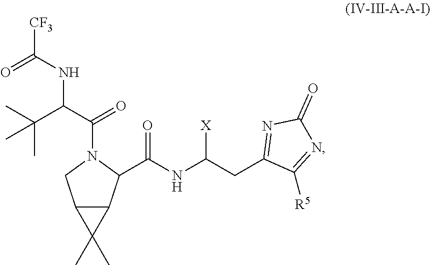

or a pharmaceutically acceptable salt, stereoisomer, solvate, prodrug, isotopic derivative, or tautomer thereof, wherein X is selected from —C(O)NH₂ and —CN and all other variables are as defined herein.

In some embodiments, the compound is of Formula (IV-III-A-A-I*):

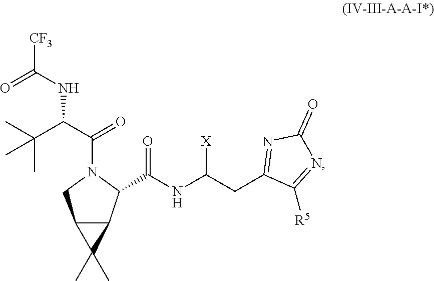

or a pharmaceutically acceptable salt, stereoisomer, solvate, prodrug, isotopic derivative, or tautomer thereof, wherein X is selected from —C(O)NH₂ and —CN and all other variables are as defined herein.

In some embodiments, the compound is of Formula (V):

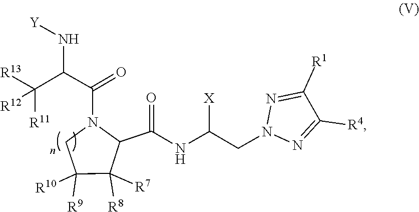

or a pharmaceutically acceptable salt, stereoisomer, solvate, prodrug, isotopic derivative, or tautomer thereof, wherein all variables are as defined herein.

In some embodiments, the compound is of Formula (V-I):

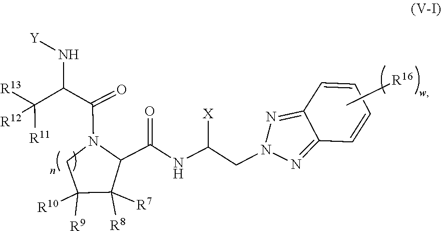

or a pharmaceutically acceptable salt, stereoisomer, solvate, prodrug, isotopic derivative, or tautomer thereof, wherein w is an integer selected from 0, 1, 2, 3, 4 and all other variables are as defined herein.

In some embodiments, the compound is of Formula (V-A):

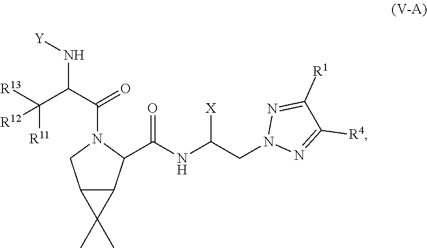

or a pharmaceutically acceptable salt, stereoisomer, solvate, prodrug, isotopic derivative, or tautomer thereof, wherein all variables are as defined herein.

In some embodiments, the compound is of Formula (V-I-A):

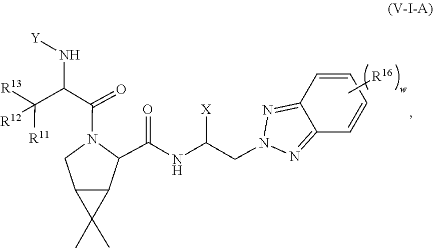

or a pharmaceutically acceptable salt, stereoisomer, solvate, prodrug, isotopic derivative, or tautomer thereof, wherein w is an integer selected from 0, 1, 2, 3, 4 and all other variables are as defined herein.

In some embodiments, the compound is of Formula (V-A-A):

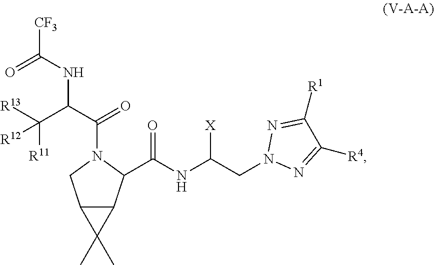

or a pharmaceutically acceptable salt, stereoisomer, solvate, prodrug, isotopic derivative, or tautomer thereof, wherein all variables are as defined herein.

In some embodiments, the compound is of Formula (V-I-A-A):

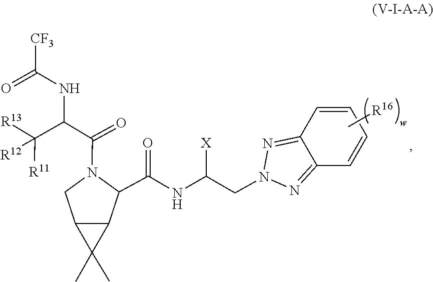

or a pharmaceutically acceptable salt, stereoisomer, solvate, prodrug, isotopic derivative, or tautomer thereof, wherein w is an integer selected from 0, 1, 2, 3, 4 and all other variables are as defined herein.

In some embodiments, the compound is of Formula (V-A-A*):

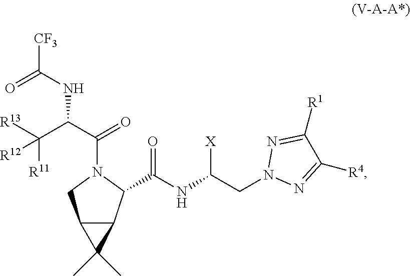

or a pharmaceutically acceptable salt, stereoisomer, solvate, prodrug, isotopic derivative, or tautomer thereof, wherein all variables are as defined herein.

In some embodiments, the compound is of Formula (V-I-A-A*):

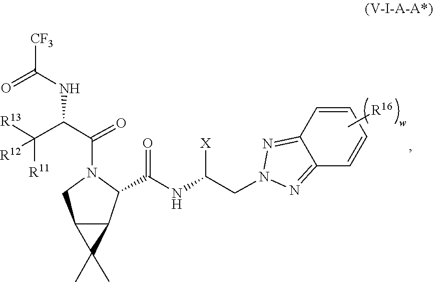

or a pharmaceutically acceptable salt, stereoisomer, solvate, prodrug, isotopic derivative, or tautomer thereof, wherein w is an integer selected from 0, 1, 2, 3, 4 and all other variables are as defined herein.

In some embodiments, the compound is of Formula (V-A-A-I):

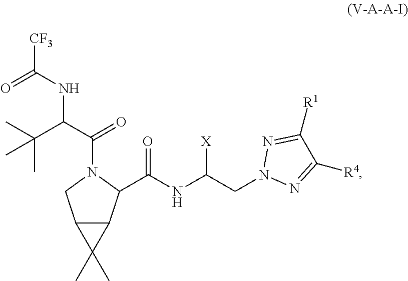

or a pharmaceutically acceptable salt, stereoisomer, solvate, prodrug, isotopic derivative, or tautomer thereof, wherein X is selected from —C(O)NH₂ and —CN and all variables are as defined herein.

In some embodiments, the compound is of Formula (V-I-A-A-I):

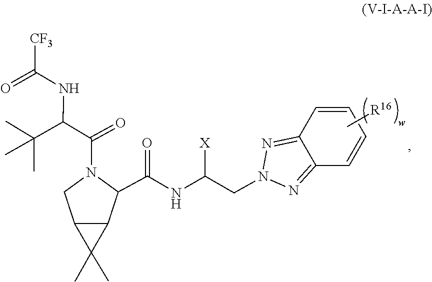

or a pharmaceutically acceptable salt, stereoisomer, solvate, prodrug, isotopic derivative, or tautomer thereof, wherein X is selected from —C(O)NH₂ and —CN; w is an integer selected from 0, 1, 2, 3, 4 and all other variables are as defined herein.

In some embodiments, the compound is of Formula (V-A-A-I*):

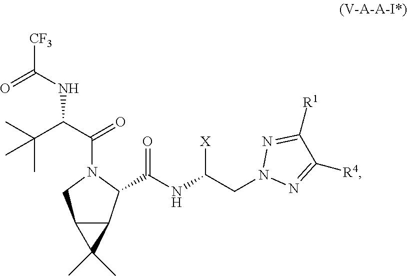

or a pharmaceutically acceptable salt, stereoisomer, solvate, prodrug, isotopic derivative, or tautomer thereof, wherein X is selected from —C(O)NH₂ and —CN and all variables are as defined herein.

In some embodiments, the compound is of Formula (V-I-A-A-I*):

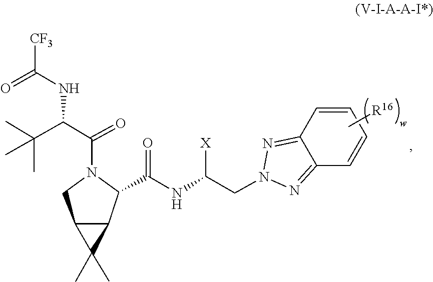

or a pharmaceutically acceptable salt, stereoisomer, solvate, prodrug, isotopic derivative, or tautomer thereof, wherein X is selected from —C(O)NH₂ and —CN; w is an integer selected from 0, 1, 2, 3, 4 and all other variables are as defined herein.

A suitable pharmaceutically acceptable salt of a compound of the disclosure is, for example, an acid-addition salt of a compound of the disclosure, which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the disclosure which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, diethylamine, trimethylamine, piperidine, morpholine or tris-(2-hydroxyethyl)amine.

It will be understood that the compounds of any one of the Formulae disclosed herein and any pharmaceutically acceptable salts thereof, comprise stereoisomers, mixtures of stereoisomers, polymorphs of all isomeric forms of said compounds.

In some embodiments, the compound is selected from the compounds described in Table 1 and pharmaceutically acceptable salts, stereoisomers, solvates, prodrugs, isotopic derivatives, or tautomers thereof.

In some embodiments, the compound is selected from the compounds described in Table 1 and prodrugs and pharmaceutically acceptable salts thereof.

In some embodiments, the compound is selected from the compounds described in Table 1 and pharmaceutically acceptable salts thereof.

In some embodiments, the compound is selected from the prodrugs of the compounds described in Table 1 and pharmaceutically acceptable salts thereof.

In some embodiments, the compound is selected from the compounds described in Table 1.

TABLE 1
Certain examples of the compound of Formula (A)

| # | IUPAC Name |
|---|---|
| 201 | (1S,2S,5S)-N-[(1S)-1-cyano-2-[(3S)-2-oxopyrrolidin-3-yl]ethyl]-3-[(2S)-2- |
| | (methanesulfonamido)-3,3-dimethyl-butanoyl]-6,6-dimethyl-3- |
| | azabicyclo[3.1.0]hexane-2-carboxamide |
| 202 | (1S,2S,5S)-N-[(1S)-2-amino-2-oxo-1-[[(3S)-2-oxopyrrolidin-3- |
| | yl]methyl]ethyl]-3-[(2S)-2-(methanesulfonamido)-3,3-dimethyl-butanoyl]-6,6- |
| | dimethyl-3-azabicyclo[3.1.0]hexane-2-carboxamide |
| 203 | (2S)-N-[(1S)-1-cyano-2-[(3S)-2-oxopyrrolidin-3-yl]ethyl]-1-[(2S)-3,3- |
| | dimethyl-2-[(2,2,2-trifluoroacetyl)amino]butanoyl]-4,4-dimethyl-piperidine-2- |
| | carboxamide |
| 204 | (1R,2S,5S)-N-[(1S)-1-cyano-2-[(3S)-2-oxopyrrolidin-3-yl]ethyl]-6,6-dimethyl- |
| | 3-[(2R)-3,3,3-trifluoro-2-[(2,2,2-trifluoroacetyl)amino]propanoyl]-3- |
| | azabicyclo[3.1.0]hexane-2-carboxamide |
| 205 | (1S,2S,5S)-N-[(1S)-1-cyano-2-[(3S)-2-oxopyrrolidin-3-yl]ethyl]-3-[(2R)-3,3- |
| | difluoro-2-[(2,2,2-trifluoroacetyl)amino]butanoyl]-6,6-dimethyl-3- |
| | azabicyclo[3.1.0]hexane-2-carboxamide |
| 206 | (7S)-N-[(1S)-1-cyano-2-[(3S)-2-oxopyrrolidin-3-yl]ethyl]-6-[(2S)-3,3- |
| | dimethyl-2-[(2,2,2-trifluoroacetyl)amino]butanoyl]-6-azaspiro[2.5]octane-7- |
| | carboxamide |
| 207 | (1S,2S,5S)-N-[(1S)-1-cyano-2-[(3S)-2-oxopyrrolidin-3-yl]ethyl]-3-[(2S)-3,3- |
| | dimethyl-2-(trifluoromethylsulfonylamino)butanoyl]-6,6-dimethyl-3- |
| | azabicyclo[3.1.0]hexane-2-carboxamide |
| 208 | (1S,2S,5S)-N-[(1S)-2-amino-2-oxo-1-[[(3S)-2-oxopyrrolidin-3- |
| | yl]methyl]ethyl]-3-[(2S)-3,3-dimethyl-2-(2,2,2- |
| | trifluoroethylsulfonylamino)butanoyl]-6,6-dimethyl-3-azabicyclo[3.1.0]hexane- |
| | 2-carboxamide |
| 209 | (1S,2S,5S)-N-[(1S)-1-cyano-2-[(3S)-2-oxopyrrolidin-3-yl]ethyl]-3-[(2S)-3,3- |
| | dimethyl-2-(2,2,2-trifluoroethylsulfonylamino)butanoyl]-6,6-dimethyl-3- |
| | azabicyclo[3.1.0]hexane-2-carboxamide |
| 212 | (1S,2S,5S)-3-[(2S)-3,3-dimethyl-2-[(2,2,2-trifluoroacetyl)amino]butanoyl]-N- |
| | [(1S)-1-(1,1-dioxo-2-thienyl)-2-(2-oxopyrrolidin-3-yl)ethyl]-6,6-dimethyl-3- |
| | azabicyclo[3.1.0]hexane-2-carboxamide |
| 214 | (1S,2S,5S)-N-[(1S)-1-cyano-2-(2-oxoimidazolidin-1-yl)ethyl]-3-[(2S)-3,3- |
| | dimethyl-2-[(2,2,2-trifluoroacetyl)amino]butanoyl]-6,6-dimethyl-3- |
| | azabicyclo[3.1.0]hexane-2-carboxamide |
| 215 | (1S,2S,5S)-N-[(1S)-1-cyano-2-(3-methyl-2-oxo-imidazolidin-1-yl)ethyl]-3- |
| | [(2S)-3,3-dimethyl-2-[(2,2,2-trifluoroacetyl)amino]butanoyl]-6,6-dimethyl-3- |
| | azabicyclo[3.1.0]hexane-2-carboxamide |
| 216 | (1S,2S,5S)-N-[(1S)-1-cyano-2-(2-oxo-1,3,3a,4,5,6,7,7a-octahydroindol-3- |
| | yl)ethyl]-3-[(2S)-3,3-dimethyl-2-[(2,2,2-trifluoroacetyl)amino]butanoyl]-6,6- |
| | dimethyl-3-azabicyclo[3.1.0]hexane-2-carboxamide |
| 217 | (1S,2S,5S)-N-[(1S)-1-cyano-2-(2-oxoindolin-3-yl)ethyl]-3-[(2S)-3,3-dimethyl- |
| | 2-[(2,2,2-trifluoroacetyl)amino]butanoyl]-6,6-dimethyl-3- |
| | azabicyclo[3.1.0]hexane-2-carboxamide |
| 218 | (1S,2S,5S)-N-[(1S)-2-(5-chloro-2-oxo-indolin-3-yl)-1-cyano-ethyl]-3-[(2S)-3,3- |
| | dimethyl-2-[(2,2,2-trifluoroacetyl)amino]butanoyl]-6,6-dimethyl-3- |
| | azabicyclo[3.1.0]hexane-2-carboxamide |
| 219 | (1S,2S,5S)-N-[(1S)-2-(6-chloro-2-oxo-indolin-3-yl)-1-cyano-ethyl]-3-[(2S)-3,3- |
| | dimethyl-2-[(2,2,2-trifluoroacetyl)amino]butanoyl]-6,6-dimethyl-3- |
| | azabicyclo[3.1.0]hexane-2-carboxamide |
| 220 | (1S,2S,5S)-N-[(1S)-1-cyano-2-(5-methyl-2-oxo-indolin-3-yl)ethyl]-3-[(2S)-3,3- |
| | dimethyl-2-[(2,2,2-trifluoroacetyl)amino]butanoyl]-6,6-dimethyl-3- |
| | azabicyclo[3.1.0]hexane-2-carboxamide |
| 221 | (1S,2S,5S)-N-[(1S)-1-cyano-2-(5-methoxy-2-oxo-indolin-3-yl)ethyl]-3-[(2S)- |
| | 3,3-dimethyl-2-[(2,2,2-trifluoroacetyl)amino]butanoyl]-6,6-dimethyl-3- |
| | azabicyclo[3.1.0]hexane-2-carboxamide |
| 222 | (1S,2S,5S)-N-[(1S)-1-cyano-2-(6-methoxy-2-oxo-indolin-3-yl)ethyl]-3-[(2S)- |
| | 3,3-dimethyl-2-[(2,2,2-trifluoroacetyl)amino]butanoyl]-6,6-dimethyl-3- |
| | azabicyclo[3.1.0]hexane-2-carboxamide |
| 223 | (1S,2S,5S)-N-[1-cyano-2-[(4S)-2,5-dioxoimidazolidin-4-yl]ethyl]-3-[(2S)-3,3- |
| | dimethyl-2-[(2,2,2-trifluoroacetyl)amino]butanoyl]-6,6-dimethyl-3- |
| | azabicyclo[3.1.0]hexane-2-carboxamide |
| 224 | (1S,2S,5S)-N-[(1S)-1-cyano-2-(5,6-dimethyl-2-oxo-indolin-3-yl)ethyl]-3-[(2S)- |
| | 3,3-dimethyl-2-[(2,2,2-trifluoroacetyl)amino]butanoyl]-6,6-dimethyl-3- |
| | azabicyclo[3.1.0]hexane-2-carboxamide |
| 225 | (1S,2S,5S)-N-[(1S)-1-cyano-2-(6-methyl-2-oxo-indolin-3-yl)ethyl]-3-[(2S)-3,3- |
| | dimethyl-2-[(2,2,2-trifluoroacetyl)amino]butanoyl]-6,6-dimethyl-3- |
| | azabicyclo[3.1.0]hexane-2-carboxamide |
| 226 | (1S,2S,5S)-N-[1-cyano-2-(2-oxoindolin-3-yl)ethyl]-3-[(2S)-3,3-dimethyl-2- |
| | [(2,2,2-trifluoroacetyl)amino]butanoyl]-6,6-dimethyl-3- |
| | azabicyclo[3.1.0]hexane-2-carboxamide |
| 227 | (1S,2S,5S)-N-[1-cyano-2-(3-fluoro-2-oxo-indolin-3-yl)ethyl]-3-[(2S)-3,3- |
| | dimethyl-2-[(2,2,2-trifluoroacetyl)amino]butanoyl]-6,6-dimethyl-3- |
| | azabicyclo[3.1.0]hexane-2-carboxamide |
| 228 | (1S,2S,5S)-N-[(1S)-1-cyano-2-(3-fluoro-2-oxo-indolin-3-yl)ethyl]-3-[(2S)-3,3- |
| | dimethyl-2-[(2,2,2-trifluoroacetyl)amino]butanoyl]-6,6-dimethyl-3- |
| | azabicyclo[3.1.0]hexane-2-carboxamide |
| 229 | (1S,2S,5S)-N-[1-cyano-2-(3-fluoro-5-methyl-2-oxo-indolin-3-yl)ethyl]-3-[(2S)- |
| | 3,3-dimethyl-2-[(2,2,2-trifluoroacetyl)amino]butanoyl]-6,6-dimethyl-3- |
| | azabicyclo[3.1.0]hexane-2-carboxamide |
| 230 | (1S,2S,5S)-N-[1-cyano-2-(5-methyl-2-oxo-indolin-3-yl)ethyl]-3-[(2S)-3,3- |
| | dimethyl-2-[(2,2,2-trifluoroacetyl)amino]butanoyl]-6,6-dimethyl-3- |
| | azabicyclo[3.1.0]hexane-2-carboxamide |
| 231 | (1S,2S,5S)-N-[(1S)-1-cyano-2-(2-oxoindolin-3-yl)ethyl]-3-[(2S)-3,3-dimethyl- |
| | 2-[(2,2,2-trifluoroacetyl)amino]butanoyl]-N,6,6-trimethyl-3- |
| | azabicyclo[3.1.0]hexane-2-carboxamide |
| 232 | (1S,2S,5S)-N-[(1S)-2-(5-bromo-2-oxo-indolin-3-yl)-1-cyano-ethyl]-3-[(2S)-3,3- |
| | dimethyl-2-[(2,2,2-trifluoroacetyl)amino]butanoyl]-6,6-dimethyl-3- |
| | azabicyclo[3.1.0]hexane-2-carboxamide |
| 233 | (1S,2S,5S)-N-[(1S)-2-(6-bromo-2-oxo-indolin-3-yl)-1-cyano-ethyl]-3-[(2S)-3,3- |
| | dimethyl-2-[(2,2,2-trifluoroacetyl)amino]butanoyl]-6,6-dimethyl-3- |
| | azabicyclo[3.1.0]hexane-2-carboxamide |
| 234 | (1S,2S,5S)-N-[(1S)-1-cyano-2-(4-methyl-2-oxo-indolin-3-yl)ethyl]-3-[(2S)-3,3- |
| | dimethyl-2-[(2,2,2-trifluoroacetyl)amino]butanoyl]-6,6-dimethyl-3- |
| | azabicyclo[3.1.0]hexane-2-carboxamide |
| 235 | (1S,2S,5S)-N-[(1S)-2-(7-chloro-2-oxo-indolin-3-yl)-1-cyano-ethyl]-3-[(2S)-3,3- |
| | dimethyl-2-[(2,2,2-trifluoroacetyl)amino]butanoyl]-6,6-dimethyl-3- |
| | azabicyclo[3.1.0]hexane-2-carboxamide |
| 236 | (1S,2S,5S)-N-[(1S)-1-cyano-2-(5-fluoro-2-oxo-indolin-3-yl)ethyl]-3-[(2S)-3,3- |
| | dimethyl-2-[(2,2,2-trifluoroacetyl)amino]butanoyl]-6,6-dimethyl-3- |
| | azabicyclo[3.1.0]hexane-2-carboxamide |
| 237 | (1S,2S,5S)-N-[(1S)-1-cyano-2-(2-oxoindolin-3-yl)ethyl]-3-[(2S)-2- |
| | (methanesulfonamido)-3,3-dimethyl-butanoyl]-6,6-dimethyl-3- |
| | azabicyclo[3.1.0]hexane-2-carboxamide |
| 238 | (1S,2S,5S)-N-[(1S)-1-cyano-2-(2-oxoindolin-3-yl)ethyl]-3-[(2S)-2- |
| | (methanesulfonamido)-3,3-dimethyl-butanoyl]-6,6-dimethyl-3- |
| | azabicyclo[3.1.0]hexane-2-carboxamide |
| 239 | (1S,2S,5S)-N-[1-cyano-2-(3-methyl-2-oxo-indolin-3-yl)ethyl]-3-[(2S)-3,3- |
| | dimethyl-2-[(2,2,2-trifluoroacetyl)amino]butanoyl]-6,6-dimethyl-3- |
| | azabicyclo[3.1.0]hexane-2-carboxamide |
| 240 | (1R,2S,5S)-N-[(1S)-1-cyano-2-(2-oxoindolin-3-yl)ethyl]-3-[(2S)-3,3-dimethyl- |
| | 2-[(2,2,2-trifluoroacetyl)amino]butanoyl]-6,6-dimethyl-N-propyl-3- |
| | azabicyclo[3.1.0]hexane-2-carboxamide |
| 241 | (1R,2S,5S)-N-[(1S)-1-cyano-2-(2-oxoindolin-3-yl)ethyl]-3-[(2S)-3,3-dimethyl- |
| | 2-[(2,2,2-trifluoroacetyl)amino]butanoyl]-N-isopentyl-6,6-dimethyl-3- |
| | azabicyclo[3.1.0]hexane-2-carboxamide |
| 242 | (1S,2S,5S)-N-[(1S)-1-cyano-2-(2-oxoindolin-3-yl)ethyl]-3-[(2S)-3,3-dimethyl- |
| | 2-[(2,2,2-trifluoroacetyl)amino]butanoyl]-6,6-dimethyl-N-(2-phenylethyl)-3- |
| | azabicyclo[3.1.0]hexane-2-carboxamide |
| 243 | methyl N-[(1S)-1-[(1S,2S,5S)-2-[[(1S)-1-cyano-2-(2-oxoindolin-3- |
| | yl)ethyl]carbamoyl]-6,6-dimethyl-3-azabicyclo[3.1.0]hexane-3-carbonyl]-2,2- |
| | dimethyl-propyl]carbamate |
| 244 | ethyl N-[(1S)-1-[(1S,2S,5S)-2-[[(1S)-1-cyano-2-(2-oxoindolin-3- |
| | yl)ethyl]carbamoyl]-6,6-dimethyl-3-azabicyclo[3.1.0]hexane-3-carbonyl]-2,2- |
| | dimethyl-propyl]carbamate |
| 245 | methyl N-[(1S)-1-[(1S,2S,5S)-2-[[(1S)-1-cyano-2-(2-oxoindolin-3- |
| | yl)ethyl]carbamoyl]-6,6-dimethyl-3-azabicyclo[3.1.0]hexane-3-carbonyl]-3- |
| | methyl-butyl]carbamate |
| 246 | ethyl N-[(1S)-1-[(1S,2S,5S)-2-[[(1S)-1-cyano-2-(2-oxoindolin-3- |
| | yl)ethyl]carbamoyl]-6,6-dimethyl-3-azabicyclo[3.1.0]hexane-3-carbonyl]-3- |
| | methyl-butyl]carbamate |
| 247 | (1R,2S,5S)-N-[(1S)-1-cyano-2-(2-oxoindolin-3-yl)ethyl]-3-[(2S)-3,3-dimethyl- |
| | 2-[(2,2,2-trifluoroacetyl)amino]butanoyl]-N-isopentyl-6,6-dimethyl-3- |
| | azabicyclo[3.1.0]hexane-2-carboxamide |
| 248 | (1R,2S,5S)-N-[(1S)-1-cyano-2-(2-oxoindolin-3-yl)ethyl]-3-[(2S)-3,3-dimethyl- |
| | 2-[(2,2,2-trifluoroacetyl)amino]butanoyl]-6,6-dimethyl-N-propyl-3- |
| | azabicyclo[3.1.0]hexane-2-carboxamide |
| 249 | (1S,2S,5S)-N-[1-cyano-2-(2-oxo-1,3-dihydropyrrolo[2,3-b]pyridin-3-yl)ethyl]- |
| | 3-[(2S)-3,3-dimethyl-2-[(2,2,2-trifluoroacetyl)amino]butanoyl]-6,6-dimethyl-3- |
| | azabicyclo[3.1.0]hexane-2-carboxamide |
| 250 | (1S,2S,5S)-N-[1-cyano-3-(2-oxoindolin-3-yl)propyl]-3-[(2S)-3,3-dimethyl-2- |
| | [(2,2,2-trifluoroacetyl)amino]butanoyl]-6,6-dimethyl-3- |
| | azabicyclo[3.1.0]hexane-2-carboxamide |
| 251 | (1S,2S,5S)-N-[(1S)-1-cyano-2-(2-oxoindolin-3-yl)ethyl]-6,6-dimethyl-3-[(2S)- |
| | 4-methyl-2-[(2,2,2-trifluoroacetyl)amino]pentanoyl]-3-azabicyclo[3.1.0]hexane- |
| | 2-carboxamide |
| 252 | 7-chloro-N-[(1S)-1-[(1S,2S,5S)-2-[[(1S)-1-cyano-2-(2-oxoindolin-3- |
| | yl)ethyl]carbamoyl]-6,6-dimethyl-3-azabicyclo[3.1.0]hexane-3-carbonyl]-2,2- |
| | dimethyl-propyl]-1H-indole-2-carboxamide |
| 253 | (1S,2S,5S)-N-[(1R)-1-cyano-2-(2-oxoindolin-3-yl)ethyl]-3-[(2S)-3,3-dimethyl- |
| | 2-[(2,2,2-trifluoroacetyl)amino]butanoyl]-6,6-dimethyl-3- |
| | azabicyclo[3.1.0]hexane-2-carboxamide |
| 301 | (1S,2S,5S)-N-[(1S)-1-cyano-2-(2-oxo-3H-benzimidazol-1-yl)ethyl]-3-[(2S)-3,3- |
| | dimethyl-2-[(2,2,2-trifluoroacetyl)amino]butanoyl]-6,6-dimethyl-3- |
| | azabicyclo[3.1.0]hexane-2-carboxamide |
| 302 | (1S,2S,5S)-N-[1-cyano-2-(3-methyl-2-oxo-benzimidazol-1-yl)ethyl]-3-[(2S)- |
| | 3,3-dimethyl-2-[(2,2,2-trifluoroacetyl)amino]butanoyl]-6,6-dimethyl-3- |
| | azabicyclo[3.1.0]hexane-2-carboxamide |
| 303 | (1S,2S,5S)-N-[(1S)-2-amino-2-oxo-1-[(2-oxo-3H-benzimidazol-1- |
| | yl)methyl]ethyl]-3-[(2S)-3,3-dimethyl-2-[(2,2,2- |
| | trifluoroacetyl)amino]butanoyl]-6,6-dimethyl-3-azabicyclo[3.1.0]hexane-2- |
| | carboxamide |
| 304 | (1S,2S,5S)-N-[(1S)-1-cyano-2-(2-oxo-1H-imidazol-3-yl)ethyl]-3-[(2S)-3,3- |
| | dimethyl-2-[(2,2,2-trifluoroacetyl)amino]butanoyl]-6,6-dimethyl-3- |
| | azabicyclo[3.1.0]hexane-2-carboxamide |
| 305 | (1S,2S,5S)-N-[(1S)-1-cyano-2-(3-methyl-2-oxo-imidazol-1-yl)ethyl]-3-[(2S)- |
| | 3,3-dimethyl-2-[(2,2,2-trifluoroacetyl)amino]butanoyl]-6,6-dimethyl-3- |
| | azabicyclo[3.1.0]hexane-2-carboxamide |
| 306 | (1S,2S,5S)-N-[(1S)-1-cyano-2-(triazol-1-yl)ethyl]-3-[(2S)-3,3-dimethyl-2- |
| | [(2,2,2-trifluoroacetyl)amino]butanoyl]-6,6-dimethyl-3- |
| | azabicyclo[3.1.0]hexane-2-carboxamide |
| 307 | (1S,2S,5S)-N-[(1S)-1-cyano-2-(4,5-dimethyltriazol-1-yl)ethyl]-3-[(2S)-3,3- |
| | dimethyl-2-[(2,2,2-trifluoroacetyl)amino]butanoyl]-6,6-dimethyl-3- |
| | azabicyclo[3.1.0]hexane-2-carboxamide |
| 308 | (1S,2S,5S)-N-[(1S)-2-(benzotriazol-1-yl)-1-cyano-ethyl]-3-[(2S)-3,3-dimethyl- |
| | 2-[(2,2,2-trifluoroacetyl)amino]butanoyl]-6,6-dimethyl-3- |
| | azabicyclo[3.1.0]hexane-2-carboxamide |
| 309 | (1S,2S,5S)-N-[(1S)-2-amino-2-oxo-1-(triazol-1-ylmethyl)ethyl]-3-[(2S)-3,3- |
| | dimethyl-2-[(2,2,2-trifluoroacetyl)amino]butanoyl]-6,6-dimethyl-3- |
| | azabicyclo[3.1.0]hexane-2-carboxamide |
| 310 | (1R,2S,5S)-N-[(1S)-2-amino-1-[(4,5-dimethyltriazol-1-yl)methyl]-2-oxo-ethyl]- |
| | 3-[(2S)-3,3-dimethyl-2-[(2,2,2-trifluoroacetyl)amino]butanoyl]-6,6-dimethyl-3- |
| | azabicyclo[3.1.0]hexane-2-carboxamide |
| 311 | (1S,2S,5S)-N-[(1S)-1-cyano-2-(4,5-dimethyltriazol-1-yl)ethyl]-3-[(2S)-3,3- |
| | dimethyl-2-[(2,2,2-trifluoroacetyl)amino]butanoyl]-6,6-dimethyl-3- |
| | azabicyclo[3.1.0]hexane-2-carboxamide |
| 312 | (1S,2S,5S)-N-[(1S)-2-(benzotriazol-1-yl)-1-cyano-ethyl]-3-[(2S)-3,3-dimethyl- |
| | 2-[(2,2,2-trifluoroacetyl)amino]butanoyl]-6,6-dimethyl-3- |
| | azabicyclo[3.1.0]hexane-2-carboxamide |
| 401 | (1S,2S,5S)-N-[1-cyano-2-[(4S)-2,5-dioxoimidazolidin-4-yl]ethyl]-3-[(2S)-3,3- |
| | dimethyl-2-[(2,2,2-trifluoroacetyl)amino]butanoyl]-6,6-dimethyl-3- |
| | azabicyclo[3.1.0]hexane-2-carboxamide |
| 402 | (1S,2S,5S)-N-[1-cyano-2-(2-oxoimidazol-4-yl)ethyl]-3-[(2S)-3,3-dimethyl-2- |
| | [(2,2,2-trifluoroacetyl)amino]butanoyl]-6,6-dimethyl-3- |
| | azabicyclo[3.1.0]hexane-2-carboxamide |
| 403 | (1S,2S,5S)-N-[1-cyano-2-(2-oxo-1,3-dihydroimidazol-4-yl)ethyl]-3-[(2S)-3,3- |
| | dimethyl-2-[(2,2,2-trifluoroacetyl)amino]butanoyl]-6,6-dimethyl-3- |
| | azabicyclo[3.1.0]hexane-2-carboxamide |
| 404 | (1S,2S,5S)-N-[1-cyano-2-(3-methyl-2-oxo-1H-imidazol-5-yl)ethyl]-3-[(2S)-3,3- |
| | dimethyl-2-[(2,2,2-trifluoroacetyl)amino]butanoyl]-6,6-dimethyl-3- |
| | azabicyclo[3.1.0]hexane-2-carboxamide |
| 501 | (1S,2S,5S)-N-[(1S)-2-(benzotriazol-2-yl)-1-cyano-ethyl]-3-[(2S)-3,3-dimethyl- |
| | 2-[(2,2,2-trifluoroacetyl)amino]butanoyl]-6,6-dimethyl-3- |
| | azabicyclo[3.1.0]hexane-2-carboxamide |

In some embodiments, the compound is a pharmaceutically acceptable salt of any one of the compounds described in Table 1.

In some embodiments, the compound is a lithium salt, sodium salt, potassium salt, calcium salt, or magnesium salt of any one of the compounds described in Table 1.

In some embodiments, the compound is a sodium salt or potassium salt ofany one of the compounds described in Table 1.

In some embodiments, the compound is a salt ofany acid described in the Table 2 and any one of the compounds described in Table 1.

TABLE 2
Pharmaceutically acceptable acid forming
salts with the Compound of Formula (A).

| | | |
|---|---|---|
| 1-hydroxy-2-naphthoic acid | ethanesulfonic acid | methanesulfonic acid |
| 2,2-dichloroacetic acid | formic acid | naphthalene-1,5-disulfonic acid |
| 2-hydroxyethanesulfonic acid | fumaric acid | naphthalene-2-sulfonic acid |
| 2-oxoglutaric acid | galactaric acid | nicotinic acid |
| 4-acetamidobenzoic acid | gentisic acid | nitric acid |
| 4-aminosalicylic acid | glucoheptonic acid (D) | oleic acid |
| acetic acid | gluconic acid (D) | oxalic acid |
| adipic acid | glucuronic acid (D) | palmitic acid |
| ascorbic acid (L) | glutamic acid | pamoic acid |
| aspartic acid (L) | glutaric acid | phosphoric acid |
| benzenesulfonic acid | glycerophosphoric acid | proprionic acid |
| benzoic acid | glycolic acid | pyroglutamic acid (−L) |
| camphoric acid (+) | hippuric acid | salicylic acid |
| camphor-10-sulfonic acid (+) | hydrobromic acid | sebacic acid |
| capric acid (decanoic acid) | hydrochloric acid | stearic acid |
| caproic acid (hexanoic acid) | isobutyric acid | succinic acid |
| caprylic acid (octanoic acid) | lactic acid (DL) | sulfuric acid |
| carbonic acid | lactobionic acid | tartaric acid (+L) |
| cinnamic acid | lauric acid | thiocyanic acid |
| citric acid | maleic acid | toluenesulfonic acid (p) |
| cyclamic acid | malic acid (−L) | undecylenic acid |
| dodecylsulfuric acid | malonic acid | |
| ethane-1,2-disulfonic acid | mandelic acid (DL) | |

In some embodiments, the compound is a salt of acetic acid and any one of the compounds described in Table 1.

In some embodiments, the compound is a salt of adipic acid and any one of the compounds described in Table 1.

In some embodiments, the compound is a salt of ascorbic acid (L) and any one of the compounds described in Table 1.

In some embodiments, the compound is a salt of hydrobromic acid and any one of the compounds described in Table 1.

In some embodiments, the compound is a salt of hydrochloric acid and any one of the compounds described in Table 1.

In some embodiments, the compound is a salt of citric acid and any one of the compounds described in Table 1.

In some embodiments, the compound is a salt of glutamic acid and any one of the compounds described in Table 1.

In some embodiments, the compound is a salt of oxalic acid and any one of the compounds described in Table 1.

In some embodiments, the compound is a salt of formic acid and any one of the compounds described in Table 1.

In some embodiments, the compound is a salt of sulfuric acid and any one of the compounds described in Table 1.

In some aspects, the present disclosure provides a compound being an isotopic derivative (e.g., isotopically labeled compound) of any one of the compounds of the Formulae disclosed herein.

In some embodiments, the compound is an isotopic derivative of any one of the compounds described in Table 1 and prodrugs and pharmaceutically acceptable salts thereof.

In some embodiments, the compound is an isotopic derivative of any one of the compounds described in Table 1 and pharmaceutically acceptable salts thereof.

In some embodiments, the compound is an isotopic derivative of any one of prodrugs of the compounds described in Table 1 and pharmaceutically acceptable salts thereof.

In some embodiments, the compound is an isotopic derivative of any one of the compounds described in Table 1.

It is understood that the isotopic derivative can be prepared using any of a variety of art-recognized techniques. For example, the isotopic derivative can generally be prepared by carrying out the procedures disclosed in the Schemes and/or in the Examples described herein, by substituting an isotopically labeled reagent for a non-isotopically labeled reagent.

In some embodiments, the isotopic derivative is a deuterium labeled compound.

In some embodiments, the isotopic derivative is a deuterium labeled compound of any one of the compounds of the Formulae disclosed herein.

The term “isotopic derivative”, as used herein, refers to a derivative of a compound in which one or more atoms are isotopically enriched or labelled. For example, an isotopic derivative of a compound of Formula (A) is isotopically enriched with regard to, or labelled with, one or more isotopes as compared to the corresponding compound of Formula (A). In some embodiments, the isotopic derivative is enriched with regard to, or labelled with, one or more atoms selected from ²H, ¹³C, ¹⁴C, ¹⁵N, ¹⁸O, ²⁹Si, ³¹P, and ³⁴S. In some embodiments, the isotopic derivative is a deuterium labeled compound (i.e., being enriched with ²H with regard to one or more atoms thereof).

In some embodiments, the compound is a deuterium labeled compound of any one of the compounds described in Table 1 and prodrugs and pharmaceutically acceptable salts thereof.

In some embodiments, the compound is a deuterium labeled compound of any one of the compounds described in Table 1 and pharmaceutically acceptable salts thereof.

In some embodiments, the compound is a deuterium labeled compound of any one of the prodrugs of the compounds described in Table 1 and pharmaceutically acceptable salts thereof.

In some embodiments, the compound is a deuterium labeled compound of any one of the compounds described in Table 1.

It is understood that the deuterium labeled compound comprises a deuterium atom having an abundance of deuterium that is substantially greater than the natural abundance of deuterium, which is 0.015%.

In some embodiments, the deuterium labeled compound has a deuterium enrichment factor for each deuterium atom of at least 3500 (52.5% deuterium incorporation at each deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). As used herein, the term “deuterium enrichment factor” means the ratio between the deuterium abundance and the natural abundance of a deuterium.

It is understood that the deuterium labeled compound can be prepared using any of a variety of art-recognized techniques. For example, the deuterium labeled compound can generally be prepared by carrying out the procedures disclosed in the Schemes and/or in the Examples described herein, by substituting a deuterium labeled reagent for a non-deuterium labeled reagent.

A compound of the disclosure or a pharmaceutically acceptable salt or solvate thereof that contains the aforementioned deuterium atom(s) is within the scope of the disclosure. Further, substitution with deuterium (i.e., ²H) may afford certain therapeutic advantages resulting from greater metabolic stability, e.g., increased in vivo half-life or reduced dosage requirements.

In some embodiments, the compound is a ¹⁸F labeled compound.

In some embodiments, the compound is a ³S labeled compound, a ³⁴S labeled compound, a ³⁵S labeled compound, a ³⁶S labeled compound, or any combination thereof.

It is understood that the ¹⁸F, ³³S, ³⁴S, ³⁵S, and/or ³⁶S labeled compound, can be prepared using any of a variety of art-recognized techniques. For example, the deuterium labeled compound can generally be prepared by carrying out the procedures disclosed in the Schemes and/or in the Examples described herein, by substituting a ¹⁸F, ³³S, ³⁴S, ³⁵S, and/or ³⁶S labeled reagent for a non-isotope labeled reagent.

A compound of the disclosure or a pharmaceutically acceptable salt or solvate thereof that contains one or more of the aforementioned ¹⁸F, ³³S, ³⁴S, ³⁵, and ³⁶S atom(s) is within the scope of the disclosure. Further, substitution with isotope (e.g, ¹⁸F, ³³S, ³⁴S, ³⁵S, and/or ³⁶S) may afford certain therapeutic advantages resulting from greater metabolic stability, e.g., increased in vivo half-life or reduced dosage requirements.

For the avoidance of doubt, it is to be understood that, where in this specification a group is qualified by “described herein”, the said group encompasses the first occurring and broadest definition as well as each and all of the particular definitions for that group.

The various functional groups and substituents making up the compounds of the Formula (A) are typically chosen such that the molecular weight of the compound does not exceed 1000 Daltons. More usually, the molecular weight of the compound will be less than 1000, for example less than 900, or less than 800, or less than 700, or less than 600, or less than 500.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral center” refers to a carbon atom bonded to four nonidentical substituents.

As used herein, the term “chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerisation is possible, a chemical equilibrium of the tautomers will be reached. The exact ratio of the tautomers depends on several factors, including temperature, solvent, and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterised by the absolute configuration of its asymmetric centre and is described by the R- and S-sequencing rules of Cahn and Prelog, or by the manner in which the molecule rotates the plane of polarised light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this disclosure may possess one or more asymmetric centres; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the disclosure may have geometric isomeric centres (E- and Z-isomers). It is to be understood that the present disclosure encompasses all optical, diastereoisomers and geometric isomers and mixtures thereof that possess inflammasome inhibitory activity.

The present disclosure also encompasses compounds of the disclosure as defined herein which comprise one or more isotopic substitutions.

It is to be understood that the compounds of any Formula described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted compound disclosed herein. Suitable cations include sodium ion, potassium ion, magnesium ion, calcium ion, and an ammonium cation such as tetramethylammonium ion or diethylamine ion. The substituted compounds disclosed herein also include those salts containing quaternary nitrogen atoms.

It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As used herein, the term “derivative” refers to compounds that have a common core structure and are substituted with various groups as described herein.

As used herein, the term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonamides, tetrazoles, sulfonates and phosphonates. See, e.g., Patani and LaVoie, Chem. Rev. 96, 3147-3176, 1996.

It is also to be understood that certain compounds of any one of the Formulae disclosed herein may exist in solvated as well as unsolvated forms such as, for example, hydrated forms. A suitable pharmaceutically acceptable solvate is, for example, a hydrate such as hemi-hydrate, a mono-hydrate, a di-hydrate or a tri-hydrate. It is to be understood that the disclosure encompasses all such solvated forms that possess inflammasome inhibitory activity.

It is also to be understood that certain compounds of any one of the Formulae disclosed herein may exhibit polymorphism, and that the disclosure encompasses all such forms, or mixtures thereof, which possess inflammasome inhibitory activity. It is generally known that crystalline materials may be analysed using conventional techniques such as X-Ray Powder Diffraction analysis, Differential Scanning Calorimetry, Thermal Gravimetric Analysis, Diffuse Reflectance Infrared Fourier Transform (DRIFT) spectroscopy, Near Infrared (NIR) spectroscopy, solution and/or solid state nuclear magnetic resonance spectroscopy. The water content of such crystalline materials may be determined by Karl Fischer analysis.

Compounds of any one of the Formulae disclosed herein may exist in a number of different tautomeric forms and references to compound of Formula (A) include all such forms. For the avoidance of doubt, where a compound can exist in one of several tautomeric forms, and only one is specifically described or shown, all others are nevertheless embraced by Formula (A). Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

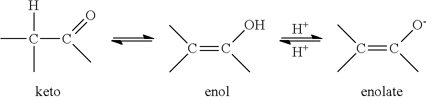

Compounds of any one of the Formulae disclosed herein containing an amine function may also form N-oxides. A reference herein to a compound of Formula (A) that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-oxides can be formed by treatment of the corresponding amine with an oxidising agent such as hydrogen peroxide or a peracid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with meta-chloroperoxybenzoic acid (mCPBA), for example, in an inert solvent such as dichloromethane.

The compounds of any one of the Formulae disclosed herein may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include derivatives containing in vivo cleavable alkyl or acyl substituents at the ester or amide group in any one of the Formulae disclosed herein.

Accordingly, the present disclosure includes those compounds of any one of the Formulae disclosed herein as defined hereinbefore when made available by organic synthesis and when made available within the human or animal body by way of cleavage of a prodrug thereof. Accordingly, the present disclosure includes those compounds of any one of the Formulae disclosed herein that are produced by organic synthetic means and also such compounds that are produced in the human or animal body by way of metabolism of a precursor compound, that is a compound of any one of the Formulae disclosed herein may be a synthetically produced compound or a metabolically-produced compound.

A suitable pharmaceutically acceptable prodrug of a compound of any one of the Formulae disclosed herein is one that is based on reasonable medical judgment as being suitable for administration to the human or animal body without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

A suitable pharmaceutically acceptable prodrug of a compound of any one of the Formulae disclosed herein that possesses a hydroxy group is, for example, an in vivo cleavable ester or ether thereof. An in vivo cleavable ester or ether of a compound of any one of the Formulae disclosed herein containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable prodrug of a compound of any one of the Formulae disclosed herein that possesses a carboxy group is, for example, an in vivo cleavable amide thereof, for example an amide formed with an amine such as ammonia, a C₁₋₄ alkylamine such as methylamine, a (C₁-C₄ alkyl)₂amine such as dimethylamine, N-ethyl-N-methylamine or diethylamine, a C₁-C₄ alkoxy-C₂-C₄ alkylamine such as 2-methoxyethylamine, a phenyl-C₁-C₄ alkylamine such as benzylamine and amino acids such as glycine or an ester thereof.

A suitable pharmaceutically acceptable prodrug of a compound of any one of the Formulae disclosed herein that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl.

The in vivo effects of a compound of any one of the Formulae disclosed herein may be exerted in part by one or more metabolites that are formed within the human or animal body after administration of a compound of any one of the Formulae disclosed herein. As stated hereinbefore, the in vivo effects of a compound of any one of the Formulae disclosed herein may also be exerted by way of metabolism of a precursor compound (a prodrug).

### Method of Synthesizing the Compounds

The compounds of the present invention may be made by a variety of methods, including standard chemistry. Suitable synthetic routes are depicted in the Schemes given below.

The compounds of Formula (A) may be prepared by methods known in the art of organic synthesis as set forth in part by the following synthetic schemes. In the schemes described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of those skilled in the art will recognize if a stereocenter exists in the compounds of Formula (A). Accordingly, the present invention includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-lnterscience, 1994).

The compounds described herein may be made from commercially available starting materials or synthesized using known organic, inorganic, and/or enzymatic processes.

### Preparation of Compounds

The compounds of the present invention can be prepared in a number of ways well known to those skilled in the art of organic synthesis. By way of example, compounds of the present invention can be synthesized using the methods described below, together with synthetic methods known in the art of synthetic organic chemistry, or variations thereon as appreciated by those skilled in the art. Suitable methods include but are not limited to those methods described below. Compounds of the present invention can be synthesized by following the steps outlined in General Procedures (General schemes I, II, III, IV) which comprise different sequences of assembling intermediates or compounds. Starting materials are either commercially available or made by known procedures in the reported literature or as illustrated below.

### General Procedure

It is understood that compound of Formula (A) described herein represents anyone of compound of Formula (II), compound of Formula (III), compound of Formula (IV) and/or compound of Formula (V).

In general, the compound of the Formula (A) can be prepared using reactions well known to those skilled in the art of organic synthesis. General scheme I showed possible synthetic route for the preparation of compound of the Formula (A) presented below:

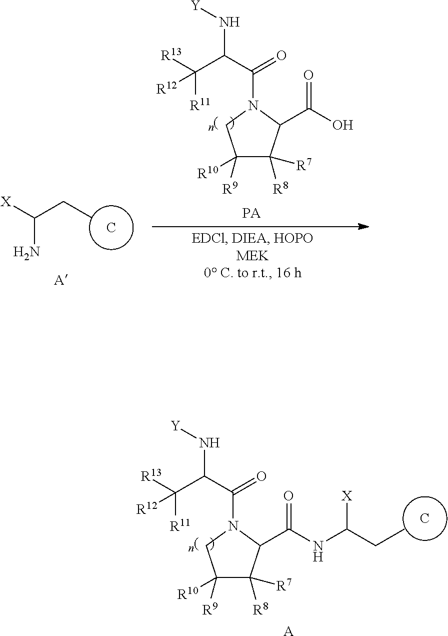

All reagents may be commercially available compounds itself or products of synthesis from commercially available reagents. For each compound in preparation may be used one step or multistep synthetic procedures, including but not limited procedures described herein in preparative part.

Possible synthetic routes for the preparation of compounds of the Formula (II)-Formula (V) presented below:

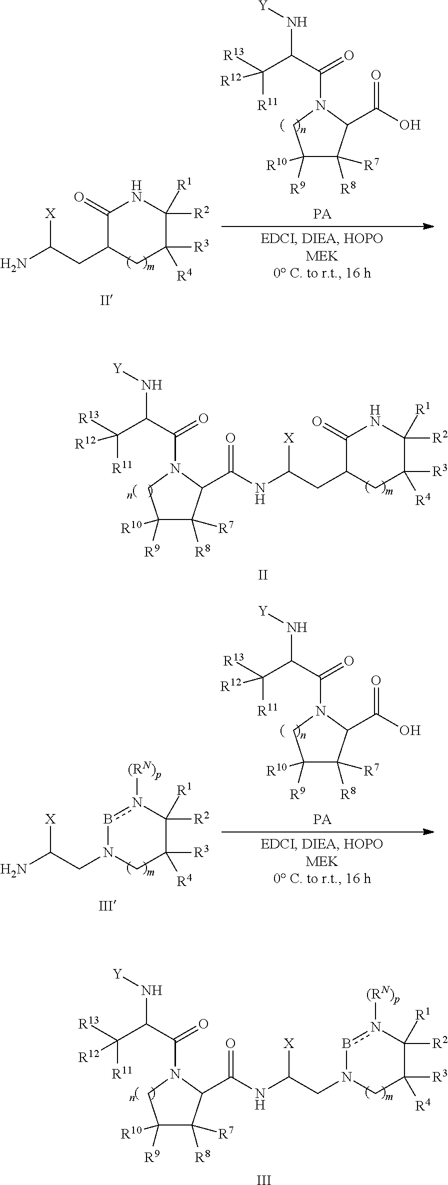

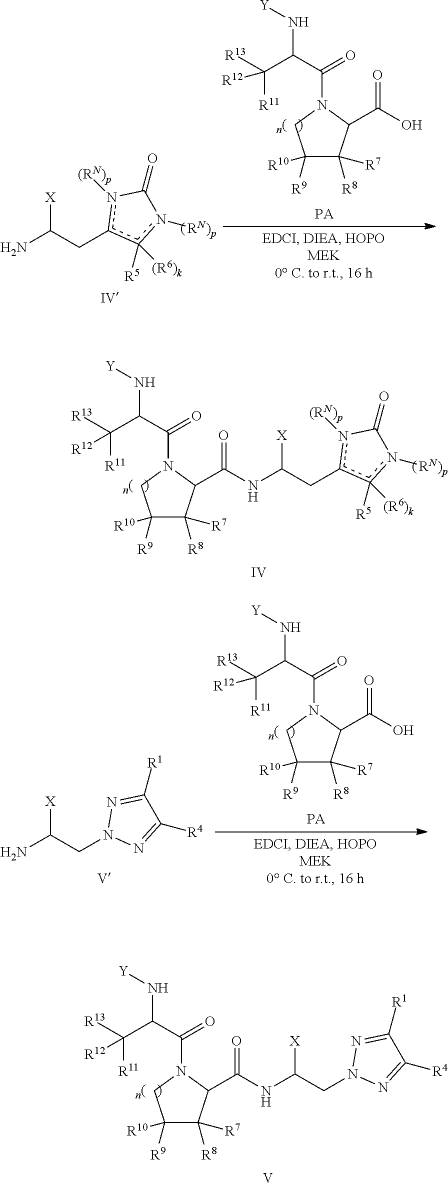

It should be obvious for specialist in this field that any of compound of Formula (A) obtained according to the procedures described above may be a subject for further transformation and modification that will led to other compound of Formula (A).

Any of described herein residue Y can be introduced into the structure of compound of Formula (A) using different approaches, some of them presented on the Scheme below.

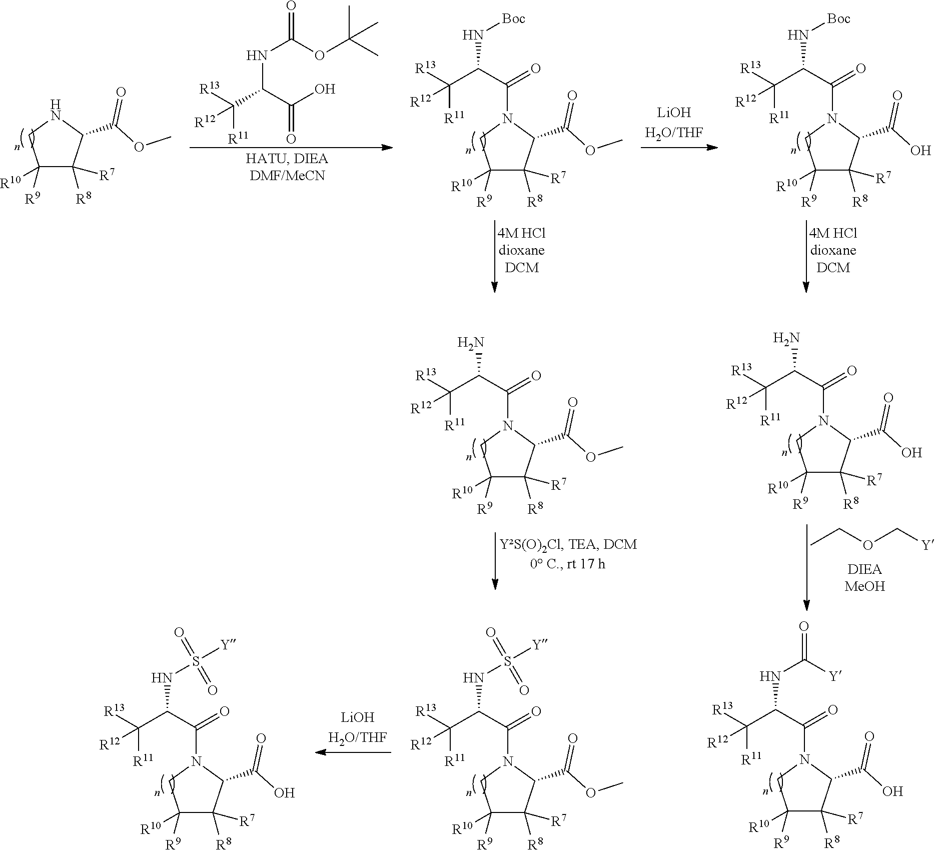

In some embodiments compound of Formula (A) can be subject for further transformations leading to other compound of Formula (A):

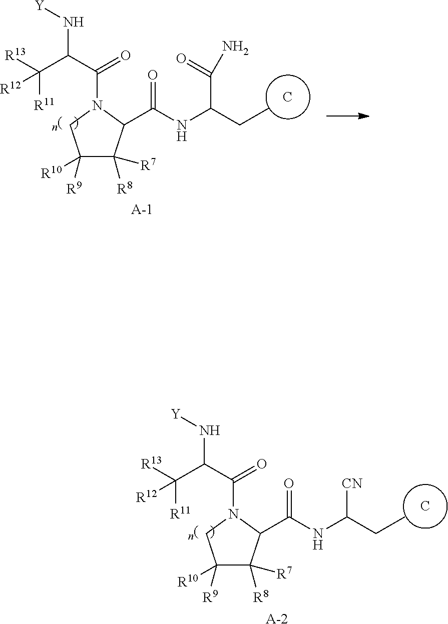

### Biological Assays

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

### Pharmaceutical Compositions

In some aspects, the present disclosure provides a pharmaceutical composition comprising a compound of the present disclosure as an active ingredient. In some embodiments, the present disclosure provides a pharmaceutical composition comprising at least one compound of each of the formulae described herein, or a pharmaceutically acceptable salt or solvate thereof, and one or more pharmaceutically acceptable carriers or excipients. In some embodiments, the present disclosure provides a pharmaceutical composition comprising at least one compound selected from Table 1.

As used herein, the term “composition” is intended to encompass a product comprising the specified ingredients in the specified amounts, as well as any product which results, directly or indirectly, from combination of the specified ingredients in the specified amounts.

The compounds of present disclosure can be formulated for oral administration in forms such as tablets, capsules (each of which includes sustained release or timed release formulations), pills, powders, granules, elixirs, tinctures, suspensions, syrups and emulsions. The compounds of present disclosure on can also be formulated for intravenous (bolus or in-fusion), intraperitoneal, topical, subcutaneous, intramuscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio)propyl-β-cyclodextrin, glucosyl-β-cyclodextrin, sulfated β-cyclodextrin (S-β-CD), maltosyl-β-cyclodextrin, β-cyclodextrin sulfobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

In some embodiments, examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof. In some embodiments, the tonicity agent is selected from the group consisting of a glycol (such as propylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols—such as Carbopol 934, Carbopol 934P, Carbopol 971, Carbopol 974 and Carbopol 974P), and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from the group of potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base—depending on the formulation, the addition of one of the acid or base may be sufficient to bring the mixture to the desired pH range.

The aqueous vehicle may also contain a buffering agent to stabilize the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and ε-aminocaproic acid, and mixtures thereof.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

According to a further aspect of the disclosure there is provided a pharmaceutical composition which comprises a compound of the disclosure as defined hereinbefore, or a pharmaceutically acceptable salt, hydrate or solvate thereof, in association with a pharmaceutically acceptable diluent or carrier.

In some embodiments, a pharmaceutical composition described herein may further comprise one or more additional pharmaceutically active agents.

Additional therapeutic agents that can be used in the methods of the invention include Diosmin, Hesperidin, MK-3207, Venetoclax, Dihydroergocristine, Bolazine, R428, Ditercalinium, Etoposide, Teniposide, UK-432097, lrinotecan, Lumacaftor, Velpatasvir, Eluxadoline, Ledipasvir, Lopinavir/Ritonavir+Ribavirin, Alferon, and prednisone. Other additional agents useful in the methods of the present invention include dexamethasone, azithromycin and remdesivir as well as boceprevir, umifenovir and favipiravir.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure may be obtained by conventional procedures using conventional pharmaceutical excipients, well known in the art. Thus, compositions intended for oral use may contain, for example, one or more coloring, sweetening, flavoring and/or preservative agents.

A therapeutically effective amount of a compound of the present disclosure for use in therapy is an amount sufficient to treat or prevent a coronavirus infection, slow its progression and/or reduce the symptoms associated with the condition.

The size of the dose for therapeutic or prophylactic purposes of a compound of Formula (II)-Formula (V) will naturally vary according to the nature and severity of the conditions, the age and sex of the animal or subject and the route of administration, according to well-known principles of medicine.

### Methods of Use

In some aspects, the present disclosure provides a method of inhibition of SARS-CoV-2-related 3C-like protease (e.g., in vitro or in vivo), comprising contacting a cell with a therapeutically effective amount of a compound of the present disclosure or a pharmaceutically acceptable salt thereof.

In some aspects, the present disclosure provides a method of treating or preventing a coronavirus infection in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a coronavirus infection in a subject in need thereof, comprising administering to the subject a therapeutically effective amount of a compound of the present disclosure or a pharmaceutically acceptable salt thereof, or a pharmaceutical composition of the present disclosure.

In some embodiments, the disease or disorder is associated with coronavirus. In some embodiments, the disease or disorder is a disease or disorder in which coronavirus is implicated.

The compounds of the invention are inhibitors of SARS-CoV-2-related 3C-like protease.

In some embodiments, the compounds, compositions, and methods disclosed herein are used in the prevention or treatment of a disease, disorder, or condition associated with coronavirus infection.

The compounds of the invention are also useful in treating diseases associated with the coronavirus infection. For example, diseases and conditions treatable according to the methods of the invention include COVID-19.

In some aspects, the present disclosure provides a method of treating or preventing a COVID-19 infection in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a COVID-19 in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a compound of the present disclosure or a pharmaceutically acceptable salt thereof for use in inhibiting of SARS-CoV-2-related 3C-like protease (e.g., in vitro or in vivo).

In some aspects, the present disclosure provides a compound of the present disclosure or a pharmaceutically acceptable salt thereof for use in treating or preventing a disease or disorder disclosed herein.

In some aspects, the present disclosure provides a compound of the present disclosure or a pharmaceutically acceptable salt thereof for use in treating a disease or disorder disclosed herein.

In some aspects, the present disclosure provides a compound of the present disclosure or a pharmaceutically acceptable salt thereof for use in treating or preventing a coronavirus infection in a subject in need thereof.

In some aspects, the present disclosure provides a compound of the present disclosure or a pharmaceutically acceptable salt thereof for use in treating or preventing a COVID-19 in a subject in need thereof.

In some aspects, the present disclosure provides use of a compound of the present disclosure or a pharmaceutically acceptable salt thereof in the manufacture of a medicament for inhibiting of SARS-CoV-2-related 3C-like protease (e.g., in vitro or in vivo).

In some aspects, the present disclosure provides use of a compound of the present disclosure or a pharmaceutically acceptable salt thereof in the manufacture of a medicament for treating or preventing a disease or disorder disclosed herein.

In some aspects, the present disclosure provides use of a compound of the present disclosure or a pharmaceutically acceptable salt thereof in the manufacture of a medicament for treating a disease or disorder disclosed herein.

In some aspects, the present disclosure provides use of a compound of the present disclosure or a pharmaceutically acceptable salt thereof in the manufacture of a medicament for treating or preventing a coronavirus infection in a subject in need thereof.

In some aspects, the present disclosure provides use of a compound of the present disclosure or a pharmaceutically acceptable salt thereof in the manufacture of a medicament for treating or preventing a COVID-19 in a subject in need thereof.

The present disclosure provides compounds that function as inhibitors of SARS-CoV-2-related 3C-like protease (e.g., in vitro or in vivo). The present disclosure therefore provides a method of inhibiting of SARS-CoV-2-related 3C-like protease in vitro or in vivo, said method comprising contacting a cell with a therapeutically effective amount of a compound, or a pharmaceutically acceptable salt thereof, as defined herein.

In some embodiments, the inhibitor of SARS-CoV-2-related 3C-like protease is a compound of the present disclosure.

Effectiveness of compounds of the disclosure can be determined by industry-accepted assays/disease models according to standard practices of elucidating the same as described in the art and are found in the current general knowledge.

The present disclosure also provides a method of treating a disease or disorder in which coronavirus infection is implicated in a subject in need of such treatment, said method comprising administering to said subject a therapeutically effective amount of a compound, or a pharmaceutically acceptable salt thereof, or a pharmaceutical composition as defined herein.

In some embodiments, the subject is a mammal. In some embodiments, the subject is a human.

### Routes of Administration

The compounds of the disclosure or pharmaceutical compositions comprising these compounds may be administered to a subject by any convenient route of administration, whether systemically/peripherally or topically (i.e., at the site of desired action).

Routes of administration include, but are not limited to, oral (e.g. by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

Abbreviations used in the following examples and elsewhere herein are:
ACN acetonitrile
AcOH acetic acid
AcCl acetyl chloride
anh. anhydrous
aq. aqueous
br. broad
Burgess reagent methyl N-(triethylammoniumsulfonyl)carbamate
calc. calculated
conc. concentrated
d duplet
DCM dichloromethane
DIPEA N,N-diisopropylethylamine
DMF dimethylformamide
DMSO dimethyl sulfoxide
ESI electrospray ionization
h hour(s)
HATU 1-[Bis(dimethylamino)methylene]-1H-1,2,3-triazolo[4,5-b]pyridinium 3-oxid hexafluorophosphate
HPLC high pressure (or performance) liquid chromatography
IPA iso-propanol
LCMS liquid chromatography mass spectrometry
m multiplet
M molar
MHz megahertz
min minutes
Ms methanesulfonyl, mesyl
MsCl methanesulfonyl chloride
NMR nuclear magnetic resonance
q quadruplet
rt room temperature
s singlet
s solid
sat. saturated
t temperature, triplet
t_{R} retention time (in chromatography)
TEA triethylamine
TFA trifluoroacetic acid
THF tetrahydrofuran
TLC thin layer chromatography

### EXAMPLES

### General Synthetical Procedures and Examples of the Compound's Preparation

### Synthesis of Building Blocks

### Synthesis of (1R,2S,5S)-3-((S)-3,3-dimethyl-2-(2,2,2-trifluoroacetamido)butanoyl)-6,6-dimethyl-3-azabicyclo[3.1.0]hexane-2-carboxylic Acid (PA.4)

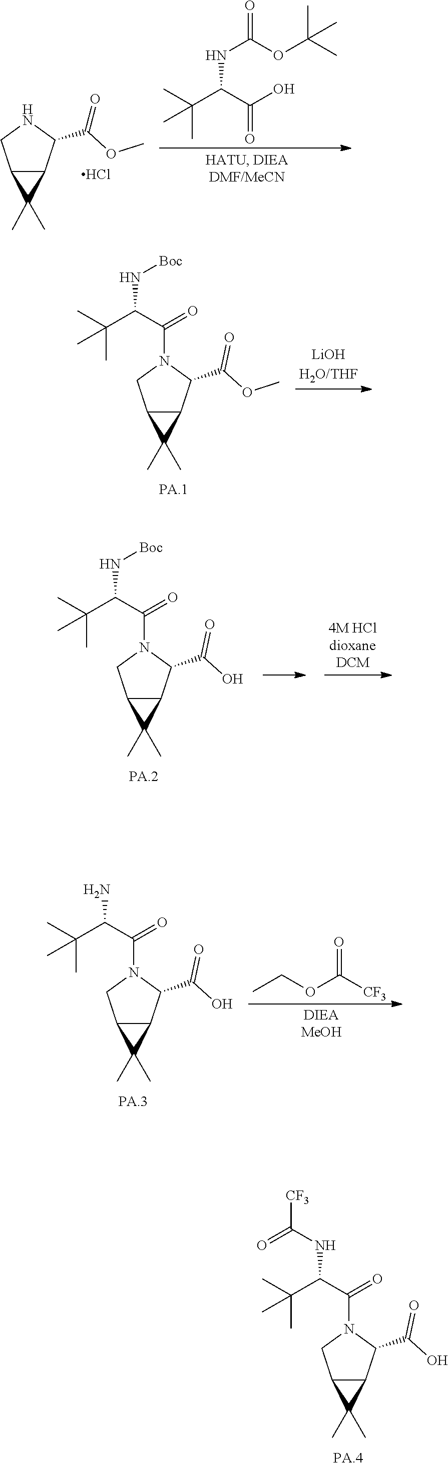

### Preparation 1. (1R,2S,5S)-3-((S)-2-((tert-Butoxycarbonyl)amino)-3,3-dimethylbutanoyl)-6,6-dimethyl-3-azabicyclo[3.1.0]hexane-2-carboxylate (PA.1)

To a solution of methyl (1R,2S,5S)-6,6-dimethyl-3-azabicyclo[3.1.0]hexane-2-carboxylate HCl salt (4.5 g, 21.9 mmol) and N-(tert-butoxycarbonyl)-3-methyl-L-valine (5.58 g, 24.1 mmol) in a mixture of N,N-dimethylformamide (9.0 ml) and acetonitrile (81 ml) was added HATU (9.09 g, 24.1 mmol) and N,N-diisopropylethylamine (8.55 g, 95.7 mmol) at 0° C. The reaction mixture was then allowed to warm to 25° C. and then stirred for 16 h. The mixture was treated with ethyl acetate, washed by water and 1M HCl_{(aq)}, and saturated aq. sodium chloride solution. The organic layer was dried over sodium sulfate, filtered, and concentrated. The residue was purified by silica gel chromatography (n-Hexane:ethyl acetate=3:1) to give the desired product PA.1 (5.6 g, 67%). ¹H NMR (400 MHz, DMSO-d₆), δ: 6.72 (d, J=9.6 Hz, 1H), 4.20 (s, 1H), 4.05-4.03 (m, 1H), 3.92 (d, J=10.4 Hz, 1H), 3.80-3.76 (m, 1H), 3.64 (s, 3H), 1.53-1.50 (m, 1H), 1.41-1.39 (m, 1H), 1.34 (s, 9H), 1.00 (s, 3H), 0.92 (s, 9H), 0.84 (s, 3H).

### Preparation 2. (1R,2S,5S)-3-((S)-2-((tert-Butoxycarbonyl)amino)-3,3-dimethylbutanoyl)-6,6-dimethyl-3-azabicyclo[3.1.0]hexane-2-carboxylic Acid (PA.2)

To a solution of PA.1 (5.6 g, 15 mmol) in THF (20 ml) was added lithium hydroxide (1.1 g, 44 mmol) and water (20 ml). After the reaction mixture was stirred at 25° C. for 17 h, it was concentrated to remove THF. The residue was then adjusted to pH 2 by addition of 1 M HCl_{(aq)}. The resulting mixture was extracted with ethyl acetate, and the combined organic layers were washed with brine, dried over sodium sulfate, filtered, and concentrated to give PA.2 (6.16 g, crude). ¹H NMR (400 MHz, DMSO-d₆), δ: 6.67 (d, J=9.2 Hz, 1H), 4.11 (s, 1H), 4.05-4.03 (m, 1H), 3.90 (d, J=10.4 Hz, 1H), 3.78-3.74 (m, 1H), 1.50-1.47 (m, 1H), 1.39-1.38 (m, 1H), 1.34 (s, 9H), 1.00 (s, 3H), 0.92 (s, 9H), 0.83 (s, 3H).

### Preparation 3. (1R,2S,5S)-3-((S)-2-Amino-3,3-dimethylbutanoyl)-6,6-dimethyl-3-azabicyclo[3.1.0]hexane-2-carboxylic Acid Hydrochloride (PA.3)

A solution of 4M HCl in 1,4-dioxane (21 ml) was added to a solution of PA.2 (6.16 g) in DCM (19 ml). After the mixture was stirred at 25° C. for 16 h, the solvent was removed under reduced pressure to give a HCl salt of PA.3 (4.7 g, crude). ¹H NMR (400 MHz, DMSO-d₆), δ: 8.31 (br. s, 2H), 4.15 (s, 1H), 3.80-3.76 (m, 2H), 3.72-3.69 (m, 1H), 1.55-1.52 (m, 1H), 1.43 (d, J=7.6 Hz, 1H), 1.02 (s, 9H), 1.01 (s, 3H), 0.95 (s, 3H).

### Preparation 4. (1R,2S,5S)-3-((S)-3,3-Dimethyl-2-(2,2,2-trifluoroacetamido)butanoyl)-6,6-dimethyl-3-azabicyclo[3.1.0]hexane-2-carboxylic Acid (PA.4)

To a solution of the HCl salt of PA.3 (4.7 g, crude) in methanol (15 ml) was added triethylamine (13 ml) and ethyl trifluoroacetate (3.13 mL, 26.3 mmol) at 0° C. The mixture was allowed to warm to 60° C., and then stirred for 16 h. The mixture was concentrated, and the residue was diluted with water and adjusted to pH 3 by addition of 1 M HCl_{(aq)}. After extraction of the aq. layer with ethyl acetate, the combined organic layers were dried over sodium sulfate, filtered, and concentrated to give PA.4 (5.5 g) which was used in next step without purification. ¹H NMR (400 MHz, DMSO-d₆), δ: 9.43 (d, J=8.6 Hz, 1H), 4.43 (d, J=8.6 Hz, 1H), 4.14 (s, 1H), 3.84 (dd, J=10.8, 5.6 Hz, 1H), 3.71 (d, J=10.8 Hz, 1H), 1.52 (dd, J=7.6, 5.2 Hz, 1H), 1.42 (d, J=7.6 Hz, 1H), 1.00 (s, 12H), 0.81 (s, 3H).

### Synthesis of (1R,2S,5S)-3-((S)-3,3-dimethyl-2-(methylsulfonamido)butanoyl)-6,6-dimethyl-3-azabicyclo[3.1.0]hexane-2-carboxylic Acid (PA.7)

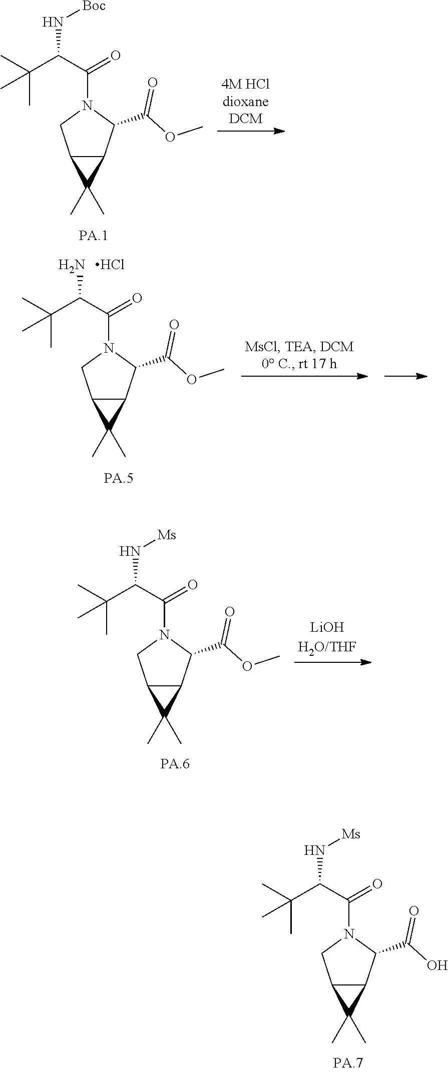 [SMILES: COC(=O)[C@@H]1[C@@H]2[C@H](CN1C(=O)[C@@H](NC(=O)OC(C)(C)C)C(C)(C)C)C2(C)C ; Cl ; COC(=O)[C@@H]1[C@@H]2[C@H](CN1C(=O)[C@@H](N)C(C)(C)C)C2(C)C ; COC(=O)[C@@H]1[C@@H]2[C@H](CN1C(=O)[C@@H](NS(C)(=O)=O)C(C)(C)C)C2(C)C ; CC(C)(C)[C@H](NS(C)(=O)=O)C(=O)N1C[C@H]2[C@@H]([C@H]1C(=O)O)C2(C)C]

### Preparation 5. Methyl (1R,2S,5S)-3-((S)-2-Amino-3,3-dimethylbutanoyl)-6,6-dimethyl-3-azabicyclo[3.1.0]hexane-2-carboxylate Hydrochloride (PA.5)

A solution of HCl in 1,4-dioxane (4 M, 20 ml) was added to a solution of PA.1 (5.0 g, 13.08 mmol) in DCM (20 ml). After the mixture was stirred at 25° C. for 16 h, the solvent was removed under reduced pressure to give a HCl salt of PA.5 (4.3 g, crude). ¹H NMR (400 MHz, DMSO-d₆), δ: 8.35 (br. s, 3H), 4.22 (s, 1H), 3.83-3.79 (m, 2H), 3.74-3.72 (m, 1H), 3.65 (s, 3H), 1.57-1.56 (m, 1H), 1.46 (s, J=7.6 Hz, 1H), 1.02-1.01 (m, 12H), 0.96 (s, 3H).

### Preparation 6. Methyl (1R,2S,5S)-3-((S)-3,3-dimethyl-2-(methylsulfonamido)butanoyl)-6,6-dimethyl-3-azabicyclo[3.1.0]hexane-2-carboxylate (PA.6)

To a solution of PA.5 (500 mg, 1.58 mmol) in DCM (10 ml) was added MsCl (270 mg, 2.36 mmol) and TEA (640 mg, 6.32 mmol) at 0° C. The reaction mixture was stirred at 25° C. for 17 h. The mixture was concentrated and purified by silica gel column chromatography (n-hexane:ethyl acetate=3:1) to give the desired product PA.6 (480 mg, 84% yield). ¹H NMR (400 MHz, DMSO-d₆), δ: 5.25 (d, J=10.0 Hz, 1H), 4.48 (s, 1H), 3.92-3.88 (m, 1H), 3.85-3.77 (m, 2H), 3.75 (s, 3H), 2.89 (s, 3H), 1.51-1.45 (m, 2H), 1.06-1.05 (m, 12H), 0.97 (s, 3H).

### Preparation 7. (1R,2S,5S)-3-((S)-3,3-Dimethyl-2-(methylsulfonamido)butanoyl)-6,6-dimethyl-3-azabicyclo[3.1.0]hexane-2-carboxylic Acid (PA.7)

To a solution of PA.6 (480 mg, 1.33 mmol) in THF (4.0 mL) was added lithium hydroxide (96 mg, 4.0 mmol) and water (4.0 mL). After the reaction mixture was stirred at 25° C. for 17 h, it was neutralized by 1 M HCl_{(aq)} and then concentrated to give PA.7 (800 mg, crude) which was used in next step. ¹H NMR (400 MHz, DMSO-d₆), δ: 7.07 (d, J=9.6 Hz, 1H), 4.12 (s, 1H), 3.82-3.76 (m, 2H), 3.40-3.67 (m, 1H), 2.83 (s, 3H), 1.54-1.51 (m, 1H), 1.40 (d, J=8.4 Hz, 1H), 1.00 (s, 3H), 0.97 (s, 9H), 0.88 (s, 3H).

### Synthesis of (1R,2S,5S)-3-[(2S)-2-(methoxycarbonylamino)-3,3-dimethyl-butanoyl]-6,6-dimethyl-3-azabicyclo[3.1.0]hexane-2-carboxylic Acid (PA.11)

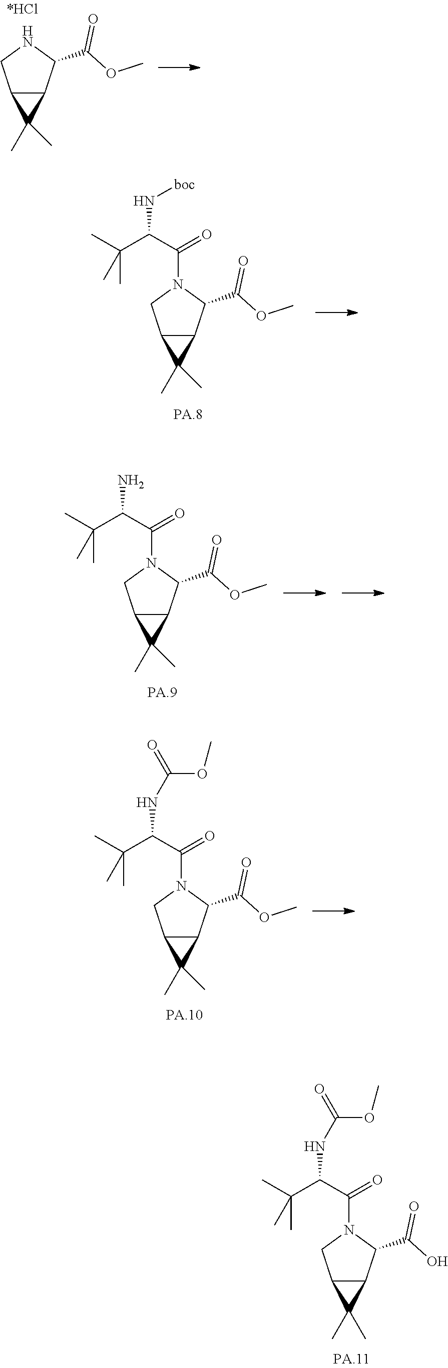

### Preparation 8. Methyl (1R,2S,5S)-6,6-dimethyl-3-[(2S)-3,3-dimethyl-2-(methylamino)butanoyl]-3-azabicyclo[3.1.0]hexane-2-carboxylate (PA.8)

To a solution of methyl (1R,2S,5S)-6,6-dimethyl-3-azabicyclo[3.1.0]hexane-2-carboxylate HCl salt (10 g, 48.7 mmol) and N-(tert-butoxycarbonyl)-3-methyl-L-valine (5.58 g, 24.1 mmol) in a mixture of DMF (20 ml) and ACN (180 ml) was added HATU (20.2 g, 53.6 mmol) and DIPEA (18.9 g, 212.7 mmol) at 0° C. The reaction mixture was then allowed to warm to 25° C. and then stirred for 16 h. The mixture was treated with ethyl acetate, washed by water and 1M HCl_{(aq)}, and saturated aqueous sodium chloride solution (18 g, 96%). ¹H NMR (400 MHz, DMSO-d₆), δ: 6.72 (d, J=9.6 Hz, 1H), 4.20 (s, 1H), 4.05 (d, J=10.4 Hz, 1H), 3.93 (d, J=10 Hz, 1H), 3.81-3.77 (m, 1H), 3.64 (s, 3H), 1.54-1.51 (m, 1H), 1.42-1.40 (m, 1H), 1.35 (s, 9H), 1.00 (s, 3H), 0.93 (s, 9H), 0.85 (s, 3H).

### Preparation 9. Methyl (1R,2S,5S)-3-[(2S)-2-amino-3,3-dimethyl-butanoyl]-6,6-dimethyl-3-azabicyclo[3.1.0]hexane-2-carboxylate (PA.9)

A solution of 4M HCl in 1,4-dioxane (20 ml) was added to a solution of PA.8 (9 g, 23.5 mmol) in DCM (20 ml). After the mixture was stirred at 25° C. for 3 h, the solvent was removed under reduced pressure to give a HCl salt of PA.9 (6 g, 90%). ¹H NMR (400 MHz, DMSO-d₆), δ: 8.21 (s, 2H), 4.25 (s, 1H), 3.83-3.79 (m, 2H), 3.72 (d, J=11.2 Hz, 1H), 3.67 (s, 3H), 3.56 (s, 3H), 1.61-1.58 (m, 1H), 1.49 (d, J=8 Hz, 1H), 1.03 (s, 9H), 0.86 (s, 3H).

### Preparation 10. Methyl (1R,2S,5S)-3-[(2S)-2-(methoxycarbonylamino)-3,3-dimethyl-butanoyl]-6,6-dimethyl-3-azabicyclo[3.1.0]hexane-2-carboxylate (PA.10)

To a solution of PA.9 (0.8 g, 2.8 mmol) in DCM was added methyl chloroformate (0.295 g, 3.1 mmol) and DIPEA (0.830 g, 6.4 mmol). After the mixture was stirred at 25° C. for 16 h. The mixture was treated with ethyl acetate, washed by aq. NaHCO₃, dried over sodium sulfate, filtered, and concentrated to give PA.10 (0.906 g, 95%). LCMS (ESI) m/z calc. for C₁₇H₂₈N₂O₅ 340.42; found, 341.6 [M+H]⁺.

### Preparation 11. (1R,2S,5S)-3-[(2S)-2-(methoxycarbonylamino)-3,3-dimethyl-butanoyl]-6,6-dimethyl-3-azabicyclo[3.1.0]hexane-2-carboxylic Acid (PA.11)

To a solution of PA.10 (0,906 g, 2.7 mmol) in THF was added lithium hydroxide (0.453 g, 10.8 mmol) and water. After the reaction mixture was stirred at 25° C. for 17 h, it was concentrated to remove THF. The residue was then adjusted to pH 2 by addition of 1 M HCl_{(aq)}. The resulting mixture was extracted with ethyl acetate, and the combined organic layers were washed with brine, dried over sodium sulfate, filtered, and concentrated to give PA.11 (0.416 g, 47.3%). ¹H NMR (400 MHz, DMSO-d₆), δ: 7.11 (d, J=10.6 Hz, 1H), 4.11 (s, 1H), 4.04 (d, J=8.4 Hz, 1H), 3.80 (s, 2H), 3.50 (s, 3H), 1.46 (s, 1H), 1.38 (d, J=7.6 Hz, 1H), 1.00 (s, 3H), 0.99 (dd, J=8.06, J=6.75, 1H), 0.95 (s, 9H), 0.85 (s, 3H).

### Synthesis of methyl N-[(1S)-1-[(1S)-2-[[(1S)-1-cyano-2-(1H-indol-3-yl)ethyl]carbamoyl]-6,6-dimethyl-3-azabicyclo[3.1.0]hexane-3-carbonyl]-2,2-dimethyl-propyl]carbamate (P2)

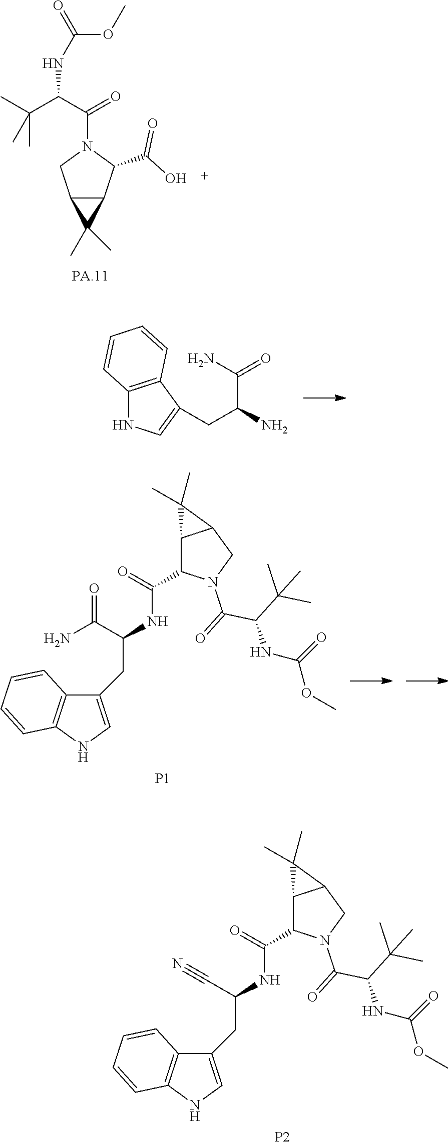

### Preparation 12. Methyl N-[(1S)-1-[(1S)-2-[[(1S)-2-amino-1-(1H-indol-3-ylmethyl)-2-oxo-ethyl]carbamoyl]-6,6-dimethyl-3-azabicyclo[3.1.0]hexane-3-carbonyl]-2,2-dimethyl-propyl]carbamate (P1)

To a solution of PA.11 (0.196, 0.6 mmol) in DMF was added (2S)-2-amino-3-(1H-indol-3-yl)propanamide (0.150 g, 0.63 mmol), 2-hydroxypyridine 1-oxide (17 mg, 0.15 mmol), DIPEA (0.310, 2.4 mmol), and 1-(3-dimethylaminopropyl)-3-ethylcarbodiimide hydrochloride (0.121 g, 0.78 mmol) at 0° C. After the reaction mixture was stirred at 25° C. for 16 h, it was diluted with ethyl acetate. The solution was washed by a mixture of water, 1 M HCl_{(aq)} and brine. The organic layer was dried over sodium sulfate, filtered, and concentrated to give a P1 (0.184 g, 62%). LCMS (ESI) m/z calc. for C₂₇H₃₇N₅O₅ 511.61; found, 512.6 [M+H]⁺.

### Preparation 13. Methyl N-[(1S)-1-[(1S)-2-[[(1S)-1-cyano-2-(1H-indol-3-yl)ethyl]carbamoyl]-6,6-dimethyl-3-azabicyclo[3.1.0]hexane-3-carbonyl]-2,2-dimethyl-propyl]carbamate (P2)

To a solution of P1 (0.184 g, 0.36 mmol) in DCM was added Burgess reagent (0.214 g, 0.9 mmol), and the resulting mixture was stirred for 16 h. The residue was diluted with a solution of EtOAc and washed with saturated NaHCO_{3(aq)}, 1N HCl_{(aq)}, and brine. The organic layer was dried over MgSO₄₍ₛ₎, filtered, and concentrated under reduced pressure to give P2 (0.105 g, 59%). LCMS (ESI) m/z calc. for C₂₇H₃₅N₅O₄ 493.59; found, 494.7 [M+H]⁺.

### Synthesis of ethyl N-[(1S)-1-[(1S)-2-[[(1S)-1-cyano-2-(1H-indol-3-yl)ethyl]carbamoyl]-6,6-dimethyl-3-azabicyclo[3.1.0]hexane-3-carbonyl]-2,2-dimethyl-propyl]carbamate (P4)

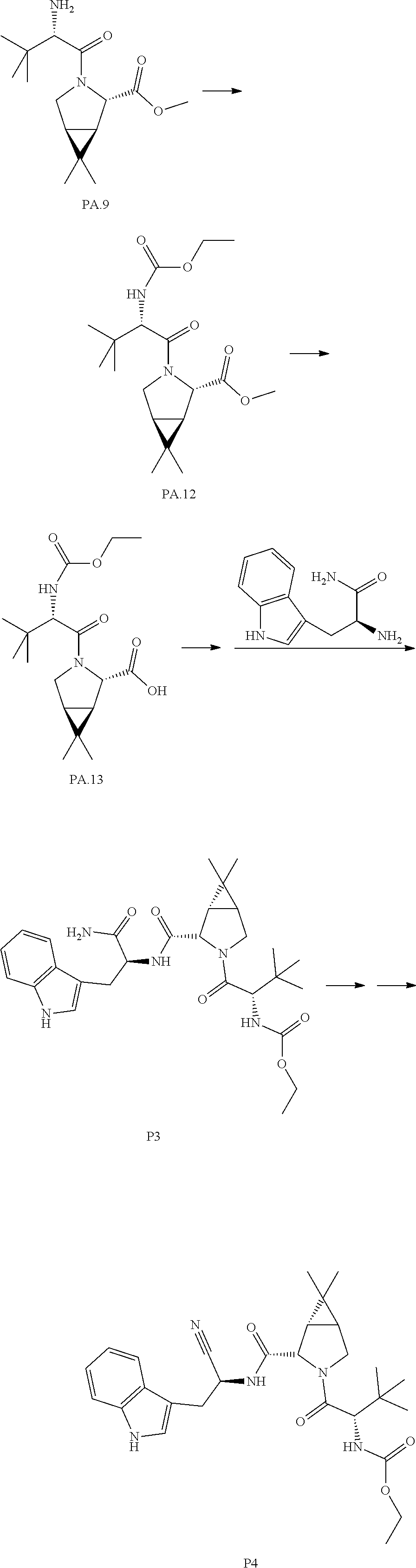

### Preparation 14. Methyl (1R,2S,5S)-3-[(2S)-2-(ethoxycarbonylamino)-3,3-dimethyl-butanoyl]-6,6-dimethyl-3-azabicyclo[3.1.0]hexane-2-carboxylate (PA.12)

To a solution of PA.9 (0.8 g, 2.8 mmol) in DCM was added ethyl chloroformate (0.334 g, 3.1 mmol) and DIPEA (0.905 g, 6.4 mmol). After the mixture was stirred at 25° C. for 16 h. The mixture was treated with ethyl acetate, washed by aq. NaHCO₃, dried over sodium sulfate, filtered, and concentrated to give PA.12 (0.813 g, 82%). LCMS (ESI) m/z calc. for C₁₈H₃₀N₂O₅ 354.44; found, 355.6 [M+H]⁺.

### Preparation 15. (1R,2S,5S)-3-[(2S)-2-(Ethoxycarbonylamino)-3,3-dimethyl-butanoyl]-6,6-dimethyl-3-azabicyclo[3.1.0]hexane-2-carboxylic Acid (PA.13)

To a solution of PA.12 (0,813 g, 2.3 mmol) in THF was added LiOH (0.386 g, 9.2 mmol) and water. After the reaction mixture was stirred at 25° C. for 17 h, it was concentrated to remove THF. The residue was then adjusted to pH 2 by addition of 1 M HCl_{(aq)}. The resulting mixture was extracted with ethyl acetate, and the combined organic layers were washed with brine, dried over sodium sulfate, filtered, and concentrated to give PA.13 (0.468 g, 60%). ¹H NMR (400 MHz, DMSO-d₆), δ: 12.61 (s, 1H), 7.06 (d, J=8.8 Hz, 1H), 4.12 (s, 1H), 4.09-3.89 (m, 5H), 3.86-3.76 (m, 3H), 1.99 (s, 1H), 1.52-1.49 (m, 1H), 1.39 (d, J=8 Hz, 1H), 1.15-1.12 (m, 4H), 0.98-0.95 (m, 10H).

### Preparation 16. Ethyl N-[(1S)-1-[(1S)-2-[[(1S)-2-amino-1-(1H-indol-3-ylmethyl)-2-oxo-ethyl]carbamoyl]-6,6-dimethyl-3-azabicyclo[3.1.0]hexane-3-carbonyl]-2,2-dimethyl-propyl]carbamate (P3)

To a solution of PA.13 (0.200, 0.59 mmol) in DMF was added (2S)-2-amino-3-(1H-indol-3-yl)propanamide (0.148 g, 0.62 mmol), 2-hydroxypyridine 1-oxide (16 mg, 0.15 mmol), DIPEA (0.301 g, 2.4 mmol), and 1-(3-dimethylaminopropyl)-3-ethylcarbodiimide hydrochloride (0.119 g, 0.77 mmol) at 0° C. After the reaction mixture was stirred at 25° C. for 16 h, it was diluted with ethyl acetate. The solution was washed by a mixture of water, 1 M HCl_{(aq)} and brine. The organic layer was dried over sodium sulfate, filtered, and concentrated to give P3 (0.201 g, 65%). LCMS (ESI) m/z calc. for C₂₈H₃₉N₅O₅ 525.64; found, 526.8 [M+H]⁺.

### Preparation 17. Ethyl N-[(1S)-1-[(1S)-2-[[(1S)-1-cyano-2-(1H-indol-3-yl)ethyl]carbamoyl]-6,6-dimethyl-3-azabicyclo[3.1.0]hexane-3-carbonyl]-2,2-dimethyl-propyl]carbamate (P4)

To a solution of P3 (0.201 g, 0.39 mmol) in DCM was added Burgess reagent (0.234 g, 0.98 mmol), and the resulting mixture was stirred for 16 h. The residue was diluted with a solution of EtOAc and washed with saturated NaHCO_{3(aq)}, 1N HCl_{(aq)}, and brine. The organic layer was dried over MgSO₄₍ₛ₎, filtered, and concentrated under reduced pressure to give P4 (0.189 g, 96%). LCMS (ESI) m/z calc. for C₂₈H₃₇N₅O₄ 507.62; found, 508.4 [M+H]⁺.

### Synthesis of (1R,2S,5S)—N-[1-cyano-3-(1H-indol-3-yl)propyl]-6,6-dimethyl-3-[(2S)-3,3-dimethyl-2-[(2,2,2-trifluoroacetyl)amino]butanoyl]-3-azabicyclo[3.1.0]hexane-2-carboxamide (P8)

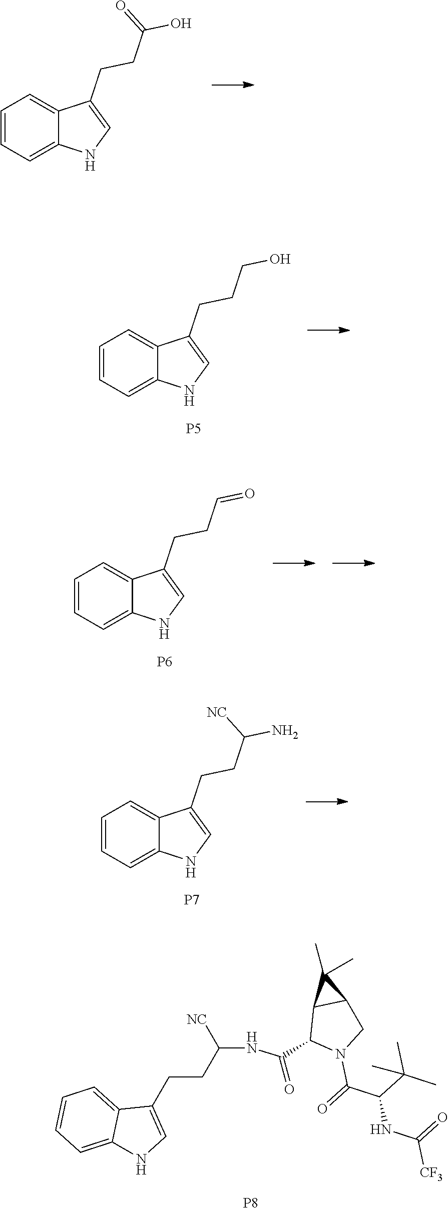

### Preparation 18. 3-(1H-Indol-3-yl)propan-1-ol (P5)

To a rapidly stirred suspension of lithium aluminium hydride (2.5 g, 66 mmol) in anh diethyl ether (100 mL) was added a solution of 3-(1H-indol-3-yl)propanoic acid (5 g, 26.4 mmol) in anh diethyl ether (100 ml) at such a rate that gentle reflux was maintained. During the course of the addition a white suspension was formed. The mixture was then stirred under reflux for a further 3 h and then at rt overnight. The excess lithium aluminium hydride was decomposed by the slow addition of water (5 mL), followed by the addition of a 1:3 mixture of sulfuric acid (95%)/water (150 mL). The ether layer was separated, and the aqueous layer was extracted with diethyl ether (3×250 mL). The combined ethereal fractions were dried, and evaporation of the solvent afforded P5 (4 g, 86%). ¹H NMR (400 MHz, DMSO-d6), δ: 10.71 (s, 1H), 7.49 (d, J=7.9 Hz, 1H), 7.35-7.28 (m, 1H), 7.05 (ddd, J=15.1, 7.8, 1.8 Hz, 2H), 6.95 (ddd, J=8.0, 6.9, 1.1 Hz, 1H), 4.42 (t, J=5.1 Hz, 1H), 3.46 (q, J=6.2 Hz, 2H), 2.70 (t, J=7.6 Hz, 2H), 1.84-1.73 (m, 2H).

### Preparation 19. 3-(1H-Indol-3-yl)propanal (P6)

To a solution of P5 (500 mg, 2.85 mmol) in EtOAc (20 ml) Dess-Martin periodinane (2.4 g, 8.55 mmol) was added. The mixture was stirred at rt for 16 h, concentrated under reduced pressure, diluted with sat. NaHCO₃ (100 ml), extracted with DCM (3×50 ml), dried over Na₂SO₄, filtered, and concentrated under reduced pressure, and the crude P6 was used in next step without purification.

### Preparation 20. 2-Amino-4-(1H-indol-3-yl)butanenitrile (P7)

To a solution of P6 (500 mg, 2.85 mmol) in 3M NH₃ in MeOH (50 ml) TMSCN (340 mg, 3.42 mmol) was added. The mixture was stirred at rt for 16 h, and concentrated under reduced pressure, and the crude P7 was used in next step without purification.

### Preparation 21. (1R,2S,5S)—N-[1-Cyano-3-(1H-indol-3-yl)propyl]-6,6-dimethyl-3-[(2S)-3,3-dimethyl-2-[(2,2,2-trifluoroacetyl)amino]butanoyl]-3-azabicyclo[3.1.0]hexane-2-carboxamide (P8)

To a mixture of the PA.4 (457 mg, 1.25 mmol) and HATU (501 mg, 1.31 mmol) in MeCN (10 ml) DIEA (195 mg, 1.5 mmol) was added. The mixture was stirred at rt for 30 min, and P7 (250 mg, 1.25 mmol) was added, and stirred overnight. Reaction mixture was diluted with ethyl acetate (50 ml) and washed with water (3×50 ml), brine (2×50 mL), dried on Na₂SO₄, and concentrated under reduced pressure, and the crude was purified by flash chromatography on silica gel (hexane/EtOAc—2:1) to give the title compound P8 (650 mg, 95%). ¹H NMR (400 MHz, DMSO-d6), δ: 10.81 (d, J=17.5 Hz, 1H), 9.41-9.31 (m, 1H), 7.53 (t, J=7.2 Hz, 1H), 7.37-7.31 (m, 1H), 7.17-7.03 (m, 2H), 6.97 (t, J=7.6 Hz, 1H), 4.81-4.64 (m, 1H), 4.41 (dd, J=8.4, 5.1 Hz, 1H), 4.26 (d, J=8.2 Hz, 1H), 3.90 (dt, J=9.7, 7.5 Hz, 1H), 3.70 (dd, J=14.3, 10.5 Hz, 1H), 2.91-2.72 (m, 1H), 2.13 (q, J=7.2 Hz, 2H), 1.61-1.49 (m, 1H), 1.31 (t, J=8.1 Hz, 1H), 1.08-0.95 (m, 11H), 0.94-0.80 (m, 3H).

### Synthesis of (S)-2-amino-3-((S)-2-oxopyrrolidin-3-yl)propanamide Hydrochloride (P2.6)

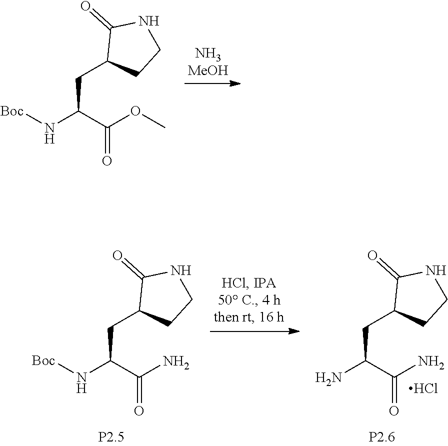 [SMILES: COC(=O)[C@H](C[C@@H]1CCNC1=O)NC(=O)OC(C)(C)C ; CC(C)(C)OC(=O)N[C@@H](C[C@@H]1CCNC1=O)C(N)=O ; NC(=O)[C@@H](N)C[C@@H]1CCNC1=O ; Cl]

### Preparation 22. tert-Butyl ((S)-1-amino-1-oxo-3-((S)-2-oxopyrrolidin-3-yl)propan-2-yl)carbamate (P2.5)

A solution of methyl (2S)-2-(tert-butoxycarbonylamino)-3-[(3S)-2-oxopyrrolidin-3-yl]propanoate (10 g, 35 mmol) and ammonia in methanol solution (90 mL) was stirred at rt for 24 h. The reaction mixture was concentrated to afford P2.5 (9.5 g, 100%) as a yellow solid which was used in the next step without further purification. ¹H NMR (400 MHz, CD₃OD), δ: 4.10 (dd, J=11.0, 4.0 Hz, 1H), 3.37-3.32 (m, 2H), 2.57-2.42 (m, 1H), 2.42-2.25 (m, 1H), 2.10-1.97 (m, 1H), 1.95-1.79 (m, 1H), 1.79-1.66 (m, 1H), 1.45 (s, 9H); LCMS (ESI+) m/z: 271.9 [M+H]⁺.

### Preparation 23. (S)-2-Amino-3-((S)-2-oxopyrrolidin-3-yl)propanamide Hydrochloride (P2.6)

A solution of P2.5 (9.0 g, 33 mmol) and HCl (3 M in methanol, 55 mL) in IPA (200 mL) was stirred at rt for 48 h. The reaction mixture was filtered and the collected solid was washed with IPA to give P2.6 (4.78 g, 70%) as a yellow solid. ¹H NMR (400 MHz, CD₃OD), δ: 4.04 (dd, J=9.2, 4.4 Hz, 1H), 3.43-3.36 (m, 2H), 2.83-2.70 (m, 1H), 2.50-2.38 (m, 1H), 2.12-1.95 (m, 2H), 1.95-1.79 (m, 1H); LRMS (ESI+) m/z: 172.0 [M+H]⁺.

### Synthesis of (1R,2S,5S)—N—((S)-1-amino-1-oxo-3-((S)-2-oxopyrrolidin-3-yl)propan-2-yl)-3-((S)-2-amino-3,3-dimethylbutanoyl)-6,6-dimethyl-3-azabicyclo[3.1.0]hexane-2-carboxamide (P2.8)

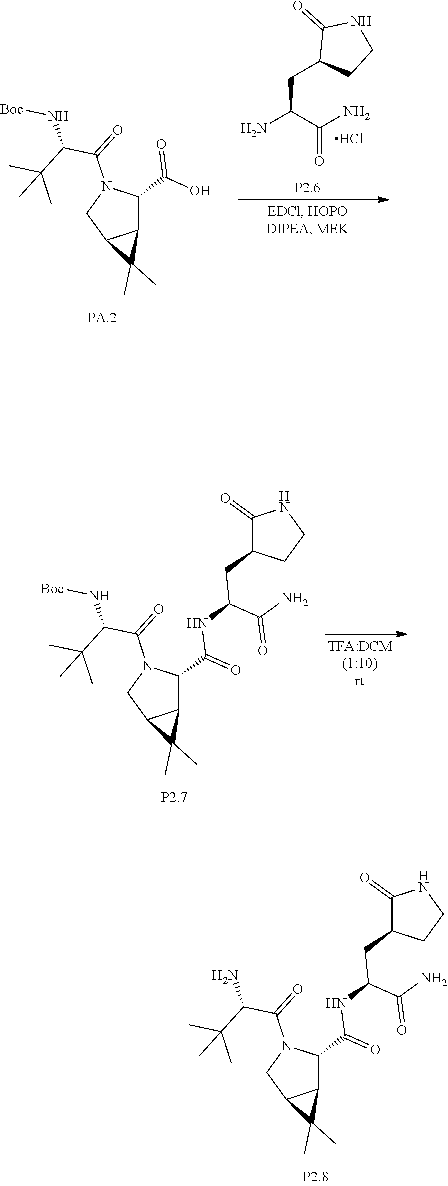 [SMILES: CC(C)(C)OC(=O)N[C@H](C(=O)N1C[C@H]2[C@@H]([C@H]1C(=O)O)C2(C)C)C(C)(C)C ; NC(=O)[C@@H](N)C[C@@H]1CCNC1=O ; Cl ; CC(C)(C)OC(=O)N[C@H](C(=O)N1C[C@H]2[C@@H]([C@H]1C(=O)N[C@@H](C[C@@H]1CCNC1=O)C(N)=O)C2(C)C)C(C)(C)C ; CC(C)(C)[C@H](N)C(=O)N1C[C@H]2[C@@H]([C@H]1C(=O)N[C@@H](C[C@@H]1CCNC1=O)C(N)=O)C2(C)C]

### Preparation 24. tert-Butyl N-[(1S)-1-[(1R,2S,5S)-2-[[(1S)-2-amino-2-oxo-1-[[(3S)-2-oxopyrrolidin-3-yl]methyl]ethyl]carbamoyl]-6,6-dimethyl-3-azabicyclo[3.1.0]hexane-3-carbonyl]-2,2-dimethyl-propyl]carbamate (P2.7)

To a solution of PA.2 (1.00 g, 2.72 mmol) in butan-2-one (8.0 mL) was added P2.6 (675 mg, 3.26 mmol, HCl salt), 2-hydroxypyridine 1-oxide (76 mg, 0.68 mmol), N,N-diisopropylethylamine (1.4 g, 10.88 mmol), and EDCI HCl (675 mg, 3.54 mmol) at 0° C. The reaction mixture was stirred at 25° C. for 16 h. The mixture was concentrated and purified by reverse phase column chromatography (5-100% of MeOH in water) to give P2.7 (1.2 g, 85% yield). ¹H NMR (400 MHz, CDCl₃), δ: 8.19 (s, 1H), 7.17 (s, 1H), 6.20 (br. s, 1H), 5.73 (br. s, 1H), 5.20 (br. d, 1H), 4.47-4.41 (m, 1H), 4.23-4.21 (m, 2H), 4.08-4.04 (m, 1H), 3.98 (d, J=10.4 Hz, 1H), 3.46 (s, 1H), 3.35-3.32 (m, 2H), 2.48-2.34 (m, 2H), 2.07-2.04 (m, 2H), 2.00-1.95 (m, 1H), 1.87-1.82 (m, 1H), 1.38 (s, 9H), 1.01-0.99 (m, 12H), 0.87 (s, 3H).

### Preparation 25. (1R,2S,5S)—N—((S)-1-Amino-1-oxo-3-((S)-2-oxopyrrolidin-3-yl)propan-2-yl)-3-((S)-2-amino-3,3-dimethylbutanoyl)-6,6-dimethyl-3-azabicyclo[3.1.0]hexane-2-carboxamide (P2.8)

To a solution of P2.7 (100 mg, 0.19 mmol) in DCM (2.0 mL) was added TFA (0.2 mL) at rt. The reaction mixture was stirred at 25° C. for 17 h. The mixture was concentrated and purified by reverse column chromatography (5-100% of MeOH in water) to give the desired product P2.8 (40 mg, 50% yield). ¹H NMR (400 MHz, CDCl₃), δ: 8.42 (s, 1H), 8.28 (br. s, 1H), 7.22 (br. s, 1H), 5.85 (br. s, 1H), 5.56 (br. s, 1H), 5.32 (br. s, 1H), 4.45-4.40 (m, 1H), 4.33-4.26 (m, 1H), 4.13-4.00 (m, 1H), 3.67-3.55 (m, 1H), 3.38-3.35 (m, 2H), 3.22-3.00 (m, 1H), 2.49-2.37 (m, 2H), 2.16-2.11 (m, 1H), 1.97-1.86 (m, 2H), 1.51-1.42 (m, 2H), 1.05-1.01 (m, 9H), 0.96-2.94 (m, 6H).

### Synthesis of (S)-2-amino-3-(2-oxo-2,3-dihydro-1H-benzo[d]imidazol-1-yl)propanamide Hydrochloride (P3.3)

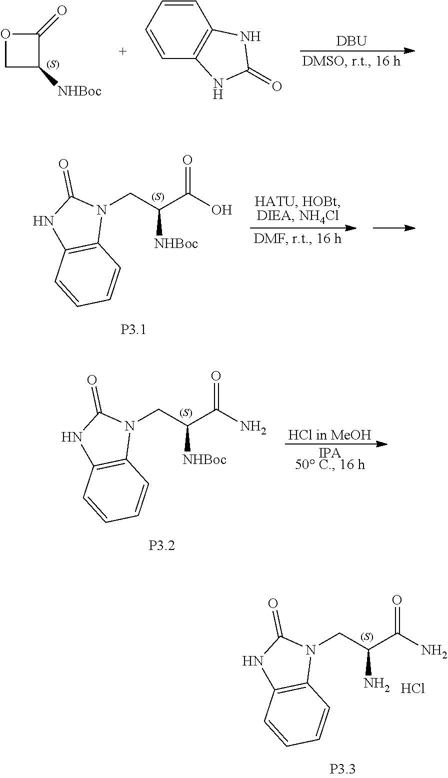

### Preparation 26. (S)-2-((tert-Butoxycarbonyl)amino)-3-(2-oxo-2,3-dihydro-1H-benzo[d]imidazol-1-yl)propanoic Acid (P3.1)

To a solution of 2-hydroxybenzimidazole (750 mg, 5.59 mmol, 1.0 eq) in DMSO (12 mL) was added 1,8-diazabicyclo[5.4.0]undec-7-ene (DBU, 1.0 mL, 6.71 mmol, 1.2 eq), and then the mixture was stirred at rt for 15 min. To the reaction mixture was added N-Boc-L-serine β-lactone (1.26 g, 6.71 mmol, 1.2 eq) in DMSO (12 mL), and the reaction mixture was stirred at rt for another 16 h. The mixture was concentrated, treated with water, adjusted pH value to 6 by HCl_{(aq)}, and extracted with ethyl acetate 3 times. The combined organic layers were washed with brine, dried over Na₂SO₄₍ₛ₎, filtered, and concentrated in vacuo. The residue was purified by C-18 reversed-phase column chromatography (mobile phase A: water, mobile phase B: methanol, UV: 214 and 254 nm, Flow rate: 35 mL/min, Gradient: 0-90% (% B)) to give P3.1 (300 mg, 17% yield) as a white solid. ¹H NMR (400 MHz, DMSO-d₆), δ: 10.85 (s, 1H), 7.17-7.10 (m, 2H), 7.00-6.93 (m, 3H), 4.31-4.24 (m, 1H), 4.19-4.10 (m, 1H), 4.02-3.99 (m, 1H), 1.22 (s, 9H).

### Preparation 27. tert-Butyl (S)-(1-amino-1-oxo-3-(2-oxo-2,3-dihydro-1H-benzo[d]imidazol-1-yl)propan-2-yl)carbamate (P3.2)

To a solution of P3.1 (300 g, 0.93 mmol, 1.0 eq) in DMF (3.5 mL) was added 1-[bis(dimethylamino)methylene]-1H-1,2,3-triazolo[4,5-b]pyridinium-3-oxide hexafluorophosphate (HATU, 426 mg, 1.12 mmol, 1.2 eq), hydroxybenzotriazole (HOBt, 71.5 mg, 0.467 mmol, 0.5 eq), ammonium chloride (75 mg, 1.40 mmol, 1.5 eq) and N,N-diisopropylethylamine (DIPEA, 0.81 mL, 4.67 mmol, 5.0 eq), and then stirred at rt for 16 h. The mixture was treated with water and extracted with ethyl acetate for 3 times. The combined organic layers were washed with brine, dried over Na₂SO₄₍ₛ₎, filtered, and concentrated in vacuo. The residue was purified by C-18 reversed-phase column chromatography (mobile phase A: water (with NH₄HCO₃), mobile phase B: methanol, UV: 214 and 254 nm, Flow rate: 35 mL/min, Gradient: 0-90% (% B)) to give P3.2 (117 mg, 39% yield) as a white solid. ¹H NMR (400 MHz, DMSO-d₆), δ: 10.83 (s, 1H), 7.44 (s, 1H), 7.22-7.13 (m, 2H), 7.01-6.92 (m, 3H), 6.79 (d, J=8.8 Hz, 1H), 4.30-4.24 (m, 1H), 4.09 (dd, J=14.4, 4.4 Hz, 1H), 3.90 (dd, J=14.0, 8.8 Hz, 1H), 1.19 (s, 9H).

### Preparation 28. (S)-2-Amino-3-(2-oxo-2,3-dihydro-1H-benzo[d]imidazol-1-yl)propanamide Hydrochloride (P3.3)

To a solution of P3.2 (128 mg, 0.400 mmol, 1.0 eq) in IPA (2.6 mL) was added 3 M HCl in methanol (0.67 mL, 2.0 mmol, 5.0 eq) dropwise. The reaction mixture was stirred at 50° C. for 16 h. The precipitation was collected by filtration, washed with IPA, and dried to give P3.3 (79 mg, 90% yield) as a white solid. ¹H NMR (400 MHz, DMSO-d₆), δ: 11.04 (s, 1H), 8.37 (s, 2H), 8.17 (s, 1H), 7.65 (s, 1H), 7.29-7.24 (m, 1H), 7.04-6.98 (m, 3H), 4.24 (dd, J=15.0, 6.8 Hz, 1H), 4.14 (dd, J=15.0, 5.2 Hz, 1H), 4.12-4.05 (m, 1H).

### Synthesis of (S)-2-amino-3-(1H-1,2,3-triazol-1-yl)propanamide (P3.7)

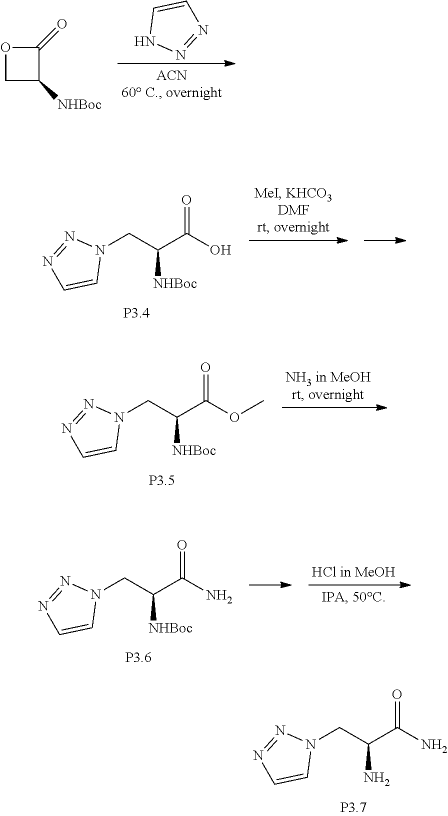 [SMILES: CC(C)(C)OC(=O)N[C@H]1COC1=O ; c1c[nH]nn1 ; CC(C)(C)OC(=O)N[C@@H](Cn1ccnn1)C(=O)O ; COC(=O)[C@H](Cn1ccnn1)NC(=O)OC(C)(C)C ; CC(C)(C)OC(=O)N[C@@H](Cn1ccnn1)C(N)=O ; NC(=O)[C@@H](N)Cn1ccnn1]

### Preparation 29. (S)-2-((tert-Butoxycarbonyl)amino)-3-(1H-1,2,3-triazol-1-yl)propanoic Acid (P3.4)

To a solution of tert-butyl (S)-(2-oxooxetan-3-yl)carbamate (1.00 g, 5.34 mmol) in ACN (20 ml) 1H-1,2,3-triazole (553 mg, 8.02 mmol) was added at rt. The reaction mixture was stirred at 60° C. for 16 h. The mixture was concentrated and purified by C18 column chromatography (5-100% of MeOH in water) to give the desired product P3.4 (460 mg, 34% yield). ¹H NMR (400 MHz, CDCl₃), δ: 7.72 (s, 1H), 7.63 (s, 1H), 5.52 (d, J=6.0 Hz, 1H), 5.02-4.89 (m. 2H), 4.69 (br. s, 1H), 1.44 (s, 9H).

### Preparation 30. Methyl (S)-2-((tert-butoxycarbonyl)amino)-3-(1H-1,2,3-triazol-1-yl)propanoate (P3.5)

To a solution of P3.4 (700 mg, 2.73 mmol) in DMF (14 ml) was added iodomethane (582 mg, 4.10 mmol) and KHCO₃ (546 mg, 5.46 mmol). After the reaction mixture was stirred at 25° C. for 17 h, the reaction was diluted by ethyl acetate and washed by water and brine. The organic layer was dried over sodium sulfate, filtered, and concentrated to give P3.5 (260 mg, 35% yield). ¹H NMR (400 MHz, CDCl₃), δ: 7.70 (d, J=1.2 Hz, 1H), 7.53 (d, J=1.2 Hz, 1H), 5.36 (d, J=6.4 Hz, 1H), 4.92-4.80 (m. 2H), 4.74-4.70 (m, 1H), 3.78 (s, 3H), 1.44 (s, 9H).

### Preparation 31. tert-Butyl (S)-(1-amino-1-oxo-3-(1H-1,2,3-triazol-1-yl)propan-2-yl)carbamate (P3.6)

To a solution of ammonia in methanol (7 M, 8.0 ml) was added P3.5 (260 mg, 0.96 mmol) and stirred at 25° C. for 16 h. The mixture was concentrated to give the desired product P3.6 (240 mg, crude). ¹H NMR (400 MHz, DMSO-d₆), δ: 7.99 (s, 1H), 7.68 (s, 1H), 7.52 (br. s, 1H), 7.26 (br. s, 1H), 7.04 (d, J=8.8 Hz, 1H), 4.71 (dd, J=4.4, 13.6 Hz, 1H), 4.47-4.34 (m, 2H), 1.30 (s, 9H).

### Preparation 32. (S)-2-Amino-3-(1H-1,2,3-triazol-1-yl)propanamide (P3.7)

To a solution of P3.6 (240 mg, crude) in IPA (12 ml) was added a solution of HCl in methanol (4 mL) at 0° C. The mixture was stirred at 50° C. for 16 h. The mixture was concentrated to give P3.7 (200 mg as a HCl salt) which was used in next step without purification. ¹H NMR (400 MHz, DMSO-d₆), δ: 8.48 (br. s, 3H), 8.13 (m, 2H), 7.78-7.77 (m, 1H), 7.73 (br. s, 1H), 4.86 (d, J=5.6 Hz, 2H), 4.32-4.29 (m, 1H).

### Synthesis of (S)-2-amino-3-(4,5-dimethyl-1H-1,2,3-triazol-1-yl)propanamide (P3.12)

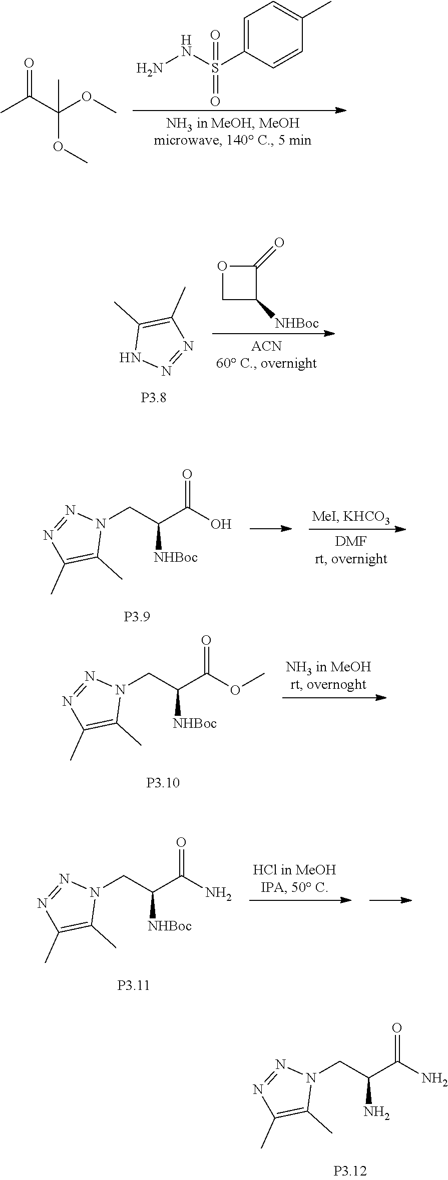

### Preparation 33. 4,5-Dimethyl-1H-1,2,3-triazole (P3.8)

To a solution of 3,3-dimethoxybutan-2-one (2.50 g, 18.9 mmol) in methanol (38 ml) was added 4-methylbenzenesulfonohydrazide (3.5 g, 18.9 mmol) and NH₃ solution (5.95 mL, 7N in methanol) at rt. The reaction mixture was stirred at 140° C. for 5 min under microwave irradiation. The mixture was concentrated and purified by silica gel column chromatography (0-55% of EtOAc in n-hexane) to give the desired product P3.8 (220 mg, 12% yield). ¹H NMR (400 MHz, CDCl₃), δ: 2.28 (s, 6H).

### Preparation 34. (S)-2-((tert-Butoxycarbonyl)amino)-3-(4,5-dimethyl-1H-1,2,3-triazol-1-yl)propanoic Acid (P3.9)

To a solution of tert-butyl (S)-(2-oxooxetan-3-yl)carbamate (290 mg, 1.54 mmol) in acetonitrile (2.0 ml) was added P3.8 (100 mg, 1.03 mmol) at rt. The reaction mixture was stirred at 60° C. for 16 h. The mixture was concentrated to give the crude product P3.9 which was used for next step without purification.

### Preparation 35. Methyl (S)-2-((tert-butoxycarbonyl)amino)-3-(1H-1,2,3-triazol-1-yl)propanoate (P3.10)

To a solution of P3.9 (1.03 mmol) in DMF (2.0 ml) was added iodomethane (210 mg, 1.03 mmol) and potassium hydrogen carbonate (200 mg, 2.06 mmol). After the reaction mixture was stirred at 25° C. for 17 h, it was treated with ethyl acetate and washed by water and brine. The organic layer was dried over sodium sulfate, filtered, concentrated, and purified by preparative TLC (5% MeOH in DCM) to give P3.10 (40 mg). ¹H NMR (400 MHz, CDCl₃), δ: 4.71-4.65 (m, 2H), 4.58-4.53 (m, 1H), 3.78 (s, 3H), 2.25 (s, 3H), 2.19 (s, 3H), 1.43 (s, 9H).

### Preparation 36. tert-Butyl (S)-(1-amino-3-(4,5-dimethyl-1H-1,2,3-triazol-1-yl)-1-oxopropan-2-yl)carbamate (P3.11)

A solution of NH₃ in methanol (7 N, 2.0 ml) was added to P3.10 (40 mg, 0.13 mmol). The solution was stirred at 25° C. for 16 h. The mixture was concentrated to give the desired product P3.11 (29 mg, crude) which was used for next step without purification.

### Preparation 37. (S)-2-Amino-3-(4,5-dimethyl-1H-1,2,3-triazol-1-yl)propanamide (P3.12)

To a solution of P3.11 (29 mg, crude) in IPA (3.0 ml) at 0° C. was added a solution of HCl in methanol (1.0 mL). The mixture was allowed to warm to 50° C. and was stirred for 16 h. The mixture was concentrated to give P3.12 (30 mg, crude, HCl salt). ¹H NMR (400 MHz, DMSO-d₆), δ: 8.52 (br. s, 2H), 8.10 (br. s, 1H), 7.66 (br. s, 1H), 4.68-4.57 (m, 2H), 4.23-4.20 (m, 1H), 2.21 (s, 3H), 2.15 (s, 3H).

### Preparation 38. (S)-2-amino-3-(1H-1,2,3-triazol-1-yl)propanamide (P3.7)

To a solution of P3.6 (240 mg, crude) in IPA (12 ml) was added a solution of HCl in methanol (4 mL) at 0° C. The mixture was stirred at 50° C. for 16 h. The mixture was concentrated to give P3.7 (200 mg as a HCl salt) which was used in next step without purification. ¹H NMR (400 MHz, DMSO-d₆), δ: 8.48 (br. s, 3H), 8.13 (m, 2H), 7.78-7.77 (m, 1H), 7.73 (br. s, 1H), 4.86 (d, J=5.6 Hz, 2H), 4.32-4.29 (m, 1H).

### Synthesis of (1R,2S,5S)—N-[1-cyano-2-(1H-pyrrolo[2,3-b]pyridin-3-yl)ethyl]-6,6-dimethyl-3-[(2S)-3,3-dimethyl-2-[(2,2,2-trifluoroacetyl)amino]butanoyl]-3-azabicyclo[3.1.0]hexane-2-carboxamide (P12)

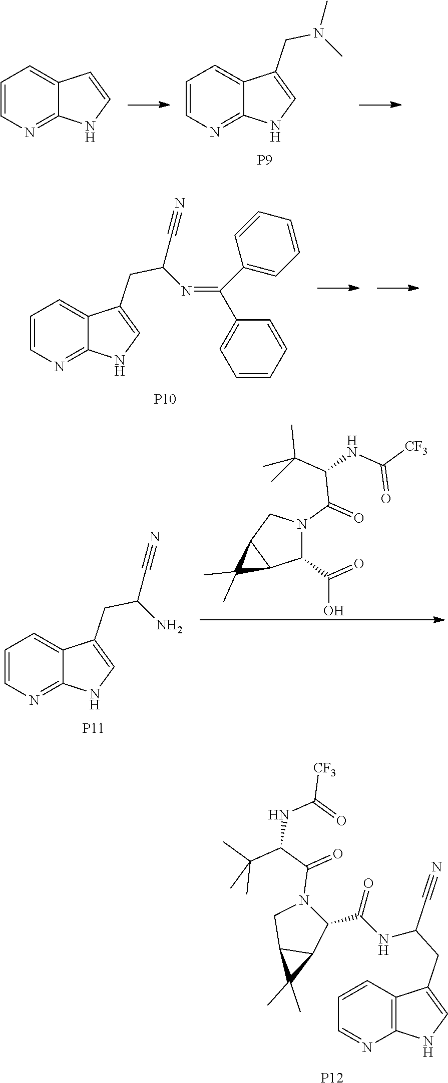

### Preparation 39. N,N-dimethyl-1-(1H-pyrrolo[2,3-b]pyridin-3-yl)methanamine (P9)

The mixture of AcOH_{(aq.)} (2.5 mL) and water (10 mL) was cooled to 5° C. and then dimethylamine (33% water solution) (8.5 mL, 63.56 mmol) and formalin (37% water solution) (3.6 mL, 50.85 mmol) were added to the reaction mixture. Reaction mixture was stirred at 5° C. about 30 min and then a solution of 1H-pyrrolo[2,3-b]pyridine (5 g, 42.37 mmol) in EtOH (5 mL) was added. The reaction mixture was warmed to rt and then was stirred under reflux overnight. The mixture was cooled to rt. The solvent was removed under reduced pressure. The residue was mixed with crushed ice and alkalized with NaOH solution to pH=11. The formed precipitate was filtered off, washed with water and air dried to give P9 (3.4 g, 46%) as a yellow solid. ¹H NMR (400 MHz, DMSO-d₆), δ: 11.42 (s, 1H), 8.18 (dd, J=4.8, 1.5 Hz, 1H), 7.98 (dd, J=7.8, 1.5 Hz, 1H), 7.32 (s, 1H), 7.02 (dd, J=7.9, 4.8 Hz, 1H), 3.51 (s, 2H), 2.13 (s, 6H).

### Preparation 40. 2-(Benzhydrylideneamino)-3-(1H-pyrrolo[2,3-b]pyridin-3-yl)propanenitrile (P10)

To a solution of P9 (3.4 g, 19.43 mmol) and 2-(benzhydrylideneamino)acetonitrile (3.5 g, 15.91 mmol) in dry DCM (250 mL) were added portion-wise 35% NaOH solution (3.1 g, 77.5 mmol) and then Me₂SO₄ (3.6 g, 28.57 mmol) over 1 h. The reaction mixture was stirred at room temperature 48 h. The insoluble material was filtered off through a Celite pad. The organic layer was separated and washed with brine. The organic layer was dried over Na₂SO₄₍ₛ₎, filtered, and then concentrated under reduced pressure. The residue was purified by silica gel column chromatography (0-20% EtOAc in n-hexane) to give P10 (0.28 g, 4%). ¹H NMR (400 MHz, DMSO-d₆), δ: 11.49 (s, 1H), 8.15 (d, J=3.2 Hz, 1H), 7.46 (m, 10H), 6.92 (m, 3H), 4.49 (t, J=7 Hz, 1H), 3.37 (m, 1H), 3.19 (m, 1H).

### Preparation 41. 2-Amino-3-(1H-pyrrolo[2,3-b]pyridin-3-yl)propanenitrile (P11)

To a solution of P10 (0.28 g, 0.80 mmol) in a mixture of THF (5 mL) and water (1 mL) was added HCl (0.5 mL) and resulting mixture was stirred at rt 30 min. The THF was removed under reduced pressure. The water and DCM were added to the residue, organic layer was separated and water layer was alkalized with NaOH solution to pH=11. The resulting mixture was diluted with DCM. The organic layer was separated and washed with brine. The organic layer was dried over Na₂SO₄₍ₛ₎, filtered, and then concentrated under reduced pressure to give P11 (0.1 g, 54%). ¹H NMR (400 MHz, DMSO-d₆), δ: 11.49 (s, 1H), 8.19 (d, J=4.6 Hz, 1H), 8.02 (d, J=7.8 Hz, 1H), 7.38 (d, J=2.3 Hz, 1H), 7.04 (m, 1H), 3.99 (m, 1H), 3.04 (d, J=7.4 Hz, 2H), 2.37 (s, 2H).

### Preparation 42. (1R,2S,5S)—N-[1-cyano-2-(1H-pyrrolo[2,3-b]pyridin-3-yl)ethyl]-6,6-dimethyl-3-[(2S)-3,3-dimethyl-2-[(2,2,2-trifluoroacetyl)amino]butanoyl]-3-azabicyclo[3.1.0]hexane-2-carboxamide (P12)

To a solution of P11 (100 mg, 0.54 mmol), PA.4 (200 mg, 0.55 mmol) in THF (3 mL) was added TBTU (140 mg, 0.60 mmol) and DIPEA (80 mg, 0.62 mmol) was added to the reaction mixture and the resulting mixture was stirred for overnight. The residue was concentrated under reduced pressure and diluted with water. The formed precipitate was filtered off and air dried. Obtained precipitate of P12 (180 mg, 63%) was used in the next step without purification. LCMS (ESI) m/z calc. for C₂₆H₃₁F₃N₆O₃ 532.41; found, 533.3 [M+H]⁺.

### Synthesis of (1R,2S,5S)—N-((2S)-1-amino-1-oxo-3-(2-oxoindolin-3-yl)propan-2-yl)-3-((S)-3,3-dimethyl-2-(2,2,2-trifluoroacetamido)butanoyl)-6,6-dimethyl-3-azabicyclo[3.1.0]hexane-2-carboxamide (P17)

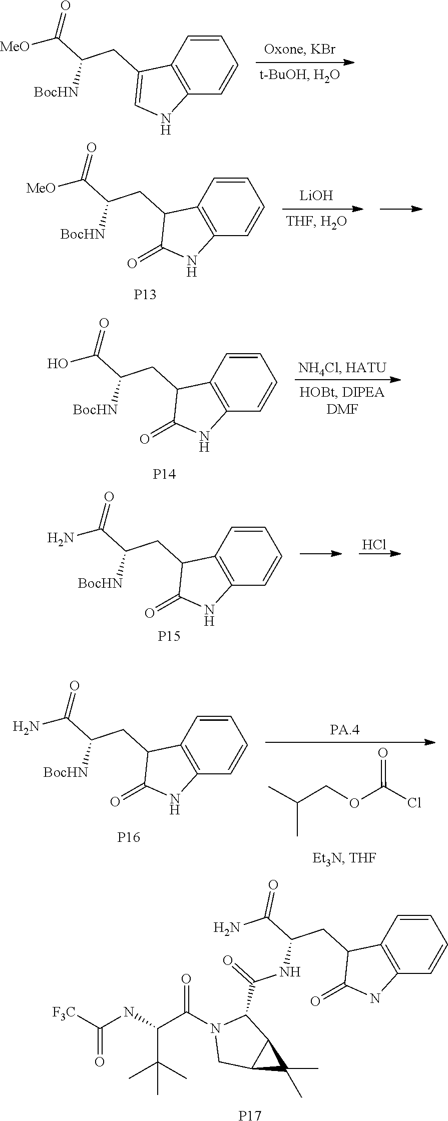

### Preparation 43. Methyl (2S)-2-((tert-butoxycarbonyl)amino)-3-(2-oxoindolin-3-yl)propanoate (P13)

A solution of methyl (2S)-2-(tert-butoxycarbonylamino)-3-(1H-indol-3-yl)propanoate (5.0 g, 16 mmol), Oxone (5.3 g, 17 mmol) and KBr (187 mg, 1.57 mmol) in tert-BuOH/H₂O (20:1 (v/v), 385 mL) was stirred at rt for 3 h. The mixture was extracted with EtOAc. The organic layers were dried over Na₂SO₄, filtered, and concentrated to give a residue. The residue was purified by silica gel column chromatography (0-10% MeOH in DCM) to give P13 (2.1 g, 40%) as a yellow solid which was a mixture of two diastereomers. ¹H NMR (400 MHz, CDCl₃), δ: 8.34 (br. s, 2H), 7.46 (d, J=7.4 Hz, 1H), 7.26-7.18 (m, 3H), 7.13-7.03 (m, 2H), 6.95-6.82 (m, 2H), 5.74 (br. s, 1H), 5.48 (d, J=8.8 Hz, 1H), 4.77-4.62 (m, 1H), 4.59-4.48 (m, 1H), 3.77-3.67 (m, 6H), 3.60-3.48 (m, 2H), 2.46-2.37 (m, 2H), 2.34-2.25 (m, 2H), 1.49-1.37 (m, 18H); LRMS (ESI) m/z 357.1 [M+Na]⁺.

### Preparation 44. (2S)-2-(tert-Butoxycarbonylamino)-3-(2-oxoindolin-3-yl)propanoic Acid (P14)

A solution of P13 (3.60 g, 10.8 mmol) and LiOH (259 mg, 10.8 mmol) in THF/H₂O (180 mL, 1:2 (v/v)) was stirred at rt for 30 min. The mixture was treated with Dowex H⁺. The mixture was filtered and the filtrate was concentrated under reduced pressure. The residue was purified by silica gel column chromatography (0-20% MeOH in DCM) and C18 column chromatography (10-50% MeOH in H₂O) to give P14 (1.9 g, 55%) as a yellow solid. ¹H NMR (400 MHz, CD₃OD), δ: 7.39-7.16 (m, 4H), 7.11-6.98 (m, 2H), 6.98-6.84 (m, 2H), 4.55-4.36 (m, 2H), 2.48-2.20 (m, 4H), 1.51-1.28 (m, 18H); LCMS (ESI) m/z 321.1 [M+H]⁺.

### Preparation 45. tert-Butyl ((2S)-1-amino-1-oxo-3-(2-oxoindolin-3-yl)propan-2-yl)carbamate (P15)

A solution of P14 (1.9 g, 5.9 mmol), HATU (2.7 g, 7.1 mmol), HOBt (0.45 g, 2.9 mmol), ammonium chloride (0.50 g, 9.4 mmol) and DIPEA (5.5 mL, 30 mmol) in DMF (25 mL) was stirred at rt for 2 h. The solution was treated with EtOAc. The mixture was washed with H₂O, dried over Na₂SO₄, filtered, and concentrated. The residue was purified by silica gel column chromatography (0-100% EtOAc in n-hexane) to give P15 (0.70 g, 37%) as a solid. ¹H NMR (400 MHz, CDCl₃), δ: 8.36-8.03 (m, 2H), 7.25-7.18 (m, 4H), 7.11-7.00 (m, 2H), 6.92-6.83 (m, 2H), 6.77-6.47 (m, 2H), 5.78-5.48 (m, 4H), 4.80-4.41 (m, 2H), 3.80-3.52 (m, 2H), 2.63-2.12 (m, 4H), 1.47-1.38 (m, 18H); LCMS (ESI) m/z 320.1 [M+H]⁺.

### Preparation 46. (2S)-2-Amino-3-(2-oxoindolin-3-yl)propanamide (P16)

To a solution of P15 (0.70 g, 2.2 mmol) in isopropanol (14 mL) was added a solution of HCl (3.7 mL, 3 M) in MeOH in ice bath. The reaction was stirred at rt for 16 h. The solvent was removed under reduced pressure. The residue was purified by C18 column chromatography (5-50% MeOH in H₂O) to give P16 (500 mg, containing isopropanol) a solid. ¹H NMR (400 MHz, CD₃OD), δ: 7.34-6.70 (m, 8H), 4.48-4.33 (m, 2H), 3.81-3.56 (m, 2H), 2.63-2.47 (m, 2H), 2.26-1.96 (m, 2H); LCMS (ESI) m/z 220.1 [M+H]⁺.

### Preparation 47. (1R,2S,5S)—N-((2S)-1-Amino-1-oxo-3-(2-oxoindolin-3-yl)propan-2-yl)-3-((S)-3,3-dimethyl-2-(2,2,2-trifluoroacetamido)butanoyl)-6,6-dimethyl-3-azabicyclo[3.1.0]hexane-2-carboxamide (P17)

A solution of PA.4 (90 mg, 0.25 mmol), isobutyl chloroformate (37 mg, 0.27 mmol) and Et₃N (27 mg, 0.27 mmol) in THF (2.0 mL) was stirred at 0° C. for 2 h. P16 (54 mg, 0.25 mmol) was added to the reaction mixture and the resulting mixture was stirred at rt for 16 h. The mixture was concentrated. The residue was purified by C18 column chromatography (10-80% MeOH in H₂O) and silica gel column (0-10% MeOH in DCM) to give P17 (30 mg, 21%) as a solid. ¹H NMR (400 MHz, CDCl₃), δ: 8.82-8.47 (m, 2H), 7.84 (br. s, 2H), 7.24-7.11 (m, 4H), 7.10-6.97 (m, 2H), 6.89-6.71 (m, 4H), 6.02 (br. s, 2H), 5.80 (br. s, 2H), 4.99-4.85 (m, 2H), 4.64-4.51 (m, 2H), 4.33 (s, 2H), 4.15-3.95 (m, 2H), 3.90-3.74 (m, 2H), 3.66-3.50 (m, 2H), 2.74-2.62 (m, 2H), 2.35-2.22 (m, 2H), 1.57-1.39 (m, 4H), 1.11-0.80 (m, 30H); LCMS (ESI) m/z 566.2 [M+H]⁺.

### Synthesis of (S)-2-amino-3-(1H-1,2,3-triazol-1-yl)propanamide (P21)

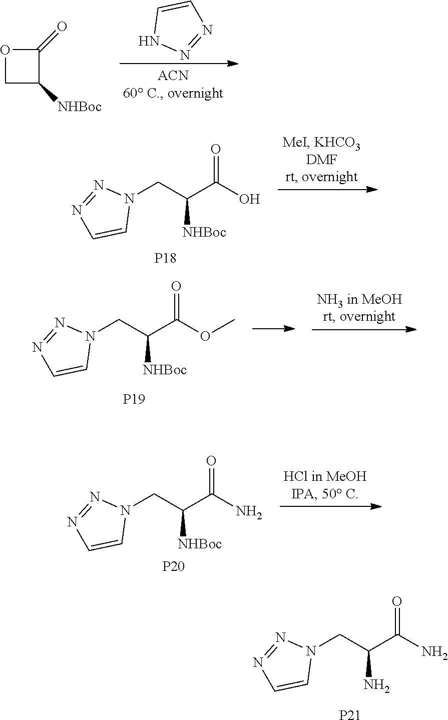

### Preparation 48. (S)-2-((tert-Butoxycarbonyl)amino)-3-(1H-1,2,3-triazol-1-yl)propanoic Acid (P18)

To a solution of tert-butyl (S)-(2-oxooxetan-3-yl)carbamate (1.00 g, 5.34 mmol) in ACN (20 ml) was added 1H-1,2,3-triazole (553 mg, 8.02 mmol) at rt. The reaction mixture was stirred at 60° C. for 16 h. The mixture was concentrated and purified by C18 column chromatography (5-100% of MeOH in water) to give the desired product P18 (460 mg, 34% yield). ¹H NMR (400 MHz, CDCl₃), δ: 7.72 (s, 1H), 7.63 (s, 1H), 5.52 (d, J=6.0 Hz, 1H), 5.02-4.89 (m. 2H), 4.69 (br. s, 1H), 1.44 (s, 9H).

### Preparation 49. Methyl (S)-2-((tert-butoxycarbonyl)amino)-3-(1H-1,2,3-triazol-1-yl)propanoate (P19)

To a solution of P18 (700 mg, 2.73 mmol) in DMF (14 ml) was added iodomethane (582 mg, 4.10 mmol) and KHCO₃ (546 mg, 5.46 mmol). After the reaction mixture was stirred at 25° C. for 17 h, the reaction was diluted by ethyl acetate and washed by water and brine. The organic layer was dried over sodium sulfate, filtered, and concentrated to give P19 (260 mg, 35% yield). ¹H NMR (400 MHz, CDCl₃), δ: 7.70 (d, J=1.2 Hz, 1H), 7.53 (d, J=1.2 Hz, 1H), 5.36 (d, J=6.4 Hz, 1H), 4.92-4.80 (m. 2H), 4.74-4.70 (m, 1H), 3.78 (s, 3H), 1.44 (s, 9H).

### Preparation 50. tert-Butyl (S)-(1-amino-1-oxo-3-(1H-1,2,3-triazol-1-yl)propan-2-yl)carbamate (P20)

To a solution of ammonia in methanol (7 M, 8.0 ml) was added P19 (260 mg, 0.96 mmol) and stirred at 25° C. for 16 h. The mixture was concentrated to give the desired product P20 (240 mg, crude). ¹H NMR (400 MHz, DMSO-d₆), δ: 7.99 (s, 1H), 7.68 (s, 1H), 7.52 (br. s, 1H), 7.26 (br. s, 1H), 7.04 (d, J=8.8 Hz, 1H), 4.71 (dd, J=4.4, 13.6 Hz, 1H), 4.47-4.34 (m, 2H), 1.30 (s, 9H).

### Preparation 51. (S)-2-Amino-3-(1H-1,2,3-triazol-1-yl)propanamide (P21)

To a solution of P20 (240 mg, crude) in isopropanol (12 ml) was added a solution of HCl in methanol (4 mL) at 0° C. The mixture was stirred at 50° C. for 16 h. The mixture was concentrated to give P21 (200 mg as a HCl salt) which was used in next step without purification. ¹H NMR (400 MHz, DMSO-d₆), δ: 8.48 (br. s, 3H), 8.13 (m, 2H), 7.78-7.77 (m, 1H), 7.73 (br. s, 1H), 4.86 (d, J=5.6 Hz, 2H), 4.32-4.29 (m, 1H).

### Synthesis of (S)-2-amino-3-(4,5-dimethyl-1H-1,2,3-triazol-1-yl)propanamide (P26)

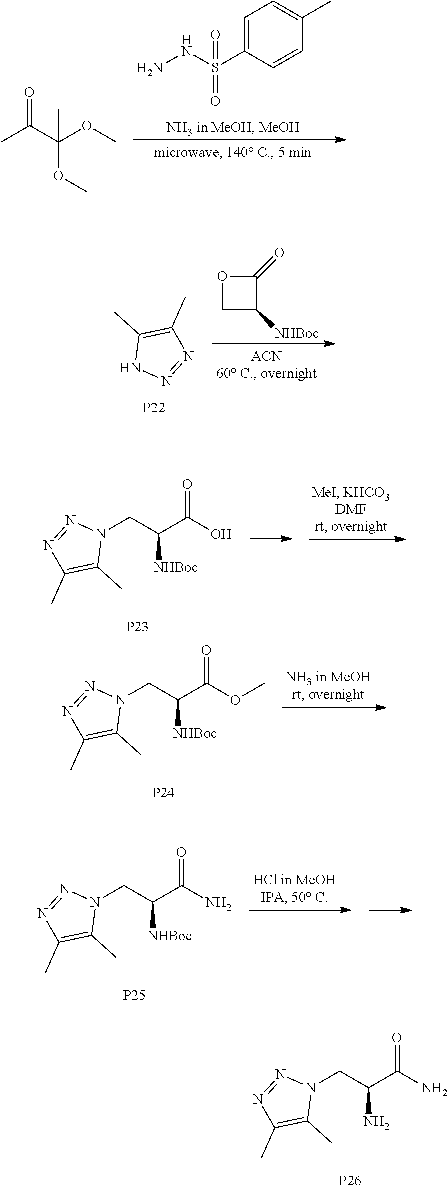

### Preparation 52. 4,5-Dimethyl-1H-1,2,3-triazole (P22)

To a solution of 3,3-dimethoxybutan-2-one (2.50 g, 18.9 mmol) in methanol (38 ml) was added 4-methylbenzenesulfonohydrazide (3.5 g, 18.9 mmol) and NH₃ solution (5.95 mL, 7N in methanol) at rt. The reaction mixture was stirred at 140° C. for 5 min under microwave irradiation. The mixture was concentrated and purified by silica gel column chromatography (0-55% of EtOAc in n-hexane) to give the desired product P22 (220 mg, 12% yield). ¹H NMR (400 MHz, CDCl₃), δ: 2.28 (s, 6H).

### Preparation 53. (S)-2-((tert-Butoxycarbonyl)amino)-3-(4,5-dimethyl-1H-1,2,3-triazol-1-yl)propanoic Acid (P23)

To a solution of tert-butyl (S)-(2-oxooxetan-3-yl)carbamate (290 mg, 1.54 mmol) in ACN (2.0 ml) was added P22 (100 mg, 1.03 mmol) at rt. The reaction mixture was stirred at 60° C. for 16 h. The mixture was concentrated to give the crude product P23 which was used for next step without purification.

### Preparation 54. Methyl (S)-2-((tert-butoxycarbonyl)amino)-3-(1H-1,2,3-triazol-1-yl)propanoate (P24)

To a solution of P23 (1.03 mmol) in DMF (2.0 ml) was added iodomethane (210 mg, 1.03 mmol) and potassium hydrogen carbonate (200 mg, 2.06 mmol). After the reaction mixture was stirred at 25° C. for 17 h, it was treated with ethyl acetate and washed by water and brine. The organic layer was dried over sodium sulfate, filtered, concentrated, and purified by preparative TLC (5% MeOH in DCM) to give P24 (40 mg). ¹H NMR (400 MHz, CDCl₃), δ: 4.71-4.65 (m, 2H), 4.58-4.53 (m, 1H), 3.78 (s, 3H), 2.25 (s, 3H), 2.19 (s, 3H), 1.43 (s, 9H).

### Preparation 55. tert-Butyl (S)-(1-amino-3-(4,5-dimethyl-1H-1,2,3-triazol-1-yl)-1-oxopropan-2-yl)carbamate (P25)

A solution of NH₃ in methanol (7 N, 2.0 ml) was added to P24 (40 mg, 0.13 mmol). The solution was stirred at 25° C. for 16 h. The mixture was concentrated to give the desired product P25 (29 mg, crude) which was used for next step without purification.

### Preparation 56. (S)-2-amino-3-(4,5-dimethyl-1H-1,2,3-triazol-1-yl)propanamide (P26)

To a solution of P25 (29 mg, crude) in isopropanol (3.0 ml) was added a solution of HCl in methanol (1.0 mL) at 0° C. The mixture was allowed to warm to 50° C., and was stirred for 16 h. The mixture was concentrated to give P26 (30 mg, crude, HCl salt). ¹H NMR (400 MHz, DMSO-d₆), δ: 8.52 (br. s, 2H), 8.10 (br. s, 1H), 7.66 (br. s, 1H), 4.68-4.57 (m, 2H), 4.23-4.20 (m, 1H), 2.21 (s, 3H), 2.15 (s, 3H).

### Synthesis of (1R,2S,5S)—N—((S)-1-amino-3-(1H-benzo[d][1,2,3]triazol-1-yl)-1-oxopropan-2-yl)-3-((S)-3,3-dimethyl-2-(2,2,2-trifluoroacetamido)butanoyl)-6,6-dimethyl-3-azabicyclo[3.1.0]hexane-2-carboxamide (P31)

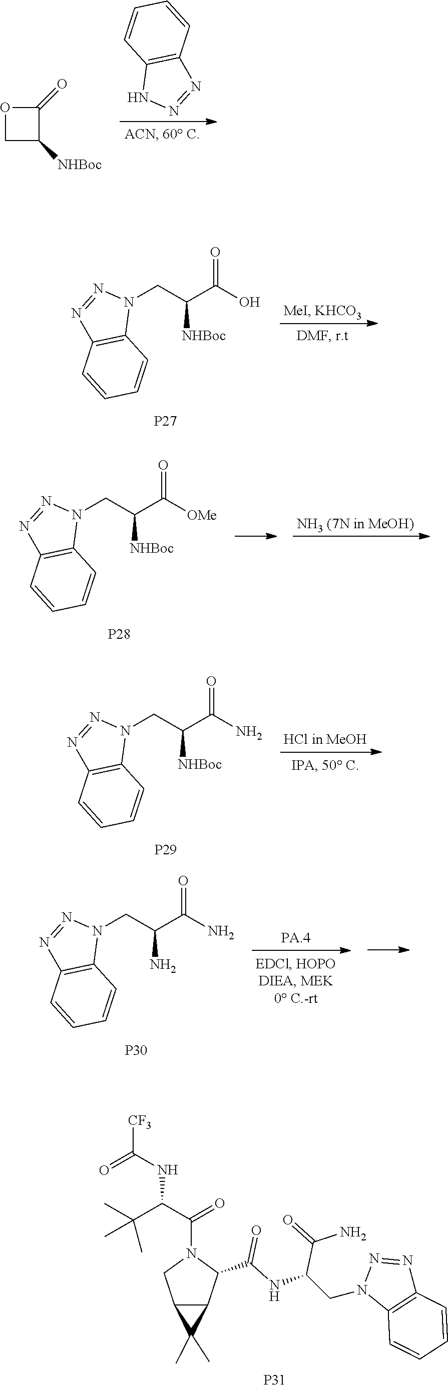

### Preparation 57. (S)-3-(1H-benzo[d][1,2,3]triazol-1-yl)-2-((tert-butoxycarbonyl)amino)propanoic Acid (P27)

To a solution of tert-butyl N-[(3S)-2-oxooxetan-3-yl]carbamate (1.00 g, 5.34 mmol) in ACN (18 mL) was added 1H-benzotriazole (954 mg, 8.01 mmol) at rt. The mixture was stirred at 60° C. for overnight. The mixture was concentrated to remover solvent. The crude was purified by C18 column chromatography (0-100% MeOH in water) to give P27 (730 mg, 45%). ¹H NMR (400 MHz, CDCl₃), δ: 8.04 (d, J=8.2 Hz, 1H), 7.61 (d, J=7.8 Hz, 1H), 7.50 (t, J=8.2, 7.0 Hz, 1H), 7.40 (dd, J=7.8, 7.0 Hz, 1H), 5.44-5.42 (m, 1H), 5.25-5.21 (m, 2H), 4.84-4.82 (m, 1H), 1.43 (s, 9H); LRMS (ESI) m/z 307.1 [M+H]⁺.

### Preparation 58. Methyl (S)-3-(1H-benzo[d][1,2,3]triazol-1-yl)-2-((tert-butoxycarbonyl)amino)propanoate (P28)

To a solution of P27 (730 mg, 2.38 mmol) and potassium bicarbonate (477 mg, 3.57 mmol) in DMF (12 mL) was added iodomethane (0.222 ml, 3.57 mmol) at rt. After the mixture was stirred at rt for overnight, the mixture was treated with water and extracted with EtOAc. The organic layers were dried over Na₂SO₄₍ₛ₎, filtered and concentrated. The residue was purified by silica gel column chromatography (0-100% EtOAc in n-hexane) to give P28 (596 mg, 78%). ¹H NMR (400 MHz, CDCl₃), δ: 8.05 (d, J=8.0 Hz, 1H), 7.54-7.47 (m, 2H), 7.39-7.35 (m, 1H), 5.35-5.33 (m, 1H), 5.11-5.10 (m, 2H), 4.83-4.79 (m, 1H), 3.76 (s, 3H), 1.41 (s, 9H).

### Preparation 59. tert-Butyl (S)-(1-amino-3-(1H-benzo[d][1,2,3]triazol-1-yl)-1-oxopropan-2-yl)carbamate (P29)

A solution of P28 (596 mg, 1.86 mmol) in NH₃ in MeOH (5 mL) was stirred at rt for 16 h. The mixture was concentrated to remove solvent to give P29 (465 mg, 82%). ¹H NMR (400 MHz, DMSO-d6), δ: 7.99 (d, J=8.4 Hz, 1H), 7.87 (d, J=8.0 Hz, 1H), 7.60 (br. s, 1H), 7.55-7.51 (m, 1H), 7.38-7.33 (m, 2H), 6.98 (d, J=8.8 Hz, 1H), 5.05-5.00 (m, 1H), 4.81-4.75 (m, 1H), 4.52-4.47 (m, 1H), 1.12 (s, 9H); LRMS (ESI) m/z 306.1 [M+H]⁺.

### Preparation 60. (S)-2-Amino-3-(1H-benzo[d][1,2,3]triazol-1-yl)propanamide (P30)

To a solution of P29 (200 mg, 0.655 mmol) in isopropanol (3.0 mL) was added 3M HCl in MeOH (1.10 ml, 3.28 mmol) at rt. The mixture was stirred at 50° C. for overnight. The resulting solid was collected by filtration and washed with isopropanol to give P30 (124 mg, 93%). ¹H NMR (400 MHz, DMSO-d6), δ: 8.33 (br. s, 1H), 8.16 (br. s, 1H), 8.06 (d, J=8.4 Hz, 1H), 7.94 (d, J=8.4 Hz, 1H), 7.73 (br. s, 1H), 7.61-7.57 (m, 1H), 7.45-7.41 (m, 1H), 5.15-5.08 (m, 2H), 4.37-4.34 (m, 1H). LCMS (ESI) m/z 206.1 [M+H]⁺.

### Preparation 61. (1R,2S,5S)—N—((S)-1-Amino-3-(1H-benzo[d][1,2,3]triazol-1-yl)-1-oxopropan-2-yl)-3-((S)-3,3-dimethyl-2-(2,2,2-trifluoroacetamido)butanoyl)-6,6-dimethyl-3-azabicyclo[3.1.0]hexane-2-carboxamide (P31)

To a solution of P30 (50 mg, 0.244 mmol) and PA.4 (74 mg, 0.203 mmol) in 2-butanone (1.0 mL) was added EDCI (50 mg, 0.264 mmol), HOPO (5.6 mg, 0.051 mmol) and DIPEA (0.141 ml, 0.812 mmol) at 0° C. The mixture was stirred at room temperature for overnight. The mixture was diluted with MTBE/EtOAc. The organic layer was washed with water, brine, and 1N HCl_{(aq)} dried over Na₂SO₄₍ₛ₎, filtered and concentrated. The residue was purified by silica gel column chromatography (0-10% MeOH in dichloromethane) to give P31 (40 mg, 36%). ¹H NMR (400 MHz, CDCl₃): δ 8.01 (d, J=8.4 Hz, 1H), 7.69 (d, J=8.4 Hz, 1H), 7.55-7.49 (m, 1H), 7.40-7.36 (m, 1H), 7.11 (d, J=7.6 Hz, 1H), 7.04 (d, J=9.2 Hz, 1H), 6.71 (br. s, 1H), 5.63 (br. s, 1H), 5.40-5.33 (m, 1H), 5.04-4.93 (m, 2H), 4.57-4.55 (m, 1H), 4.20 (s, 1H), 4.06-4.02 (m, 1H), 3.91-3.83 (m, 1H), 1.56-1.50 (m, 1H), 1.28-1.24 (m, 1H), 1.08-0.85 (m, 15H); LCMS (ESI) m/z 552.3 [M+H]⁺.

### Synthesis of (1R,2S,5S)—N-[1-cyano-2-(1H-indol-3-yl)ethyl]-3-[(2S)-3,3-dimethyl-2-[(2,2,2-trifluoroacetyl)amino]butanoyl]-6,6-dimethyl-3-azabicyclo[3.1.0]hexane-2-carboxamide (P35)

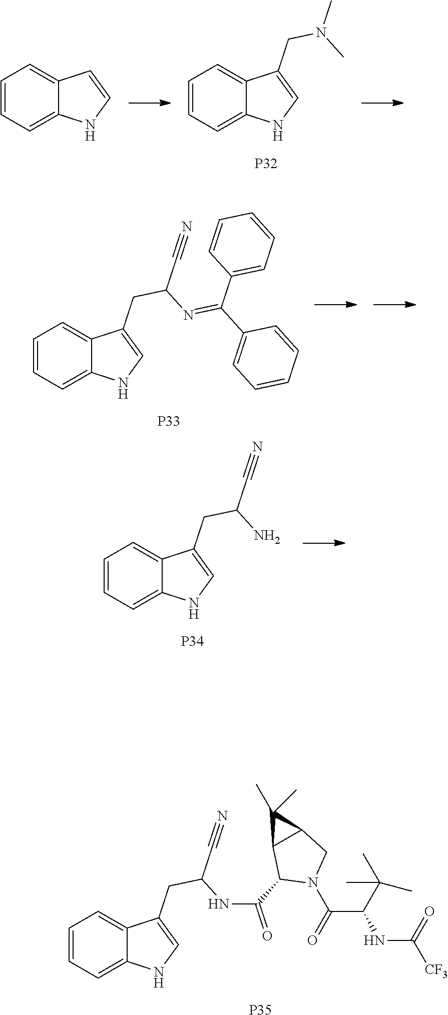

### Preparation 62. 1-(1H-indol-3-yl)-N,N-dimethyl-methanamine (P32)

Acetic acid (5.7 ml) was added slowly to 33% (w/v) aq. dimethylamine (5.7 ml), cooled in a freezing mixture, at such a rate that the temperature did not rise above 5° C. then 40% formaldehyde solution (2.85 ml) was added, the temperature being still kept below 5° C. This solution was then poured on indole (5 g, 42.7 mmol) and the mixture shaken. The indole dissolved, with evolution of heat, in the course of a minute or two and the mixture was kept at rt for at least 4 h, then the reaction mixture was poured into 2N-sodium hydroxide, and the solid gramine collected after the mixture had been kept overnight in the refrigerator. Yield 6.31 g, 84.8%. ¹H NMR (400 MHz, DMSO-d6), δ: 10.87 (s, 1H), 7.59 (d, J=7.9 Hz, 1H), 7.33 (d, J=8.1 Hz, 1H), 7.19 (d, J=2.4 Hz, 1H), 7.06 (ddd, J=8.2, 7.0, 1.3 Hz, 1H), 6.96 (td, J=7.4, 1.1 Hz, 1H), 3.51 (s, 2H), 2.14 (s, 6H).

### Preparation 63. 2-(Benzhydrylideneamino)-3-(1H-indol-3-yl)propanenitrile (P33)

To a water-cooled solution of P32 (4.36 g, 25 mmol) and 2-(benzhydrylideneamino)acetonitrile (4.6 g, 20.9 mmol) in DCM (125 ml) were added portionwise 35% NaOH (12 ml, 108.5 mmol) and DMSO (4.74 g, 37.6 mmol) over 1 h. The mixture was stirred at rt for 45 h after which the insoluble material was filtered off through a Celite pad. The organic layer was separated, washed with brine, dried (MgSO₄) and concentrated under reduced pressure. The residue was purified by column chromatography (hexane-AcOEt, 8:1) to give the title compound P33 (4.22 g, 57.8%). ¹H NMR (400 MHz, CDCl₃), δ: 8.05 (s, 1H), 7.65 (dt, J=7.2, 1.4 Hz, 2H), 7.49-7.31 (m, 7H), 7.21-7.08 (m, 3H), 6.99 (ddd, J=7.9, 7.0, 1.0 Hz, 1H), 6.87 (d, J=7.4 Hz, 2H), 4.58 (t, J=7.2 Hz, 1H), 3.47 (dd, J=14.2, 7.0 Hz, 1H), 3.29 (dd, J=14.2, 7.3 Hz, 1H).

### Preparation 64. 2-Amino-3-(1H-indol-3-yl)propanenitrile (P34)

To a solution of P33 (4.22 g, 12.07 mmol) in THF (100 ml) were added 9% HCl (20 ml). The mixture was stirred at rt for 30 min, concentrated under reduced pressure, diluted with water (50 ml), extracted with DCM (3×50 ml). Aqueous phase basified with 2N NaOH to pH 10, extracted with MTBE (2×50 ml). The combined organic fractions were dried over Na₂SO₄, filtered, and concentrated under reduced pressure to give the title compound P34 (1.4 g, 62.6%). ¹H NMR (400 MHz, DMSO-d6), δ: 10.93 (s, 1H), 7.57 (d, J=7.9 Hz, 1H), 7.36 (d, J=8.1 Hz, 1H), 7.26 (d, J=2.4 Hz, 1H), 7.08 (ddd, J=8.1, 6.9, 1.2 Hz, 1H), 6.99 (ddd, J=7.9, 6.9, 1.1 Hz, 1H), 3.99 (s, 1H), 3.05 (d, J=7.2 Hz, 2H), 2.34 (s, 2H).

### Preparation 65. (1R,2S,5S)—N-[1-Cyano-2-(1H-indol-3-yl)ethyl]-6,6-dimethyl-3-[(2S)-3,3-dimethyl-2-[(2,2,2-trifluoroacetyl)amino]butanoyl]-3-azabicyclo[3.1.0]hexane-2-carboxamide (P35)

To a mixture of the PA.4 (413 mg, 1.13 mmol) and HATU (452 mg, 1.19 mmol) in ACN (5 ml) DIPEA (168 mg, 1.3 mmol) was added. The mixture was stirred at rt for 30 min, and P34 (200 mg, 1.08 mmol) was added, and stirred overnight. Reaction mixture was diluted with ethyl acetate (50 ml) and washed with water (3×50 ml), brine (2×50 mL), dried on Na₂SO₄, and concentrated under reduced pressure, and the crude was purified by flash chromatography on silica gel (hexane/EtOAc—2:1) to give the title compound P35 (543 mg, 94.50%). ¹H NMR (400 MHz, DMSO-d6), δ: 10.99 (s, 1H), 9.37 (t, J=7.6 Hz, 1H), 8.96 (dd, J=12.6, 7.9 Hz, 1H), 7.59 (t, J=7.5 Hz, 1H), 7.35 (s, 2H), 7.36-7.25 (m, 1H), 7.13-6.93 (m, 3H), 4.97 (dq, J=14.9, 7.6 Hz, 1H), 4.40 (dd, J=11.1, 8.4 Hz, 1H), 4.21 (d, J=12.6 Hz, 1H), 3.97-3.88 (m, 1H), 3.82 (dd, J=10.4, 5.5 Hz, 0H), 3.67 (t, J=11.2 Hz, 1H), 3.31-3.10 (m, 2H), 1.56-1.45 (m, 1H), 1.41-1.33 (m, 1H), 1.24 (s, 2H), 1.00 (d, J=8.3 Hz, 13H), 0.96-0.78 (in, 8H).

In the Table 3 presented certain non-limiting examples of the compound of Formula (II)-(V).

TABLE 3
Selected examples of the compound of Formula (II)-(V)

| | | [MH]⁺ | [MH]⁺ |
|---|---|---|---|
| # | Structure | Calc. | Found |
| 201 | 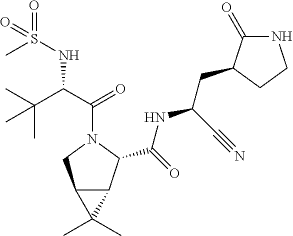 [SMILES: CC(C)(C)[C@H](NS(C)(=O)=O)C(=O)N1C[C@H]2[C@H]([C@H]1C(=O)N[C@H](C#N)C[C@@H]1CCNC1=O)C2(C)C] | 482.2437 | 482 |
| 202 | 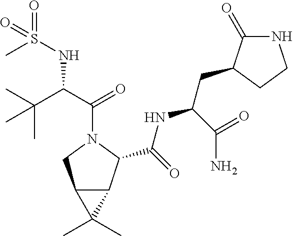 [SMILES: CC(C)(C)[C@H](NS(C)(=O)=O)C(=O)N1C[C@H]2[C@H]([C@H]1C(=O)N[C@@H](C[C@@H]1CCNC1=O)C(N)=O)C2(C)C] | 500.2543 | 500 |
| 203 | 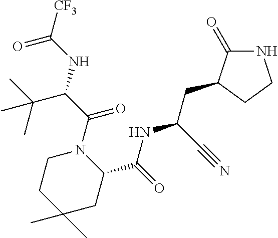 [SMILES: CC1(C)CCN(C(=O)[C@@H](NC(=O)C(F)(F)F)C(C)(C)C)[C@H](C(=O)N[C@H](C#N)C[C@@H]2CCNC2=O)C1] | 502.2641 | 502 |
| 204 | 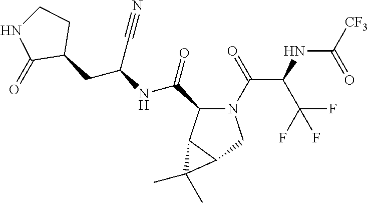 [SMILES: CC1(C)[C@@H]2[C@@H](C(=O)N[C@H](C#N)C[C@@H]3CCNC3=O)N(C(=O)[C@@H](NC(=O)C(F)(F)F)C(F)(F)F)C[C@@H]21] | 512.1733 | 512 |
| 205 | 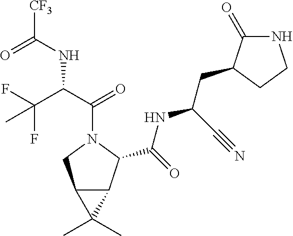 [SMILES: CC(F)(F)[C@H](NC(=O)C(F)(F)F)C(=O)N1C[C@H]2[C@H]([C@H]1C(=O)N[C@H](C#N)C[C@@H]1CCNC1=O)C2(C)C] | 508.1983 | 508 |
| 206 | 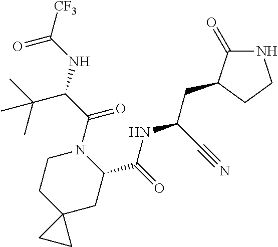 [SMILES: CC(C)(C)[C@H](NC(=O)C(F)(F)F)C(=O)N1CCC2(CC2)C[C@H]1C(=O)N[C@H](C#N)C[C@@H]1CCNC1=O] | 500.2485 | 500 |
| 207 | 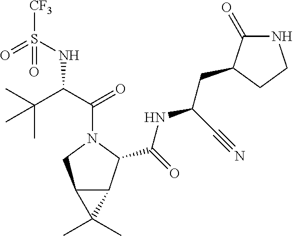 [SMILES: CC(C)(C)[C@H](NS(=O)(=O)C(F)(F)F)C(=O)N1C[C@H]2[C@H]([C@H]1C(=O)N[C@H](C#N)C[C@@H]1CCNC1=O)C2(C)C] | 536.2155 | 536 |
| 208 | 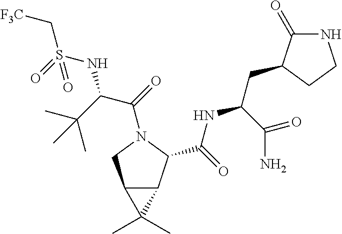 [SMILES: CC(C)(C)[C@H](NS(=O)(=O)CC(F)(F)F)C(=O)N1C[C@H]2[C@H]([C@H]1C(=O)N[C@@H](C[C@@H]1CCNC1=O)C(N)=O)C2(C)C] | 568.2417 | 568 |
| 209 | 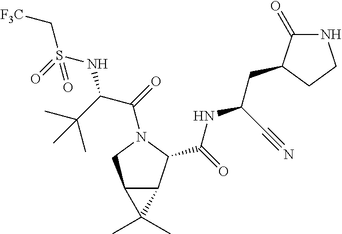 [SMILES: CC(C)(C)[C@H](NS(=O)(=O)CC(F)(F)F)C(=O)N1C[C@H]2[C@H]([C@H]1C(=O)N[C@H](C#N)C[C@@H]1CCNC1=O)C2(C)C] | 550.2311 | 550 |
| 212 | 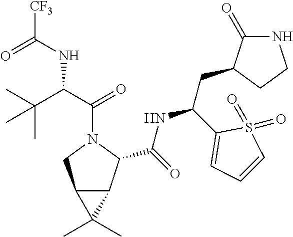 [SMILES: CC(C)(C)[C@H](NC(=O)C(F)(F)F)C(=O)N1C[C@H]2[C@H]([C@H]1C(=O)N[C@@H](C[C@@H]1CCNC1=O)C1=CC=CS1(=O)=O)C2(C)C] | 589.2308 | 589 |
| 214 | 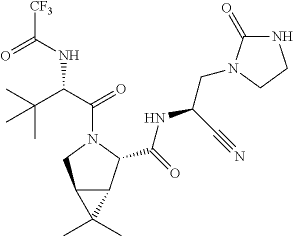 [SMILES: CC(C)(C)[C@H](NC(=O)C(F)(F)F)C(=O)N1C[C@H]2[C@H]([C@H]1C(=O)N[C@H](C#N)CN1CCNC1=O)C2(C)C] | 501.2437 | 501 |
| 215 | 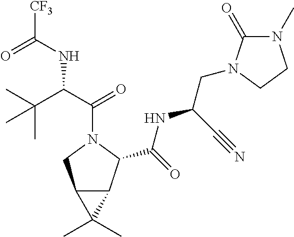 [SMILES: CN1CCN(C[C@@H](C#N)NC(=O)[C@@H]2[C@H]3[C@H](CN2C(=O)[C@@H](NC(=O)C(F)(F)F)C(C)(C)C)C3(C)C)C1=O] | 515.2594 | 515 |
| 216 | 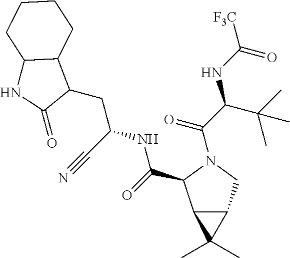 [SMILES: CC(C)(C)[C@H](NC(=O)C(F)(F)F)C(=O)N1C[C@H]2[C@H]([C@H]1C(=O)N[C@H](C#N)CC1C(=O)NC3CCCCC31)C2(C)C] | 554.2954 | 555 |
| 217 | 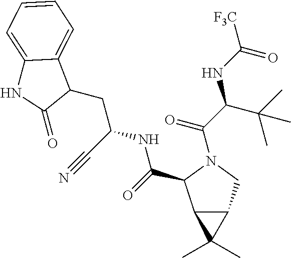 [SMILES: CC(C)(C)[C@H](NC(=O)C(F)(F)F)C(=O)N1C[C@H]2[C@H]([C@H]1C(=O)N[C@H](C#N)CC1C(=O)Nc3ccccc31)C2(C)C] | 548.2485 | 548 |
| 218 | 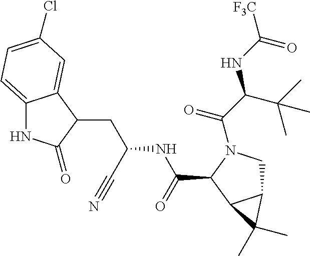 [SMILES: CC(C)(C)[C@H](NC(=O)C(F)(F)F)C(=O)N1C[C@H]2[C@H]([C@H]1C(=O)N[C@H](C#N)CC1C(=O)Nc3ccc(Cl)cc31)C2(C)C] | 582.2095 | 582 |
| 219 | 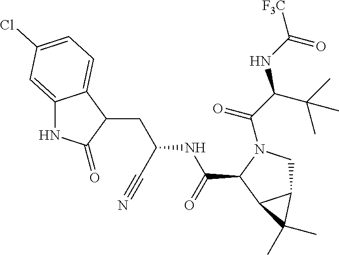 [SMILES: CC(C)(C)[C@H](NC(=O)C(F)(F)F)C(=O)N1C[C@H]2[C@H]([C@H]1C(=O)N[C@H](C#N)CC1C(=O)Nc3cc(Cl)ccc31)C2(C)C] | 582.2095 | 582 |
| 220 | 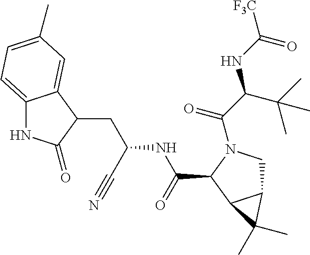 [SMILES: Cc1ccc2c(c1)C(C[C@@H](C#N)NC(=O)[C@@H]1[C@H]3[C@H](CN1C(=O)[C@@H](NC(=O)C(F)(F)F)C(C)(C)C)C3(C)C)C(=O)N2] | 562.2641 | 562 |
| 221 | 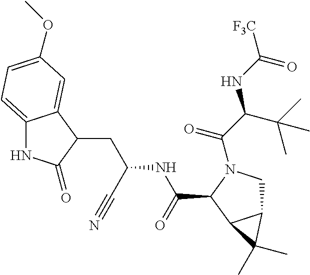 [SMILES: COc1ccc2c(c1)C(C[C@@H](C#N)NC(=O)[C@@H]1[C@H]3[C@H](CN1C(=O)[C@@H](NC(=O)C(F)(F)F)C(C)(C)C)C3(C)C)C(=O)N2] | 578.2591 | 578 |
| 222 | 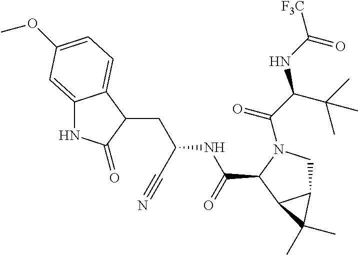 [SMILES: COc1ccc2c(c1)NC(=O)C2C[C@@H](C#N)NC(=O)[C@@H]1[C@H]2[C@H](CN1C(=O)[C@@H](NC(=O)C(F)(F)F)C(C)(C)C)C2(C)C] | 578.2591 | 578 |
| 223 | 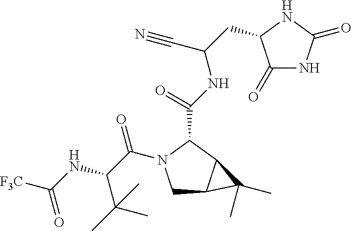 [SMILES: CC(C)(C)[C@H](NC(=O)C(F)(F)F)C(=O)N1C[C@H]2[C@@H]([C@H]1C(=O)NC(C#N)C[C@@H]1NC(=O)NC1=O)C2(C)C] | 515.223 | 515 |
| 224 | 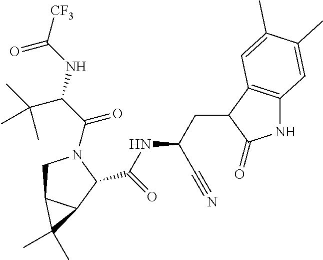 [SMILES: Cc1cc2c(cc1C)C(C[C@@H](C#N)NC(=O)[C@@H]1[C@@H]3[C@H](CN1C(=O)[C@@H](NC(=O)C(F)(F)F)C(C)(C)C)C3(C)C)C(=O)N2] | 576.2797 | 576 |
| 225 | 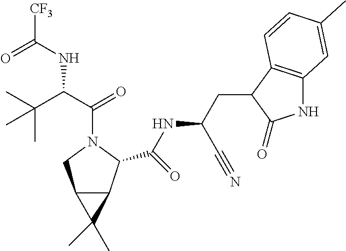 [SMILES: Cc1ccc2c(c1)NC(=O)C2C[C@@H](C#N)NC(=O)[C@@H]1[C@@H]2[C@H](CN1C(=O)[C@@H](NC(=O)C(F)(F)F)C(C)(C)C)C2(C)C] | 562.2641 | 562 |
| 226 | 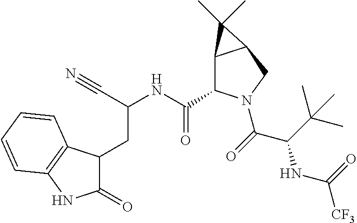 [SMILES: CC(C)(C)[C@H](NC(=O)C(F)(F)F)C(=O)N1C[C@H]2[C@@H]([C@H]1C(=O)NC(C#N)CC1C(=O)Nc3ccccc31)C2(C)C] | 548.2485 | 548 |
| 227 | 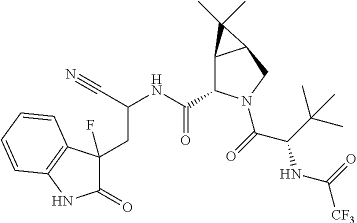 [SMILES: CC(C)(C)[C@H](NC(=O)C(F)(F)F)C(=O)N1C[C@H]2[C@@H]([C@H]1C(=O)NC(C#N)CC1(F)C(=O)Nc3ccccc31)C2(C)C] | 566.239 | 566 |
| 228 | 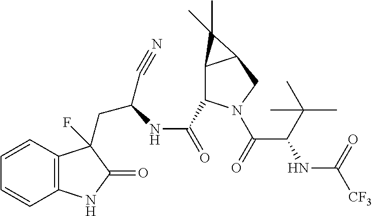 | 566.239 | 566 |
| 229 | 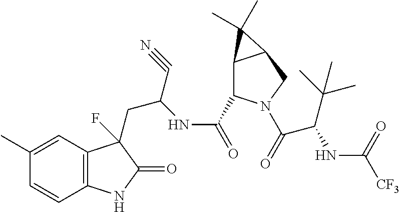 | 580.2547 | 580 |
| 230 | 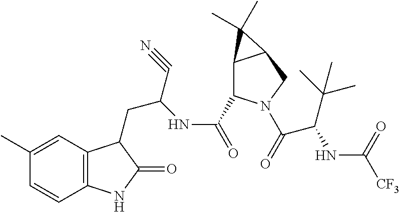 | 562.2641 | 562 |
| 231 | 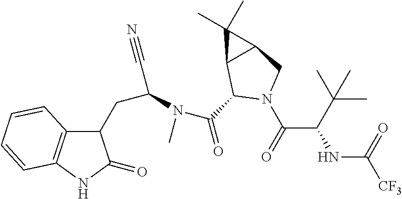 [SMILES: CN(C(=O)[C@@H]1[C@@H]2[C@H](CN1C(=O)[C@@H](NC(=O)C(F)(F)F)C(C)(C)C)C2(C)C)[C@H](C#N)CC1C(=O)Nc2ccccc21] | 562.2641 | 562 |
| 232 | 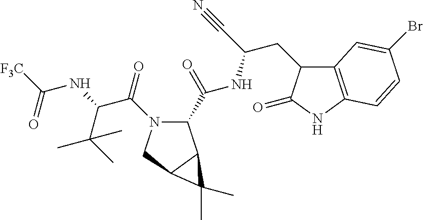 [SMILES: CC(C)(C)[C@H](NC(=O)C(F)(F)F)C(=O)N1C[C@H]2[C@@H]([C@H]1C(=O)N[C@H](C#N)CC1C(=O)Nc3ccc(Br)cc31)C2(C)C] | 626.1589 | 626 |
| 233 | 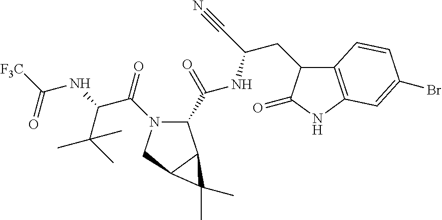 [SMILES: CC(C)(C)[C@H](NC(=O)C(F)(F)F)C(=O)N1C[C@H]2[C@@H]([C@H]1C(=O)N[C@H](C#N)CC1C(=O)Nc3cc(Br)ccc31)C2(C)C] | 626.1589 | 626 |
| 234 | 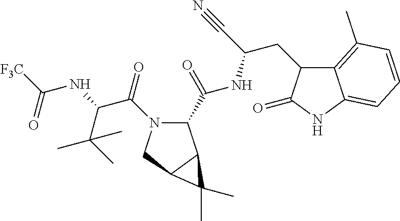 [SMILES: Cc1cccc2c1C(C[C@@H](C#N)NC(=O)[C@@H]1[C@@H]3[C@H](CN1C(=O)[C@@H](NC(=O)C(F)(F)F)C(C)(C)C)C3(C)C)C(=O)N2] | 562.2641 | 562 |
| 235 | 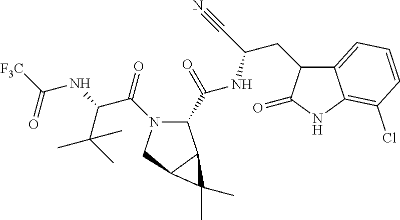 [SMILES: CC(C)(C)[C@H](NC(=O)C(F)(F)F)C(=O)N1C[C@H]2[C@@H]([C@H]1C(=O)N[C@H](C#N)CC1C(=O)Nc3c(Cl)cccc31)C2(C)C] | 582.2095 | 582 |
| 236 | 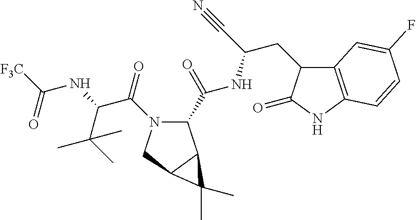 [SMILES: CC(C)(C)[C@H](NC(=O)C(F)(F)F)C(=O)N1C[C@H]2[C@@H]([C@H]1C(=O)N[C@H](C#N)CC1C(=O)Nc3ccc(F)cc31)C2(C)C] | 566.239 | 566 |
| 237 | 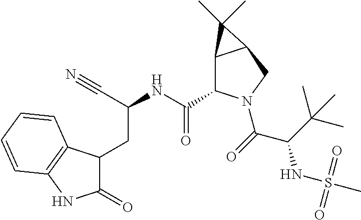 [SMILES: CC(C)(C)[C@H](NS(C)(=O)=O)C(=O)N1C[C@H]2[C@@H]([C@H]1C(=O)N[C@H](C#N)CC1C(=O)Nc3ccccc31)C2(C)C] | 530.2437 | 530 |
| 238 | 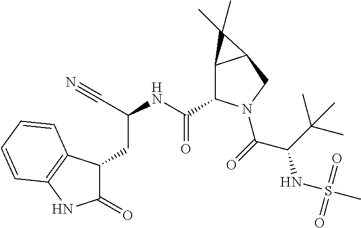 [SMILES: CC(C)(C)[C@H](NS(C)(=O)=O)C(=O)N1C[C@H]2[C@@H]([C@H]1C(=O)N[C@H](C#N)C[C@@H]1C(=O)Nc3ccccc31)C2(C)C] | 530.2437 | 530 |
| 239 | 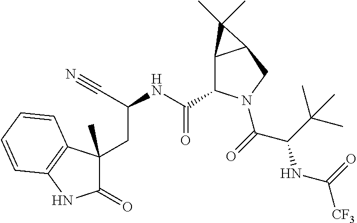 [SMILES: CC(C)(C)[C@H](NC(=O)C(F)(F)F)C(=O)N1C[C@H]2[C@@H]([C@H]1C(=O)N[C@H](C#N)C[C@]1(C)C(=O)Nc3ccccc31)C2(C)C] | 562.2641 | 562 |
| 240 | 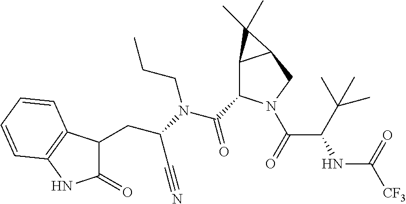 [SMILES: CCCN(C(=O)[C@@H]1[C@@H]2[C@H](CN1C(=O)[C@@H](NC(=O)C(F)(F)F)C(C)(C)C)C2(C)C)[C@H](C#N)CC1C(=O)Nc2ccccc21] | 590.2954 | 590 |
| 241 | 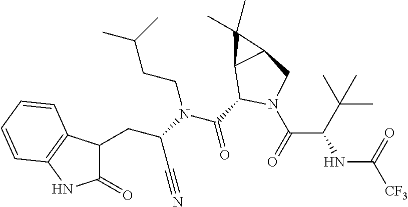 [SMILES: CC(C)CCN(C(=O)[C@@H]1[C@@H]2[C@H](CN1C(=O)[C@@H](NC(=O)C(F)(F)F)C(C)(C)C)C2(C)C)[C@H](C#N)CC1C(=O)Nc2ccccc21] | 618.3267 | 618 |
| 242 | 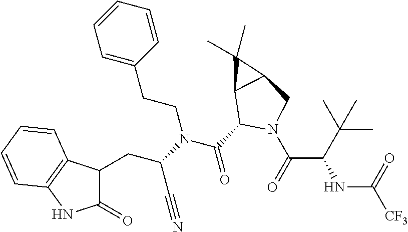 [SMILES: CC(C)(C)[C@H](NC(=O)C(F)(F)F)C(=O)N1C[C@H]2[C@@H]([C@H]1C(=O)N(CCc1ccccc1)[C@H](C#N)CC1C(=O)Nc3ccccc31)C2(C)C] | 652.311 | 652 |
| 243 | 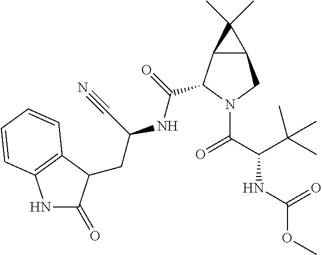 [SMILES: COC(=O)N[C@H](C(=O)N1C[C@H]2[C@@H]([C@H]1C(=O)N[C@H](C#N)CC1C(=O)Nc3ccccc31)C2(C)C)C(C)(C)C] | 510.2716 | 510 |
| 244 | 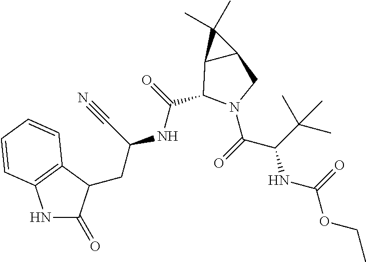 [SMILES: CCOC(=O)N[C@H](C(=O)N1C[C@H]2[C@@H]([C@H]1C(=O)N[C@H](C#N)CC1C(=O)Nc3ccccc31)C2(C)C)C(C)(C)C] | 524.2873 | 524 |
| 245 | 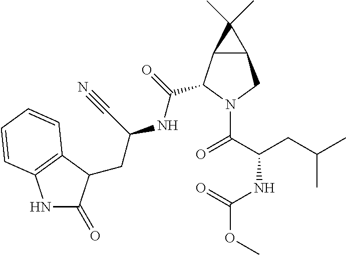 [SMILES: COC(=O)N[C@@H](CC(C)C)C(=O)N1C[C@H]2[C@@H]([C@H]1C(=O)N[C@H](C#N)CC1C(=O)Nc3ccccc31)C2(C)C] | 510.2716 | 510 |
| 246 | 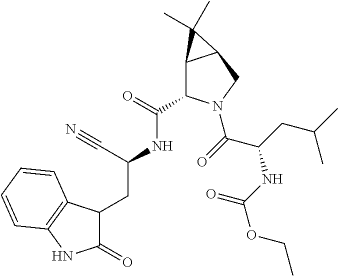 [SMILES: CCOC(=O)N[C@@H](CC(C)C)C(=O)N1C[C@H]2[C@@H]([C@H]1C(=O)N[C@H](C#N)CC1C(=O)Nc3ccccc31)C2(C)C] | 524.2873 | 524 |
| 247 | 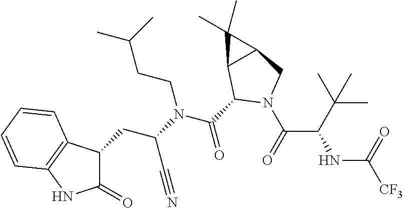 [SMILES: CC(C)CCN(C(=O)[C@@H]1[C@@H]2[C@H](CN1C(=O)[C@@H](NC(=O)C(F)(F)F)C(C)(C)C)C2(C)C)[C@H](C#N)C[C@@H]1C(=O)Nc2ccccc21] | 618.3267 | 618 |
| 248 | 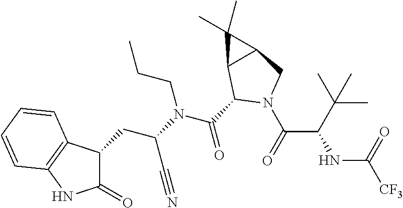 [SMILES: CCCN(C(=O)[C@@H]1[C@@H]2[C@H](CN1C(=O)[C@@H](NC(=O)C(F)(F)F)C(C)(C)C)C2(C)C)[C@H](C#N)C[C@@H]1C(=O)Nc2ccccc21] | 590.2954 | 590 |
| 249 | 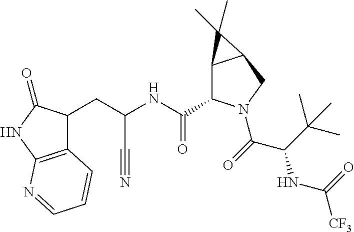 [SMILES: CC(C)(C)[C@H](NC(=O)C(F)(F)F)C(=O)N1C[C@H]2[C@@H]([C@H]1C(=O)NC(C#N)CC1C(=O)Nc3ncccc31)C2(C)C] | 549.2437 | 549 |
| 250 | 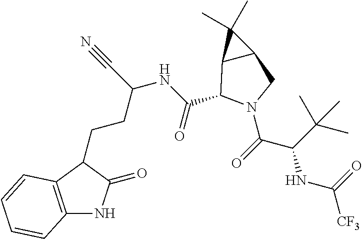 [SMILES: CC(C)(C)[C@H](NC(=O)C(F)(F)F)C(=O)N1C[C@H]2[C@@H]([C@H]1C(=O)NC(C#N)CCC1C(=O)Nc3ccccc31)C2(C)C] | 562.2641 | 562 |
| 251 | 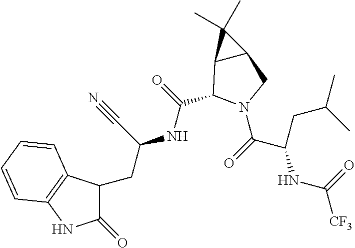 [SMILES: CC(C)C[C@H](NC(=O)C(F)(F)F)C(=O)N1C[C@H]2[C@@H]([C@H]1C(=O)N[C@H](C#N)CC1C(=O)Nc3ccccc31)C2(C)C] | 548.2485 | 548 |
| 252 | 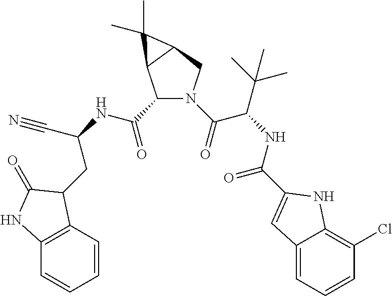 [SMILES: CC(C)(C)[C@H](NC(=O)c1cc2cccc(Cl)c2[nH]1)C(=O)N1C[C@H]2[C@@H]([C@H]1C(=O)N[C@H](C#N)CC1C(=O)Nc3ccccc31)C2(C)C] | 629.2643 | 629 |
| 253 | 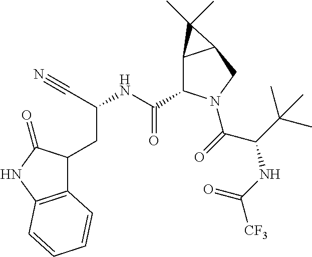 [SMILES: CC(C)(C)[C@H](NC(=O)C(F)(F)F)C(=O)N1C[C@H]2[C@@H]([C@H]1C(=O)N[C@@H](C#N)CC1C(=O)Nc3ccccc31)C2(C)C] | 548.2485 | 548 |
| 301 | 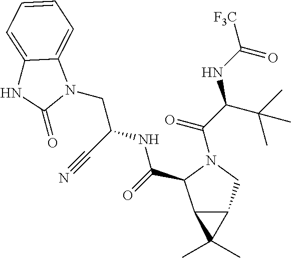 [SMILES: CC(C)(C)[C@H](NC(=O)C(F)(F)F)C(=O)N1C[C@H]2[C@H]([C@H]1C(=O)N[C@H](C#N)Cn1c(=O)[nH]c3ccccc31)C2(C)C] | 549.2437 | 549 |
| 302 | 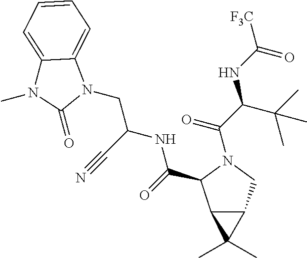 [SMILES: Cn1c(=O)n(CC(C#N)NC(=O)[C@@H]2[C@H]3[C@H](CN2C(=O)[C@@H](NC(=O)C(F)(F)F)C(C)(C)C)C3(C)C)c2ccccc21] | 563.2594 | 563 |
| 303 | 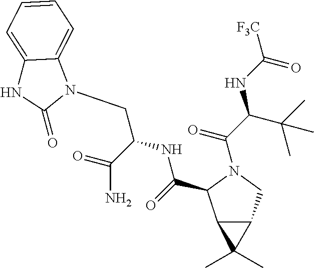 [SMILES: CC(C)(C)[C@H](NC(=O)C(F)(F)F)C(=O)N1C[C@H]2[C@H]([C@H]1C(=O)N[C@@H](Cn1c(=O)[nH]c3ccccc31)C(N)=O)C2(C)C] | 567.2543 | 567 |
| 304 | 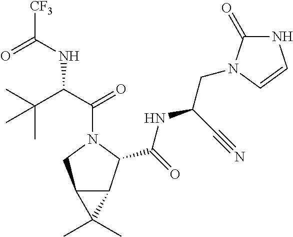 [SMILES: CC(C)(C)[C@H](NC(=O)C(F)(F)F)C(=O)N1C[C@H]2[C@H]([C@H]1C(=O)N[C@H](C#N)Cn1cc[nH]c1=O)C2(C)C] | 499.2281 | 499 |
| 305 | 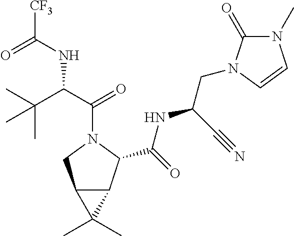 [SMILES: Cn1ccn(C[C@@H](C#N)NC(=O)[C@@H]2[C@H]3[C@H](CN2C(=O)[C@@H](NC(=O)C(F)(F)F)C(C)(C)C)C3(C)C)c1=O] | 513.2437 | 513 |
| 306 | 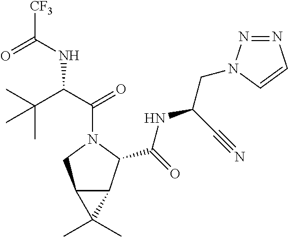 [SMILES: CC(C)(C)[C@H](NC(=O)C(F)(F)F)C(=O)N1C[C@H]2[C@H]([C@H]1C(=O)N[C@H](C#N)Cn1ccnn1)C2(C)C] | 484.2284 | 484 |
| 307 | 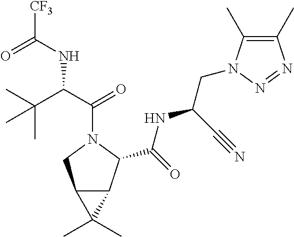 [SMILES: Cc1nnn(C[C@@H](C#N)NC(=O)[C@@H]2[C@H]3[C@H](CN2C(=O)[C@@H](NC(=O)C(F)(F)F)C(C)(C)C)C3(C)C)c1C] | 512.2597 | 512 |
| 308 | 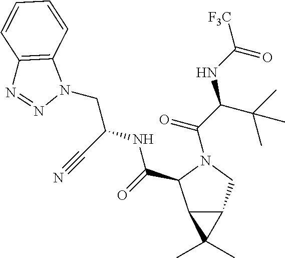 [SMILES: CC(C)(C)[C@H](NC(=O)C(F)(F)F)C(=O)N1C[C@H]2[C@H]([C@H]1C(=O)N[C@H](C#N)Cn1nnc3ccccc31)C2(C)C] | 534.244 | 534 |
| 309 | 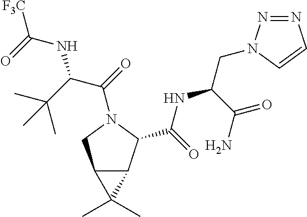 [SMILES: CC(C)(C)[C@H](NC(=O)C(F)(F)F)C(=O)N1C[C@H]2[C@H]([C@H]1C(=O)N[C@@H](Cn1ccnn1)C(N)=O)C2(C)C] | 502.2389 | 502 |
| 310 | 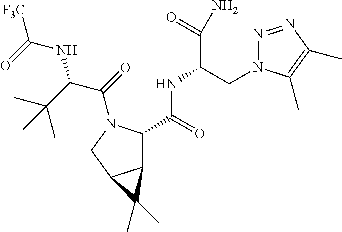 [SMILES: Cc1nnn(C[C@H](NC(=O)[C@@H]2[C@@H]3[C@H](CN2C(=O)[C@@H](NC(=O)C(F)(F)F)C(C)(C)C)C3(C)C)C(N)=O)c1C] | 530.2703 | 530 |
| 311 | 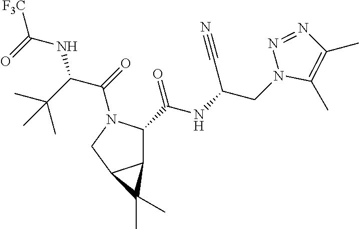 [SMILES: Cc1nnn(C[C@@H](C#N)NC(=O)[C@@H]2[C@@H]3[C@H](CN2C(=O)[C@@H](NC(=O)C(F)(F)F)C(C)(C)C)C3(C)C)c1C] | 512.2597 | 512 |
| 312 | 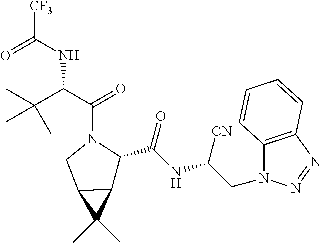 [SMILES: CC(C)(C)[C@H](NC(=O)C(F)(F)F)C(=O)N1C[C@H]2[C@@H]([C@H]1C(=O)N[C@H](C#N)Cn1nnc3ccccc31)C2(C)C] | 534.244 | 534 |
| 401 | 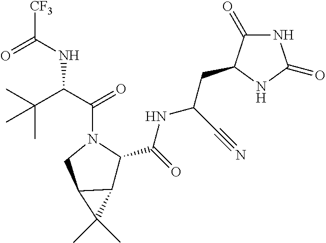 [SMILES: CC(C)(C)[C@H](NC(=O)C(F)(F)F)C(=O)N1C[C@H]2[C@H]([C@H]1C(=O)NC(C#N)C[C@@H]1NC(=O)NC1=O)C2(C)C] | 515.2229 | 515 |
| 402 | 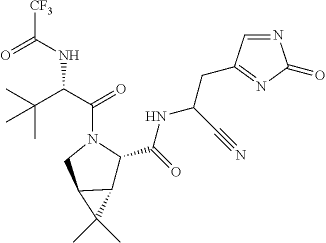 [SMILES: CC(C)(C)[C@H](NC(=O)C(F)(F)F)C(=O)N1C[C@H]2[C@H]([C@H]1C(=O)NC(C#N)CC1=NC(=O)N=C1)C2(C)C] | 497.2124 | 497 |
| 403 | 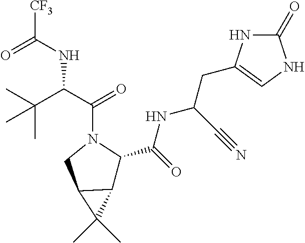 [SMILES: CC(C)(C)[C@H](NC(=O)C(F)(F)F)C(=O)N1C[C@H]2[C@H]([C@H]1C(=O)NC(C#N)Cc1c[nH]c(=O)[nH]1)C2(C)C] | 499.228 | 499 |
| 404 | 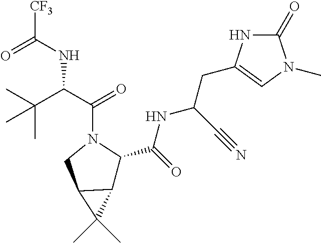 [SMILES: Cn1cc(CC(C#N)NC(=O)[C@@H]2[C@H]3[C@H](CN2C(=O)[C@@H](NC(=O)C(F)(F)F)C(C)(C)C)C3(C)C)[nH]c1=O] | 513.2437 | 513 |
| 501 | 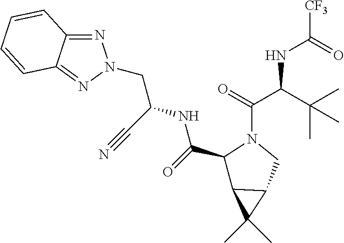 [SMILES: CC(C)(C)[C@H](NC(=O)C(F)(F)F)C(=O)N1C[C@H]2[C@H]([C@H]1C(=O)N[C@H](C#N)Cn1nc3ccccc3n1)C2(C)C] | 534.244 | 534 |

### Synthesis of the Representative Examples of the Compound

### Example 1. Methyl N-[(1S)-1-[(1S)-2-[[(1S)-1-cyano-2-(2-oxoindolin-3-yl)ethyl]carbamoyl]-6,6-dimethyl-3-azabicyclo[3.1.0]hexane-3-carbonyl]-2,2-dimethyl-propyl]carbamate (Compound 243)

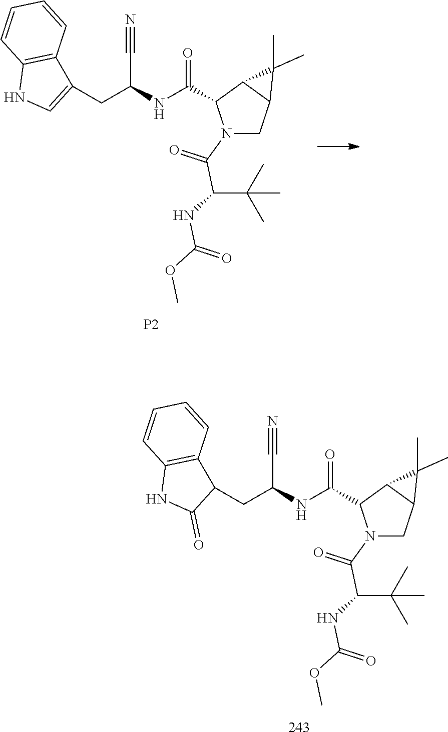

To a solution of P2 (0.105 g, 0.21 mmol) in the mixture of tert-butanol and water was added Oxone (0.077 g, 0.25 mmol) and KBr (0.025 g, 0.21 mmol). The resulting mixture was stirred overnight. The reaction mixture was diluted with a EtOAc and washed with saturated NaHCO_{3(aq)} and brine. The organic layer was dried over Na₂SO₄₍ₛ₎, filtered, and concentrated under reduced pressure. The product was purified by prep RP-HPLC to give compound 243 (0.023 g, 21.3%). ¹H NMR (400 MHz, DMSO-d₆), δ: 10.57-10.40 (m, 1H), 9.25 (d, J=8.2 Hz, 1H), 7.38-7.25 (m, 1H), 7.25-6.87 (m, 3H), 6.82 (d, J=7.8 Hz, 1H), 5.27-5.11 (m, 1H), 4.20 (s, 1H), 4.06-3.72 (m, 3H), 3.64-3.41 (m, 4H), 2.38-2.23 (m, 2H), 1.63-1.48 (m, 1H), 1.32 (d, J=7.6 Hz, 1H), 1.10-0.72 (m, 15H). LCMS (ESI) m/z calc. for C₂₇H₃₅N₅O₅ 509.59; found, 510.4 [M+H]⁺.

### Example 2. Ethyl N-[(1S)-1-[(1S)-2-[[(1S)-1-cyano-2-(2-oxoindolin-3-yl)ethyl]carbamoyl]-6,6-dimethyl-3-azabicyclo[3.1.0]hexane-3-carbonyl]-2,2-dimethyl-propyl]carbamate (Compound 244)

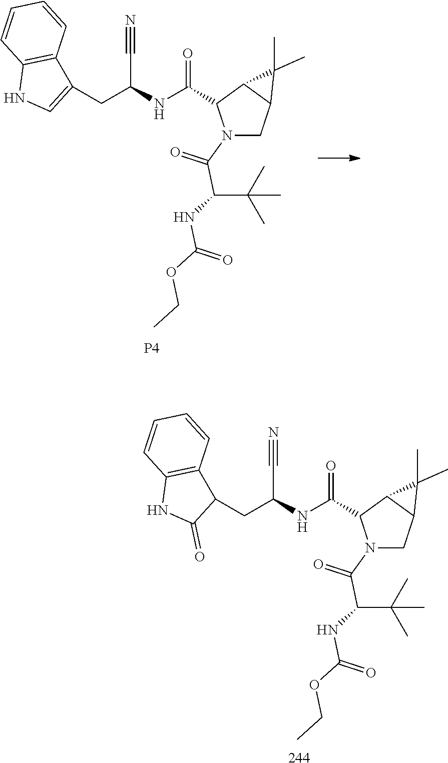

To a solution of P4 (0.189 g, 0.37 mmol) in the mixture of tert-butanol and water was added Oxone (0.137 mg, 0.44 mmol) and KBr (0.044 g, 0.37 mmol). The resulting mixture was stirred overnight. The reaction mixture was diluted with a EtOAc and washed with saturated NaHCO_{3(aq)} and brine. The organic layer was dried over Na₂SO₄₍ₛ₎, filtered, and concentrated under reduced pressure. The product was purified by prep RP-HPLC. Yield of compound 244: 0.004 g, 2%. LCMS (ESI) m/z calc. for C₂₈H₃₇N₅O₅ 523.62; found, 524.6 [M+H]⁺.

### Example 3. (1R,2S,5S)—N-[1-Cyano-3-(2-oxoindolin-3-yl)propyl]-6,6-dimethyl-3-[(2S)-3,3-dimethyl-2-[(2,2,2-trifluoroacetyl)amino]butanoyl]-3-azabicyclo[3.1.0]hexane-2-carboxamide (Compound 250)

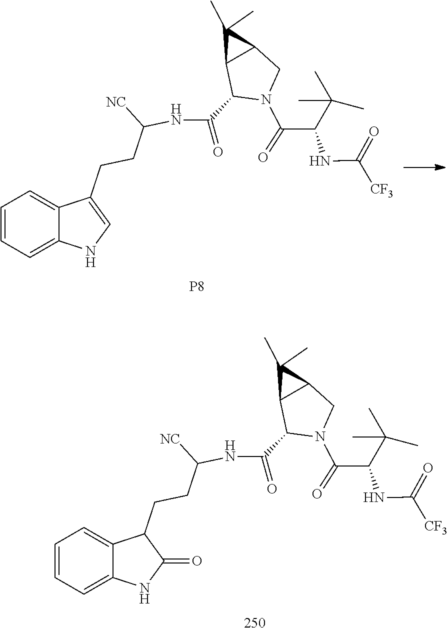

To a solution of P8 (1.0 eq, 650 mg, 1.2 mmol) and KBr (10 mol %, 14 mg) in tert-BuOH (12 ml)/H₂O (0.6 ml) (20/1, 0.1 M) at rt was added oxone (1.2 eq, MW=307, 439 mg), and was stirred overnight. The reaction was quenched by addition of aq. sat. Na₂SO₃ and then diluted with EtOAc. The organic fractions were collected and the aq. phase was extracted with EtOAc three times. The combined organic fractions were washed with brine, dried over Na₂SO₄, filtered, and concentrated under reduced pressure. The residue was purified by prep RP-HPLC on a C18 column eluting with a gradient MeCN—H₂O+0.1% TFA. Yield of compound 250: 160 mg, 24%. ¹H NMR (400 MHz, DMSO-d6), δ: 10.39 (d, J=4.5 Hz, 1H), 9.35 (dd, J=15.4, 8.4 Hz, 1H), 8.84 (dd, J=13.6, 7.9 Hz, 1H), 8.31 (s, 0H), 7.27-7.14 (m, 2H), 6.96 (s, 1H), 6.83 (d, J=7.8 Hz, 1H), 4.80 (dq, J=14.4, 7.2 Hz, 1H), 4.39 (d, J=8.8 Hz, 1H), 4.19 (s, 1H), 3.88 (dd, J=10.3, 5.3 Hz, 1H), 3.67 (t, J=10.9 Hz, 1H), 2.54 (s, 1H), 1.93 (dt, J=10.7, 5.5 Hz, 1H), 1.77 (s, 2H), 1.56-1.48 (m, 1H), 1.34-1.21 (m, 1H), 1.06-0.93 (m, 10H), 0.93-0.81 (m, 3H).

### Example 4. (1R,2S,5S)—N-[1-Cyano-2-(2-oxo-1,3-dihydropyrrolo[2,3-b]pyridin-3-yl)ethyl]-6,6-dimethyl-3-[(2S)-3,3-dimethyl-2-[(2,2,2-trifluoroacetyl)amino]butanoyl]-3-azabicyclo[3.1.0]hexane-2-carboxamide (Compound 249)

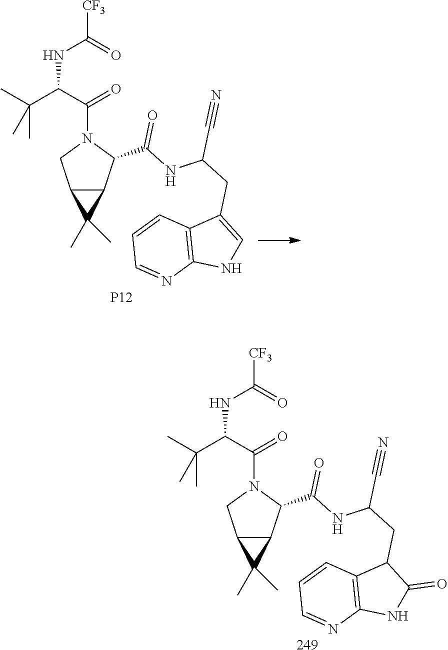

To a solution of P12 (180 mg, 0.34 mmol) in the mixture of tert-butanol (3 mL) and water (1 mL) was added Oxone (125 mg, 0.41 mmol) and KBr (4 mg, 0.034 mmol). The resulting mixture was stirred overnight. The reaction mixture was diluted with a EtOAc and washed with saturated NaHCO_{3(aq.)} and brine. The organic layer was dried over Na₂SO₄₍ₛ₎, filtered, and concentrated under reduced pressure. The residue was purified by silica gel column chromatography (0-5% methanol in DCM) to give compound 249 (17 mg, 9%) as a solid. ¹H NMR (400 MHz, DMSO-d₆), δ: 12.39 (s, 1H), 9.44-9.30 (m, 1H), 9.01-8.83 (m, 1H), 8.12 (s, 1H), 7.81-7.69 (m, 1H), 7.50-7.34 (m, 1H), 7.14-7.03 (m, 1H), 5.10-4.96 (m, 1H), 4.51-4.32 (m, 1H), 4.18 (s, 1H), 3.99-3.73 (m, 1H), 3.71-3.53 (m, 1H), 3.28-3.11 (m, 2H), 1.56-1.46 (m, 1H), 1.41-1.31 (m, 1H), 1.15-0.72 (m, 15H). LCMS (ESI) m/z calc. for C₂₆H₃₁F₃N₆O₄ 548.57; found, 549.8 [M+H]⁺.

### Example 5. (1R,2S,5S)—N-((1S)-1-Cyano-2-(2-oxoindolin-3-yl)ethyl)-3-((S)-3,3-dimethyl-2-(2,2,2-trifluoroacetamido)butanoyl)-6,6-dimethyl-3-azabicyclo[3.1.0]hexane-2-carboxamide (Compound 217)

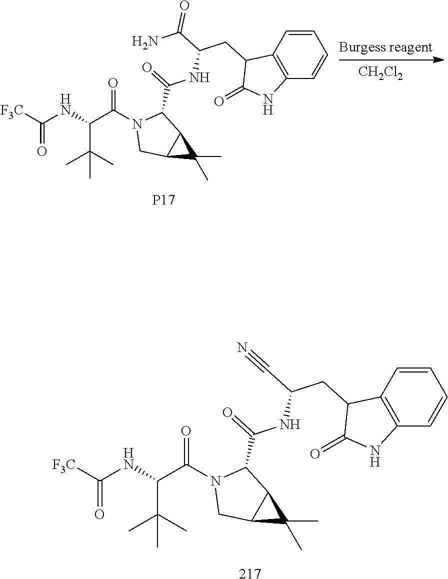

To a solution of P17 (30 mg, 0.053 mmol) in DCM (1.0 mL) was added Burgess reagent (32 mg, 0.13 mmol) at 0° C., and the resulting mixture was stirred at rt for 2 h. The mixture was concentrated under reduced pressure. The residue was purified by silica gel column chromatography (0-20% methanol in DCM) and C18 column chromatography (10-80% methanol in H₂O) to give compound 217 (6 mg, 21%) as a white solid. ¹H NMR (400 MHz, CD₃OD), δ: 7.46-7.28 (m, 2H), 7.27-7.18 (m, 2H), 7.08-6.86 (m, 4H), 5.43-5.23 (m, 2H), 4.56-4.49 (m, 2H), 4.33-4.27 (m, 2H), 4.07-3.98 (m, 2H), 3.90-3.83 (m, 2H), 3.81-3.45 (m, 2H), 2.58-2.13 (m, 4H), 1.72-1.54 (m, 2H), 1.48-1.38 (m, 2H), 1.11-0.99 (m, 12H), 0.97-0.88 (m, 18H); LCMS (ESI) m/z calc. for C₂₇H₃₂F₃N₅O₄ 547.24; found, 548.3 [M+H]⁺.

### Example 6. (1R,2S,5S)—N—((S)-1-amino-1-oxo-3-(1H-1,2,3-triazol-1-yl)propan-2-yl)-3-((S)-3,3-dimethyl-2-(2,2,2-trifluoroacetamido)butanoyl)-6,6-dimethyl-3-azabicyclo[3.1.0]hexane-2-carboxamide (309)

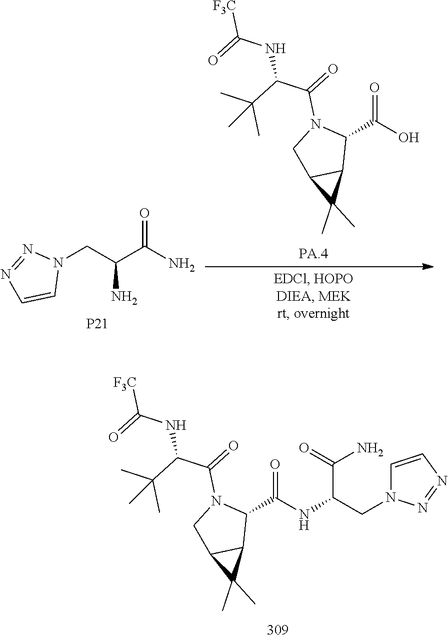

To a solution of PA.4 (174 mg, 0.49 mmol) in butan-2-one (1.5 ml) was added P21 (100 mg, 0.52 mol, HCl salt), 2-hydroxypyridine 1-oxide (14 mg, 0.12 mmol), DIPEA (252 mg, 1.96 mmol), and EDCI (121 mg, 0.64 mmol) at 0° C. The reaction mixture was stirred at 25° C. for 16 h. The mixture was concentrated and purified by C18 column chromatography (5-100% of MeOH in water) to give compound 309 (50 mg, 21% yield). ¹H NMR (400 MHz, CDCl₃), δ: 7.77 (d, J=0.8 Hz, 1H), 7.67 (d, J=0.8 Hz, 1H), 7.28 (d, J=8.0 Hz, 1H), 7.07 (d, J=9.2 Hz, 1H), 6.86 (br. s, 1H), 5.78 (br. s, 1H). 5.04 (dd, J=5.2, 14.0 Hz, 1H), 4.97-4.63 (m, 1H), 4.73 (dd, J=14.0, 4.4 Hz, 1H), 4.57 (d, J=9.6 Hz, 1H), 4.26 (s, 1H), 4.04 (dd, J=10.4, 5.6 Hz, 1H), 3.88 (d, J=10.4 Hz, 1H), 1.64-1.62 (m, 1H), 1.45 (d, J=7.6 Hz, 1H), 1.05 (s, 3H), 0.98 (s, 9H), 0.86 (s, 3H); LCMS (ESI) m/z calc. for C₂₁H₃₀F₃N₇O₄ 501.51; found, 502.7 [M+H]⁺; HPLC purity: 97.80%, t_{R}=16.250 min.

### Example 7. (1R,2S,5S)—N—((S)-1-amino-1-oxo-3-(2-oxo-2,3-dihydro-1H-benzo[d]imidazol-1-yl)propan-2-yl)-3-((S)-3,3-dimethyl-2-(2,2,2-trifluoroacetamido)butanoyl)-6,6-dimethyl-3-azabicyclo[3.1.0]hexane-2-carboxamide (Compound 303)

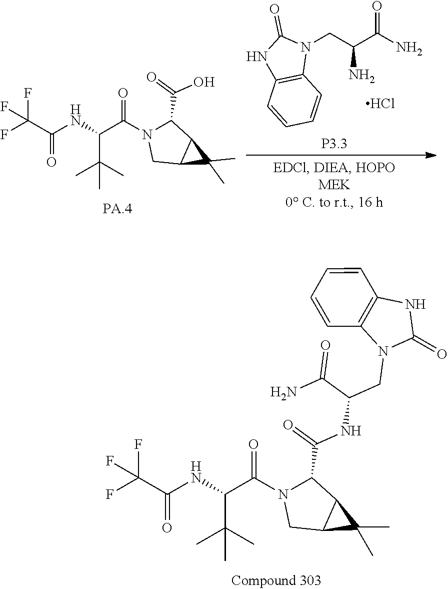 [SMILES: CC(C)(C)[C@H](NC(=O)C(F)(F)F)C(=O)N1C[C@H]2[C@@H]([C@H]1C(=O)O)C2(C)C ; NC(=O)[C@@H](N)Cn1c(=O)[nH]c2ccccc21 ; Cl ; CC(C)(C)[C@H](NC(=O)C(F)(F)F)C(=O)N1C[C@H]2[C@@H]([C@H]1C(=O)N[C@@H](Cn1c(=O)[nH]c3ccccc31)C(N)=O)C2(C)C]

To a solution of PA.4 (100 mg, 0.274 mmol, 1.0 eq) and P3.3 (79 mg, 0.31 mmol, 1.13 eq) in methyl ethyl ketone (MEK, 1.1 mL) was added 2-hydroxypyridine-N-oxide (HOPO, 7.6 mg, 0.069 mmol, 0.25 eq). After cooled to 0° C., the reaction mixture was added N,N-diisopropylethylamine (DIPEA, 0.14 mL, 0.82 mmol, 3.0 eq) and 1-ethyl-3-(3-dimethylaminopropyl)carbodiimide hydrochloride (EDCI, 63.1 mg, 0.33 mmol, 1.2 eq). The reaction mixture was stirred at rt for 16 h. The reaction mixture was treated with water and extracted with a mixture of ethyl acetate and methyl tert-butyl ether (1:1) three times. The combined organic layers were washed with 1 M HCl_{(aq)} and brine, then dried over Na₂SO₄₍ₛ₎, filtered, and concentrated in vacuo. The residue was purified by C-18 reversed-phase column chromatography (mobile phase A: water (with NH₄HCO₃), mobile phase B: methanol, UV: 214 and 254 nm, Flow rate: 35 mL/min, Gradient: 3-90% (% B)) to give ZE83-0154 (91 mg, 59% yield) as a white solid. ¹H NMR (400 MHz, DMSO-d₆), δ: 10.93 (s, 1H), 9.50 (d, J=8.4 Hz, 1H), 8.22 (d, J=7.2 Hz, 1H), 7.28-7.25 (m, 1H), 7.14-7.10 (m, 2H), 7.01-6.94 (m, 3H), 4.45-4.39 (m, 2H), 4.15-4.10 (m, 1H), 4.05 (s, 1H), 3.98-3.92 (m, 1H), 3.60 (d, J=11.6 Hz, 1H), 1.48-1.44 (m, 1H), 1.04-0.96 (m, 10H), 0.92 (s, 3H), 0.78 (s, 3H); LCMS (ESI+) m/z: calc. for C₂₆H₃₃F₃N₆O₅ 566.25; found, 567.3 [M+H]⁺; HPLC purity: 100%, t_{R}=17.916 min.

### Example 8. (1R,2S,5S)—N—((S)-1-cyano-2-(2-oxo-2,3-dihydro-1H-benzo[d]imidazol-1-yl)ethyl)-3-((S)-3,3-dimethyl-2-(2,2,2-trifluoroacetamido)butanoyl)-6,6-dimethyl-3-azabicyclo[3.1.0]hexane-2-carboxamide (Compound 301)

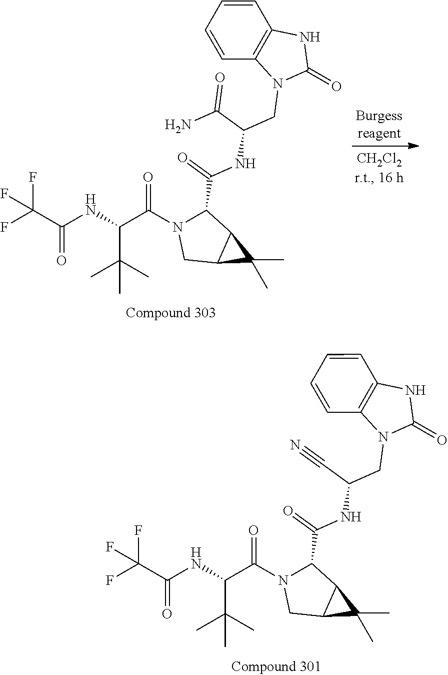 [SMILES: CC(C)(C)[C@H](NC(=O)C(F)(F)F)C(=O)N1C[C@H]2[C@@H]([C@H]1C(=O)N[C@@H](Cn1c(=O)[nH]c3ccccc31)C(N)=O)C2(C)C ; CC(C)(C)[C@H](NC(=O)C(F)(F)F)C(=O)N1C[C@H]2[C@@H]([C@H]1C(=O)N[C@H](C#N)Cn1c(=O)[nH]c3ccccc31)C2(C)C]

To a solution of compound 303 (40 mg, 0.071 mmol, 1.0 eq) in DCM (0.36 mL) was added methyl N-(triethylammoniumsulfonyl)carbamate (Burgess reagent, 42.1 mg, 0.177 mmol, 2.5 eq), then stirred at rt for 16 h. The mixture was treated with water, extracted with DCM for 3 times. The combined organic layers were dried over Na₂SO₄₍ₛ₎, filtered, and concentrated in vacuo. The residue was purified by C-18 reversed-phase column chromatography (mobile phase A: water (with NH₄HCO₃), mobile phase B: acetonitrile, UV: 214 and 254 nm, Flow rate: 35 mL/min, Gradient: 3-90% (% B)) to give compound 301 (29 mg, 75% yield) as a white solid. ¹H NMR (400 MHz, DMSO-d₆), δ: 11.02 (s, 1H), 9.49-9.43 (m, 1H), 8.84 (d, J=8.0 Hz, 1H), 7.22-7.18 (m, 1H), 7.05-6.97 (m, 3H), 5.13-5.08 (m, H), 4.43-4.35 (m, 1H), 4.27-4.16 (m, 2H), 4.10 (s, 1H), 3.95 (dd, J=10.4, 5.6 Hz, 1H), 3.61 (d, J=10.4 Hz, 1H), 1.47-1.44 (m, 1H), 1.00 (s, 9H), 0.97-0.94 (m, 1H), 0.92 (s, 3H), 0.78 (s, 3H); LCMS (ESI+) m/z: calc. for C₂₆H₃₁F₃N₆O₄ 548.24; found, 549.2 [M+H]⁺; HPLC purity: 100%, t_{R}=20.627 min.

### Example 9. (1R,2S,5S)—N—((S)-1-Cyano-2-(1H-1,2,3-triazol-1-yl)ethyl)-3-((S)-3,3-dimethyl-2-(2,2,2-trifluoroacetamido)butanoyl)-6,6-dimethyl-3-azabicyclo[3.1.0]hexane-2-carboxamide (306)

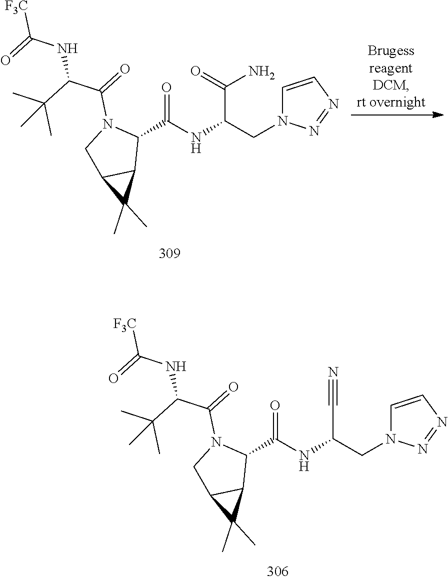 [SMILES: CC(C)(C)[C@H](NC(=O)C(F)(F)F)C(=O)N1C[C@H]2[C@@H]([C@H]1C(=O)N[C@@H](Cn1ccnn1)C(N)=O)C2(C)C ; CC(C)(C)[C@H](NC(=O)C(F)(F)F)C(=O)N1C[C@H]2[C@@H]([C@H]1C(=O)N[C@H](C#N)Cn1ccnn1)C2(C)C]

To a solution of 309 (45 mg, 0.090 mmol) in DCM (2.0 ml) was added Burgess reagent (54 mg, 0.23 mmol) at 25° C. After the reaction mixture was stirred at 25° C. for 16 h, the reaction mixture was concentrated and then purified by C18 column chromatography (5-100% of MeOH in water) to give compound 306 (40 mg, 83% yield). ¹H NMR (400 MHz, DMSO-d₆), δ: 9.41 (br. s, 1H), 9.11 (d, J=7.6 Hz, 1H), 8.23 (d, J=1.0 Hz, 1H), 7.76 (d, J=1.0 Hz, 1H), 5.33-5.28 (m, 1H), 4.87-4.82 (m, 1H), 4.78-4.73 (m, 1H), 4.40 (br. s, 1H), 4.18 (s, 1H), 3.91-3.88 (m, 1H), 3.68 (d, J=10.8 Hz, 1H), 1.55-1.54 (m, 1H), 1.26 (d, J=7.6 Hz, 1H), 1.00 (s, 3H), 0.98 (s, 9H), 0.82 (s, 3H); LCMS (ESI) m/z calc. for C₂₁H₂₈F₃N₇O₃ 483.50; found, 484.3 [M+H]⁺; HPLC purity: 100.00%, t_{R}=19.123 min.

### Example 10. (1R,2S,5S)—N—((S)-1-amino-3-(4,5-dimethyl-1H-1,2,3-triazol-1-yl)-1-oxopropan-2-yl)-3-((S)-3,3-dimethyl-2-(2,2,2-trifluoroacetamido)butanoyl)-6,6-dimethyl-3-azabicyclo[3.1.0]hexane-2-carboxamide (Compound 310)

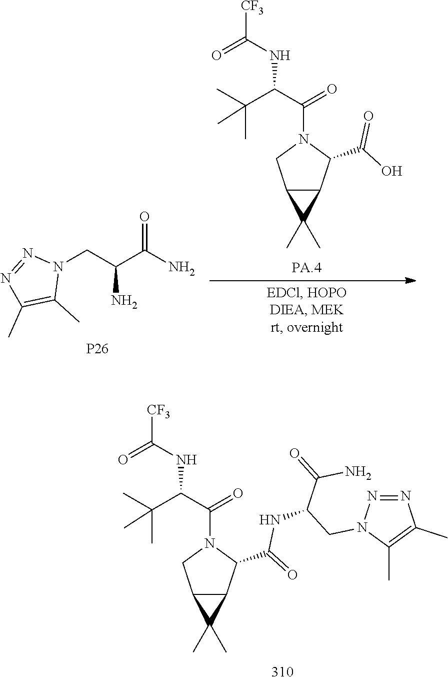

To a solution of PA.4 (65 mg, 0.18 mmol) in butan-2-one (1.0 ml) was added P26 (30 mg, 0.16 mmol, HCl salt), 2-hydroxypyridine 1-oxide (5.0 mg, 0.04 mmol), DIPEA (82 mg, 0.64 mmol), and EDCI (40 mg, 0.21 mmol) at 0° C. The reaction mixture was stirred at 25° C. for 16 h. The mixture was concentrated and purified by reverse column chromatography (5-100% of MeOH in water) to give compound 310 (25 mg, 29% yield). ¹H NMR (400 MHz, CDCl₃), δ: 7.66 (d, J=8.4 Hz, 1H), 7.08 (s, d=9.6 Hz, 1H), 6.91 (br. s, 1H), 5.62 (br. s, 1H), 4.98-4.94 (m, 1H), 4.83-4.78 (m, 1H), 4.61 (d, J=9.6 Hz, 1H), 4.42-4.37 (m, 1H), 4.28 (s, 1H), 4.13-4.06 (m, 1H), 3.90 (d, J=10.8 Hz, 1H), 2.24 (s, 3H), 2.23 (s, 3H), 1.63-1.60 (m, 1H), 1.46 (d, J=7.6 Hz, 1H), 1.05 (s, 3H), 0.99 (s, 9H), 0.87 (s, 3H).

### Example 11. (1R,2S,5S)—N—((S)-1-Cyano-2-(4,5-dimethyl-1H-1,2,3-triazol-1-yl)ethyl)-3-((S)-3,3-dimethyl-2-(2,2,2-trifluoroacetamido)butanoyl)-6,6-dimethyl-3-azabicyclo[3.1.0]hexane-2-carboxamide (Compound 311)

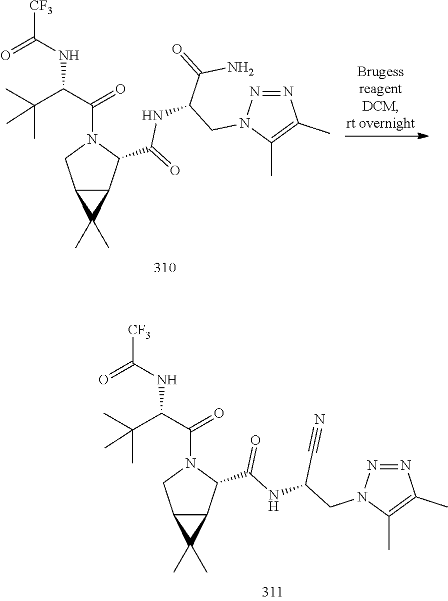

To a solution of compound 310 (20 mg, 0.04 mmol) in DCM (2.0 mL) was added Burgess reagent (23 mg, 0.10 mmol) at 25° C. After the reaction mixture had been stirred at 25° C. for 16 h. The reaction mixture was concentrated and purified by reverse column chromatography (5-100% of MeOH in water) to give compound 311 (4.0 mg, 21% yield). ¹H NMR (400 MHz, CDCl₃), δ: 7.73-7.70 (m, 1H), 6.95-6.89 (m, 1H), 5.36-5.30 (m, 1H), 5.03-4.99 (m, 0.3H), 4.61-4.54 (m, 2.4H), 4.36-4.34 (m, 1H), 4.14-3.54 (m, 2H), 2.34-2.29 (m, 6H), 1.60-1.51 (m, 1H), 1.44-1.34 (m, 1H), 1.07-1.03 (m, 10H), 0.96-0.87 (m, 5H); LCMS (ESI) m/z calc. for C₂₃H₃₂F₃N₇O₃ 511.55; found, 512.3 [M+H]⁺; HPLC purity: 100%, t_{R}=19.974 min.

### Example 12. (1R,2S,5S)—N—((S)-2-(1H-Benzo[d][1,2,3]triazol-1-yl)-1-cyanoethyl)-3-((S)-3,3-dimethyl-2-(2,2,2-trifluoroacetamido)butanoyl)-6,6-dimethyl-3-azabicyclo[3.1.0]hexane-2-carboxamide (Compound 312)

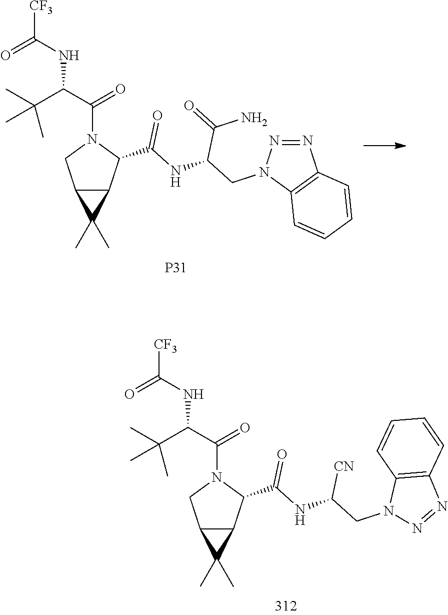

To a solution of P31 (400 mg, 0.85 mmol) in DCM (0.40 mL) was added methyl N-(triethylammoniosulfonyl)carbamate (44 mg, 0.183 mmol). The reaction was stirred at rt for 2 h. The reaction was quenched with saturated NaHCO_{3(aq)}/brine (2:1) and treated with a solution of MTBE/EtOAc (1:1). The organic layer was washed with NaHCO_{3(aq)}, brine and 1N HCl, dried over Na₂SO₄₍ₛ₎, filtered and concentrated. The residue was purified by silica gel column chromatography (0-10% MeOH in DCM) to give compound 312 (14 mg, 37%). ¹H NMR (400 MHz, CDCl₃), δ: 8.07 (d, J=8.8 Hz, 1H), 7.81 (d, J=8.0 Hz, 1H), 7.62 (m, J=8.4 Hz, 1H), 7.58-7.54 (m, 1H), 7.46-7.45 (m, 1H), 6.90 (d, J=9.2 Hz, 1H), 5.40-5.33 (m, 1H), 5.22-5.17 (m, 1H), 5.08-4.96 (m, 2H), 4.50 (d, J=9.2 Hz, 1H), 4.40 (d, J=8.0 Hz, 1H), 4.24 (s, 1H), 3.96-3.92 (m, 1H), 3.84-3.81 (m, 1H), 1.55-1.50 (m, 1H), 1.25-1.20 (m, 2H), 1.03-0.78 (m, 15H); LCMS (ESI) m/z 534.7 [M+H]⁺; HPLC purity: 99.3%, t_{R}=21.02 min.

### Example 13. (1R,2S,5S)—N—((S)-1-Amino-1-oxo-3-((S)-2-oxopyrrolidin-3-yl)propan-2-yl)-3-((S)-3,3-dimethyl-2-(methylsulfonamido)butanoyl)-6,6-dimethyl-3-azabicyclo[3.1.0]hexane-2-carboxamide (Compound 202)

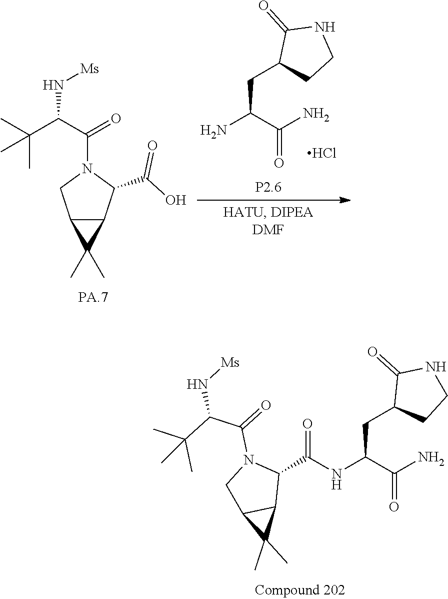 [SMILES: CC(C)(C)[C@H](NS(C)(=O)=O)C(=O)N1C[C@H]2[C@@H]([C@H]1C(=O)O)C2(C)C ; NC(=O)[C@@H](N)C[C@@H]1CCNC1=O ; Cl ; CC(C)(C)[C@H](NS(C)(=O)=O)C(=O)N1C[C@H]2[C@@H]([C@H]1C(=O)N[C@@H](C[C@@H]1CCNC1=O)C(N)=O)C2(C)C]

To a solution of PA.7 (100 mg, 0.29 mmol) in DMF (2.0 mL) was added P2.6 (73 mg, 0.35 mmol, HCl salt), HATU (121 mg, 0.32 mmol) and N,N-diisopropylethylamine (112 mg, 0.87 mmol) at 0° C. The reaction mixture was stirred at 25° C. for 16 h. The reaction mixture was purified by reverse phase column chromatography (5-100% MeOH in water) to give compound 202 (50 mg, 35% yield). ¹H NMR (400 MHz, CDCl₃), δ: 8.18 (d, J=7.2 Hz, 1H), 7.06 (br. s, 1H), 6.20 (br. s, 1H), 6.08 (br. s, 2H), 4.55-4.50 (m, 1H), 4.32 (s, 1H), 4.05-4.01 (m, 1H), 3.89-3.87 (m, 1H), 3.79 (d, J=10.0 Hz, 1H), 3.35-3.29 (m, 2H), 2.91 (s, 3H), 2.57-2.49 (m, 1H), 2.40-2.32 (m, 1H), 2.06-1.95 (m, 2H), 1.86-1.83 (m, 1H), 1.57-1.54 (m, 1H), 1.45 (d, J=7.6 Hz, 1H), 1.04-1.02 (m, 12H), 0.96 (s, 3H).

### Example 14. (1R,2S,5S)—N—((S)-1-Cyano-2-((S)-2-oxopyrrolidin-3-yl)ethyl)-3-((S)-3,3-dimethyl-2-(methylsulfonamido)butanoyl)-6,6-dimethyl-3-azabicyclo[3.1.0]hexane-2-carboxamide (Compound 201)

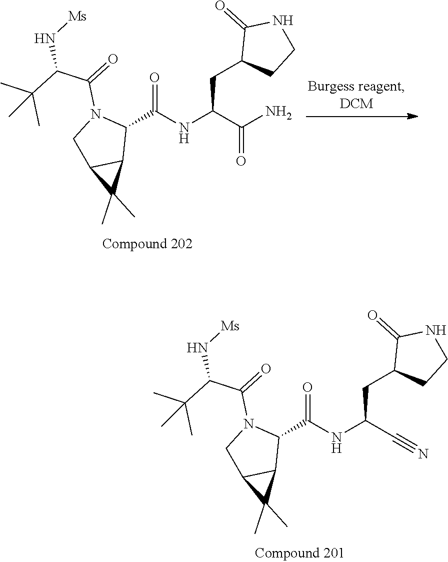 [SMILES: CC(C)(C)[C@H](NS(C)(=O)=O)C(=O)N1C[C@H]2[C@@H]([C@H]1C(=O)N[C@@H](C[C@@H]1CCNC1=O)C(N)=O)C2(C)C ; CC(C)(C)[C@H](NS(C)(=O)=O)C(=O)N1C[C@H]2[C@@H]([C@H]1C(=O)N[C@H](C#N)C[C@@H]1CCNC1=O)C2(C)C]

To a solution of compound 202 (45 mg, 0.09 mmol) in dichloromethane (2.0 mL) was added Burgess reagent (54 mg, 0.23 mmol) at 25° C. After the reaction mixture had been stirred at 25° C. for 16 h. The reaction mixture was concentrated and purified by reverse phase column chromatography (5-100% MeOH in water) to give compound 201 (40 mg, 92% yield). ¹H NMR (400 MHz, DMSO-d₆), δ: 9.04 (d, J=8.8 Hz, 1H), 7.64 (s, 1H), 7.11 (d, J=10.0 Hz, 1H), 4.99-4.93 (m, 1H), 4.15 (s, 1H), 3.86-3.80 (m, 2H), 3.67 (d, J=10.4 Hz, 1H), 3.14-3.10 (m, 1H), 3.05-2.98 (m, 1H), 2.82 (s, 3H), 2.45-2.37 (m, 1H), 2.18-2.00 (m, 2H), 1.73-1.65 (m, 1H), 1.57-1.54 (m, 1H), 1.29 (d, J=7.6 Hz, 1H), 1.02 (s, 3H), 0.94 (s, 9H), 0.91 (s, 3H); LCMS (ESI+) m/z: calc. for C₂₂H₃₅N₅O₅S 481.61; found, 482.2 [M+H]⁺; HPLC purity: 100.00%, t_{R}=15.287 min.

### Example 15. (1R,2S,5S)—N-[(1S)-2-amino-2-oxo-1-[[(3S)-2-oxopyrrolidin-3-yl]methyl]ethyl]-3-[(2S)-3,3-dimethyl-2-(2,2,2-trifluoroethylsulfonylamino)butanoyl]-6,6-dimethyl-3-azabicyclo[3.1.0]hexane-2-carboxamide (compound 208)

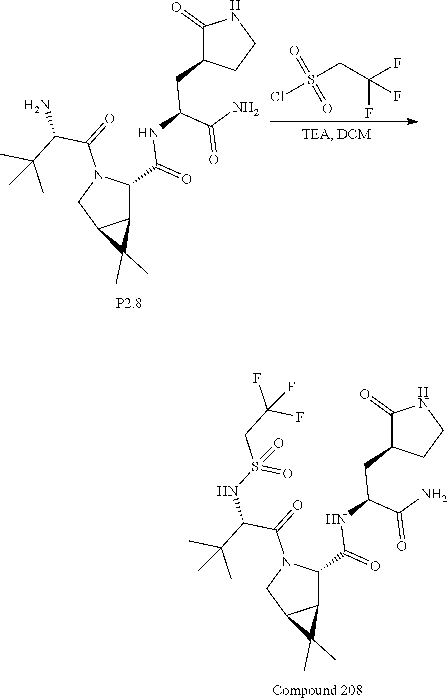

To a solution of P2.8 (40 mg, 0.095 mmol) in DCM (2.0 mL) was added 2,2,2-trifluoroethane-1-sulfonyl chloride (20 mg, 0.114 mmol) and TEA (40 mg, 0.38 mmol) at 0° C. The reaction mixture was stirred at 0° C. for 2 h. The mixture was concentrated and purified by reverse phase column chromatography (5-100% of MeOH in water) to give the desired product compound 208 (30 mg, 56% yield). ¹H NMR (400 MHz, CDCl₃), δ: 8.14 (d, J=6.4 Hz, 1H), 7.06 (s, 1H), 6.06 (br. s, 1H), 5.77 (br. s, 1H), 5.68 (br. s, 1H), 4.54-4.49 (m, 1H), 4.3 (s, 1H), 4.06-4.04 (m, 1H), 3.95 (d, J=8.8 Hz, 1H), 3.83-3.69 (m, 3H), 3.42-3.33 (m, 2H), 2.57-2.48 (m, 1H), 2.42-2.35 (m, 1H), 2.05-2.02 (m, 2H), 1.89-1.84 (m, 1H), 1.55-1.52 (m, 1H), 1.47-1.44 (m, 1H), 1.05 (s, 12H), 0.95 (s, 3H).

### Example 16. (1R,2S,5S)—N-[(1S)-1-cyano-2-[(3S)-2-oxopyrrolidin-3-yl]ethyl]-3-[(2S)-3,3-dimethyl-2-(2,2,2-trifluoroethylsulfonylamino)butanoyl]-6,6-dimethyl-3-azabicyclo[3.1.0]hexane-2-carboxamide (Compound 209)

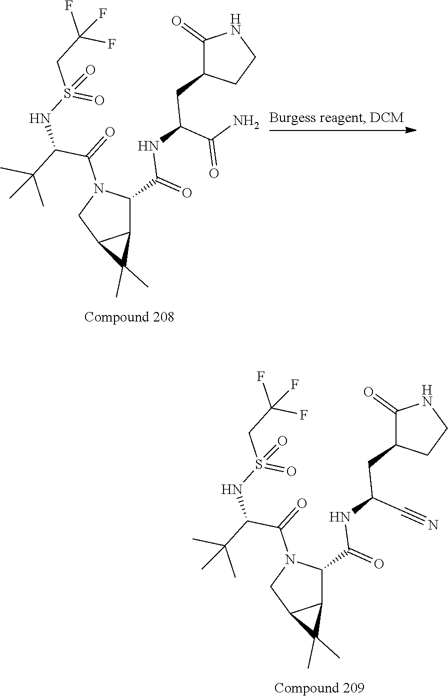 [SMILES: CC(C)(C)[C@H](NS(=O)(=O)CC(F)(F)F)C(=O)N1C[C@H]2[C@@H]([C@H]1C(=O)N[C@@H](C[C@@H]1CCNC1=O)C(N)=O)C2(C)C ; CC(C)(C)[C@H](NS(=O)(=O)CC(F)(F)F)C(=O)N1C[C@H]2[C@@H]([C@H]1C(=O)N[C@H](C#N)C[C@@H]1CCNC1=O)C2(C)C]

To a solution of compound 208 (30 mg, 0.053 mmol) in DCM (2.0 mL) was added Burgess reagent (32 mg, 0.132 mmol) at 25° C. After the reaction mixture had been stirred at 25° C. for 16 h. The reaction mixture was concentrated and purified by reverse phase column chromatography (5-100% of MeOH in water) to give compound 209 (15 mg, 52% yield). ¹H NMR (400 MHz, DMSO-d₆), δ: 8.36 (d, J=6.8 Hz, 1H), 5.96 (d, J=9.2 Hz, 1H), 5.70 (br. s, 1H), 4.95-4.89 (m, 1H), 4.25 (s, 1H), 3.99-3.91 (m, 2H), 3.84-3.67 (m, 3H), 3.50-3.33 (m, 3H), 2.57-2.28 (m, 3H), 2.10-2.04 (m, 1H), 1.90-1.84 (m, 1H), 1.62-1.59 (m, 1H), 1.51-1.50 (m, 1H), 1.07-1.03 (m, 12H), 0.95 (s, 3H); LCMS (ESI+) m/z: calc. for C₂₃H₃₄F₃N₅O₅S 549.61; found, 550.2 [M+H]⁺; HPLC purity: 97.21%, t_{R}=18.592 min.

### Example 17. (1R,2S,5S)—N-[(1S)-1-Cyano-2-[(3S)-2-oxopyrrolidin-3-yl]ethyl]-3-[(2S)-3,3-dimethyl-2-(2,2,2-trifluoroethylsulfonylamino)butanoyl]-6,6-dimethyl-3-azabicyclo[3.1.0]hexane-2-carboxamide (Compound 227)

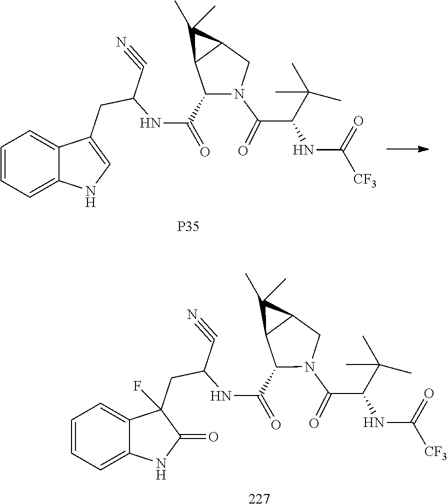

Selectfluor (3.0 eq, 200 mg) was added to a stirred solution of P35 (1.0 eq, 100 mg, 0.188 mmol) in ACN/water (1/1, 2 mL) at rt. After overnight stirring, the reaction mixture was diluted with ethyl acetate (50 mL), washed with water (10 mL), 4% HCl (10 mL), a saturated solution of sodium bicarbonate (10 mL), and brine (10 mL), and dried over Na₂SO₄. The solvent was removed under reduced pressure to give a residue that was purified by prep RP-HPLC on a C18 column eluting with a gradient MeCN—H₂O+0.1% TFA to give compound 227 (26 mg, 24.5%). ¹H NMR (400 MHz, DMSO) δ 11.20-10.72 (m, 1H), 9.36 (t, J=7.2 Hz, 1H), 9.03 (d, J=7.9 Hz, 1H), 7.61-7.45 (m, 1H), 7.42-7.34 (m, 1H), 7.14-7.01 (m, 1H), 6.92 (d, J=7.8 Hz, 1H), 5.15-4.98 (m, 1H), 4.39 (t, J=8.3 Hz, 1H), 4.21-4.11 (m, 1H), 3.94-3.79 (m, 1H), 3.72 (s, 8H), 3.67 (d, J=10.8 Hz, 1H), 2.87-2.75 (m, 1H), 1.58-1.43 (m, 1H), 1.35-1.16 (m, 1H), 1.06-0.90 (m, 13H), 0.88 (d, J=5.4 Hz, 1H), 0.86-0.77 (m, 3H).

### Analytical Data of Certain Final Compounds

### Compound 214. (1R,2S,5S)—N—((S)-1-cyano-2-(2-oxoimidazolidin-1-yl)ethyl)-3-((S)-3,3-dimethyl-2-(2,2,2-trifluoroacetamido)butanoyl)-6,6-dimethyl-3-azabicyclo[3.1.0]hexane-2-carboxamide

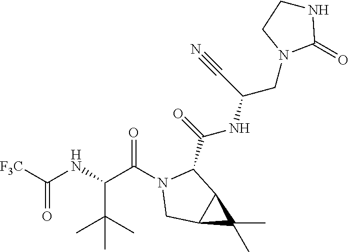 [SMILES: CC(C)(C)[C@H](NC(=O)C(F)(F)F)C(=O)N1C[C@H]2[C@@H]([C@H]1C(=O)N[C@H](C#N)CN1CCNC1=O)C2(C)C]

White solid. ¹H NMR (400 MHz, CD₃OD), δ: 5.06-5.03 (m, 1H), 4.57 (m, 1H), 4.29 (s, 1H), 4.05-4.01 (m, 1H), 3.86-3.84 (m, 1H), 3.64-3.55 (m, 4H), 3.43-3.39 (m, 2H), 1.63-1.60 (m, 1H), 1.41 (d, J=7.6 Hz, 1H), 1.08-0.97 (m, 12H), 0.92 (s, 3H); LCMS (ESI) m/z calc. for C₂₂H₃₁F₃N₆O₄ 500.24; found, 501.7 [M+H]⁺. HPLC purity: 98.7%, t_{R}=17.992 min.

### Compound 215. (1R,2S,5S)—N—((S)-1-cyano-2-(3-methyl-2-oxoimidazolidin-1-yl)ethyl)-3-((S)-3,3-dimethyl-2-(2,2,2-trifluoroacetamido)butanoyl)-6,6-dimethyl-3-azabicyclo[3.1.0]hexane-2-carboxamide

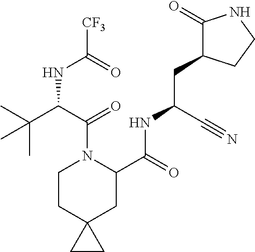 [SMILES: CC(C)(C)[C@H](NC(=O)C(F)(F)F)C(=O)N1CCC2(CC2)CC1C(=O)N[C@H](C#N)C[C@@H]1CCNC1=O]

White solid. ¹H NMR (400 MHz, CDCl₃), δ: 8.03 (d, J=6.8 Hz, 1H), 6.89 (d, J=9.6 Hz, 1H), 4.86-4.82 (m, 1H), 4.55 (d, J=9.6 Hz, 1H), 4.33 (s, 1H), 4.02-3.98 (m, 1H), 3.78 (d, J=10.4 Hz, 1H), 3.67-3.62 (m, 2H), 3.54-3.46 (m, 1H), 3.45-3.41 (m, 3H), 2.83 (s, 3H), 1.57-1.50 (m, 1H), 1.47-1.44 (m, 1H), 1.05-1.03 (m, 9H), 0.94 (s, 3H), 0.86 (s, 3H); LCMS (ESI) m/z calc. for C₂₃H₃₃F₃N₆O₄ 514.25; found, 515.2 [M+H]⁺. HPLC purity: 100.00%, t_{R}=19.426 min.

### Compound 302. (1R,2S,5S)—N—((S)-1-Cyano-2-(3-methyl-2-oxo-2,3-dihydro-1H-benzo[d]imidazol-1-yl)ethyl)-3-((S)-3,3-dimethyl-2-(2,2,2-trifluoroacetamido)butanoyl)-6,6-dimethyl-3-azabicyclo[3.1.0]hexane-2-carboxamide

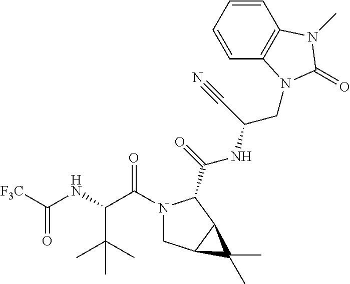 [SMILES: Cn1c(=O)n(C[C@@H](C#N)NC(=O)[C@@H]2[C@@H]3[C@H](CN2C(=O)[C@@H](NC(=O)C(F)(F)F)C(C)(C)C)C3(C)C)c2ccccc21]

White solid. ¹H NMR (400 MHz, CDCl₃), δ: 8.91-8.04 (m, 1H), 7.25-6.97 (m, 5H), 5.31-5.00 (m, 1H), 4.63-4.51 (m, 1H), 4.43-4.17 (m, 3H), 4.05-3.74 (m, 2H), 3.58-3.38 (m, 3H), 1.59-1.23 (m, 2H), 1.06-0.79 (m, 15H); LCMS (ESI) m/z calc. for C₂₇H₃₃F₃N₆O₄ 562.25; found, 563.4 [M+H]⁺; HPLC purity: 97.0%, t_{R}=22.195 min.

### Compound 206. N—((S)-1-Cyano-2-((S)-2-oxopyrrolidin-3-yl)ethyl)-6-((S)-3,3-dimethyl-2-(2,2,2-trifluoroacetamido)butanoyl)-6-azaspiro[2.5]octane-5-carboxamide

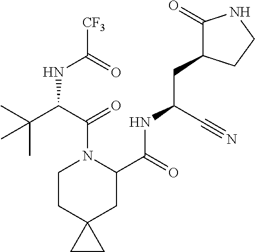 [SMILES: CC(C)(C)[C@H](NC(=O)C(F)(F)F)C(=O)N1CCC2(CC2)CC1C(=O)N[C@H](C#N)C[C@@H]1CCNC1=O]

White solid. ¹H NMR (400 MHz, CDCl₃), δ: 9.47 (s, 0.5H), 8.91-8.80 (m, 1H), 7.41-7.09 (m, 1H), 5.83-5.73 (m, 1H), 5.35-5.27 (m, 1H), 5.01-4.99 (m, 1H), 4.71-4.42 (m, 2H), 4.07-3.97 (m, 1H), 3.81-3.55 (m, 1H), 3.45-3.03 (m, 2H), 2.63-2.39 (m, 2H), 2.34-2.18 (m, 2H), 2.13-1.86 (m, 4H), 1.76-1.53 (m, 1H), 1.07-1.01 (m, 9H), 0.97-0.91 (m, 1H), 0.66-0.55 (m, 1H), 0.45-0.35 (m, 3H); LCMS (ESI) m/z calc. for C₂₃H₃₂F₃N₅O₄ 499.24; found, 500.3 [M+H]⁺. HPLC purity: 66.3% and 30.3%, t_{R}=18.690 and 19.167 min. The desired product is a mixture of two diastereoisomers.

### Compound 226. (1S,2S,5S)—N-[1-cyano-2-(2-oxoindolin-3-yl)ethyl]-3-[(2S)-3,3-dimethyl-2-[(2,2,2-trifluoroacetyl)amino]butanoyl]-6,6-dimethyl-3-azabicyclo[3.1.0]hexane-2-carboxamide

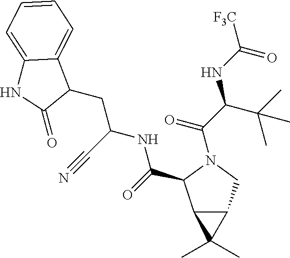 [SMILES: CC(C)(C)[C@H](NC(=O)C(F)(F)F)C(=O)N1C[C@H]2[C@H]([C@H]1C(=O)NC(C#N)CC1C(=O)Nc3ccccc31)C2(C)C]

¹H NMR (400 MHz, DMSO-d6), δ: 10.55-10.45 (m, 1H), 9.37 (d, J=8.4 Hz, 1H), 9.30 (dd, J=8.3, 3.7 Hz, 1H), 9.05 (q, J=7.8 Hz, 1H), 7.37-7.20 (m, 1H), 7.19 (d, J=7.8 Hz, 1H), 7.03-6.94 (m, 1H), 6.93-6.79 (m, 1H), 5.19-5.03 (m, 1H), 4.44-4.32 (m, 1H), 4.24-4.16 (m, 1H), 3.96-3.84 (m, 1H), 3.74-3.63 (m, 1H), 2.33-2.19 (m, 2H), 1.63-1.46 (m, 1H), 1.41-1.32 (m, 1H), 1.08-0.86 (m, 11H), 0.88-0.81 (m, 6H).

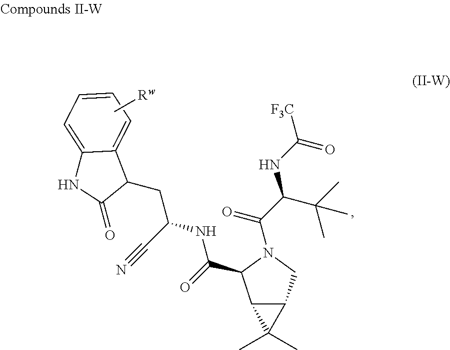

| Compound | R^{W} | NMR |
|---|---|---|
| 218 | 5-Cl | ¹H-NMR (400 MHz, DMSO-d6), δ: 10.51-10.62 (m, 1H), 9.22- |
| | | 9.39 (m, 1.5H), 8.87-9.00 (m, 0.2H), 7.36-7.49 (m, 1H), 7.18- |
| | | 7.28 (m, 1H), 6.75-6.89 (m, 1H), 5.25-5.35 (m, 0.7H), 5.07- |
| | | 5.17 (m, 0.23H), 4.32-4.44 (m, 1H), 4.14-4.23 (m, 1H), 3.83- |
| | | 3.97 (m, 1H), 3.64-3.76 (m, 1H), 3.51-3.63 (m, 1H), 2.28- |
| | | 2.39 (m, 1.27H), 2.06-2.15 (m, 0.7H), 1.54-1.64 (m, 1H), 1.34- |
| | | 1.37 (m, 1H), 1.02-1.06 (m, 2.8H), 0.95-1.01 (m, 2H) 0.90- |
| | | 0.94 (s, 2H), 0.83-0.89 (m, 3.7H), 0.73-0.81 (s, 6.3H) |
| 219 | 5-Br | ¹H-NMR (400 MHz, DMSO-d6), δ: 10.47-10.72 (m, 1H), 9.20- |
| | | 9.41 (m, 1.43H), 8.90-9.06 (m, 0.68H), 7.23-7.38 (m, 1H), |
| | | 7.00-7.05 (m, 0.68H), 6.90-6.94 (m, 0.4H), 6.80-6.86 (m, |
| | | 1H), 4.97-5.26 (m, 1H), 4.30-4.45 (m, 1H), 4.12-4.24 (m, |
| | | 1H), 3.81-3.95 (m, 1H), 3.61-3.75 (m, 1H), 3.47-3.59 (m, |
| | | 1H), 2.20-2.36 (m, 1.64H), 2.04-2.20 (m, 0.45H), 1.45-1.63 |
| | | (m, 1.2H), 1.29-1.41 (m, 0.88H), 1.18-1.26 (m, 0.5H), 0.92- |
| | | 1.05 (m, 10.5H), 0.79-0.90 (m, 7.4H) |
| 220 | 5-CH₃ | ¹H-NMR (400 MHz, CD₃OD), δ: 6.98-7.27 (m, 2H), 6.70-6.85 |
| | | (m, 1H), 5.33-5.41 (m, 0.31H), 5.14-5.28 (m, 0.4H), 5.06- |
| | | 5.12 (m, 0.3H), 4.46-4.61 (m, 1.46H), 4.25-4.34 (m, 0.93H), |
| | | 3.92-4.08 (m, 1H), 3.79-3.88 (m, 1H), 3.59-3.70 (m, 0.5H), |
| | | 2.12-2.68 (m, 5.7H), 1.46-1.69 (m, 1.3H), 1.40-1.45 (m, |
| | | 0.8H), 0.99-1.11 (m, 10H), 0.85-0.98 (m, 7H) |
| 221 | 5-OCH₃ | ¹H-NMR (400 MHz, DMSO-d6), δ: 10.20-10.38 (m, 1H), 9.18- |
| | | 9.37 (m, 1.28H), 8.89-9.06 (m, 0.6H), 6.87-7.11 (m, 1H), |
| | | 6.69-6.79 (m, 2H), 4.99-5.37 (m, 1H), 4.28-4.46 (m, 1H), |
| | | 4.13-4.26 (m, 1H), 3.63-3.73 (m, 4H), 3.44-3.54 (m, 0.36H), |
| | | 2.16-2.38 (m, 1.59H), 2.04-2.14 (m, 0.39H), 1.44-1.63 (m, |
| | | 1H), 1.31-1.39 (m, 0.78H), 1.18-1.27 (m, 0.4H), 0.91-1.07 |
| | | (m, 9H), 0.78-0.90 (m, 7H) |
| 222 | 6-OCH₃ | ¹H-NMR (400 MHz, DMSO-d6), δ: 10.31-10.51 (m, 1H), 9.16- |
| | | 9.40 (m, 1.25H), 8.87-9.09 (m, 0.52H), 7.07-7.30 (m, 1H), |
| | | 6.33-6.60 (m, 2H), 4.97-5.24 (m, 1H), 4.29-4.49 (m, 1H), |
| | | 4.10-4.27 (m, 1H), 3.83-3.99 (m, 1H), 3.57-3.77 (m, 4H), |
| | | 3.36-3.50 (m, 0.79H), 2.16-2.34 (m, 1.53H), 2.04-2.14 (m, |
| | | 0.42H), 1.43-1.65 (m, 1H), 1.29-1.41 (m, 0.85H), 1.20- |
| | | 1.29 (m, 0.36H), 0.91-1.07 (m, 9H), 0.78-0.90 (m, 7H) |
| 225 | 6-CH₃ | ¹H-NMR (400 MHz, DMSO-d6), δ: 10.35-10.44 (m, 1H), 9.20- |
| | | 9.36 (m, 1.65H), 8.95-9.01 (m, 0.5H), 7.12-7.20 (m, 1H), |
| | | 6.67-6.81 (m, 1H), 6.61-6.67 (m, 1H), 5.10-5.22 (m, 1H), |
| | | 4.32-4.45 (m, 1H), 4.15-4.26 (m, 1H), 3.84-3.97 (m, 1H), |
| | | 3.64-3.74 (m, 1H), 3.39-3.54 (m, 1H), 2.17-2.33 (m, 5H), |
| | | 2.03-2.15 (m, 0.67H), 1.72-1.78 (m, 0.15H), 1.52-1.64 (m, |
| | | 1H), 1.29-1.38 (m, 1H), 1.00-1.06 (m, 3.6H), 0.91-0.97 (m, |
| | | 4.8H), 0.81-0.89 (m, 9.2H) |
| 232 | 6-Cl | ¹H-NMR (400 MHz, DMSO-d6), δ: 10.50-10.69 (m, 1H), 9.23- |
| | | 9.38 (m, 1.25H), 8.89-9.08 (m, 0.67H), 7.32-7.45 (m, |
| | | 1.23H), 6.72-6.83 (m, 1H), 5.23-5.36 (m, 0.3H), 4.95-5.16 |
| | | (m, 0.75H), 4.29-4.45 (m, 1H), 4.10-4.24 (m, 1H), 3.81-3.94 |
| | | (m, 1H), 3.52-3.74 (m, 2H), 2.20-2.38 (m, 1.5H), 2.04-2.16 |
| | | (m, 0.3H), 1.43-1.64 (m, 1H), 1.30-1.40 (m, 0.66H), 1.15- |
| | | 1.20 (m, 0.3H) 0.95-1.05 (m, 8H), 0.86-0.94 (m, 1.6H), 0.81- |
| | | 0.86 (m, 2.6H), 0.74-0.8 (m, 2H) |
| 233 | 7-Cl | ¹H-NMR (400 MHz, DMSO-d6), δ: 10.52-10.70 (m, 1H), 9.24- |
| | | 9.39 (m, 1H), 8.92-9.06 (m, 0.78H), 7.13-7.31 (m, 2H), 6.94- |
| | | 6.99 (m, 1H), 4.96-5.13 (m, 1H), 4.32-4.44 (m, 1H), 4.13- |
| | | 4.23 (m, 1H), 3.81-3.94 (m, 1H), 3.62-3.72 (m, 1H), 3.43- |
| | | 3.54 (m, 1H), 2.21-2.36 (m, 1.5H), 1.44-1.55 (m, 1H), 1.31- |
| | | 1.38 (m, 0.6H), 1.19-1.26 (m, 0.6H), 0.91-1.09 (m, 12H), |
| | | 0.77-0.90 (m, 4.6H) |
| 234 | 4-CH₃ | ¹H-NMR (400 MHz, CD₃OD), δ: 7.04-7.20 (m, 1H), 6.78-6.90 |
| | | (m, 1H), 6.63-6.78 (m, 1H), 5.12-5.17 (m, 0.32H), 5.03-5.10 |
| | | (m, 0.31 H), 4.94-4.99 (m, 0.27H), 4.50-4.59 (m, 1H), 4.26- |
| | | 4.35 (m, 0.68H), 4.18-4.25 (m, 0.3H), 3.89-4.07 (m, 1H), 3.79- |
| | | 3.86 (m, 1H), 3.64-3.75 (m, 0.7H), 2.85-2.96 (m, 0.3H), 2.62- |
| | | 2.82 (m, 0.7H), 2.31-2.40 (m, 3H), 2.17-2.31 (m, 1H), 1.50- |
| | | 1.64 (m, 1H), 1.39-1.48 (m, 0.64H), 1.32-1.37 (m, 0.18H), |
| | | 1.19-1.25 (m, 0.18H), 1.05-1.11 (m, 2.7H), 0.98-1.05 (m, |
| | | 8.7H), 0.96-0.98 (s, 0.4H), 0.93-0.95 (s, 0.4H), 0.89-0.93 |
| | | (m, 2.3H), 0.86-0.88 (s, 0.47H) |
| 235 | 6-Br | ¹H-NMR (400 MHz, DMSO-d6), δ: 10.77-11.02 (m, 1H), 9.22- |
| | | 9.42 (m, 1.32H), 8.89-9.14 (m, 0.6H), 7.15-7.39 (m, 2H), |
| | | 6.75-7.08 (m, 1H), 4.92-5.37 (m, 1H), 4.30-4.45 (m, 1H), |
| | | 4.13-4.24 (m, 1H), 3.80-3.96 (m, 1H), 3.57-3.74 (m, 2H), |
| | | 2.25-2.39 (m, 1.45H), 2.13-2.25 (m, 0.44H), 1.55-1.61 (m, |
| | | 0.48H), 1.45-1.55 (m, 0.5H), 1.32-1.40 (m, 0.69H), 1.20- |
| | | 1.25 (m, 0.28H), 0.91-1.07 (m, 8H), 0.78-0.90 (m, 6H) |
| 236 | 5-F | ¹H-NMR (400 MHz, DMSO-d₆), δ: 10.42-10.55 (m, 1H), 9.21- |
| | | 9.40 (m, 1.4H), 8.91-9.06 (m, 0.53H), 7.10-7.31 (m, 1H), |
| | | 6.94-7.06 (m, 1H), 6.70-6.88 (m, 1H), 5.24-5.35 (m, 0.4H), |
| | | 5.07-5.16 (m, 0.4H), 4.96-5.06 (m, 025H), 4.29-4.45 (m, |
| | | 1H), 4.13-4.23 (m, 1H), 3.81-3.95 (m, 1H), 3.61-3.75 (m, |
| | | 1H), 3.46-3.59 (m, 1H), 2.03-2.14 (m, 0.45H), 1.45-1.62 (m, |
| | | 1H), 1.32-1.38 (m, 0.73H), 1.17-1.22 (m, 0.36H), 0.95-1.06 |
| | | (m, 7.3H), 0.91-0.94 (s, 1H), 0.80-0.86 (m, 2.8H), 0.75-0.80 |
| | | (s, 3H) |

### Biological Assays

### Example A. Primary Assay Used to Determine Potency of SARS-CoV2-Mpro Enzymatic Activity Inhibition

(1) Deliver 2× Enzyme
(2) Deliver buffer into No Enzyme wells
(3) Deliver compounds in 100% DMSO by using Acoustic technology (Echo550; nanoliter range)
(4) Incubate for 20 min
(5) Deliver 2× Substrate to initiate the reaction
(6) Spin & shake, start measuring in EnVision at rt; 5 min interval for 25 (2 hours)
(7) Analyze data by taking slope (signal/time) of linear portion of measurement
(8) Slope is calculated by using Excel, and curve fits are performed using Prism software.

The % inhibition was then used to calculate the IC₅₀ values. The IC₅₀ values are shown in Table A, wherein “A” corresponds to IC₅₀<10.0 nm, “B” corresponds to 10.0 nm≤IC₅₀<50.0 nm, “C” corresponds to 50.0 nm≤IC₅₀<100.0 nm, “D” corresponds to 100.0 nm≤IC₅₀<500.0 nm, and “E” corresponds to 500.0 nm≤IC₅₀<1000.0 nm, and “F” corresponds to 1000.0 nm≤IC₅₀.

TABLE A
SARS-CoV2-Mpro enzymatic activity Inhibition

| Compound Number | IC₅₀, nM* |
|---|---|
| 201 | B |
| 206 | B |
| 209 | B |
| 217 | A |
| 218 | B |
| 219 | B |
| 220 | B |
| 221 | A |
| 222 | B |
| 226 | A |
| 227 | B |
| 228 | B |
| 229 | B |
| 230 | B |
| 231 | C |
| 232 | B |
| 233 | C |
| 234 | E |
| 235 | A |
| 236 | B |
| 237 | A |
| 238 | A |
| 239 | F |
| 243 | A |
| 244 | B |
| 246 | B |
| 247 | F |
| 248 | F |
| 249 | E |
| 250 | E |
| 301 | F |
| 308 | F |

*IC50 is a quantitative measure that indicates how much of a particular inhibitory substance (e.g. drug) is needed to inhibit, in vitro, a given biological process or biological component by 50%; wherein “A” corresponds to IC50 < 10.0 nm, “B” corresponds to 10.0 nm ≤ IC50 < 50.0 nm, “C” corresponds to 50.0 nm ≤ IC50 < 100.0 nm, “D” corresponds to 100.0 nm ≤ IC50 < 500.0 nm, and “E” corresponds to 500.0 nm ≤ IC50 < 1000.0 nm, and “F” corresponds to 1000.0 nm ≤ IC50.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A compound of Formula (A): 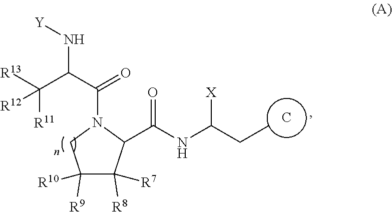
or a pharmaceutically acceptable salt, stereoisomer, solvate, prodrug, or tautomer thereof, wherein:
C is a heterocyclic ring selected from: 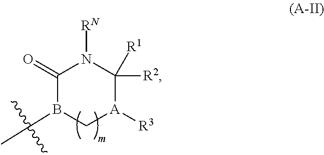
wherein: m is an integer selected from 0, 1, 2; A is selected from CR⁴ and N; B is selected from CR⁶ and N; 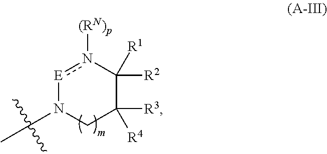
wherein: the bond [symbol] is selected from a single bond and a double bond; p is an integer selected from 0, 1; E is selected from C(O) and N; provided that when the bond [symbol] is a single bond p is 1 and E is C(O), and when the bond [symbol] is a double bond p is 0 and E is N; m is an integer selected from 0, 1, 2; 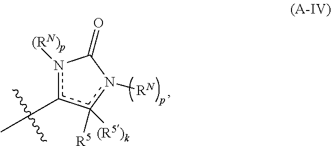
wherein: each bond [symbol] and [symbol] is a single bond or double bond; when [symbol] is a double bond each bond [symbol] is a single bond, when [symbol] is a single bond each bond [symbol] is independently selected from a single bond or double bond; each p is an integer independently selected from 0 and 1; provided that when [symbol] is a single bond then p is 1 and when [symbol] is a double bond then p is 0; k is an integer selected from 0, 1; provided that when the bond 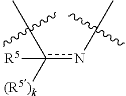
is a single bond and the bond [symbol] is a single bond then k is 1 and when the bond 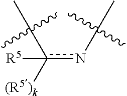
or the bond [symbol] is a double bond then k is 0;
and 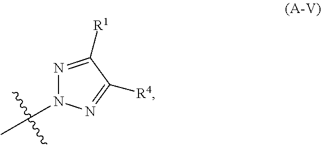
wherein:
R^{N} is selected from H, C₁-C₆ alkyl, cycloalkyl;
R¹ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R² is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R¹ and R² together is oxo;
or R² and R³ together form a bond;
R³ is selected from hydrogen, C₁-C₆ alkyl, C₂-C₆ alkenyl, C₂-C₆ alkynyl, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R⁴ is selected from hydrogen, halogen, —OH, —CN, —NO₂, —NR¹³R¹⁴, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R¹ and R⁴ together with the atoms to which they are attached and any intervening atoms, form a 3-10 membered cycloalkyl, a 6-10 membered aryl, a 3-14 membered heterocycle, or a 5-6 membered heteroaryl, wherein the cycloalkyl, aryl, heterocycle or heteroaryl is optionally substituted with one or more R¹⁶;
R⁵ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹³, —C(O)N—NR¹⁴R¹⁵, cycloalkyl, —O-cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R^{5′} is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)N—NR¹⁴R¹⁵, cycloalkyl, —O-cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R⁵ and R^{5′} form oxo;
R⁶ is selected from H, C₁-C₆ alkyl, cycloalkyl, halogen, —CN, —OH;
R⁷, R⁸, R⁹ and R¹⁰ are each independently selected from H, alkyl;
or R⁸ and R⁹ together with the atoms to which they are attached and any intervening atoms, form cycloalkyl optionally substituted with one or more halogen, —OH, —CN, C₁-C₆ alkyl;
or R⁹ and R¹⁰ together with the atoms to which they are attached and any intervening atoms, form cycloalkyl optionally substituted with one or more halogen, —OH, —CN, C₁-C₆ alkyl;
R¹¹, R¹², R¹³ are each independently selected from C₁-C₆ alkyl, cycloalkyl, halogen, —CN;
R¹⁴ and R¹⁵ are each independently selected from H, C₁-C₆ alkyl;
or R¹⁴ and R¹⁵ together with the atoms to which they are attached and any intervening atoms, form heterocyclyl optionally substituted with one or more halogen, —OH, —CN, C₁-C₆ alkyl;
R¹⁶ is independently selected from halogen, —OH, —CN, —NO₂, —NR¹³R¹⁴, C₁-C₆ alkyl, C₂-C₆ alkenyl, C₂-C₆ alkynyl, C₁-C₆ alkoxy, —C(O)OR¹³, —C(O)N—NR¹³R¹⁴, cycloalkyl, —O-cycloalkyl, aryl, heterocyclyl, and heteroaryl;
X is selected from hydrogen, halogen, OH, CN, NO₂, CONH₂, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl wherein alkyl optionally substituted with cycloalkyl, heterocyclyl, aryl, and heteroaryl;
Y is selected from C₁-C₆ alkyl-S(O)ᵤ—, C₁-C₆ alkyl-C(O)—, heteroaryl-C(O)—, wherein alkyl or heteroaryl is optionally substituted with one or more halogen, —OH, —CN;
n is integer selected from 0, 1, 2;
u is integer selected from 0, 1, 2;
wherein,
cycloalkyl is a mono or polycyclic saturated carbon rings containing 3-18 carbon atoms;
aryl is a cyclic, aromatic hydrocarbon groups that have 1 to 3 aromatic rings;
heterocyclyl is a saturated or partially unsaturated 3-10 membered monocyclic, 7-12 membered bicyclic (fused, bridged, or spiro rings), or 11-14 membered tricyclic ring system (fused, bridged, or spiro rings) having one or more heteroatoms selected from O, N, S, P, Se, or B;
heteroaryl is a monovalent monocyclic or a polycyclic aromatic radical of 5 to 24 ring atoms, containing one or more ring heteroatoms selected from N, O, S, P, or B, the remaining ring atoms being C;
provided that when in heterocycle (A-II) m is 0; A and B are CH; R¹ and R² are CH₃, H, deuterium, or R¹ is OH, or R¹ and R² together is oxo; n is 1; R⁸ and R⁹ are C₁-C₆ alkyl or R⁸ and R⁹ together with the atoms to which they are attached and any intervening atoms, form cyclopropyl or cyclohexyl, or R⁹ and R¹⁰ together with the atoms to which they are attached and any intervening atoms, form cyclobutyl, cyclohexyl, or R⁹ is cyclopentyl; R¹¹, R¹², R¹³ are each CH₃; and X is CN then Y is not CF₃C(O)—, (CH₃)₃CS(O)₂—, ClCH₂C(O)—, (CH₃)₂CHC(O)—, CH₃CH₂C(O)—, CH₃C(O)—;
provided that when in heterocycle (A-II) m is 1; A and B are CH; R¹ and R² are CH₃ or H; n is 1; R⁸ and R⁹ together with the atoms to which they are attached and any intervening atoms, form cyclopropyl, cyclopentyl or cyclohexyl, or R⁹ and R¹⁰ together with the atoms to which they are attached and any intervening atoms, form cyclobutyl, cyclohexyl, or R⁹ is cyclopentyl, then Y is not CF₃C(O)—;
and provided that when in heterocycle (A-II) m is 0; A and B are CH; R¹ and R² are both H; n is 2; R⁹ or R¹⁰ is C₁-C₆ alkyl, then Y is not CF₃C(O)—, CH₃S(O)₂.

2. The compound of claim 1, wherein the compound is of Formula (II): 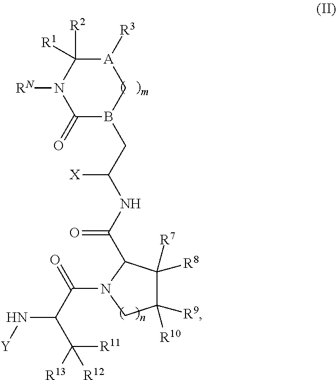
or a pharmaceutically acceptable salt, stereoisomer, solvate, prodrug, or tautomer thereof,
wherein:
A is selected from CR⁴ and N;
B is selected from CR⁶ and N;
R^{N} is selected from H, C₁-C₆ alkyl, cycloalkyl;
R¹ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R² is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R¹ and R² together is oxo;
or R² and R³ together form a bond;
R³ is selected from hydrogen, C₁-C₆ alkyl, C₂-C₆ alkenyl, C₂-C₆ alkynyl, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R⁴ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R¹ and R⁴ together with the atoms to which they are attached and any intervening atoms, form a 3-10 membered cycloalkyl, a 6-10 membered aryl, a 3-14 membered heterocycle, or a 5-6 membered heteroaryl, wherein the cycloalkyl, aryl, heterocycle or heteroaryl is optionally substituted with one or more R¹⁶;
or R³ and R⁴ together is oxo;
R⁶ is selected from H, C₁-C₆ alkyl, cycloalkyl, halogen, —CN, —OH;
R⁷, R⁸, R⁹ and R¹⁰ are each independently selected from H, C₁-C₆ alkyl, cycloalkyl;
or R⁸ and R⁹ together with the atoms to which they are attached and any intervening atoms, form cycloalkyl optionally substituted with one or more halogen, —OH, —CN, C₁-C₆ alkyl;
or R⁹ and R¹⁰ together with the atoms to which they are attached and any intervening atoms, form cycloalkyl optionally substituted with one or more halogen, —OH, —CN, C₁-C₆ alkyl;
R¹¹, R¹², R¹³ are each independently selected from C₁-C₆ alkyl, cycloalkyl, halogen, —CN;
R¹⁴ and R¹⁵ are each independently selected from H, C₁-C₆ alkyl;
or R¹⁴ and R¹⁵ together with the atoms to which they are attached and any intervening atoms, form heterocyclyl optionally substituted with one or more halogen, —OH, —CN, C₁-C₆ alkyl;
R¹⁶ is independently selected from halogen, —OH, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)N—NR¹⁴R¹⁵, cycloalkyl, —O-cycloalkyl, aryl, heterocyclyl, and heteroaryl;
X is selected from hydrogen, halogen, OH, CN, NO₂, CONH₂, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl wherein alkyl optionally substituted with cycloalkyl, heterocyclyl, aryl, and heteroaryl;
Y is selected from C₁-C₆ alkyl-S(O)ᵤ—, C₁-C₆ alkyl-C(O)—, heteroaryl-C(O)—, wherein alkyl or heteroaryl is optionally substituted with one or more halogen, —OH, —CN;
m is an integer selected from 0, 1, 2;
n is an integer selected from 0, 1, 2;
u is integer selected from 0, 1, 2;
wherein,
cycloalkyl is a mono or polycyclic saturated carbon rings containing 3-18 carbon atoms;
aryl is a cyclic, aromatic hydrocarbon groups that have 1 to 3 aromatic rings;
heterocyclyl is a saturated or partially unsaturated 3-10 membered monocyclic, 7-12 membered bicyclic (fused, bridged, or spiro rings), or 11-14 membered tricyclic ring system (fused, bridged, or spiro rings) having one or more heteroatoms selected from O, N, S, P, Se, or B;
heteroaryl is a monovalent monocyclic or a polycyclic aromatic radical of 5 to 24 ring atoms, containing one or more ring heteroatoms selected from N, O, S, P, or B, the remaining ring atoms being C;
provided that when m is 0; A and B are CH; R¹ and R² are CH₃, H, deuterium, or R¹ is OH, or R¹ and R² together is oxo; n is 1; R⁸ and R⁹ are C₁-C₆ alkyl or R⁸ and R⁹ together with the atoms to which they are attached and any intervening atoms, form cyclopropyl or cyclohexyl, or R⁹ and R¹⁰ together with the atoms to which they are attached and any intervening atoms, form cyclobutyl, cyclohexyl, or R⁹ is cyclopentyl; R¹¹, R¹², R¹³ are each CH₃; and X is CN then Y is not CF₃C(O)—, (CH₃)₃CS(O)₂—, ClCH₂C(O)—, (CH₃)₂CHC(O)—, CH₃CH₂C(O)—, CH₃C(O)—;
provided that when m is 1; A and B are CH; R¹ and R² are CH₃ or H; n is 1; R⁸ and R⁹ together with the atoms to which they are attached and any intervening atoms, form cyclopropyl, cyclopentyl or cyclohexyl, or R⁹ and R¹⁰ together with the atoms to which they are attached and any intervening atoms, form cyclobutyl, cyclohexyl, or R⁹ is cyclopentyl, then Y is not CF₃C(O)—;
and provided that when m is 0; A and B are CH; R¹ and R² are both H; n is 2; R⁹ or R¹⁰ is C₁-C₆ alkyl, then Y is not CF₃C(O)—, CH₃S(O)₂.

3. The compound of claim 1, wherein the compound is of Formula (III): 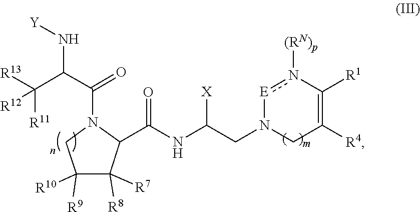
or a pharmaceutically acceptable salt, stereoisomer, solvate, prodrug, or tautomer thereof,
wherein:
the bond [symbol] is selected from a single bond and a double bond;
p is an integer selected from 0, 1;
E is selected from C(O) and N;
provided that when the bond [symbol] is a single bond p is 1, E is C(O) and when the bond [symbol] is a double bond p is 0, E is N;
R^{N} is selected from H, C₁-C₆ alkyl, cycloalkyl;
R¹ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R⁴ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R¹ and R⁴ together with the atoms to which they are attached and any intervening atoms, form a 3-10 membered cycloalkyl, a 6-10 membered aryl, a 3-14 membered heterocycle, or a 5-6 membered heteroaryl, wherein the cycloalkyl, aryl, heterocycle or heteroaryl is optionally substituted with one or more R¹⁶;
R⁷, R⁸, R⁹ and R¹⁰ are each independently selected from H, C₁-C₆ alkyl, cycloalkyl;
or R⁸ and R⁹ together with the atoms to which they are attached and any intervening atoms, form cycloalkyl optionally substituted with one or more halogen, —OH, —CN, C₁-C₆ alkyl;
or R⁹ and R¹⁰ together with the atoms to which they are attached and any intervening atoms, form cycloalkyl optionally substituted with one or more halogen, —OH, —CN, C₁-C₆ alkyl;
R¹¹, R¹², R¹³ are each independently selected from C₁-C₆ alkyl, cycloalkyl, halogen, —CN;
R¹⁴ and R¹⁵ are each independently selected from H, C₁-C₆ alkyl;
or R¹⁴ and R¹⁵ together with the atoms to which they are attached and any intervening atoms, form heterocyclyl optionally substituted with one or more halogen, —OH, —CN, C₁-C₆ alkyl;
R¹⁶ is independently selected from halogen, —OH, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)N—NR¹⁴R¹⁵, cycloalkyl, —O-cycloalkyl, aryl, heterocyclyl, and heteroaryl;
X is selected from hydrogen, halogen, OH, CN, NO₂, CONH₂, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl wherein alkyl optionally substituted with cycloalkyl, heterocyclyl, aryl, and heteroaryl;
Y is selected from C₁-C₆ alkyl-S(O)ᵤ—, C₁-C₆ alkyl-C(O)—, heteroaryl-C(O)—, wherein alkyl or heteroaryl is optionally substituted with one or more halogen, —OH, —CN;
m is an integer selected from 0, 1, 2;
n is an integer selected from 0, 1, 2;
u is integer selected from 0, 1, 2;
wherein,
cycloalkyl is a mono or polycyclic saturated carbon rings containing 3-18 carbon atoms;
aryl is a cyclic, aromatic hydrocarbon groups that have 1 to 3 aromatic rings;
heterocyclyl is a saturated or partially unsaturated 3-10 membered monocyclic, 7-12 membered bicyclic (fused, bridged, or spiro rings), or 11-14 membered tricyclic ring system (fused, bridged, or spiro rings) having one or more heteroatoms selected from O, N, S, P, Se, or B;
heteroaryl is a monovalent monocyclic or a polycyclic aromatic radical of 5 to 24 ring atoms, containing one or more ring heteroatoms selected from N, O, S, P, or B, the remaining ring atoms being C.

4. The compound of claim 1, wherein the compound is of Formula (IV): 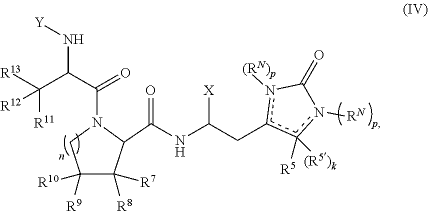
or a pharmaceutically acceptable salt, stereoisomer, solvate, prodrug, or tautomer thereof,
wherein:
each bond [symbol] and [symbol] is a single bond or double bond;
when [symbol] is a double bond each bond [symbol] is a single bond, when [symbol] is a single bond each bond [symbol] is independently selected from a single bond or double bond;
each p is an integer independently selected from 0 and 1;
provided that when [symbol] is a single bond then p is 1 and when [symbol] is a double bond then p is 0;
k is an integer selected from 0, 1;
provided that when the bond 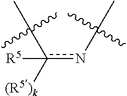
is a single bond and the bond [symbol] is a single k is 1 and when the bond 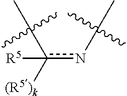
or the bond [symbol] is a double bond k is 0;
each R^{N} is independently selected from H, C₁-C₆ alkyl, cycloalkyl;
R⁵ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹³, —C(O)N—NR¹⁴R¹⁵, cycloalkyl, —O-cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R^{5′} is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)N—NR¹⁴R¹⁵, cycloalkyl, —O-cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R⁵ and R^{5′} form oxo;
R⁷, R⁸, R⁹ and R¹⁰ are each independently selected from H, C₁-C₆ alkyl, cycloalkyl;
or R⁸ and R⁹ together with the atoms to which they are attached and any intervening atoms, form cycloalkyl optionally substituted with one or more halogen, —OH, —CN, C₁-C₆ alkyl;
or R⁹ and R¹⁰ together with the atoms to which they are attached and any intervening atoms, form cycloalkyl optionally substituted with one or more halogen, —OH, —CN, C₁-C₆ alkyl;
R¹¹, R¹², R¹³ are each independently selected from C₁-C₆ alkyl, cycloalkyl, halogen, —CN;
R¹⁴ and R¹⁵ are each independently selected from H, C₁-C₆ alkyl;
or R¹⁴ and R¹⁵ together with the atoms to which they are attached and any intervening atoms, form heterocyclyl optionally substituted with one or more halogen, —OH, —CN, C₁-C₆ alkyl;
R¹⁶ is independently selected from halogen, —OH, —OH, —CN, —NO₂, —NR¹⁴R¹⁵, C₁-C₆ alkyl, C₂-C₆ alkenyl, C₂-C₆ alkynyl, C₁-C₆ alkoxy, —C(O)OR¹⁴, —C(O)N—NR¹⁴R¹⁵, cycloalkyl, —O-cycloalkyl, aryl, heterocyclyl, and heteroaryl;
X is selected from hydrogen, halogen, OH, CN, NO₂, CONH₂, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl wherein alkyl optionally substituted with cycloalkyl, heterocyclyl, aryl, and heteroaryl;
Y is selected from C₁-C₆ alkyl-S(O)ᵤ—, C₁-C₆ alkyl-C(O)—, heteroaryl-C(O)—, wherein alkyl or heteroaryl is optionally substituted with one or more halogen, —OH, —CN;
n is an integer selected from 0, 1, 2;
u is an integer selected from 0, 1, 2;
wherein,
cycloalkyl is a mono or polycyclic saturated carbon rings containing 3-18 carbon atoms;
aryl is a cyclic, aromatic hydrocarbon groups that have 1 to 3 aromatic rings;
heterocyclyl is a saturated or partially unsaturated 3-10 membered monocyclic, 7-12 membered bicyclic (fused, bridged, or spiro rings), or 11-14 membered tricyclic ring system (fused, bridged, or spiro rings) having one or more heteroatoms selected from O, N, S, P, Se, or B;
heteroaryl is a monovalent monocyclic or a polycyclic aromatic radical of 5 to 24 ring atoms, containing one or more ring heteroatoms selected from N, O, S, P, or B, the remaining ring atoms being C.

5. The compound of claim 1, wherein the compound is of Formula (V): 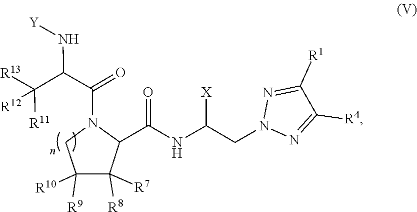
or a pharmaceutically acceptable salt, stereoisomer, solvate, prodrug, or tautomer thereof,
wherein:
R¹ is selected from hydrogen, halogen, —OH, —CN, —NO₂, —NR¹⁴R¹⁵, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
R⁴ is selected from hydrogen, halogen, —OH, —CN, —NO₂, —NR¹³R¹⁴, C₁-C₆ alkyl, C₂-C₆ alkenyl, C₂-C₆ alkynyl, C₁-C₆ alkoxy, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹⁶;
or R¹ and R⁴ together with the atoms to which they are attached and any intervening atoms, form a 3-10 membered cycloalkyl, a 6-10 membered aryl, a 3-14 membered heterocycle, or a 5-6 membered heteroaryl, wherein the cycloalkyl, aryl, heterocycle or heteroaryl is optionally substituted with one or more R¹⁶;
R⁷, R⁸, R⁹ and R¹⁰ are each independently selected from H, C₁-C₆ alkyl, cycloalkyl;
or R⁸ and R⁹ together with the atoms to which they are attached and any intervening atoms, form cycloalkyl optionally substituted with one or more halogen, —OH, —CN, C₁-C₆ alkyl;
or R⁹ and R¹⁰ together with the atoms to which they are attached and any intervening atoms, form cycloalkyl optionally substituted with one or more halogen, —OH, —CN, C₁-C₆ alkyl;
R¹¹, R¹², R¹³ are each independently selected from C₁-C₆ alkyl, cycloalkyl, halogen, —CN;
R¹⁴ and R¹⁵ are each independently selected from H, C₁-C₆ alkyl;
or R¹⁴ and R¹⁵ together with the atoms to which they are attached and any intervening atoms, form heterocyclyl optionally substituted with one or more halogen, —OH, —CN, C₁-C₆ alkyl;
R¹⁶ is independently selected from halogen, —OH, —OH, —CN, —NO₂, —NR¹³R¹⁴, C₁-C₆ alkyl, C₂-C₆ alkenyl, C₂-C₆ alkynyl, C₁-C₆ alkoxy, —C(O)OR¹³, —C(O)N—NR¹³R¹⁴, cycloalkyl, —O-cycloalkyl, aryl, heterocyclyl, and heteroaryl;
X is selected from hydrogen, halogen, OH, CN, NO₂, CONH₂, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl wherein alkyl optionally substituted with cycloalkyl, heterocyclyl, aryl, and heteroaryl;
Y is selected from C₁-C₆ alkyl-S(O)ᵤ—, C₁-C₆ alkyl-C(O)—, heteroaryl-C(O)—, wherein alkyl or heteroaryl is optionally substituted with one or more halogen, —OH, —CN;
n is integer selected from 0, 1, 2;
u is integer selected from 0, 1, 2;
wherein,
cycloalkyl is a mono or polycyclic saturated carbon rings containing 3-18 carbon atoms;
aryl is a cyclic, aromatic hydrocarbon groups that have 1 to 3 aromatic rings;
heterocyclyl is a saturated or partially unsaturated 3-10 membered monocyclic, 7-12 membered bicyclic (fused, bridged, or spiro rings), or 11-14 membered tricyclic ring system (fused, bridged, or spiro rings) having one or more heteroatoms selected from O, N, S, P, Se, or B;
heteroaryl is a monovalent monocyclic or a polycyclic aromatic radical of 5 to 24 ring atoms, containing one or more ring heteroatoms selected from N, O, S, P, or B, the remaining ring atoms being C.

6. The compound of claim 2, wherein the compound is of Formula (II-I-A-A-A-I), (II-I-A-A-A-II), (II-I-A-A-A-III), (II-I-A-A-B-I), (II-I-A-A-C-I), (II-I-A-A-D-I), (II-I-A-B-A-I), (II-I-B-A-A-I), (II-II-A-A-A-I), (II-II-A-B-A-I), or (II-II-A-C-A-I): 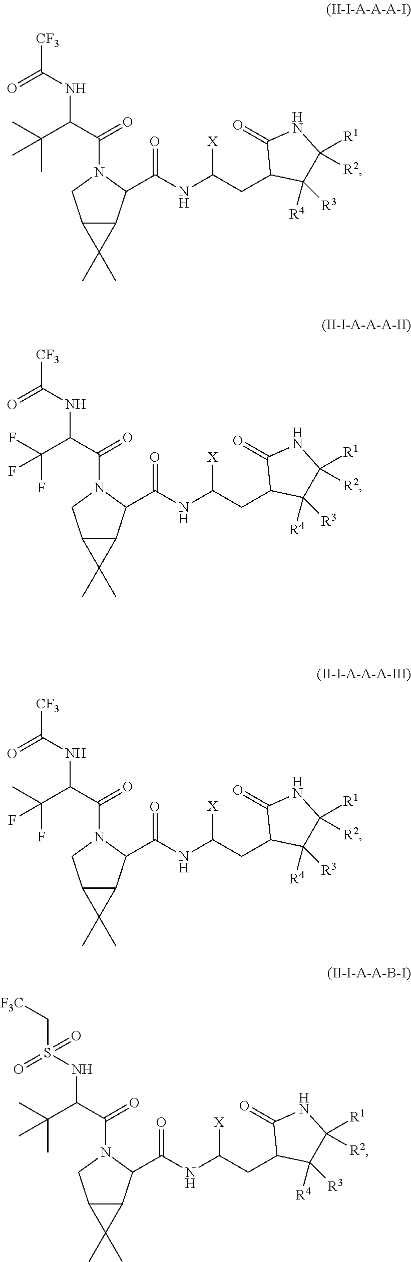 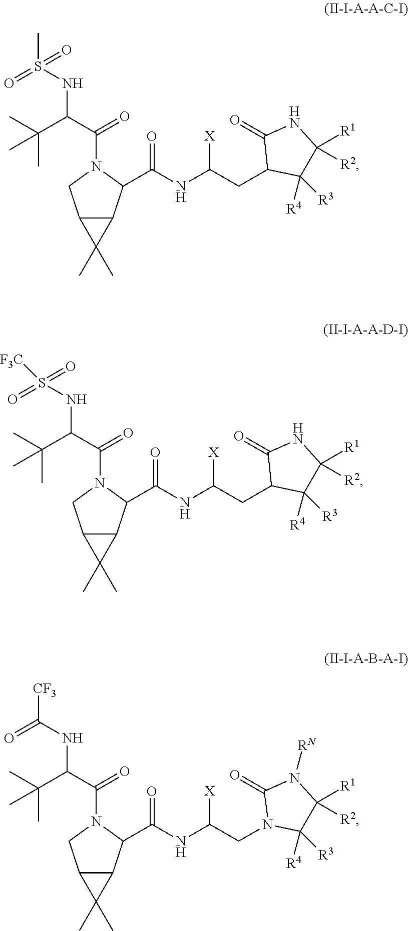 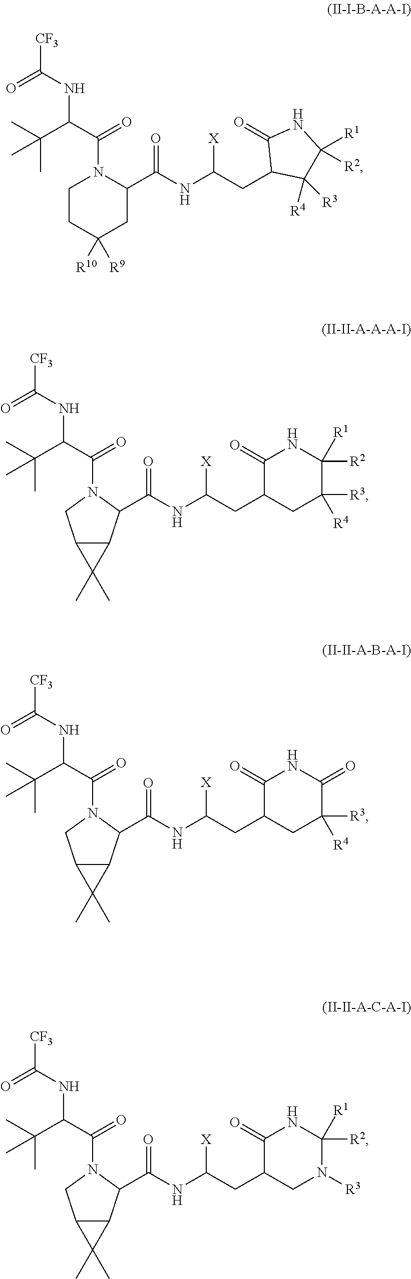
or a pharmaceutically acceptable salt, stereoisomer, solvate, or tautomer thereof.

7. The compound of claim 2, wherein the compound is of Formula (II-I-A-A-A-I-A) or (II-I-A-A-A-I-B): 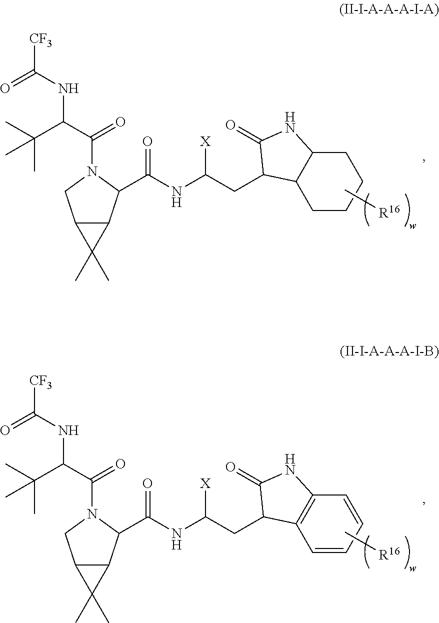
or a pharmaceutically acceptable salt, stereoisomer, solvate, or tautomer thereof, wherein w is an integer selected from 0, 1, 2, 3, and 4.

8. The compound of claim 2, wherein the compound is of Formula (II-I-W): 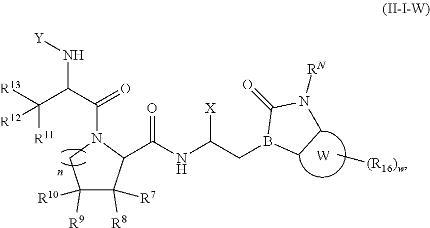
or a pharmaceutically acceptable salt, stereoisomer, solvate, prodrug, isotopic derivative, or tautomer thereof, wherein ring W is selected from 3-10 membered cycloalkyl, a 6-10 membered aryl, a 3-14 membered heterocycle, or a 5-6 membered heteroaryl; w is an integer selected from 0, 1, 2, 3, and 4; and all other variables are as defined herein.

9. The compound of claim 3, wherein the compound is of Formula (III-I-I-A-A-A-I) or (III-II-I-A-A-I): 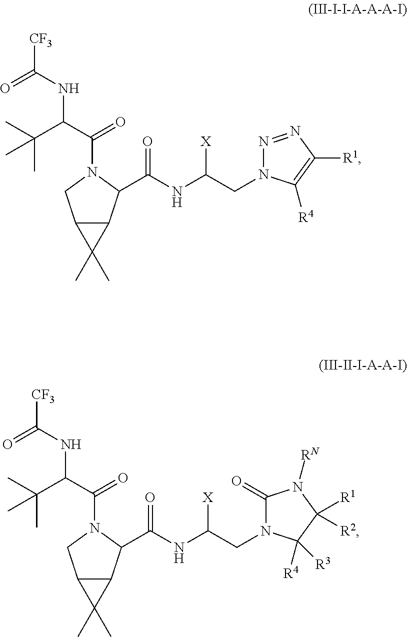
or a pharmaceutically acceptable salt, stereoisomer, solvate, or tautomer thereof.

10. The compound of claim 4, wherein the compound is of Formula (IV-I-A-A-I), (IV-II-A-A-I), or (IV-III-A-A-I): 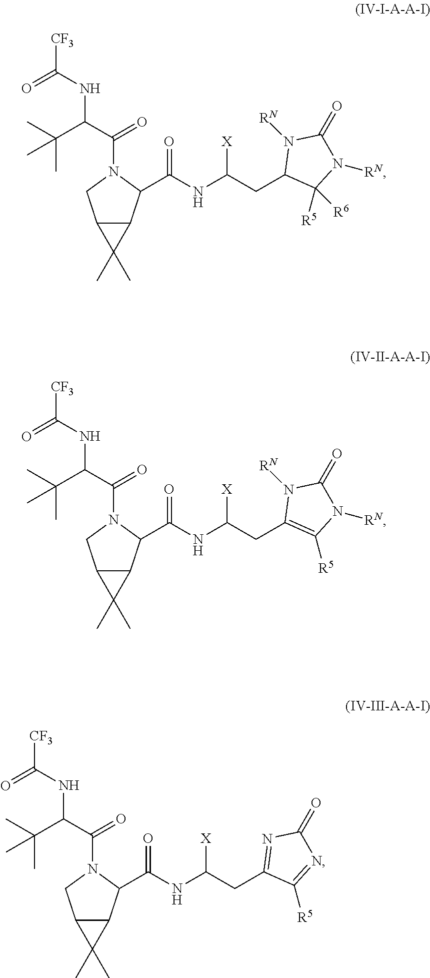
or a pharmaceutically acceptable salt, stereoisomer, solvate, or tautomer thereof.

11. The compound of claim 5, wherein the compound is of Formula (V-I-A-A-I): 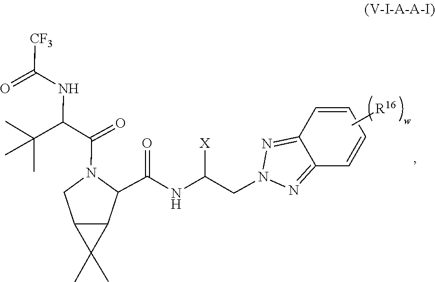
or a pharmaceutically acceptable salt, stereoisomer, solvate, or tautomer thereof, wherein w is an integer selected from 0, 1, 2, 3, 4.

12. A compound selected from:

| # | Structure | IUPAC Name |
|---|---|---|
| 201 | 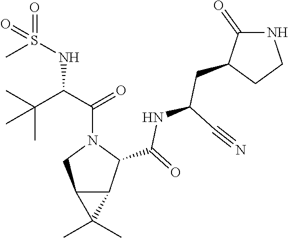 [SMILES: CC(C)(C)[C@H](NS(C)(=O)=O)C(=O)N1C[C@H]2[C@H]([C@H]1C(=O)N[C@H](C#N)C[C@@H]1CCNC1=O)C2(C)C] | (1S,2S,5S)-N-[(1S)-1-cyano- 2-[(3S)-2-oxopyrrolidin-3- yl]ethyl]-3-[(2S)-2- (methanesulfonamido)-3,3- dimethyl-butanoyl]-6,6- dimethyl-3- azabicyclo[3.1.0]hexane-2- carboxamide |
| 202 | 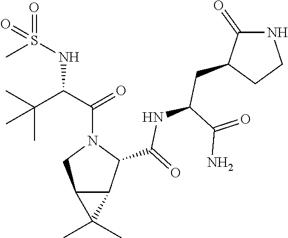 [SMILES: CC(C)(C)[C@H](NS(C)(=O)=O)C(=O)N1C[C@H]2[C@H]([C@H]1C(=O)N[C@@H](C[C@@H]1CCNC1=O)C(N)=O)C2(C)C] | (1S,2S,5S)-N-[(1S)-2-amino- 2-oxo-1-[[(3S)-2- oxopyrrolidin-3- yl]methyl]ethyl]-3-[(2S)-2- (methanesulfonamido)-3,3- dimethyl-butanoyl]-6,6- dimethyl-3- azabicyclo[3.1.0]hexane-2- carboxamide |
| 203 | 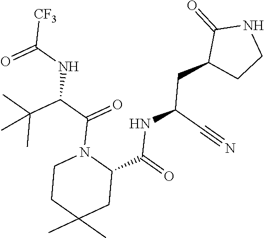 [SMILES: CC1(C)CCN(C(=O)[C@@H](NC(=O)C(F)(F)F)C(C)(C)C)[C@H](C(=O)N[C@H](C#N)C[C@@H]2CCNC2=O)C1] | (2S)-N-[(1S)-1-cyano-2- [(3S)-2-oxopyrrolidin-3- yl]ethyl]-1-[(2S)-3,3- dimethyl-2-[(2,2,2- trifluoroacetyl)amino]butanoyl]- 4,4-dimethyl-piperidine- 2-carboxamide |
| 204 | 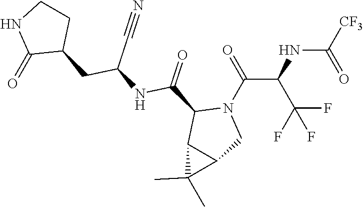 [SMILES: CC1(C)[C@@H]2[C@@H](C(=O)N[C@H](C#N)C[C@@H]3CCNC3=O)N(C(=O)[C@@H](NC(=O)C(F)(F)F)C(F)(F)F)C[C@@H]21] | (1R,2S,5S)-N-[(1S)-1-cyano- 2-[(3S)-2-oxopyrrolidin-3- yl]ethyl]-6,6-dimethyl-3- [(2R)-3,3,3-trifluoro-2- [(2,2,2- trifluoroacetyl)amino] propanoyl]-3- azabicyclo[3.1.0]hexane-2- carboxamide |
| 205 | 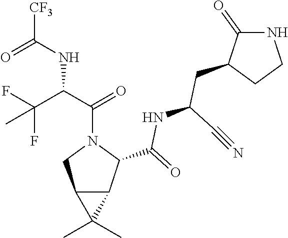 [SMILES: CC(F)(F)[C@H](NC(=O)C(F)(F)F)C(=O)N1C[C@H]2[C@H]([C@H]1C(=O)N[C@H](C#N)C[C@@H]1CCNC1=O)C2(C)C] | (1S,2S,5S)-N-[(1S)-1-cyano- 2-[(3S)-2-oxopyrrolidin-3- yl]ethyl]-3-[(2R)-3,3- difluoro-2-[(2,2,2- trifluoroacetyl)amino]butanoyl]- 6,6-dimethyl-3- azabicyclo[3.1.0]hexane-2- carboxamide |
| 206 | 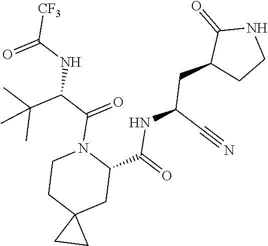 [SMILES: CC(C)(C)[C@H](NC(=O)C(F)(F)F)C(=O)N1CCC2(CC2)C[C@H]1C(=O)N[C@H](C#N)C[C@@H]1CCNC1=O] | (7S)-N-[(1S)-1-cyano-2- [(3S)-2-oxopyrrolidin-3- yl]ethyl]-6-[(2S)-3,3- dimethyl-2-[(2,2,2- trifluoroacetyl)amino]butanoyl]- 6-azaspiro[2.5]octane-7- carboxamide |
| 207 | 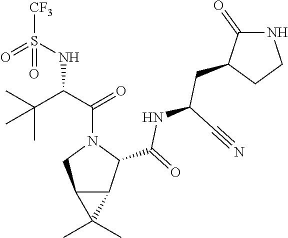 [SMILES: CC(C)(C)[C@H](NS(=O)(=O)C(F)(F)F)C(=O)N1C[C@H]2[C@H]([C@H]1C(=O)N[C@H](C#N)C[C@@H]1CCNC1=O)C2(C)C] | (1S,2S,5S)-N-[(1S)-1-cyano- 2-[(3S)-2-oxopyrrolidin-3- yl]ethyl]-3-[(2S)-3,3- dimethyl-2- (trifluoromethylsulfonylamino) butanoyl]-6,6-dimethyl-3- azabicyclo[3.1.0]hexane-2- carboxamide |
| 208 | 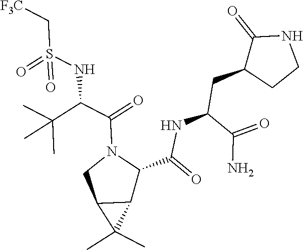 [SMILES: CC(C)(C)[C@H](NS(=O)(=O)CC(F)(F)F)C(=O)N1C[C@H]2[C@H]([C@H]1C(=O)N[C@@H](C[C@@H]1CCNC1=O)C(N)=O)C2(C)C] | (1S,2S,5S)-N-[(1S)-2-amino- 2-oxo-1-[[(3S)-2- oxopyrrolidin-3- yl]methyl]ethyl]-3-[(2S)-3,3- dimethyl-2-(2,2,2- trifluoroethylsulfonylamino) butanoyl]-6,6-dimethyl-3- azabicyclo[3.1.0]hexane-2- carboxamide |
| 209 | 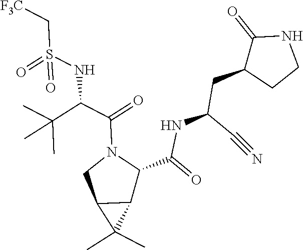 [SMILES: CC(C)(C)[C@H](NS(=O)(=O)CC(F)(F)F)C(=O)N1C[C@H]2[C@H]([C@H]1C(=O)N[C@H](C#N)C[C@@H]1CCNC1=O)C2(C)C] | (1S,2S,5S)-N-[(1S)-1-cyano- 2-[(3S)-2-oxopyrrolidin-3- yl]ethyl]-3-[(2S)-3,3- dimethyl-2-(2,2,2- trifluoroethylsulfonylamino) butanoyl]-6,6-dimethyl-3- azabicyclo[3.1.0]hexane-2- carboxamide |
| 212 | 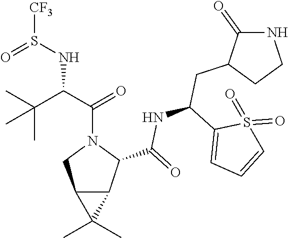 [SMILES: CC(C)(C)[C@H](NS(=O)C(F)(F)F)C(=O)N1C[C@H]2[C@H]([C@H]1C(=O)N[C@@H](CC1CCNC1=O)C1=CC=CS1(=O)=O)C2(C)C] | (1S,2S,5S)-3-[(2S)-3,3- dimethyl-2-[(2,2,2- trifluoroacetyl)amino]butanoyl]- N-[(1S)-1-(1,1-dioxo-2- thienyl)-2-(2-oxopyrrolidin- 3-yl)ethyl]-6,6-dimethyl-3- azabicyclo[3.1.0]hexane-2- carboxamide |
| 214 | 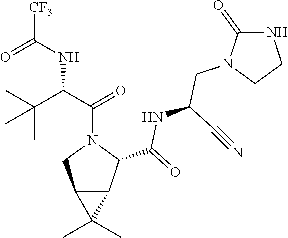 [SMILES: CC(C)(C)[C@H](NC(=O)C(F)(F)F)C(=O)N1C[C@H]2[C@H]([C@H]1C(=O)N[C@H](C#N)CN1CCNC1=O)C2(C)C] | (1S,2S,5S)-N-[(1S)-1-cyano- 2-(2-oxoimidazolidin-1- yl)ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 215 | 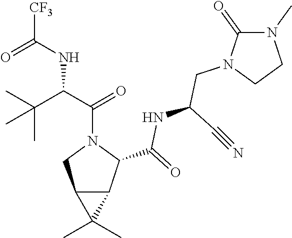 [SMILES: CN1CCN(C[C@@H](C#N)NC(=O)[C@@H]2[C@H]3[C@H](CN2C(=O)[C@@H](NC(=O)C(F)(F)F)C(C)(C)C)C3(C)C)C1=O] | (1S,2S,5S)-N-[(1S)-1-cyano- 2-(3-methyl-2-oxo- imidazolidin-1-yl)ethyl]-3- [(2S)-3,3-dimethyl-2-[(2,2,2- trifluoroacetyl)amino]butanoyl]- 6,6-dimethyl-3- azabicyclo[3.1.0]hexane-2- carboxamide |
| 216 | 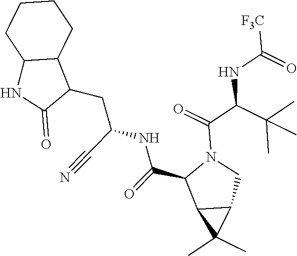 [SMILES: CC(C)(C)[C@H](NC(=O)C(F)(F)F)C(=O)N1C[C@H]2[C@H]([C@H]1C(=O)N[C@H](C#N)CC1C(=O)NC3CCCCC31)C2(C)C] | (1S,2S,5S)-N-[(1S)-1-cyano- 2-(2-oxo-1,3,3a,4,5,6,7,7a- octahydroindol-3-yl)ethyl]-3- [(2S)-3,3-dimethyl-2-[(2,2,2- trifluoroacetyl)amino]butanoyl]- 6,6-dimethyl-3- azabicyclo[3.1.0]hexane-2- carboxamide |
| 217 | 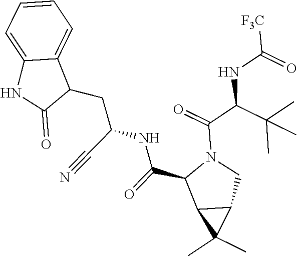 [SMILES: CC(C)(C)[C@H](NC(=O)C(F)(F)F)C(=O)N1C[C@H]2[C@H]([C@H]1C(=O)N[C@H](C#N)CC1C(=O)Nc3ccccc31)C2(C)C] | (1S,2S,5S)-N-[(1S)-1-cyano- 2-(2-oxoindolin-3-yl)ethyl]- 3-[(2S)-3,3-dimethyl-2- [(2,2,2- trifluoroacetyl)amino]butanoyl]- 6,6-dimethyl-3- azabicyclo[3.1.0]hexane-2- carboxamide |
| 218 | 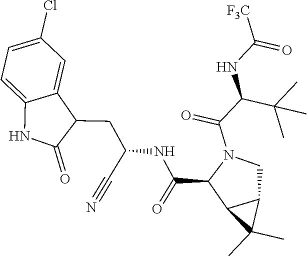 [SMILES: CC(C)(C)[C@H](NC(=O)C(F)(F)F)C(=O)N1C[C@H]2[C@H]([C@H]1C(=O)N[C@H](C#N)CC1C(=O)Nc3ccc(Cl)cc31)C2(C)C] | (1S,2S,5S)-N-[(1S)-2-(5- chloro-2-oxo-indolin-3-yl)-1- cyano-ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 219 | 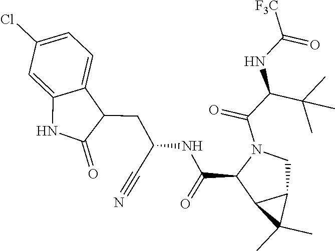 [SMILES: CC(C)(C)[C@H](NC(=O)C(F)(F)F)C(=O)N1C[C@H]2[C@H]([C@H]1C(=O)N[C@H](C#N)CC1C(=O)Nc3cc(Cl)ccc31)C2(C)C] | (1S,2S,5S)-N-[(1S)-2-(6- chloro-2-oxo-indolin-3-yl)-1- cyano-ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 220 | 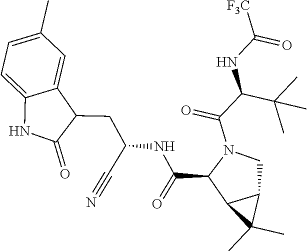 [SMILES: Cc1ccc2c(c1)C(C[C@@H](C#N)NC(=O)[C@@H]1[C@H]3[C@H](CN1C(=O)[C@@H](NC(=O)C(F)(F)F)C(C)(C)C)C3(C)C)C(=O)N2] | (1S,2S,5S)-N-[(1S)-1-cyano- 2-(5-methyl-2-oxo-indolin-3- yl)ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 221 | 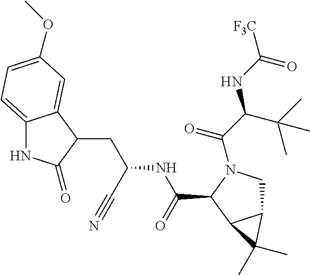 [SMILES: COc1ccc2c(c1)C(C[C@@H](C#N)NC(=O)[C@@H]1[C@H]3[C@H](CN1C(=O)[C@@H](NC(=O)C(F)(F)F)C(C)(C)C)C3(C)C)C(=O)N2] | (1S,2S,5S)-N-[(1S)-1-cyano- 2-(5-methoxy-2-oxo-indolin- 3-yl)ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 222 | 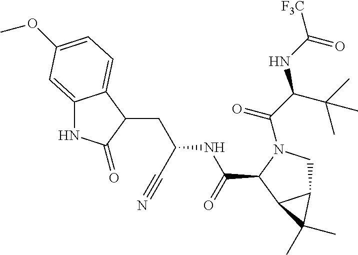 [SMILES: COc1ccc2c(c1)NC(=O)C2C[C@@H](C#N)NC(=O)[C@@H]1[C@H]2[C@H](CN1C(=O)[C@@H](NC(=O)C(F)(F)F)C(C)(C)C)C2(C)C] | (1S,2S,5S)-N-[(1S)-1-cyano- 2-(6-methoxy-2-oxo-indolin- 3-yl)ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 223 | 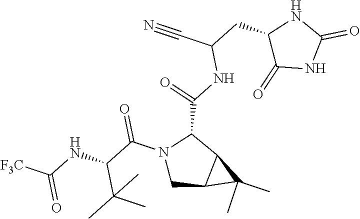 [SMILES: CC(C)(C)[C@H](NC(=O)C(F)(F)F)C(=O)N1C[C@H]2[C@@H]([C@H]1C(=O)NC(C#N)C[C@@H]1NC(=O)NC1=O)C2(C)C] | (1S,2S,5S)-N-[1-cyano-2- [(4S)-2,5-dioxoimidazolidin- 4-yl]ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 224 | 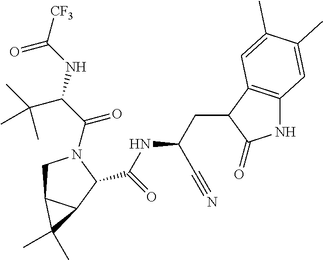 [SMILES: Cc1cc2c(cc1C)C(C[C@@H](C#N)NC(=O)[C@@H]1[C@@H]3[C@H](CN1C(=O)[C@@H](NC(=O)C(F)(F)F)C(C)(C)C)C3(C)C)C(=O)N2] | (1S,2S,5S)-N-[(1S)-1-cyano- 2-(5,6-dimethyl-2-oxo- indolin-3-yl)ethyl]-3-[(2S)- 3,3-dimethyl-2-[(2,2,2- trifluoroacetyl)amino]butanoyl]- 6,6-dimethyl-3- azabicyclo[3.1.0]hexane-2- carboxamide |
| 225 | 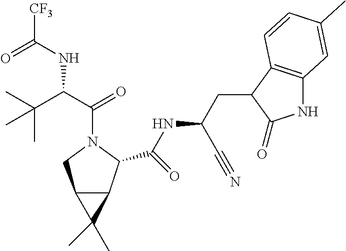 [SMILES: Cc1ccc2c(c1)NC(=O)C2C[C@@H](C#N)NC(=O)[C@@H]1[C@@H]2[C@H](CN1C(=O)[C@@H](NC(=O)C(F)(F)F)C(C)(C)C)C2(C)C] | (1S,2S,5S)-N-[(1S)-1-cyano- 2-(6-methyl-2-oxo-indolin-3- yl)ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 226 | 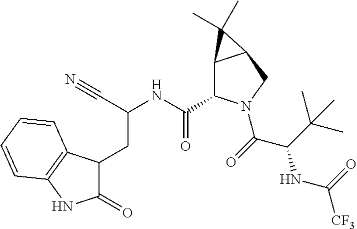 [SMILES: CC(C)(C)[C@H](NC(=O)C(F)(F)F)C(=O)N1C[C@H]2[C@@H]([C@H]1C(=O)NC(C#N)CC1C(=O)Nc3ccccc31)C2(C)C] | (1S,2S,5S)-N-[1-cyano-2-(2- oxoindolin-3-yl)ethyl]-3- [(2S)-3,3-dimethyl-2-[(2,2,2- trifluoroacetyl)amino]butanoyl]- 6,6-dimethyl-3- azabicyclo[3.1.0]hexane-2- carboxamide |
| 227 | 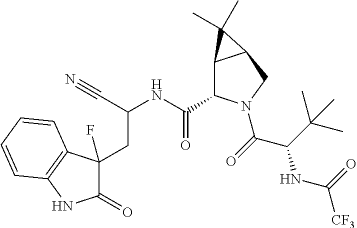 [SMILES: CC(C)(C)[C@H](NC(=O)C(F)(F)F)C(=O)N1C[C@H]2[C@@H]([C@H]1C(=O)NC(C#N)CC1(F)C(=O)Nc3ccccc31)C2(C)C] | (1S,2S,5S)-N-[1-cyano-2-(3- fluoro-2-oxo-indolin-3- yl)ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 228 | 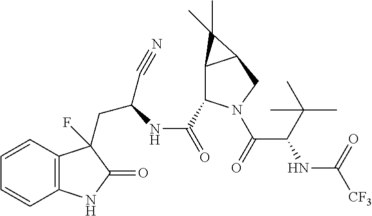 [SMILES: CC(C)(C)[C@H](NC(=O)C(F)(F)F)C(=O)N1C[C@H]2[C@@H]([C@H]1C(=O)N[C@H](C#N)CC1(F)C(=O)Nc3ccccc31)C2(C)C] | (1S,2S,5S)-N-[(1S)-1-cyano- 2-(3-fluoro-2-oxo-indolin-3- yl)ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 229 | 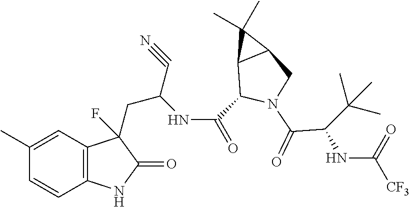 [SMILES: Cc1ccc2c(c1)C(F)(CC(C#N)NC(=O)[C@@H]1[C@@H]3[C@H](CN1C(=O)[C@@H](NC(=O)C(F)(F)F)C(C)(C)C)C3(C)C)C(=O)N2] | (1S,2S,5S)-N-[1-cyano-2-(3- fluoro-5-methyl-2-oxo- indolin-3-yl)ethyl]-3-[(2S)- 3,3-dimethyl-2-[(2,2,2- trifluoroacetyl)amino]butanoyl]- 6,6-dimethyl-3- azabicyclo[3.1.0]hexane-2- carboxamide |
| 230 | 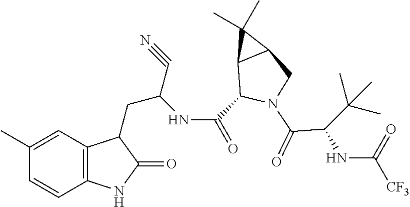 [SMILES: Cc1ccc2c(c1)C(CC(C#N)NC(=O)[C@@H]1[C@@H]3[C@H](CN1C(=O)[C@@H](NC(=O)C(F)(F)F)C(C)(C)C)C3(C)C)C(=O)N2] | (1S,2S,5S)-N-[1-cyano-2-(5- methyl-2-oxo-indolin-3- yl)ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 231 | 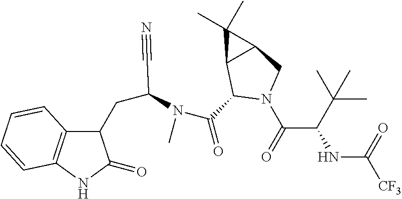 [SMILES: CN(C(=O)[C@@H]1[C@@H]2[C@H](CN1C(=O)[C@@H](NC(=O)C(F)(F)F)C(C)(C)C)C2(C)C)[C@H](C#N)CC1C(=O)Nc2ccccc21] | (1S,2S,5S)-N-[(1S)-1-cyano- 2-(2-oxoindolin-3-yl)ethyl]- 3-[(2S)-3,3-dimethyl-2- [(2,2,2- trifluoroacetyl)amino]butanoyl]- N,6,6-trimethyl-3- azabicyclo[3.1.0]hexane-2- carboxamide |
| 232 | 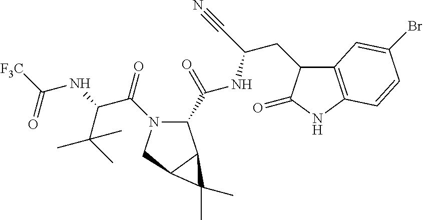 [SMILES: CC(C)(C)[C@H](NC(=O)C(F)(F)F)C(=O)N1C[C@H]2[C@@H]([C@H]1C(=O)N[C@H](C#N)CC1C(=O)Nc3ccc(Br)cc31)C2(C)C] | (1S,2S,5S)-N-[(1S)-2-(5- bromo-2-oxo-indolin-3-yl)- 1-cyano-ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 233 | 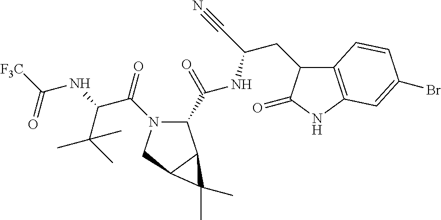 [SMILES: CC(C)(C)[C@H](NC(=O)C(F)(F)F)C(=O)N1C[C@H]2[C@@H]([C@H]1C(=O)N[C@H](C#N)CC1C(=O)Nc3cc(Br)ccc31)C2(C)C] | (1S,2S,5S)-N-[(1S)-2-(6- bromo-2-oxo-indolin-3-yl)- 1-cyano-ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 234 | 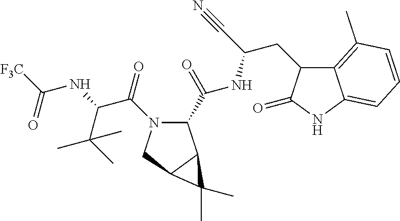 [SMILES: Cc1cccc2c1C(C[C@@H](C#N)NC(=O)[C@@H]1[C@@H]3[C@H](CN1C(=O)[C@@H](NC(=O)C(F)(F)F)C(C)(C)C)C3(C)C)C(=O)N2] | (1S,2S,5S)-N-[(1S)-1-cyano- 2-(4-methyl-2-oxo-indolin-3- yl)ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 235 | 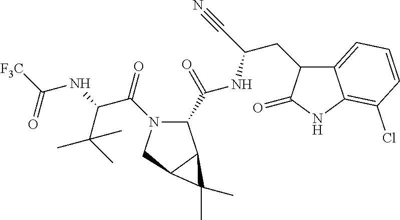 [SMILES: CC(C)(C)[C@H](NC(=O)C(F)(F)F)C(=O)N1C[C@H]2[C@@H]([C@H]1C(=O)N[C@H](C#N)CC1C(=O)Nc3c(Cl)cccc31)C2(C)C] | (1S,2S,5S)-N-[(1S)-2-(7- chloro-2-oxo-indolin-3-yl)-1- cyano-ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 236 | 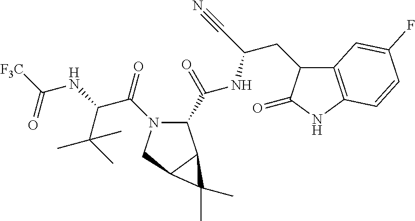 [SMILES: CC(C)(C)[C@H](NC(=O)C(F)(F)F)C(=O)N1C[C@H]2[C@@H]([C@H]1C(=O)N[C@H](C#N)CC1C(=O)Nc3ccc(F)cc31)C2(C)C] | (1S,2S,5S)-N-[(1S)-1-cyano- 2-(5-fluoro-2-oxo-indolin-3- yl)ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 237 | 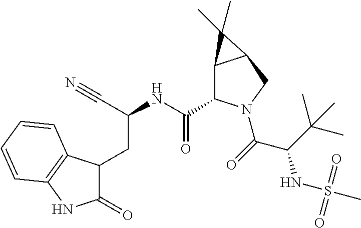 [SMILES: CC(C)(C)[C@H](NS(C)(=O)=O)C(=O)N1C[C@H]2[C@@H]([C@H]1C(=O)N[C@H](C#N)CC1C(=O)Nc3ccccc31)C2(C)C] | (1S,2S,5S)-N-[(1S)-1-cyano- 2-(2-oxoindolin-3-yl)ethyl]- 3-[(2S)-2- (methanesulfonamido)-3,3- dimethyl-butanoyl]-6,6- dimethyl-3- azabicyclo[3.1.0]hexane-2- carboxamide |
| 238 | 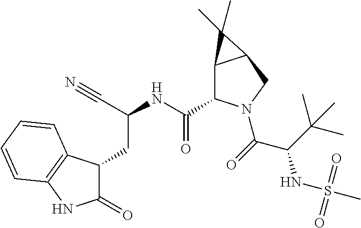 [SMILES: CC(C)(C)[C@H](NS(C)(=O)=O)C(=O)N1C[C@H]2[C@@H]([C@H]1C(=O)N[C@H](C#N)C[C@@H]1C(=O)Nc3ccccc31)C2(C)C] | (1S,2S,5S)-N-[(1S)-1-cyano- 2-(2-oxoindolin-3-yl)ethyl]- 3-[(2S)-2- (methanesulfonamido)-3,3- dimethyl-butanoyl]-6,6- dimethyl-3- azabicyclo[3.1.0]hexane-2- carboxamide |
| 239 | 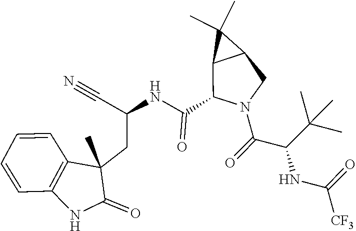 [SMILES: CC(C)(C)[C@H](NC(=O)C(F)(F)F)C(=O)N1C[C@H]2[C@@H]([C@H]1C(=O)N[C@H](C#N)C[C@]1(C)C(=O)Nc3ccccc31)C2(C)C] | (1S,2S,5S)-N-[1-cyano-2-(3- methyl-2-oxo-indolin-3- yl)ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 240 | 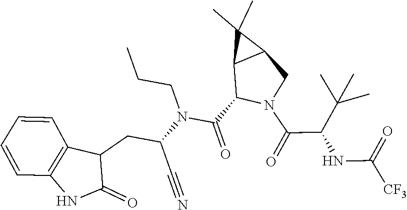 [SMILES: CCCN(C(=O)[C@@H]1[C@@H]2[C@H](CN1C(=O)[C@@H](NC(=O)C(F)(F)F)C(C)(C)C)C2(C)C)[C@H](C#N)CC1C(=O)Nc2ccccc21] | (1R,2S,5S)-N-[(1S)-1-cyano- 2-(2-oxoindolin-3-yl)ethyl]- 3-[(2S)-3,3-dimethyl-2- [(2,2,2- trifluoroacetyl)amino]butanoyl]- 6,6-dimethyl-N-propyl-3- azabicyclo[3.1.0]hexane-2- carboxamide |
| 241 | 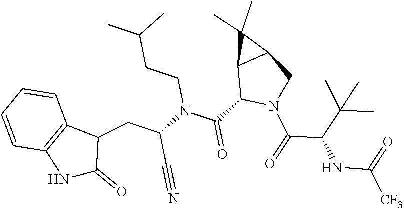 [SMILES: CC(C)CCN(C(=O)[C@@H]1[C@@H]2[C@H](CN1C(=O)[C@@H](NC(=O)C(F)(F)F)C(C)(C)C)C2(C)C)[C@H](C#N)CC1C(=O)Nc2ccccc21] | (1R,2S,5S)-N-[(1S)-1-cyano- 2-(2-oxoindolin-3-yl)ethyl]- 3-[(2S)-3,3-dimethyl-2- [(2,2,2- trifluoroacetyl)amino]butanoyl]- N-isopentyl-6,6- dimethyl-3- azabicyclo[3.1.0]hexane-2- carboxamide |
| 242 | 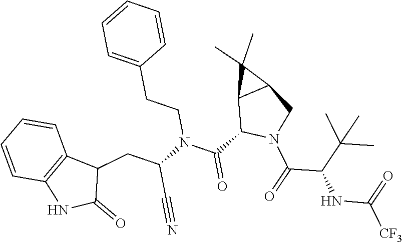 [SMILES: CC(C)(C)[C@H](NC(=O)C(F)(F)F)C(=O)N1C[C@H]2[C@@H]([C@H]1C(=O)N(CCc1ccccc1)[C@H](C#N)CC1C(=O)Nc3ccccc31)C2(C)C] | (1S,2S,5S)-N-[(1S)-1-cyano- 2-(2-oxoindolin-3-yl)ethyl]- 3-[(2S)-3,3-dimethyl-2- [(2,2,2- trifluoroacetyl)amino]butanoyl]- 6,6-dimethyl-N-(2- phenylethyl)-3- azabicyclo[3.1.0]hexane-2- carboxamide |
| 243 | 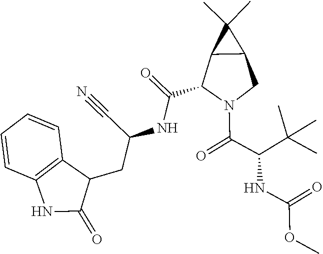 [SMILES: COC(=O)N[C@H](C(=O)N1C[C@H]2[C@@H]([C@H]1C(=O)N[C@H](C#N)CC1C(=O)Nc3ccccc31)C2(C)C)C(C)(C)C] | methyl N-[(1S)-1- [(1S,2S,5S)-2-[[(1S)-1- cyano-2-(2-oxoindolin-3- yl)ethyl]carbamoyl]-6,6- dimethyl-3- azabicyclo[3.1.0]hexane-3- carbonyl]-2,2-dimethyl- propyl]carbamate |
| 244 | 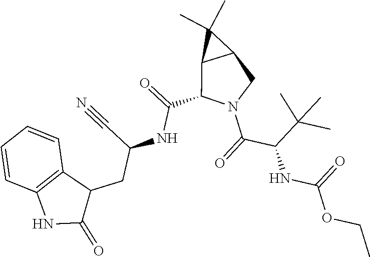 [SMILES: CCOC(=O)N[C@H](C(=O)N1C[C@H]2[C@@H]([C@H]1C(=O)N[C@H](C#N)CC1C(=O)Nc3ccccc31)C2(C)C)C(C)(C)C] | ethyl N-[(1S)-1-[(1S,2S,5S)- 2-[[(1S)-1-cyano-2-(2- oxoindolin-3- yl)ethyl]carbamoyl]-6,6- dimethyl-3- azabicyclo[3.1.0]hexane-3- carbonyl]-2,2-dimethyl- propyl]carbamate |
| 245 | 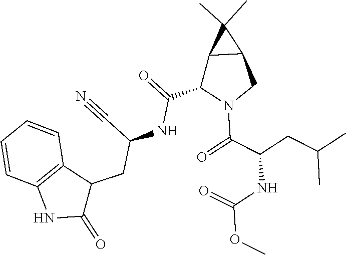 [SMILES: COC(=O)N[C@@H](CC(C)C)C(=O)N1C[C@H]2[C@@H]([C@H]1C(=O)N[C@H](C#N)CC1C(=O)Nc3ccccc31)C2(C)C] | methyl N-[(1S)-1- [(1S,2S,5S)-2-[(1S)-1- cyano-2-(2-oxoindolin-3- yl)ethyl]carbamoyl]-6,6- dimethyl-3- azabicyclo[3.1.0]hexane-3- carbonyl]-3-methyl- butyl]carbamate |
| 246 | 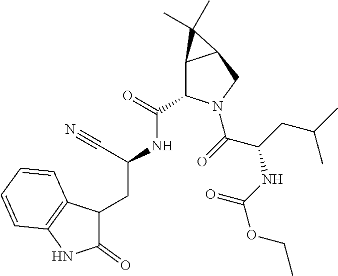 [SMILES: CCOC(=O)N[C@@H](CC(C)C)C(=O)N1C[C@H]2[C@@H]([C@H]1C(=O)N[C@H](C#N)CC1C(=O)Nc3ccccc31)C2(C)C] | ethyl N-[(1S)-1-[(1S,2S,5S)- 2-[[(1S)-1-cyano-2-(2- oxoindolin-3- yl)ethyl]carbamoyl]-6,6- dimethyl-3- azabicyclo[3.1.0]hexane-3- carbonyl]-3-methyl- butyl]carbamate |
| 247 | 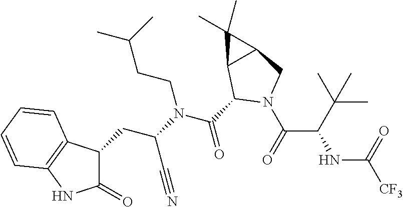 [SMILES: CC(C)CCN(C(=O)[C@@H]1[C@@H]2[C@H](CN1C(=O)[C@@H](NC(=O)C(F)(F)F)C(C)(C)C)C2(C)C)[C@H](C#N)C[C@@H]1C(=O)Nc2ccccc21] | (1R,2S,5S)-N-[(1S)-1-cyano- 2-(2-oxoindolin-3-yl)ethyl]- 3-[(2S)-3,3-dimethyl-2- [(2,2,2- trifluoroacetyl)amino]butanoyl]- N-isopentyl-6,6- dimethyl-3- azabicyclo[3.1.0]hexane-2- carboxamide |
| 248 | 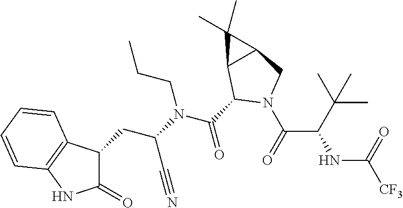 [SMILES: CCCN(C(=O)[C@@H]1[C@@H]2[C@H](CN1C(=O)[C@@H](NC(=O)C(F)(F)F)C(C)(C)C)C2(C)C)[C@H](C#N)C[C@@H]1C(=O)Nc2ccccc21] | (1R,2S,5S)-N-[(1S)-1-cyano- 2-(2-oxoindolin-3-yl)ethyl]- 3-[(2S)-3,3-dimethyl-2- [(2,2,2- trifluoroacetyl)amino]butanoyl]- 6,6-dimethyl-N-propyl-3- azabicyclo[3.1.0]hexane-2- carboxamide |
| 249 | 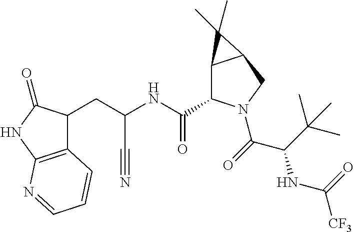 [SMILES: CC(C)(C)[C@H](NC(=O)C(F)(F)F)C(=O)N1C[C@H]2[C@@H]([C@H]1C(=O)NC(C#N)CC1C(=O)Nc3ncccc31)C2(C)C] | (1S,2S,5S)-N-[1-cyano-2-(2- oxo-1,3-dihydropyrrolo[2,3- b]pyridin-3-yl)ethyl]-3- [(2S)-3,3-dimethyl-2-[(2,2,2- trifluoroacetyl)amino]butanoyl]- 6,6-dimethyl-3- azabicyclo[3.1.0]hexane-2- carboxamide |
| 250 | 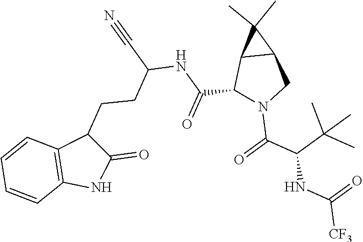 [SMILES: CC(C)(C)[C@H](NC(=O)C(F)(F)F)C(=O)N1C[C@H]2[C@@H]([C@H]1C(=O)NC(C#N)CCC1C(=O)Nc3ccccc31)C2(C)C] | (1S,2S,5S)-N-[1-cyano-3-(2- oxoindolin-3-yl)propyl]-3- [(2S)-3,3-dimethyl-2-[(2,2,2- trifluoroacetyl)amino]butanoyl]- 6,6-dimethyl-3- azabicyclo[3.1.0]hexane-2- carboxamide |
| 251 | 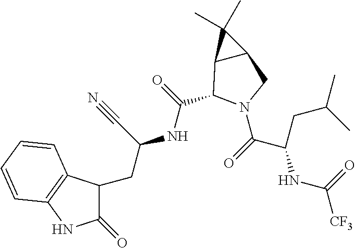 [SMILES: CC(C)C[C@H](NC(=O)C(F)(F)F)C(=O)N1C[C@H]2[C@@H]([C@H]1C(=O)N[C@H](C#N)CC1C(=O)Nc3ccccc31)C2(C)C] | (1S,2S,5S)-N-[(1S)-1-cyano- 2-(2-oxoindolin-3-yl)ethyl]- 6,6-dimethyl-3-[(2S)-4- methyl-2-[(2,2,2- trifluoroacetyl)amino] pentanoyl]-3- azabicyclo[3.1.0]hexane-2- carboxamide |
| 252 | 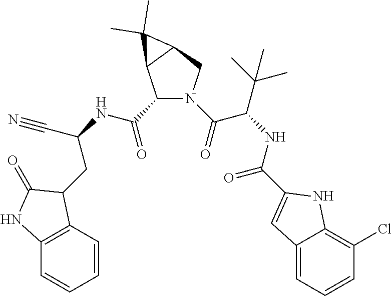 [SMILES: CC(C)(C)[C@H](NC(=O)c1cc2cccc(Cl)c2[nH]1)C(=O)N1C[C@H]2[C@@H]([C@H]1C(=O)N[C@H](C#N)CC1C(=O)Nc3ccccc31)C2(C)C] | 7-chloro-N-[(1S)-1- [(1S,2S,5S)-2-[(1S)-1- cyano-2-(2-oxoindolin-3- yl)ethyl]carbamoyl]-6,6- dimethyl-3- azabicyclo[3.1.0]hexane-3- carbonyl]-2,2-dimethyl- propyl]-1H-indole-2- carboxamide |
| 253 | 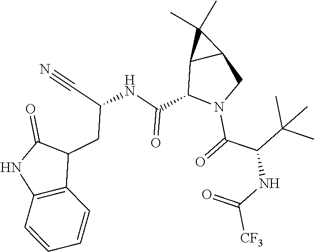 [SMILES: CC(C)(C)[C@H](NC(=O)C(F)(F)F)C(=O)N1C[C@H]2[C@@H]([C@H]1C(=O)N[C@@H](C#N)CC1C(=O)Nc3ccccc31)C2(C)C] | (1S,2S,5S)-N-[(1R)-1-cyano- 2-(2-oxoindolin-3-yl)ethyl]- 3-[(2S)-3,3-dimethyl-2- [(2,2,2- trifluoroacetyl)amino]butanoyl]- 6,6-dimethyl-3- azabicyclo[3.1.0]hexane-2- carboxamide |
| 301 | 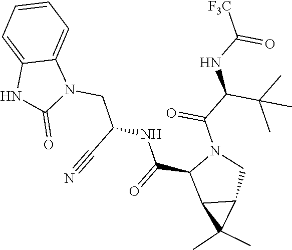 [SMILES: CC(C)(C)[C@H](NC(=O)C(F)(F)F)C(=O)N1C[C@H]2[C@H]([C@H]1C(=O)N[C@H](C#N)Cn1c(=O)[nH]c3ccccc31)C2(C)C] | (1S,2S,5S)-N-[(1S)-1-cyano- 2-(2-oxo-3H-benzimidazol- 1-yl)ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 302 | 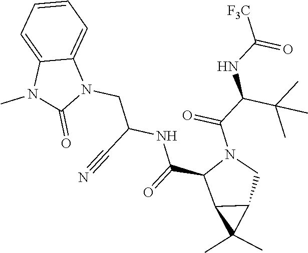 [SMILES: Cn1c(=O)n(CC(C#N)NC(=O)[C@@H]2[C@H]3[C@H](CN2C(=O)[C@@H](NC(=O)C(F)(F)F)C(C)(C)C)C3(C)C)c2ccccc21] | (1S,2S,5S)-N-[1-cyano-2-(3- methyl-2-oxo-benzimidazol- 1-yl)ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 303 | 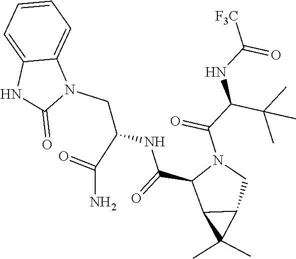 [SMILES: CC(C)(C)[C@H](NC(=O)C(F)(F)F)C(=O)N1C[C@H]2[C@H]([C@H]1C(=O)N[C@@H](Cn1c(=O)[nH]c3ccccc31)C(N)=O)C2(C)C] | (1S,2S,5S)-N-[(1S)-2-amino- 2-oxo-1-[(2-oxo-3H- benzimidazol-1- yl)methyl]ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 304 | 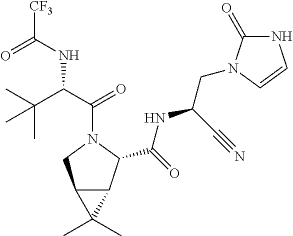 [SMILES: CC(C)(C)[C@H](NC(=O)C(F)(F)F)C(=O)N1C[C@H]2[C@H]([C@H]1C(=O)N[C@H](C#N)Cn1cc[nH]c1=O)C2(C)C] | (1S,2S,5S)-N-[(1S)-1-cyano- 2-(2-oxo-1H-imidazol-3- yl)ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 305 | 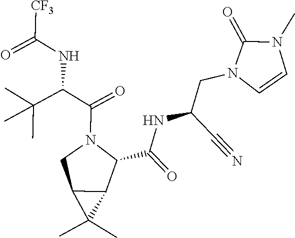 [SMILES: Cn1ccn(C[C@@H](C#N)NC(=O)[C@@H]2[C@H]3[C@H](CN2C(=O)[C@@H](NC(=O)C(F)(F)F)C(C)(C)C)C3(C)C)c1=O] | (1S,2S,5S)-N-[(1S)-1-cyano- 2-(3-methyl-2-oxo-imidazol- 1-yl)ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 306 | 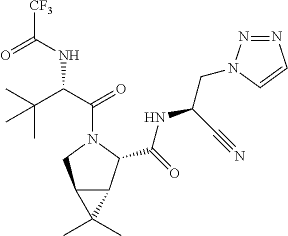 [SMILES: CC(C)(C)[C@H](NC(=O)C(F)(F)F)C(=O)N1C[C@H]2[C@H]([C@H]1C(=O)N[C@H](C#N)Cn1ccnn1)C2(C)C] | (1S,2S,5S)-N-[(1S)-1-cyano- 2-(triazol-1-yl)ethyl]-3- [(2S)-3,3-dimethyl-2-[(2,2,2- trifluoroacetyl)amino]butanoyl]- 6,6-dimethyl-3- azabicyclo[3.1.0]hexane-2- carboxamide |
| 307 | 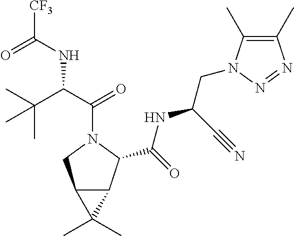 [SMILES: Cc1nnn(C[C@@H](C#N)NC(=O)[C@@H]2[C@H]3[C@H](CN2C(=O)[C@@H](NC(=O)C(F)(F)F)C(C)(C)C)C3(C)C)c1C] | (1S,2S,5S)-N-[(1S)-1-cyano- 2-(4,5-dimethyltriazol-1- yl)ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 308 | 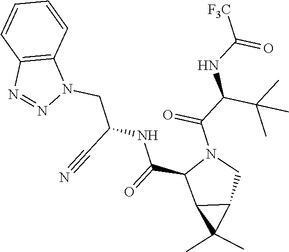 [SMILES: CC(C)(C)[C@H](NC(=O)C(F)(F)F)C(=O)N1C[C@H]2[C@H]([C@H]1C(=O)N[C@H](C#N)Cn1nnc3ccccc31)C2(C)C] | (1S,2S,5S)-N-[(1S)-2- (benzotriazol-1-yl)-1-cyano- ethyl]-3-[(2S)-3,3-dimethyl- 2-[(2,2,2- trifluoroacetyl)amino]butanoyl]- 6,6-dimethyl-3- azabicyclo[3.1.0]hexane-2- carboxamide |
| 309 | 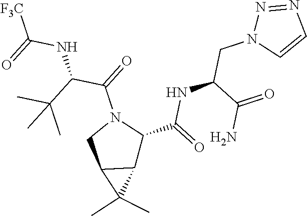 [SMILES: CC(C)(C)[C@H](NC(=O)C(F)(F)F)C(=O)N1C[C@H]2[C@H]([C@H]1C(=O)N[C@@H](Cn1ccnn1)C(N)=O)C2(C)C] | (1S,2S,5S)-N-[(1S)-2-amino- 2-oxo-1-(triazol-1- ylmethyl)ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 310 | 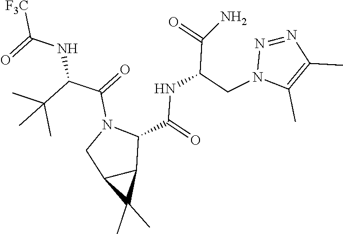 [SMILES: Cc1nnn(C[C@H](NC(=O)[C@@H]2[C@@H]3[C@H](CN2C(=O)[C@@H](NC(=O)C(F)(F)F)C(C)(C)C)C3(C)C)C(N)=O)c1C] | (1R,2S,5S)-N-[(1S)-2- amino-1-[(4,5- dimethyltriazol-1-yl)methyl]- 2-oxo-ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 311 | 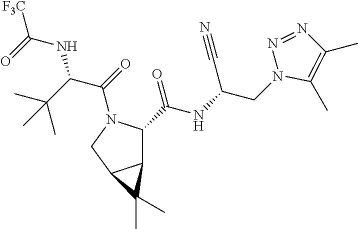 [SMILES: Cc1nnn(C[C@@H](C#N)NC(=O)[C@@H]2[C@@H]3[C@H](CN2C(=O)[C@@H](NC(=O)C(F)(F)F)C(C)(C)C)C3(C)C)c1C] | (1S,2S,5S)-N-[(1S)-1-cyano- 2-(4,5-dimethyltriazol-1- yl)ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 312 | 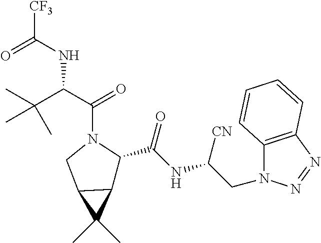 [SMILES: CC(C)(C)[C@H](NC(=O)C(F)(F)F)C(=O)N1C[C@H]2[C@@H]([C@H]1C(=O)N[C@H](C#N)Cn1nnc3ccccc31)C2(C)C] | (1S,2S,5S)-N-[(1S)-2- (benzotriazol-1-yl)-1-cyano- ethyl]-3-[(2S)-3,3-dimethyl- 2-[(2,2,2- trifluoroacetyl)amino]butanoyl]- 6,6-dimethyl-3- azabicyclo[3.1.0]hexane-2- carboxamide |
| 401 | 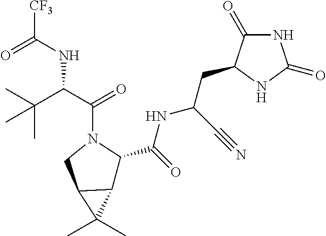 [SMILES: CC(C)(C)[C@H](NC(=O)C(F)(F)F)C(=O)N1C[C@H]2[C@H]([C@H]1C(=O)NC(C#N)C[C@@H]1NC(=O)NC1=O)C2(C)C] | (1S,2S,5S)-N-[1-cyano-2- [(4S)-2,5-dioxoimidazolidin- 4-yl]ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 402 | 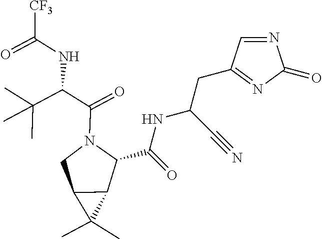 [SMILES: CC(C)(C)[C@H](NC(=O)C(F)(F)F)C(=O)N1C[C@H]2[C@H]([C@H]1C(=O)NC(C#N)CC1=NC(=O)N=C1)C2(C)C] | (1S,2S,5S)-N-[1-cyano-2-(2- oxoimidazol-4-yl)ethyl]-3- [(2S)-3,3-dimethyl-2-[(2,2,2- trifluoroacetyl)amino]butanoyl]- 6,6-dimethyl-3- azabicyclo[3.1.0]hexane-2- carboxamide |
| 403 | 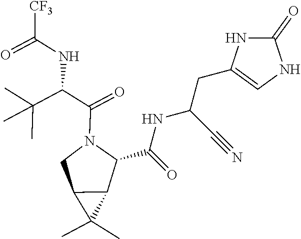 [SMILES: CC(C)(C)[C@H](NC(=O)C(F)(F)F)C(=O)N1C[C@H]2[C@H]([C@H]1C(=O)NC(C#N)Cc1c[nH]c(=O)[nH]1)C2(C)C] | (1S,2S,5S)-N-[1-cyano-2-(2- oxo-1,3-dihydroimidazol-4- yl)ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 404 | 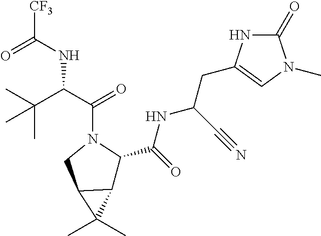 [SMILES: Cn1cc(CC(C#N)NC(=O)[C@@H]2[C@H]3[C@H](CN2C(=O)[C@@H](NC(=O)C(F)(F)F)C(C)(C)C)C3(C)C)[nH]c1=O] | (1S,2S,5S)-N-[1-cyano-2-(3- methyl-2-oxo-1H-imidazol- 5-yl)ethyl]-3-[(2S)-3,3- dimethyl-2-[(2,2,2- trifluoroacetyl)amino]butanoyl]- 6,6-dimethyl-3- azabicyclo[3.1.0]hexane-2- carboxamide |
| 501 | 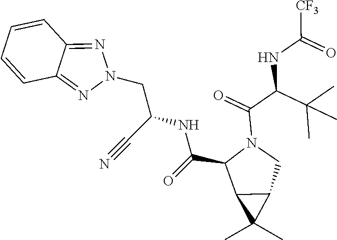 [SMILES: CC(C)(C)[C@H](NC(=O)C(F)(F)F)C(=O)N1C[C@H]2[C@H]([C@H]1C(=O)N[C@H](C#N)Cn1nc3ccccc3n1)C2(C)C] | (1S,2S,5S)-N-[(1S)-2- (benzotriazol-2-yl)-1-cyano- ethyl]-3-[(2S)-3,3-dimethyl- 2-[(2,2,2- trifluoroacetyl)amino]butanoyl]- 6,6-dimethyl-3- azabicyclo[3.1.0]hexane-2- carboxamide |

or a pharmaceutically acceptable salt, stereoisomer, solvate, or tautomer thereof.

13. A pharmaceutical composition comprising the compound or pharmaceutically acceptable salt, solvate, hydrate, stereoisomer, or tautomer thereof of claim 1, and a pharmaceutically acceptable carrier.

14. The pharmaceutical composition of claim 13, further comprising an additional pharmaceutically active agent.

15. A method of inhibiting of coronavirus replication, comprising of administering to a subject a compound of claim 1.

16. A method of treating a disease or disorder associated with coronavirus infection, comprising of administering to a subject a compound of claim 1.

17. A method of treating coronavirus infection comprising of administering to a subject in need of a treatment a compound of claim 1.

18. The method of claim 17, wherein coronavirus infection is COVID-19.

19. The method of any one of claims 15-18, wherein the subject is a mammal.

20. The method of claim 19, wherein the subject is a human.
